(19) United States
(12) Patent Application Publication
(10) Pub. No.: US 2023/0266330 A1
(43) Pub. Date: Aug. 24, 2023

(54) EXOSOMAL TUMOR BIOMARKERS AND COLLECTIONS THEREOF

(71) Applicant: CORNELL UNIVERSITY, Ithaca, NY (US)

(72) Inventors: David LYDEN, New York, NY (US); Ayuko HOSHINO, New York, NY (US); Han Sang KIM, New York, NY (US); Linda BOJMAR, New York, NY (US)

(21) Appl. No.: 18/004,149

(22) PCT Filed: Jul. 6, 2021
(86) PCT No.: PCT/US2021/040543

§ 371 Date: Jan. 3, 2023

Related U.S. Application Data
(60) Provisional application No. 63/048,406, filed on Jul. 6, 2020.
(60) Provisional application No. 63/064,825, filed on Aug. 12, 2020.

(51) Int. Cl. G01N 33/574; G01N 33/68

(52) U.S. Cl. CPC G01N 33/57488; G01N 33/6854; G01N 33/57438; G01N 33/57419; G01N 33/57423

(57) ABSTRACT

The present disclosure relates to methods that involve obtaining a tissue or liquid biopsy sample from a subject. Extracellular vesicles and particles are separated from the sample, and protein from the separated extracellular vesicles and particles is isolated to form an extracellular vesicle and particle protein sample. The extracellular vesicle and particle protein sample is subjected to a detection assay suitable for detecting the sample's protein signature, and the presence, absence, status, and/or type of cancer in the subject is identified based the detected protein signature.

This application claims the priority benefit of U.S. Provisional Patent Application Ser. No. 63/048,406, filed Jul. 6, 2020, and U.S. Provisional Patent Application Ser. No. 63/064,825, filed Aug. 12, 2020, which are hereby incorporated by reference in their entirety.

### FIELD OF INVENTION

The present disclosure relates to exosomal tumor biomarkers and collections thereof.

### BACKGROUND

Pathologists employ tissue biopsies, when accessible, to diagnose cancer, cancer spread, and, more recently, to measure treatment response. The number of biopsies is limited due to the invasive and specialized nature of the procedure. On the other hand, liquid biopsies derived from a patient's blood sample are minimally invasive, far more easily procured, and can be obtained repeatedly. In addition, liquid biopsies may provide the advantage of detecting cancers at their earliest, most curable stage, even before radiographically occult tumors. As expectations for the potential of liquid biopsies in cancer diagnosis, prognosis and therapeutic response grow, extracellular vesicles (EVs), particularly exosomes, are attracting considerable interest as a valuable resource in this endeavor.

Exosomes are 50-150 nm nanovesicles of endosomal origin that are enriched in nucleic acids, lipids and proteins (O'Driscoll, L., “Expanding on Exosomes and Ectosomes in Cancer,” N Engl J Med 372:2359-2362 (2015); Thakur et al., “Double-Stranded DNA in Exosomes: A Novel Biomarker in Cancer Detection,” Cell Res 24:766-769 (2014)). Initially thought to be “cell debris” (Johnstone, R. M., “The Jeanne Manery-Fisher Memorial Lecture 1991. Maturation of Reticulocytes: Formation of Exosomes as a Mechanism for Shedding Membrane Proteins,” Biochem Cell Biol 70:179-190. (1992)) and a means of eliminating unneeded material from the cell, exosomes are now considered critical and active mediators of intercellular communication with physiological and pathologic relevance (Becker et al., “Extracellular Vesicles in Cancer: Cell-to-Cell Mediators of Metastasis,” Cancer cell 30:836-848 (2016); Johnstone et al., “Vesicle Formation during Reticulocyte Maturation. Association of Plasma Membrane Activities with Released Vesicles (exosomes),” The Journal of Biological Chemistry 262:9412-9420 (1987); Maas et al., “Extracellular Vesicles: Unique Intercellular Delivery Vehicles,” Trends in Cell Biology 27:172-188 (2017); Skog et al., “Glioblastoma Microvesicles Transport RNA and Proteins that Promote Tumour Growth and Provide Diagnostic Biomarkers,” Nature Cell Biology 10:1470-1476 (2008); Yanez-Mo et al., “Biological Properties of Extracellular Vesicles and their Physiological Functions,” Journal of Extracellular Vesicles 4:27066 (2015)). Previously, the prognostic and functional importance of selective protein cargo packaged in tumor-derived exosomes was reported in the context of tumor progression, immune regulation and metastasis (Costa-Silva et al., “Pancreatic Cancer Exosomes Initiate Pre-Metastatic Niche Formation in the Liver,” Nature Cell Biology 17:816-826 (2015); Hoshino et al., “Tumour Exosome Integrins Determine Organotropic Metastasis,” Nature 527:329-335 (2015); Peinado et al., “Melanoma Exosomes Educate Bone Marrow Progenitor Cells Toward a Pro-Metastatic Phenotype through MET,” Nat Med 18:883-891 (2012)). Moreover, the heterogeneity of extracellular nano-particle populations isolated by sequential ultracentrifugation was deconvoluted from a diversity of murine and human samples, defining three distinct subpopulations, small exosomes (Exo-S), large exosomes (Exo-L) and the newly identified exomeres (Zhang et al., “Identification of Distinct Nanoparticles and Subsets of Extracellular Vesicles by Asymmetric Flow Field-Flow Fractionation,” Nature Cell Biology 20:332-343 (2018); Zhang et al., “Asymmetric-Flow Field-Flow Fractionation Technology for Exomere and Small Extracellular Vesicle Separation and Characterization,” Nat Protoc 14:1027-1053 (2019)). For the purposes of this study, these three sub-populations will be collectively referred to as extracellular vesicles and particles (EVPs). As such, characterization of EVP proteins obtained from blood liquid biopsies can offer valuable information for cancer diagnosis, prognosis and for monitoring therapeutic outcomes. Since exosomes are actively released into the peripheral circulation from both tumor and normal cells, resulting in exosome concentrations of >109 vesicles/mL in the plasma, ample material can be isolated for downstream analyses (Colombo et al., “Biogenesis, Secretion, and Intercellular Interactions of Exosomes and other Extracellular Vesicles,” Annual Review of Cell and Developmental Biology 30:255-289 (2014)). Accumulating evidence suggests that exosome-based disease markers can be identified in early-stage disease (Chen et al., “Phosphoproteins in Extracellular Vesicles as Candidate Markers for Breast Cancer,” Proc Natl Acad Sci 114:3175-3180 (2017)) and could thus be used for early detection as well as prognosis and therapy guidance.

Mass spectrometry-based proteomic profiling represents an emerging strategy to gain better insight into the biology and clinical potential of circulating exosomes (Choi et al., “Proteomics of Extracellular Vesicles: Exosomes and Ectosomes,” Mass Spectrom Rev 34:474-490 (2015)). Although identifying common and/or tumor-derived exosomal proteins is crucial for biomarker development, little is known about proteomic composition across different tissue- and tumor type14 specific exosomes, despite the public availability of several exosome protein databases (e.g., Vesiclepedia, EVpedia, ExoCarta) (Kalra et al., “Vesiclepedia: A Compendium for Extracellular Vesicles with Continuous Community Annotation,” PLoS biology 10:e1001450 (2012); Kim et al., “EVpedia: A Community Web Portal for Extracellular Vesicles Research,” Bioinformatics 31:933-939 (2015); Mathivanan et al., “ExoCarta: A Compendium of Exosomal Proteins and RNA,” Proteomics 9:4997-5000 (2009)). Exosomes from a plethora of sources (e.g., cell lines, tissues and bodily fluids from humans and mice) have now been characterized. However, a comprehensive analysis to determine the extent to which exosome-specific proteins are conserved across species and tissues has yet to be performed. In addition, identification of exosome markers that are detected with high frequency and abundance throughout samples will improve exosome isolation methodologies for exosome enriched liquid biopsies. Conversely, to date, exosomal proteins that can be used to unequivocally distinguish normal exosomes from cancer exosomes have not been identified, mainly due to a paucity of data on the cargo of exosomes isolated from normal cells and tissues. Therefore, proof of principle analyses demonstrating that exosomal proteomes are a useful liquid biopsy tool are needed.

The present disclosure is directed to overcome these and other deficiencies in the art.

### SUMMARY

A first aspect of the present disclosure is directed to a method for screening a subject for the presence of cancer that involves obtaining a liquid biopsy sample from a subject. Extracellular vesicles and particles are separated from the sample, and protein from the separated extracellular vesicles and particles is isolated to form an extracellular vesicle and particle protein sample. The extracellular vesicle and particle protein sample is subjected to a detection assay suitable for detecting: (i) a protein selected from the group consisting of ferritin light chain, von Willebrand factor, immunoglobulin lambda constant 2, keratin 17, immunoglobulin heavy constant gamma 1, keratin 6B, radixin, cofilin 1, protease, serine 1, tubulin alpha 1c, ADAM metallopeptidase with thrombospondin type 1 motif 13, immunoglobulin kappa variable 6D-21, tyrosine 3-monooxygenase/tryptophan 5-monooxygenase activation protein theta, POTE ankyrin domain family member I, POTE ankyrin domain family member F, and combinations thereof; and (ii) a protein selected from the group consisting of actin gamma 1, immunoglobulin lambda variable 3-27, immunoglobulin kappa variable 1D-12, coagulation factor XI, complement C1r subcomponent like, attractin, butyrylcholinesterase, immunoglobulin heavy variable 3-35, immunoglobulin kappa variable 1-17, C1q and TNF related 3, immunoglobulin heavy variable 3-20, immunoglobulin heavy variable 3/OR15-7, collectin subfamily member 11, immunoglobulin heavy constant delta, immunoglobulin kappa variable 3D-11, immunoglobulin heavy variable 3/OR16-10, immunoglobulin kappa variable 2D-24, immunoglobulin kappa variable 2-40, immunoglobulin kappa variable 1-27, immunoglobulin heavy variable 3/OR16-9, immunoglobulin lambda variable 5-45, immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6, and combinations thereof, thereby detecting the presence or absence of the protein of (i) and the protein of (ii) in the extracellular vesicle and particle protein sample.

Another aspect of the present disclosure is directed to a method for screening a subject for the presence of cancer that involves obtaining a liquid biopsy sample from a subject. Extracellular vesicles and particles are separated from the sample, and protein from the separated extracellular vesicles and particles is isolated to form an extracellular vesicle and particle protein sample. The extracellular vesicle and particle protein sample is subjected to a detection assay suitable for detecting: (i) a protein selected from the group consisting of Ferritin light chain (FTL), ABC-type oligopeptide transporter ABCB9 (ABCB9), Protein Z-dependent protease inhibitor (SERPINA10), Coagulation factor VIII (F8), Lactotransferrin (LTF), Basement membrane-specific heparan sulfate proteoglycan core protein (HSPG2), Protein disulfide-isomerase (P4HB), Trypsin-1 (PRSS1), Keratin, type II cytoskeletal 1b (KRT77), Endoplasmic reticulum chaperone BiP (HSPA5), and combinations thereof; and (ii) a protein selected from the group consisting of Complement C1q tumor necrosis factor-related protein 3 (C1QTNF3) and Immunoglobulin heavy constant delta (IGHD), or a combination thereof, thereby detecting the presence or absence of the protein of (i) and the protein of (ii) in the extracellular vesicle and particle protein sample.

In another aspect, the present disclosure is directed to a method for screening a subject for the presence of cancer that involves obtaining a tissue sample from a subject. Extracellular vesicles and particles are separated from the tissue sample, and protein from the separated extracellular vesicles and particles is isolated to form an extracellular vesicle and particle protein sample. The extracellular vesicle and particle protein sample is subjected to a detection assay suitable for detecting: (i) a protein selected from the group consisting of thrombospondin 2, versican, serrate, RNA effector molecule, tenascin C, dihydropyrimidinase like 2, adenosylhomocysteinase, DnaJ heat shock protein family (Hsp40) member A1, phosphoglycerate kinase 1, EH domain containing 2, and combinations thereof, and (ii) a protein selected from the group consisting of alcohol dehydrogenase 1B (class I), beta polypeptide, caveolae associated protein 1, FGGY carbohydrate kinase domain containing, ATP binding cassette subfamily A member 3, syntaxin 11, caveolae associated protein 2, CD36 molecule, and combinations thereof, thereby detecting the presence or absence of the protein of (i) and the protein of (ii) in the extracellular vesicle and particle protein sample.

In another aspect, the present disclosure is directed to a method for screening a subject for the presence of cancer that involves obtaining a tissue sample from a subject. Extracellular vesicles and particles are separated from the tissue sample, and protein from the separated extracellular vesicles and particles is isolated to form an extracellular vesicle and particle protein sample. The extracellular vesicle and particle protein sample is subjected to a detection assay suitable for detecting: (i) a protein selected from the group consisting of tenacin (TNC), Periostin (POSTN), Versican core protein (VCAN), signal recognition particle 9 kDa protein (SRP9), Nucleophosmin (NPM1), Serrate RNA effector molecule homolog (SRRT), ELAV-like protein 1 (ELAVL1), Cytosolic acyl coenzyme A thioester hydrolase (ACOT7), 5′-3′ exoribonuclease 2 (XRN2), Flap endonuclease 1 (FEN1), ADP-ribosylation factor-like protein 1 (ARL1), Heat shock protein 105 kDa (HSPH1), Nucleolar RNA helicase 2 (DDX21), Src-associated in mitosis 68 kDa protein (KHDRBS1), Importin subunit alpha-1 (KPNA2), SLIT-ROBO Rho GTPase-activating protein 1 (SRGAP1), WD repeat-containing protein 3 (WDR3), and combinations thereof, and (ii) a protein selected from the group consisting of Voltage-dependent calcium channel subunit alpha-2/delta-2 (CACNA2D2), Specifically androgen-regulated gene protein (C1orf116), Caveolin-2 (CAV2), Syntaxin-11 (STX11), Caveolae-associated protein 2 (CAVIN2), and combinations thereof, thereby detecting the presence or absence of the protein of (i) and the protein of (ii) in the extracellular vesicle and particle protein sample.

Another aspect of the present disclosure is directed to a method of determining the presence of lung cancer in a subject. The method involves obtaining a tissue sample from the subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting (i) a protein selected from the group consisting of four and a half LIM domains protein 2 (FHL2), 5′-3′ exoribonuclease 2 (XRN2), glutaredoxin-3 (GLRX), vigilin (High density lipoprotein-binding protein, HDL-binding protein) (HDLBP), serrate RNA effector molecule homolog (SRRT), regulator of chromosome condensation (RCC1), AP-3 complex subunit sigma-1 (AP3S1), small nuclear ribonucleoprotein Sm D3, Sm-D3 (SNRPD3), NOP2, 60S ribosomal protein L22 (RPL22), DnaJ homolog subfamily C member 7 (DNAJC7), STE20/SPS1-related proline-alanine-rich protein kinase, Ste-20-related kinase (STK39), signal recognition particle 54 kDa protein (SRP54), ATP-dependent DNA/RNA helicase DHX36 (DHX36), ELAV-like protein 1 (ELAVL1), thrombospondin-2 (THBS2), aconitate hydratase, mitochondrial, Aconitase (ACO2), acyl-CoA-binding domain-containing protein 3 (ACBD3), signal recognition particle 9 kDa protein (SRP9), THO complex subunit 2 (THOC2), heterogeneous nuclear ribonucleoproteins C1/C2 (HNRNPC), eukaryotic translation initiation factor 5B (EIF5B), RNA-binding protein Raly (RALY), ubiquitin carboxyl-terminal hydrolase isozyme L5 (UCHL5), KH domain-containing, RNA-binding, signal transduction-associated protein 1 (KHDRBS1), splicing factor 3B subunit 6 (SF3B6), WD repeat-containing protein 44 (WDR44), BRISC and BRCA1-A complex member 2 (BABAM2), cleavage stimulation factor subunit 3 (CSTF3), HIV-1 Tat interactive protein 2 (HTATIP2), methyltransferase like 1 (METTL1), and combinations thereof, and (ii) a protein selected from the list in Table 8, and any combination thereof; thereby detecting the presence or absence of the protein of (i) and the protein of (ii) in the extracellular vesicle and particle protein sample.

Another aspect of the present disclosure is directed to a method of determining the presence of lung cancer in a subject. The method involves obtaining a tissue sample from the subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from the group consisting of Small nuclear ribonucleoprotein Sm D3 (SNRPD3), Four and a half LIM domains protein 2 (FHL2), 60S ribosomal protein L26 (RPL26), 60S ribosomal protein L22 (RPL22), ELAV-like protein 1 (ELAVL1), 5′-3′ exoribonuclease 2 (XRN2), ATP-dependent DNA/RNA helicase DHX36 (DHX36), DnaJ homolog subfamily C member 7 (DNAJC7), Oxidoreductase HTATIP2 (HTATIP2), Amidophosphoribosyltransferase (PPAT), and combinations thereof, and (ii) a protein selected from the group consisting of Caveolae-associated protein 2 (CAVIN2), Na(+)/H(+) exchange regulatory cofactor NHE-RF2 (SLC9A3R2), Protein mab-21-like 4 (MAB21L4), Fructose-1,6-bisphosphatase 1 (FBP1), Heat shock 70 kDa protein 12B (HSPA12B), Sciellin (SCEL), Pulmonary surfactant-associated protein C (SFTPC), Caveolin-2 (CAV2), F-actin-uncapping protein LRRC16A (CARMIL1), Advanced glycosylation end product-specific receptor (AGER), Protein XRP2 (RP2), Specifically androgen-regulated gene protein (C1orf116), and combinations thereof, thereby detecting the presence or absence of the protein of (i) and the protein of (ii) in the extracellular vesicle and particle protein sample.

Another aspect of the present disclosure is directed to a method of determining the presence of lung cancer in a subject that involves obtaining a liquid biopsy sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting: (i) a protein selected from the group consisting of selenoprotein P (SELENOP), rho-related GTP binding protein RhoV (RHOV), roquin-2 (RC3H2), claudin-5 (CLDN5), dematin (DMTN), serine/threonine-protein kinase/endoribonuclease IRE1 (ERN1), IGCL2, radixin (RDX), complement factor B (CFB), trypsin-1, EC 3.4.21.4 (PRSS1), leukocyte surface antigen CD53 (CD53), charged multivesicular body protein 4b (CHMP4B), proteasome subunit beta type-1 (PSMB1), actin aortic smooth muscle (ACTA2), guanine nucleotide-binding protein (GNG5), histone H2A.Z (H2AFZ), histone H2A type 1-C (HISTIH2AC), POTE ankyrin domain family member E (POTEE), POTE ankyrin domain family member I (POTEI) and combinations thereof; and (ii) a protein selected from immunoglobulin heavy constant delta (IGHD), collectin-11 (COLEC11), immunoglobulin lambda variable 4-69 (IGLV4-69), thrombospondin-2 (THBS2), immunoglobulin kappa variable 1-27 (IGKV1-27), immunoglobulin lambda variable 4-60 (IGLV4-60), complement C1q tumor necrosis factor-related protein 3 (C1QTNF3), probable non-functional immunoglobulin heavy variable 3-35 (IGHV3-35), immunoglobulin lambda variable 2-18 (IGLV2-18), immunoglobulin kappa variable 3D-15 (IGKV3D-15), immunoglobulin kappa variable 3D-11 (IGKV3D-11), immunoglobulin kappa variable 1-6 (IGKV1-6), immunoglobulin kappa variable 1-17 (IGKV1-17), attractin (ATRN), immunoglobulin kappa variable 3/OR2-268 (non-functional) (IGKV30R2-268), immunoglobulin lambda variable 3-27 (IGLV3-27), cholinesterase (BCHE), immunoglobulin heavy variable 3/OR15-7 (IGHV3OR15-7), thrombospondin-1 (Glycoprotein G) (THBS1), immunoglobulin kappa variable 1-8 (IGKV1-8), multimerin-1 (MMRN1), probable non-functional immunoglobulin kappa variable 3-7 (IGKV3-7), immunoglobulin lambda variable 3-16 (IGLV3-16), immunoglobulin lambda variable 9-49 (IGLV9-49), apolipoprotein M (APOM), immunoglobulin kappa variable 2-29 (IGKV2-29), immunoglobulin lambda variable 1-44 (IGLV1-44), sushi, von Willebrand factor type A (SVEP1), collectin-10 (COLEC10), integrin alpha-IIb (ITGA2B), complement C1r subcomponent-like protein (C1RL), immunoglobulin kappa variable 1-39 (IGKV1-39), immunoglobulin lambda variable 5-45 (IGLV5-45), insulin-like growth factor-binding protein complex acid labile subunit (IGFALS), HY1, mannose-binding protein C (MBL2), platelet factor 4, PF-4 (PF4), coagulation factor XI, FXI, EC 3.4.21.27 (F11), transforming growth factor beta-1 proprotein (TGFB1), probable non-functional immunoglobulin kappa variable 2D-24 (IGKV2D-24), immunoglobulin kappa variable 2-24 (IGKV2-24), immunoglobulin kappa variable 2D-29 (IGKV2D-29), mannosyl-oligosaccharide 1,2-alpha-mannosidase IC (MAN1C1), charged multivesicular body protein 4a (CHMP4A), SERPIN4A, C-type lectin domain family 3 member B (CLEC3B), platelet factor 4 variant (PF4V1), immunoglobulin kappa variable 1-16 (IGKV1-16), immunoglobulin kappa variable 1-12 (IGKV1-12), Immunoglobulin heavy variable 3/OR16-12 (non-functional) (IGHV3OR16-12) and any combination thereof; thereby detecting the presence or absence of the protein of (i) and the protein of (ii) in the extracellular vesicle and particle protein sample.

Another aspect of the present disclosure is directed to a method of determining the presence of lung cancer in a subject that involves obtaining a liquid biopsy sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting: (i) a protein selected from the group consisting of Putative alpha-1-antitrypsin-related protein (SERPINA2), Immunoglobulin kappa joining 1 (IGKJ1), Protein 4.2 (EPB42), Histone H2A type 1-D (H2AC7), Proteasome subunit alpha type-2 (PSMA2), Nebulette (NEBL), Tripeptidyl-peptidase 2 (TPP2), Monocyte differentiation antigen CD14 (CD14), Fc receptor-like protein 3 (FCRL3), Charged multivesicular body protein 4b (CHMP4B), Rho-related GTP-binding protein RhoV (RHOV), Leukocyte surface antigen CD53 (CD53), Basement membrane-specific heparan sulfate proteoglycan core protein (HSPG2), Trypsin-1 (PRSS1), and combinations therefore, and (ii) transforming growth factor-beta-induced protein ig-h3 (TGFBI), thereby detecting the presence or absence of the protein of (i) and the protein of (ii) in the extracellular vesicle and particle protein sample.

Another aspect of the present disclosure is directed to method of determining the presence of pancreatic cancer in a subject. The method involves obtaining a tissue sample from a subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from, Myosin light polypeptide 6 (MYL6), EH domain-containing protein 1 (EHD1), Myosin-10 (MYH10), Fibronectin (FN1), Tropomyosin alpha-4 chain (TPM4), Flotillin-2 (FLOT2), Apolipoprotein A-I (APOA1), Thrombospondin-1 (THBS1), Tropomyosin alpha-3 chain (TPM3), Versican (VCAN), Dihydropyrimidinase-related protein 3 (DPYSL3), Actin-related protein 2/3 complex subunit 3 (ARPC3), Cathepsin B (CTSB), Thrombospondin-2 (THBS2), Coagulation factor XIII A chain (F13A1), Rho-related GTP-binding protein (RHOG), Myosin-9 (MYH9), Actin-related protein 2 (ACTR2), F-actin-capping protein subunit alpha-1 (CAPZA1), Actin-related protein 3 (ACTR3), Annexin A3 (ANXA3), Vimentin (VIM), Transitional endoplasmic reticulum ATPase (VCP), AP-2 complex subunit beta (AP2B1), Cytoplasmic dynein 1 heavy chain 1 (DYNC1H1), Vacuolar protein sorting-associated protein 35 (VPS35), High affinity immunoglobulin epsilon receptor subunit gamma (FCER1G), TB/POZ domain-containing protein KCTD12 (KCTD12), Guanine nucleotide-binding protein G(q) subunit alpha (GNAQ), Serpin H1 (SERPINH1), Ras-related protein Rab-31 (RAB31), Cytochrome b-245 heavy chain (CYBB), Protein S100-A13 (S100A13), Tropomyosin beta chain (TPM2), Milk fat globule-EGF factor 8 (MFGE8), Periostin (POSTN), Platelet-derived growth factor receptor beta, PDGF-R-beta (PDGFRB), Histidine-rich glycoprotein (HRG), Interferon-induced GTP-binding protein Mx1 (MX1), LIM and senescent cell antigen-like-containing domain protein 1 (LIMS1), Acyl-protein thioesterase 2 (LYPLA2), Inactive tyrosine-protein kinase 7 (PTK7), Ras-related protein Rab-22A (RAB22A), IST1 homolog (IST1), Raftlin (RFTN1), Plexin-B2 (PLXNB2), Vacuolar protein sorting-associated protein 28 homolog (VPS28), C-type mannose receptor 2 (MRC2), Neutrophil elastase (ELANE), Formin-like protein 1 (FMNL1), Cyclin-dependent kinase 4 (CDK4), Cyclin-dependent kinase 2 (CDK2), AP-2 complex subunit sigma (AP2S1), Prolyl endopeptidase FAP (FAP), Basigin (BSG), NADH-cytochrome b5 reductase 3 (CYB5R3), Fibulin-2 (FBLN2), Beta-hexosaminidase subunit beta (HEXB), Cyclin-dependent kinase 17 (CDK17), Tyrosine-protein kinase Lck (LCK), Retinoid-inducible serine carboxypeptidase (SCPEP1), Integrin alpha-X (ITGAX), Complement C1q subcomponent subunit B (C1QB), Macrophage-capping protein (CAPG), Osteoclast-stimulating factor 1 (OSTF1), Syntaxin-7 (STX7), Ectonucleoside triphosphate diphosphohydrolase 1 (ENTPD1), Neutrophil cytosol factor 2 (NCF2), Intercellular adhesion molecule 1 (ICAM1), Kinesin light chain 1 (KLC1), S-phase kinase-associated protein 1 (SKP1), Polyunsaturated fatty acid 5-lipoxygenase (ALOX5), Anoctamin-6 (ANO6), Metalloproteinase inhibitor 1 (TIMP1), 5′-AMP-activated protein kinase subunit gamma-1 (PRKAG1), Unconventional myosin-If (MYO1F), Mucin-5B (MUC5B), Alpha-1-antitrypsin (SERPINA1), and any combination thereof; and (ii) a protein selected from the list in Table 9 and any combination thereof, thereby detecting the presence or absence of the protein of (i) and the protein of (ii) in the extracellular vesicle and particle protein sample.

Another aspect of the present disclosure is directed to method of determining the presence of pancreatic cancer in a subject. The method involves obtaining a tissue sample from a subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from the group consisting of Protein S100-A9 (S100A9), Protein S100-A 11 (S100A11), Protein S100-A13 (S100A13), Integrin alpha-6 (ITGA6), Integrin alpha-V (ITGAV), Versican (VCAN), Fibronectin (FN1), Annexin A1 (ANXA1), Annexin A3 (ANXA3), Cathepsin B (CTSB), Protein-glutamine gamma-glutamyltransferase 2 (TGM2), Complement decay-accelerating factor (CD55), Thymosin beta-10 (TMSB10), Syntenin-2 (SDCBP2), Fermitin family homolog 3 (FERMT3), Myosin-10 (MYH10), Myosin-14 (MYH14), Dihydropyrimidinase-related protein 3 (DPYSL3), Lactadherin (MFGE8), Inactive tyrosine-protein kinase 7 (PTK7), Dipeptidyl peptidase 1 (CTSC), Serpin B5 (SERPINB5), Epidermal growth factor receptor kinase substrate 8-like protein 1 (EPS8L1), Neutrophil cytosol factor 2 (NCF2), Metalloproteinase inhibitor 1 (TIMP1), Cathepsin S (CTSS), Glutamine synthetase (GLUL), Integrin alpha-L (ITGAL), Formin-like protein 1 (FMNL1), Intercellular adhesion molecule 1 (ICAM1), Vascular endothelial growth factor receptor 3 (FLT4), Platelet-derived growth factor receptor alpha (PDGFRA), Integrin alpha-X (ITGAX), Sequestosome-1 (SQSTM1), Retinoic acid-induced protein 3 (GPRC5A), Disintegrin and metalloproteinase domain-containing protein 9 (ADAM9), and combinations thereof, and (ii) a protein selected from the group consisting of Syncollin (SYCN), Pancreatic lipase-related protein 2 (PNLIPRP2), Inactive pancreatic lipase-related protein 1 (PNLIPRP1), Phospholipase A2 (PLA2G1B), Chymotrypsin-like elastase family member 2B (CELA2B), Stress-70 protein, mitochondrial (HSPA9), Very long-chain specific acyl-CoA dehydrogenase, mitochondrial (ACADVL), and combinations thereof, thereby detecting the presence or absence of the protein of (i) and the protein of (ii) in the extracellular vesicle and particle protein sample.

Another aspect of the present disclosure is directed to a method determining presence of pancreatic cancer in a subject that involves obtaining a liquid biopsy sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting: (i) a protein selected from the group consisting of HSPA2, CA2, RAB1A, RAB8B, RAP1A, BAIAP2L1, CD55, GLIPR2, KRAS, LRRC26, LTF, P4HB, PEBP1, RDX, ABCB1, ABCB11, ABCB4, ADGRG6, ADH1A, ALPL, ITGA1, PACSIN2, PTPRJ, RAP2B, SRI, XPNPEP2, ADH1C, ADH4, ANXA11, CCT6A, CPNE1, DSC1, DSG1, DSP, ENPEP, FABP1, FCER1G, FLNB, GNG5, KRT8, KRT81, KRT85, MPP1, PLGLB1, PRDX6, PSMA4, PSMA5, SFN, SNX18, TGM2, cell surface hyaluronidase (TMEM2), and combinations thereof, and (ii) a protein selected from IGHD, collectin-11 (COLEC11), IGLV4-69, Thrombospondin-2 (THBS2), IGKV1-27, IGLV4-60, C1QTNF3, IGHV3-35, IGLV2-18, IGKV3D-15, IGKV3D-11, IGKV1-6, IGKV1-17, ATRN, IGKV3OR2-268, IGLV3-27, BCHE, IGHV3OR15-7, THBS1, IGKV1-8, MMRN1, IGKV3-7, IGLV3-16, IGLV9-49, APOM, IGKV2-29, IGLV1-44, SVEP1, COLEC10, ITGA2B, C1RL, IGKV1-39, IGLV5-45, IGFALS, HY1, MBL2, PF4, F11, TGFB1, IGKV2D-24, IGKV2-24, IGKV2D-29, MAN1C1, CHMP4A, SERPIN4A, CLEC3B, PF4V1, IGKV1-16, IGKV1-12, IGHV3OR16-12 and any combination thereof, thereby detecting the presence or absence of the protein of (i) and the protein of (ii) in the extracellular vesicle and particle protein sample.

Another aspect of the present disclosure is directed to a method determining presence of pancreatic cancer in a subject that involves obtaining a liquid biopsy sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting: (i) a protein selected from the group consisting of Calmodulin-like protein 5 (CALML5), Carboxypeptidase N subunit 2 (CPN2), Carbonic anhydrase 2 (CA2), Heat shock-related 70 kDa protein 2 (HSPA2), Lactotransferrin (LTF), GTPase KRas (KRAS), Complement decay-accelerating factor (CD55), Brain-specific angiogenesis inhibitor 1-associated protein 2-like protein 1 (BAIAP2L1), Phosphatidylethanolamine-binding protein 1 (PEBP1), Ras-related protein Rab-1A (RAB1A), Ras-related protein Rab-8B (RAB8B), Desmoplakin (DSP), Leucine-rich repeat-containing protein 26 (LRRC26), and combinations therefore, and (ii) a protein selected from the group consisting of Thrombospondin-1 (THBS1), Complement C1r subcomponent-like protein (C1RL), Immunoglobulin kappa variable 1-6 (IGKV1.6), Immunoglobulin kappa variable 1-17 (IGKV1.17), Immunoglobulin kappa variable 1-39 (IGKV1.39), Immunoglobulin kappa variable 1-27 (IGKV1.27), Immunoglobulin kappa variable 1-12 (IGKV1.12), and Immunoglobulin kappa variable 1D-33 (IGKV1D.33), and combinations thereof, thereby detecting the presence or absence of the protein of (i) and the protein of (ii) in the extracellular vesicle and particle protein sample.

Another aspect of the present disclosure is directed to a method of determining the presence of neuroblastoma in a subject. The method involves obtaining a liquid biopsy sample from the subject, separating extracellular vesicles and particles from the liquid biopsy sample, and isolating protein from the separated extracellular vesicles and particles to form an extracellular vesicle and particle protein sample. The extracellular vesicle and particle protein sample are subjected to a detection assay suitable for detecting (i) a protein selected from the group consisting of ferritin heavy chain (FTH1), keratin, type I cytoskeletal 17 (KRT17), histone H3.3 (H3F3A), ATP-binding cassette sub-family B member 9 (ABCB9), a disintegrin and metalloproteinase with thrombospondin motifs 13 (ADAMTS13), CD14, erythrocyte membrane protein band 4.2 (EPB42), hepatocyte growth factor activator (HGFAC), keratin, type I cytoskeletal 13 (KRT13), and KRT8, and combinations thereof and (ii) a protein selected from the list in Table 3 (FIG. 25) or any combination of proteins thereof; thereby detecting the presence or absence of the protein of (i) and the protein of (ii) in the extracellular vesicle and particle protein sample.

In another aspect, the present disclosure relates to a method of determining the presence of osteosarcoma in a subject. The method involves obtaining a liquid biopsy sample from a subject, separating extracellular vesicles and particles from the liquid biopsy sample, and isolating protein from the separated extracellular vesicles and particles to form an extracellular vesicle and particle protein sample. The extracellular vesicle and particle protein sample are subjected to a detection assay suitable for detecting a protein (i) selected from the group consisting of actin, alpha skeletal muscle (ACTA1), actin, gamma-enteric smooth muscle (ACTG2), ADAMTS13, HGFAC, neprilysin (MME), and TNC, and combinations thereof and (ii) a protein from the list in Table 4 (FIG. 26) or any combination of proteins thereof.

Another aspect of the present disclosure is directed to a method of cancer sub-type identification that involves obtaining a liquid biopsy sample from a subject, separating extracellular vesicles and particles from the sample, and isolating protein from the separated extracellular vesicles and particles to form an extracellular vesicle and particle protein sample. The extracellular vesicle and particle protein sample is subjected to a detection assay suitable for detecting levels of at least three proteins selected from the group consisting of Fibrinogen beta chain (FGB), FGA (Fibrinogen alpha chain), Fibrinogen gamma chain (FGG), Complement factor H (CFH), Plasminogen (PLG), Immunoglobulin heavy variable 3-53 (IGHV3-53), Serum amyloid P-component, SAP (APCS), Complement factor H-related protein 1 (CFHR1), Immunoglobulin heavy variable 3-48 (IGHV3-48), Immunoglobulin heavy variable 3-74 (IGHV3-74), Immunoglobulin heavy variable 3-72 (IGHV3-72), Immunoglobulin heavy variable 3-43 (IGHV3-43), Immunoglobulin heavy variable 5-10-1 (IGHV5-10-1), Immunoglobulin lambda variable 7-46 (IGLV7-46), Immunoglobulin kappa variable 3D-20 (IGKV3D-20), Immunoglobulin kappa variable 2-24 (IGKV2-24), Complement factor H-related protein 2 (CFHR2), Immunoglobulin heavy variable 4-59 (IGHV4-59), Immunoglobulin heavy variable 3-20 (IGHV3-20), Immunoglobulin heavy variable 3-64 (IGHV3-64), Probable non-functional immunoglobulin heavy variable 3-16 (IGHV3-16), Immunoglobulin heavy variable 3-11 (IGHV3-11), Immunoglobulin heavy variable 3/OR16-9 (IGHV3OR16-9), Probable non-functional immunoglobulin kappa variable 2D-24 (IGKV2D-24), Immunoglobulin lambda constant 3 (IGLC3), Immunoglobulin heavy variable 3/OR16-13 (IGHV3OR16-13), Complement factor H-related protein 3 (CFHR3), Immunoglobulin heavy constant gamma 3 (IGHG3), Immunoglobulin lambda constant 2 (IGLC2), and Immunoglobulin kappa variable 1-8 (IGKV1-8).

Another aspect of the present disclosure is directed to a method of cancer sub-type identification that involves obtaining a tissue sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting at least three proteins selected from the group consisting of Apolipoprotein D (APOD), Polyubiquitin-C (UBC), Transaldolase (TALDO1), Thymidine phosphorylase (TYMP), Aminopeptidase B (RNPEP), Transgelin (TAGLN), Septin (SEPT7), Histone H2A type 2-B (HIST2H2AB), Gamma-enolase (ENO2), NADH-cytochrome b5 reductase 3 (CYB5R3), Actin-related protein 2/3 complex subunit 4 (ARPC4), Interleukin enhancer-binding factor 2 (ILF2), Protein transport protein Sec23B (SEC23B), COMM domain-containing protein 3 (COMMD3), Ankyrin-3 (ANK3), Glycogen phosphorylase, muscle form (PYGM), Putative histone H2B type 2-D (HIST2H2BD), Keratin, type I cytoskeletal 19 (KRT19), Sulfotransferase 1A2 (SULT1A2), Desmin (DES), Histone H2B (HIST1H2BD), Histone H2B type 1-A (HIST1H2BA), Histone H3.It (HIST3H3), Tubulin beta-1 chain (TUBB1), Retinal dehydrogenase 2 (ALDH1A2), HLA class II histocompatibility antigen, DP beta 1 chain (HLA-DPB1), Bifunctional epoxide hydrolase 2 (EPHX2), Mitochondrial-processing peptidase subunit alpha (PMPCA), and Xylulose kinase (XYLB).

Another aspect of the present disclosure is directed to a method of identifying a primary tumor of unknown origin. The method involves obtaining a tissue sample from a subject, wherein the tissue sample is from a primary tumor of unknown origin, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting one or more proteins independently selected from the proteins of Table 12, 13, 14, and 15.

Another aspect of the present disclosure is directed to a method of identifying a pancreatic lesion in a subject. The method involves obtaining a liquid biopsy sample from a subject, separating extracellular vesicles and particles from the biopsy sample, and isolating protein from the separated extracellular vesicles and particles to form an extracellular vesicle and particle protein sample. The extracellular vesicle and particle protein sample is subjected to a detection assay suitable for detecting one or more proteins selected from PSMA2, CA2, EPB41, CD59, CNP, RAB8A, TPI1, GGT1, GGT3P, GGT2, PEBP1, IGLV8-61, C6, PON1, CPN2, ECM1, Ig kappa chain V-I region AG, IGKV4-1, IG lambda chain V-1 region, CFP, TUBB, TUBB4B, TUBB2B, TUBB2A, VCL, RSU1, FERMT3, and ADAMTS13.

Another aspect of the present disclosure is directed to a method of isolating extracellular vesicles and particles from a biological sample that involves obtaining a biological sample from a subject and contacting the sample with one or more binding molecules, wherein each binding molecule is capable of binding to a target extracellular vesicle and particle protein selected from the group consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin. The sample, after said contacting, is subjected to conditions effective for the one or more binding molecules to bind to its respective target extracellular vesicle and particle protein in the sample to form one or more binding molecule-target protein complexes. The one or more binding molecule-target protein complexes are separated from the sample, thereby isolating extracellular vesicles and particles from the sample.

Another aspect of the present disclosure is directed to a kit suitable for detecting, in a liquid biopsy sample from a subject, the presence of cancer in the subject. The kit includes reagents suitable for detecting: (i) one or more proteins selected from the group consisting of ferritin light chain, von Willebrand factor, immunoglobulin lambda constant 2, keratin 17, immunoglobulin heavy constant gamma 1, keratin 6B, radixin, cofilin 1, protease, serine 1, tubulin alpha 1c, ADAM metallopeptidase with thrombospondin type 1 motif 13, immunoglobulin kappa variable 6D-21, tyrosine 3-monooxygenase/tryptophan 5-monooxygenase activation protein theta, POTE ankyrin domain family member I, POTE ankyrin domain family member F, and immunoglobulin kappa variable 2D-30, and combinations thereof, and (ii) one or more proteins selected from the group consisting of actin gamma 1, immunoglobulin lambda variable 3-27, immunoglobulin kappa variable 1D-12, coagulation factor XI, complement C1r subcomponent like, attractin, butyrylcholinesterase, immunoglobulin heavy variable 3-35, immunoglobulin kappa variable 1-17, C1q and TNF related 3, immunoglobulin heavy variable 3-20, immunoglobulin heavy variable 3/OR15-7, collectin subfamily member 11, immunoglobulin heavy constant delta, immunoglobulin kappa variable 3D-11, immunoglobulin heavy variable 3/OR16-10, immunoglobulin kappa variable 2D-24, immunoglobulin kappa variable 2-40, immunoglobulin kappa variable 1-27, immunoglobulin heavy variable 3/OR16-9, immunoglobulin lambda variable 5-45, immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6, and combinations thereof.

Another aspect of the present disclosure is directed to a kit suitable for detecting, in a liquid biopsy sample from a subject, the presence of cancer in the subject. The kit includes reagents suitable for detecting: (i) one or more proteins selected from the group consisting of Ferritin light chain (FTL), ABC-type oligopeptide transporter ABCB9 (ABCB9), Protein Z-dependent protease inhibitor (SERPINA10), Coagulation factor VIII (F8), Lactotransferrin (LTF), Basement membrane-specific heparan sulfate proteoglycan core protein (HSPG2), Protein disulfide-isomerase (P4HB), Trypsin-1 (PRSS1), Keratin, type II cytoskeletal 1b (KRT77), Endoplasmic reticulum chaperone BiP (HSPA5); and (ii) one or both proteins selected from the group consisting of Complement C1q tumor necrosis factor-related protein 3 (C1QTNF3) and Immunoglobulin heavy constant delta (IGHD). In some embodiments, the kit includes at least reagent for detecting the presence of one or more of LTF, HSPG2, P4HB, and PRSS1.

Another aspect of the present disclosure is directed to a kit suitable for detecting, in a tissue derived exosomal sample from a subject, the presence of cancer in the subject. The kit includes reagents suitable for detecting: (i) one or more proteins selected from the group consisting of thrombospondin 2, versican, serrate, RNA effector molecule, tenascin C, dihydropyrimidinase like 2, adenosylhomocysteinase, DnaJ heat shock protein family (Hsp40) member A1, phosphoglycerate kinase 1, EH domain containing 2, and combinations thereof, and (ii) one or more proteins selected from the group consisting of alcohol dehydrogenase 1B (class I), beta polypeptide, caveolae associated protein 1, FGGY carbohydrate kinase domain containing, ATP binding cassette subfamily A member 3, syntaxin 11, caveolae associated protein 2, CD36 molecule, and combinations thereof.

Another aspect of the present disclosure is directed to a kit suitable for detecting, in a tissue derived exosomal, sample from a subject, the presence of cancer in the subject. The kit includes reagents suitable for detecting: (i) one or more proteins selected from the group consisting of tenacin (TNC), Periostin (POSTN), Versican core protein (VCAN), signal recognition particle 9 kDa protein (SRP9), Nucleophosmin (NPM1), Serrate RNA effector molecule homolog (SRRT), ELAV-like protein 1 (ELAVL1), Cytosolic acyl coenzyme A thioester hydrolase (ACOT7), 5′-3′ exoribonuclease 2 (XRN2), Flap endonuclease 1 (FEN1), ADP-ribosylation factor-like protein 1 (ARL1), Heat shock protein 105 kDa (HSPH1), Nucleolar RNA helicase 2 (DDX21), Src-associated in mitosis 68 kDa protein (KHDRBS1), Importin subunit alpha-1 (KPNA2), SLIT-ROBO Rho GTPase-activating protein 1 (SRGAP1), WD repeat-containing protein 3 (WDR3), and (ii) one or more proteins selected from the group consisting of Voltage-dependent calcium channel subunit alpha-2/delta-2 (CACNA2D2), Specifically androgen-regulated gene protein (C1orf116), Caveolin-2 (CAV2), Syntaxin-11 (STX11), Caveolae-associated protein 2 (CAVIN2). In some embodiments, the kit includes at least reagent for detecting the presence of one or more of KPNA2, SRGAP1, WDR3.

Another aspect of the present disclosure is directed to a kit suitable for identifying the origin of a tumor from a liquid biopsy. The kit includes reagents suitable for detecting at least three proteins selected from the group consisting of fibrinogen beta chain (FGB), fibrinogen alpha chain (FGA), fibrinogen gamma chain (FGG), complement factor H (CFH), plasminogen (PLG), immunoglobulin heavy variable 3-53 (IGHV3-53), serum amyloid P-component (APCS), complement factor H-related protein 1 (CFHR1), immunoglobulin heavy variable 3-48 (IGHV3-48), immunoglobulin heavy variable 3-74 (IGHV3-74), immunoglobulin heavy variable 3-72 (IGHV3-72), immunoglobulin heavy variable 3-43 (IGHV3-43), immunoglobulin heavy variable 5-10-1 (IGHV5-10-1), immunoglobulin lambda variable 7-46 (IGLV7-46), immunoglobulin kappa variable 3D-20 (IGKV3D-20), immunoglobulin kappa variable 2-24 (IGKV2-24), complement factor H-related protein 2 (CFHR2), immunoglobulin heavy variable 4-59 (IGHV4-59), immunoglobulin heavy variable 3-20 (IGHV3-20), immunoglobulin heavy variable 3-64 (IGHV3-64), probable non-functional immunoglobulin heavy variable 3-16 (IGHV3-16), immunoglobulin heavy variable 3-11 (IGHV3-11), Immunoglobulin heavy variable 3/OR16-9 (IGHV3OR16-9), probable non-functional immunoglobulin kappa variable 2D-24 (IGKV2D-24), immunoglobulin lambda constant 3 (IGLC3), Immunoglobulin heavy variable 3/OR16-13 (IGHV3OR16-13), complement factor H-related protein 3 (CFHR3), immunoglobulin heavy constant gamma 3 (IGHG3), immunoglobulin lambda constant 2 (IGLC2), and immunoglobulin kappa variable 1-8 (IGKV1-8).

Another aspect of the present disclosure is directed to a kit suitable for detecting, in a liquid biopsy sample from a subject, the presence of cancer in the subject. The kit includes reagents suitable for detecting: (i) one or more proteins selected from the group consisting of Ferritin light chain (FTL), ABC-type oligopeptide transporter ABCB9 (ABCB9), Protein Z-dependent protease inhibitor (SERPINA10), Coagulation factor VIII (F8), Lactotransferrin (LTF), Basement membrane-specific heparan sulfate proteoglycan core protein (HSPG2), Protein disulfide-isomerase (P4HB), Trypsin-1 (PRSS1), Keratin, type II cytoskeletal 1b (KRT77), Endoplasmic reticulum chaperone BiP (HSPA5); and (ii) one or both proteins selected from the group consisting of Complement C1q tumor necrosis factor-related protein 3 (C1QTNF3) and Immunoglobulin heavy constant delta (IGHD). In some embodiments, the kit includes at least reagent for detecting the presence of one or more proteins selected from LTF, HSPG2, P4HB, and PRSS1.

Another aspect of the present disclosure is directed to a kit suitable for identifying the origin of a metastatic tumor from a tissue biopsy. The kit includes reagents suitable for detecting at least one or more proteins selected from the proteins listed in Tables 12, 13, 14, and 15.

Another aspect of the present disclosure is directed to a kit suitable for isolating exosome from a human sample. The kit includes at least one binding molecule capable of binding a protein selected from the group consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin.

Another aspect of the present disclosure is directed to a kit for identifying a pancreatic lesion. The kit includes reagents suitable for detecting at least one or more proteins selected from PSMA2, CA2, EPB41, CD59, CNP, RAB8A, TPI1, GGT1, GGT3P, GGT2, PEBP1, IGLV8-61, C6, PON1, CPN2, ECM1, Ig kappa chain V-I region AG, IGKV4-1, IG lambda chain V-1 region, CFP, TUBB, TUBB4B, TUBB2B, TUBB2A, VCL, RSU1, FERMT3, and ADAMTS13.

Another aspect of the present disclosure is directed to a kit suitable for detecting, in a liquid biopsy sample from a subject, the presence of pancreatic cancer in the subject. The kit includes reagents suitable for detecting: (i) one or more proteins selected from the group consisting of Calmodulin-like protein 5 (CALML5), Carboxypeptidase N subunit 2 (CPN2), Carbonic anhydrase 2 (CA2), Heat shock-related 70 kDa protein 2 (HSPA2), Lactotransferrin (LTF), GTPase KRas (KRAS), Complement decay-accelerating factor (CD55), Brain-specific angiogenesis inhibitor 1-associated protein 2-like protein 1 (BAIAP2L1), Phosphatidylethanolamine-binding protein 1 (PEBP1), Ras-related protein Rab-1A (RAB1A), Ras-related protein Rab-8B (RAB8B), Desmoplakin (DSP), Leucine-rich repeat-containing protein 26 (LRRC26), and (ii) one or more proteins selected from the group consisting of Thrombospondin-1 (THBS1), Complement C1r subcomponent-like protein (C1RL), Immunoglobulin kappa variable 1-6 (IGKV1.6), Immunoglobulin kappa variable 1-17 (IGKV1.17), Immunoglobulin kappa variable 1-39 (IGKV1.39), Immunoglobulin kappa variable 1-27 (IGKV1.27), Immunoglobulin kappa variable 1-12 (IGKV1.12), and Immunoglobulin kappa variable 1D-33 (IGKV1D.33). In some embodiments, the kit includes at least reagents for detecting the presence of one or more proteins selected from LTF, KRAS, CD55, BAIAP2L1, PEBP1, DSP, and LRRC26.

Another aspect of the present disclosure is directed to a kit suitable for detecting, in a tissue derived exosomal sample from a subject, the presence of pancreatic cancer in the subject. The kit includes reagents suitable for detecting: (i) one or more proteins selected from the group consisting of Protein S100-A9 (S100A9), Protein S100-A11 (S100A11), Protein S100-A13 (S100A13), Integrin alpha-6 (ITGA6), Integrin alpha-V (ITGAV), Versican (VCAN), Fibronectin (FN1), Annexin A1 (ANXA1), Annexin A3 (ANXA3), Cathepsin B (CTSB), Protein-glutamine gamma-glutamyltransferase 2 (TGM2), Complement decay-accelerating factor (CD55), Thymosin beta-10 (TMSB10), Syntenin-2 (SDCBP2), Fermitin family homolog 3 (FERMT3), Myosin-10 (MYH10), Myosin-14 (MYH14), Dihydropyrimidinase-related protein 3 (DPYSL3), Lactadherin (MFGE8), Inactive tyrosine-protein kinase 7 (PTK7), Dipeptidyl peptidase 1 (CTSC), Serpin B5 (SERPINB5), Epidermal growth factor receptor kinase substrate 8-like protein 1 (EPS8L1), Neutrophil cytosol factor 2 (NCF2), Metalloproteinase inhibitor 1 (TIMP1), Cathepsin S (CTSS), Glutamine synthetase (GLUL), Integrin alpha-L (ITGAL), Formin-like protein 1 (FMNL1), Intercellular adhesion molecule 1 (ICAM1), Vascular endothelial growth factor receptor 3 (FLT4), Platelet-derived growth factor receptor alpha (PDGFRA), Integrin alpha-X (ITGAX), Sequestosome-1 (SQSTM1), Retinoic acid-induced protein 3 (GPRC5A), Disintegrin and metalloproteinase domain-containing protein 9 (ADAM9), and (ii) one or more proteins selected from the group consisting of Syncollin (SYCN), Pancreatic lipase-related protein 2 (PNLIPRP2), Inactive pancreatic lipase-related protein 1 (PNLIPRP1), Phospholipase A2 (PLA2G1B), Chymotrypsin-like elastase family member 2B (CELA2B), Stress-70 protein, mitochondrial (HSPA9), Very long-chain specific acyl-CoA dehydrogenase, mitochondrial (ACADVL). In some embodiments, the kit includes at least reagents for detecting the presence of one or more proteins selected from CTSC, SERPINB5, EPS8L1, NCF2, TIMP1, CTSS, GLUL, ITGAL, FMNL1, ICAM1, FLT4, PDGFRA, ITGAX, SQSTM1, GPRC5A, ADAM9, HSPA9, and ACADVL.

Another aspect of the present disclosure is directed to a kit suitable for detecting, in a liquid biopsy sample from a subject, the presence of lung cancer in the subject. The kit includes reagents suitable for detecting: (i) one or more proteins selected from the group consisting of Putative alpha-1-antitrypsin-related protein (SERPINA2), Immunoglobulin kappa joining 1 (IGKJ1), Protein 4.2 (EPB42), Histone H2A type 1-D (H2AC7), Proteasome subunit alpha type-2 (PSMA2), Nebulette (NEBL), Tripeptidyl-peptidase 2 (TPP2), Monocyte differentiation antigen CD14 (CD14), Fc receptor-like protein 3 (FCRL3), Charged multivesicular body protein 4b (CHMP4B), Rho-related GTP-binding protein RhoV (RHOV), Leukocyte surface antigen CD53 (CD53), Basement membrane-specific heparan sulfate proteoglycan core protein (HSPG2), Trypsin-1 (PRSS1), and (ii) Transforming growth factor-beta-induced protein ig-h3 (TGFBI). In some embodiments, the kit includes at least reagents for detecting the presence of one or more proteins selected from CHMP4B, RHOV, CD53, HSPG2, and PRSS1.

Another aspect of the present disclosure is directed to a kit suitable for detecting, in a tissue derived exosomal sample from a subject, the presence of lung cancer in the subject. The kit includes reagents suitable for detecting: (i) one or more proteins selected from the group consisting of Small nuclear ribonucleoprotein Sm D3 (SNRPD3), Four and a half LIM domains protein 2 (FHL2), 60S ribosomal protein L26 (RPL26), 60S ribosomal protein L22 (RPL22), ELAV-like protein 1 (ELAVL1), 5′-3′ exoribonuclease 2 (XRN2), ATP-dependent DNA/RNA helicase DHX36 (DHX36), DnaJ homolog subfamily C member 7 (DNAJC7), Oxidoreductase HTATIP2 (HTATIP2), Amidophosphoribosyltransferase (PPAT), and (ii) one or more proteins selected from the group consisting of Caveolae-associated protein 2 (CAVIN2), Na(+)/H(+) exchange regulatory cofactor NHE-RF2 (SLC9A3R2), Protein mab-21-like 4 (MAB21L4), Fructose-1,6-bisphosphatase 1 (FBP1), Heat shock 70 kDa protein 12B (HSPA12B), Sciellin (SCEL), Pulmonary surfactant-associated protein C (SFTPC), Caveolin-2 (CAV2), F-actin-uncapping protein LRRC16A (CARMIL1), Advanced glycosylation end product-specific receptor (AGER), Protein XRP2 (RP2), Specifically androgen-regulated gene protein (C1orf116). In some embodiments, the kit includes at least reagents for detecting the presence of one or more proteins selected from HTATIP2 and PPAT

Another aspect of the present disclosure is directed to a method of determining a treatment regimen for a subject having a tumor. The method involves obtaining, from the subject having the tumor, a biopsy of tumor tissue and a biopsy of tissue adjacent to the tumor, and separating extracellular vesicles and particles from the obtained samples. Protein from the separated extracellular vesicle and particles is isolated to form extracellular vesicle and particle protein samples, and the extracellular vesicle and particle protein samples is subjected to a detection assay suitable for detecting proteins differentially expressed in the tumor tissue versus adjacent, non-tumor tissue. A treatment regimen for the subject is identified based on said subjecting.

Another aspect of the present disclosure is directed to a method of identifying drug targets for cancer therapy. The method involves obtaining, from each of a plurality of subjects having a particular tumor, a biopsy of tumor tissue and a biopsy of tissue adjacent to said tumor, and separating extracellular vesicles and particles from the obtained samples. Protein from the separated extracellular vesicle and particles is isolated to form extracellular vesicle and particle protein samples, and the extracellular vesicle and particle protein samples is subjected to proteomic analysis to identify proteins differentially expressed in the tumor tissue versus tissue adjacent said tumor. Drug targets for cancer therapy are identified based on said subjecting.

Another aspect of the present disclosure is directed to a method of treating a subject having cancer. The method involves selecting a subjecting having a tumor, wherein exosomes from tumor tissue express Src-associated in mitosis 68 kDa protein (Sam68; KHDRBS1) and administering to said subject a Sam68 inhibitor.

Another aspect of the present disclosure is directed to a method of treating a subject having a tumor. The method involves selecting a subjecting having a tumor, wherein exosomes from the tumor tissue express nucleolin (NCL), and administering to said subject a nucleolin inhibitor.

Another aspect of the present disclosure is directed to a method of treating a subject having a tumor. The method involves selecting a subjecting having a tumor, wherein exosomes from the tumor tissue express tenacin (TNC), and administering to said subject a tenacin inhibitor.

Another aspect of the present disclosure is directed to a method of treating a subject having a tumor. The method involves selecting a subjecting having a tumor, wherein exosomes from the tumor tissue express inosine-5′-monophosphate dehydrogenase 2 (IMPDH2), and administering to said subject an inosine-5′-monophosphate dehydrogenase 2 inhibitor.

Another aspect of the present disclosure is directed to a method of treating a subject having a tumor. The method involves selecting a subjecting having a tumor, wherein exosomes from the tumor tissue express GMP synthase (GMPS), and administering to said subject a glutamine amidotransferase inhibitor.

Another aspect of the present disclosure is directed to a method of treating a subject having a tumor. The method involves selecting a subjecting having a tumor, wherein exosomes from the tumor tissue express DNA topoisomerase I (TOP1MT), and administering to said subject a DNA topoisomerase I inhibitor.

Another aspect of the present disclosure is directed to a method of treating a subject having a tumor. The method involves selecting a subjecting having a tumor, wherein exosomes from the tumor tissue express bifunctional purine biosynthesis protein ATIC (ATIC), and administering to said subject an ATIC inhibitor.

Another aspect of the present disclosure is directed to a method of treating a subject having a tumor. The method involves selecting a subjecting having a tumor, wherein exosomes from the tumor tissue express aldo-keto reductase family 1 member B1 (AKR1B1), and administering to said subject an aldo-keto reductase family 1 member B1 inhibitor.

Another aspect of the present disclosure is directed to a method of treating a subject having a tumor. The method involves selecting a subjecting having a tumor, wherein plasma tumor derived exosomes of the subject express cytokeratin-2e (KRT2), and administering to said subject a cytokeratin-2e inhibitor.

Another aspect of the present disclosure is directed to a method of treating a subject having a tumor. The method involves selecting a subjecting having a tumor, wherein plasma tumor derived exosomes of the subject express coagulation factor VIII (F8), and administering to said subject a coagulation factor VIII inhibitor.

Another aspect of the present disclosure is directed to a method of treating a subject having a tumor. The method involves selecting a subjecting having a tumor, wherein plasma tumor derived exosomes of the subject express peptidyl-prolyl cis-trans isomerase A (PPIA), and administering to said subject a peptidyl-prolyl cis-trans isomerase A inhibitor.

Another aspect of the present disclosure is directed to a method of treating a subject having a tumor. The method involves selecting a subjecting having a tumor, wherein plasma tumor derived exosomes of the subject express carbonic anhydrase I (CA1), and administering to said subject a carbonic anhydrase I inhibitor.

To identify universal exosomal markers and improve the isolation of human exosomes, a total of 497 human and murine samples were analyzed by exosome proteomic profiling. Among the conventional exosome markers evaluated, heat shock cognate 71 kDa protein (HSPA8), heat shock protein HSP 90-beta (HSP90AB1), CD9 and programmed cell death 6-interacting protein (ALIX) were the most prominent markers found in human-derived exosomes isolated from cells, tissues, and biofluids (except for bile duct fluid and plasma for CD9 and ALIX, respectively). Importantly, in human cell lines, these markers were shared by all particle sub-populations, including exomeres, Exo-S and Exo-L, thus representing pan-exosome/exomere markers (Zhang et al., “Identification of Distinct Nanoparticles and Subsets of Extracellular Vesicles by Asymmetric Flow Field-Flow Fractionation,” Nature Cell Biology 20:332-343 (2018); Zhang et al., “Asymmetric-Flow Field-Flow Fractionation Technology for Exomere and Small Extracellular Vesicle Separation and Characterization,” Nat Protoc 14:1027-1053 (2019)). Thirteen additional proteins shared by >50% of the human samples analyzed were identified, thus drastically expanding the panel of representative human exosome markers. To identify cancer-specific exosomal protein signatures, the exosomal proteomes of paired tumor and adjacent tissue from freshly resected surgical specimens of patients with various cancers as described herein were characterized and compared.

Tumor-associated exosomal proteins expressed in the plasma of cancer patients but never detected in non-cancer samples were identified. By comparing the exosome proteomes of matched tissue-derived and plasma-derived exosomes for each cancer type, it was determined that exosomal proteins in plasma were derived from a variety of sources, including tumor tissue, distant organs, as well as the immune system, emphasizing the importance of using non-cancer cell-derived exosomal signatures to identify cancer-associated alterations and define tumor-associated biomarkers. Thus, stage I-IV cancers were analyzed from 12 pediatric and 6 adult cancer types for tissue (n=85 patient tissue) and 10 pediatric and 6 adult cancer types for plasma (n=77 patient plasma), respectively, and were compared to the exosomal proteomes of non-tumor tissues and plasma. Random forest classification based on exosomal proteomes revealed 90% sensitivity and 94% specificity in cancer detection for tissues, and 95% sensitivity and 90% specificity in cancer detection for plasma, respectively. Importantly, by comparing plasma-derived exosome cargo from different cancers, cancer types were able to be distinguished in patients. These data suggest that tumor-associated exosomal proteins could be used as biomarkers for early stage cancer detection and potentially for diagnosing tumors of unknown primary origin.

### BRIEF DESCRIPTION OF THE DRAWINGS

FIGS. 1A-1F show proteomic characterization of EVPs obtained from 497 samples, from seven different sources. FIG. 1A is a description of samples analyzed. A total of 426 human samples and 71 murine samples were collected for EVP proteomics analysis by liquid chromatography tandem-mass spectrometry (LC-MS/MS). FIG. 1B shows the centrifugation protocol and general workflow for EVP enrichment for LC-MS/MS analysis (left), representative nanoparticle tracking analysis (NTA) (middle), and transmission electron microscopy (TEM) imaging (right) of EVPs from human control plasma. Scale bar, 200 nm. FIG. 1C shows Pearson correlation of EVP protein expression among tissue types. Larger size and darker shading depict a higher correlation between samples. FIG. 1D shows positivity for 11 conventional EVP protein markers across different tissue types. Noted in each box is the frequency (%) of samples from each source in which the respective protein was present. Darker shading depicts higher frequency. FIG. 1E shows the frequency (%) of samples from each source positive for the 13 newly defined EVP markers. Proteins found in more than 50% of all human samples were identified. Noted in each box is the frequency (%) of samples positive for the protein. Darker shading depicts higher frequency. FIG. 1F shows gene ontology analysis using Metascape for the 13 common exosomal proteins listed in FIG. E.

FIGS. 2A-2B show representative TEM images and Nanosight profiles. FIG. 2A shows TEM images for EVPs isolated from: a human cell line (Panc-1), human plasma (colon cancer), human explant (melanoma), human lymphatic fluid, human bile duct fluid, a mouse cell line (B16-F10), and mouse explant (lung tissue). FIG. 2B shows Nanosight profiles showing size distribution for EVP isolated from: a human cell line (HCT116), human plasma (melanoma), human explant (pancreatic cancer), human lymphatic fluid, human bile duct fluid, a mouse cell line (4T1), mouse plasma (B16-F10), and mouse explant (lung tissue). Scale bar, 200 nm.

FIGS. 3A-3C show the average number of EVP proteins detected by mass spectrometry for each source. FIG. 3A shows results from all samples (n=497), FIG. 3B shows results from tumor samples (n=310), and FIG. 3C shows results from non-tumor samples (n=187).

FIGS. 4A-4F show the EVP protein cargo correlation between tumor and non-tumor samples. FIGS. 4A-4B show Pearson correlation of EVP protein levels among source types. Larger size and darker shading depict a higher correlation between samples. FIG. 4A shows results from tumor samples (n=274 human, (n=36 mouse), and FIG. 4B shows results from non-tumor samples (n=152 human, n=35 mouse). FIGS. 4C-4D show positivity of conventional exosome markers across different sources. FIG. 4C shows results from tumor samples (n=310), and FIG. 4D shows results from non-tumor samples (n=187). The number in each box is the percentage of positivity in each tissue type. The colors in each box were represented from white for 0% to red for 100%. FIGS. 4E-4F show positivity for the newly identified 13 EVP proteins found in more than 50% of all human samples. FIG. 4E shows results from tumor samples (n=310), and FIG. 4F shows results from non-tumor samples (n=187).

FIG. 5 is a heatmap illustration of the conventional exosome markers and the newly identified 13 EVP proteins in exomeres, Exo-S and Exo-L. EVP subpopulations (exomeres, <50 nm with an average of 35 nm in diameter; Exo-S, 60-80 nm in diameter; Exo-L, 90-120 nm in diameter and small exosome vesicles) were separated using asymmetric-flow field flow fractionation (AF4). The relative abundances of conventional exosome marker and newly identified 13 EVP proteins are shown in the heatmaps. Scale shown is intensity (area) subtracted by mean and divided by row standard deviation (i.e. Δ [expression-mean]/SD).

FIGS. 6A-6E show the identification of tumor tissue-specific EVP protein cargo in surgically removed tissue explants. FIG. 6A is a schematic diagram of the explant culture method used for pairwise comparison of tumor and non-tumor tissue-derived EVPs from the same group of patients. EVPs were isolated from paired tumor tissue (TT) and adjacent tissues (AT) and were cultured in serum-free media for 24 hours. For lung cancer (LuCa), distant tissues (DT) from the same patients were also cultured for EVP isolation. FIG. 6B is a heatmap showing the top 30 proteins highly represented in PaCa TT compared to AT (top) and LuCa TT compared to AT and DT (bottom). The heatmap is based on proteins found in more than 50% of TT and whose levels were at least 10-fold higher in PaCa and LuCa TT than in AT (FDR<0.1%). FIG. 6C is a heatmap showing the top 50 proteins never found in AT but found in more than 50% of PaCa (left). Two proteins were found in more than 50% of LuCa TT and never found in AT or DT 1 (right). FIG. 6D is a heatmap of tumor (n=85) and non-tumor (n=66) samples based on the random forest algorithm depicting proteins found to have the highest predictive values in this classification. FIG. 6E shows a classification error matrix result using random forest classifier of 75% training set and 25% test set. Sample numbers identified are noted in each box. Sensitivity, specificity, positive predictive value and negative predictive value are specified.

FIGS. 7A-7I show pathway analysis of the complete PaCa Tissue explant EVP protein dataset. FIG. 7A shows a hallmark enrichment plot, heatmap and protein list for EMT. FIG. 7B shows a hallmark enrichment plot, heatmap and protein list for coagulation. FIG. 7C shows a hallmark enrichment plot, heatmap and protein list for the TGF-beta pathway. FIG. 7D shows a KEGG enrichment plot, heatmap and protein list for cardiac muscle contraction. FIG. 7E shows a KEGG enrichment plot, heatmap and protein list for actin cytoskeleton. FIG. 7F shows a KEGG enrichment plot, heatmap and protein list for ECM receptor interaction. FIG. 7G shows a GO enrichment plot, heatmap and protein list for endothelial cell apoptosis. FIG. 7H shows a GO enrichment plot, heatmap and protein list for actin filament bundle. FIG. 7I shows a GO enrichment plot, heatmap and protein list for peptide cross linking.

FIGS. 8A-8G show pathway analysis of the complete LuCa Tissue explant EVP protein dataset. FIG. 8A shows a hallmark enrichment plot, heatmap and protein for E2F targets. FIG. 8B shows a hallmark enrichment plot, heatmap and protein list for the G2M checkpoint. FIG. 8C shows a hallmark enrichment plot, heatmap and protein list for MYC targets. FIG. 8D shows a KEGG enrichment plot, heatmap and protein list for the spliceosome. FIG. 8E shows a KEGG enrichment plot, heatmap and protein list for RNA degradation. FIG. 8F shows a KEGG enrichment plot, heatmap and protein list for KEGG for purine metabolism. FIG. 8G shows a GO enrichment plot, heatmap and protein list for RNA processing.

FIGS. 9A-9B show analysis of highly enriched EVP DAMP molecules in PaCa and LuCa. FIG. 9A is a heatmap of EVP proteins significantly enriched in PaCa TT found in more than 50% of TT with more than 10-fold differences and 0.1<FDR. FIG. 9B is a heatmap of EVP proteins significantly enriched in LuCa TT or AT/DT found in more than 50% of TT or AT/DT, respectively, with more than 10-fold differences and 0.1<FDR. Two sample t-test was used to calculate FDR.

FIGS. 10A-10D show the identification of EVP protein cargo in cancer patient plasma. FIG. 10A is a heatmap showing proteins exclusively found in more than 30% of PaCa patient plasma-derived EVP samples but never found in 28 healthy control plasma-derived EVP samples (left). A heatmap of EVP proteins exclusively found in PaCa patient plasma relative to PaCa TT and AT EVP cargo is shown on the right. FIG. 10B is a heatmap showing proteins exclusively found in more than 30% of LuCa patient plasma-derived EVPs but never found in 28 healthy control plasma exosome samples (left). A heatmap of EVPs proteins exclusively found in LuCa patient plasma relative to LuCa TT, AT and DT EVP cargo is shown on the right. FIG. 10C is a heatmap of tumor (n=77) and non-tumor (n=43) plasma samples based on the random forest algorithm depicting EVP proteins found to have the highest predictive values in this classification. FIG. 10D shows the classification error matrix result using random forest classifier of 75% training set and 25% test set. Sample numbers identified are noted in each box. Sensitivity, specificity, positive predictive value and negative predictive value are specified.

FIGS. 11A-11B show identification of neuroblastoma and osteosarcoma patient plasma EVP protein cargo. FIG. 11A shows EVP proteins uniquely found in neuroblastoma patient plasma that were analyzed. EVP proteins found in more than 30% of patient samples but never in 15 healthy control samples were defined as unique to neuroblastoma patients. These EVP protein lists were then compared to patient tissue explant samples to examine the origin of the EVPs. FIG. 11B shows EVP proteins uniquely found in osteosarcoma patient plasma that were analyzed. EVP proteins found in more than 30% of patient samples but never in 15 healthy control samples were defined as unique to osteosarcoma patients. These EVP protein lists were then compared to patient tissue explant samples to examine the origin of the EVPs.

FIGS. 12A-12G show tumor-derived EVP profiles classify primary tumor of origin. FIG. 12A shows a classification error matrix result for training (75% of samples) and test (25% of samples) sets from explant tissue derived EVPs. FIG. 12B is a heatmap showing 29 proteins identified as having the highest predictive value by the random forest algorithm based on primary tumor tissue-derived EVPs. Tumors from primary tissue or tumor-positive draining lymph nodes were used for this analysis. Samples included colorectal cancer (n=3, stage 0=1, stage 3=2), lung cancer (n=14, stage 1=7, stage 2=5, stage 3=2), melanoma (n=5, stage 3=5) and pancreatic cancer (n=21, stage 1=1, stage 2=15, stage 3=5) used in this analysis. FIG. 12C shows supervised three-dimensional images by tSNE plot representing FIG. 12B. FIG. 12D shows the classification error matrix for the training (75% of samples) and test (25% of samples) sets from plasma-derived EVPs. All samples were assigned to the correct cancer type with a classification error of zero. FIG. 12E is a heatmap showing the top 30 proteins analyzed to have the highest predictive value as determined by random forest algorithm based on plasma-derived EVP differences relative to primary tumor type. Breast cancer (n=8, stage 1=1, stage 2=2, stage 4=5), colorectal cancer (n=3, stage 0=1, stage 3=2), lung cancer (n=12, stage 1=6, stage 2=5, stage 3=1), pancreatic cancer (n=9, stage 2=7, stage 3=2), and mesothelioma (n=15, stage 1=2, stage 3=1, stage 4=1, not available [NA]=11). FIG. 12F shows supervised three dimensional images by tSNE plot representing FIG. 12E. FIG. 12G is a model showing that the sources of EVPs in the plasma are a combination of TT, AT/DT and DO.

FIGS. 13A-13C are heat maps showing proteins expression in healthy tissue versus cancer tissue for lung tissue (FIG. 13A), pancreatic tissue (FIG. 13B), mammary gland tissue (FIG. 13C), and colon tissue (FIG. 13D).

FIG. 14 is a heat map showing proteins expressed in plasma exosomes of a healthy child versus plasma exosomes of a child having neuroblastoma.

FIG. 15 is a heat map showing proteins expressed in plasma exosomes of a healthy young adult versus plasma exosomes of a young adult having osteosarcoma.

FIG. 16 is heat map showing proteins expressed in plasma exosomes of patients having various stages of pancreatic lesions including benign cysts, intraductal papillary mucinous neoplasm (IPMN), and pancreatic ductal adenocarcinoma (PDAC).

FIG. 17 show the expression of mucins and serpins in pancreatic adenocarcinoma vs. non-tumor adjacent tissue-derived exosomes. Human pancreatic adenocarcinoma tissue-derived exosomal proteomes (n=21) and non-tumor adjacent tissue-derived exosomal proteomes (n=16) were analyzed by liquid chromatography with tandem mass spectrometry (LC-MS/MS). Proteins found in >15% of pancreatic cancer exosomes were compared to pancreatic adjacent tissue-derived exosomes. Log 2 protein expression of the indicated proteins is presented. P values were calculated by Welch's t-test for the comparison of expression level and Fisher's exact test for the comparison of positivity. Data are expressed as mean±SEM.

FIG. 18 shows that expression of ficolin-3 with plasma-derived extracellular vesicles and particles is significantly higher in control (i.e., healthy subjects) that in subjects having breast cancer and subjects having stage 3 or 4 melanoma. Protein expression in samples was detected using an ELISA assay.

FIG. 19A is a heatmap showing the top 22 protein markers found in control and tumor tissue derived extracellular vesicles and particles. The identified 22 protein markers are utilized as described herein in a method to detect the presence of cancer in a subject. FIG. 19B shows the sensitivity and specificity of the markers in detecting cancer in a sample (AUC 0.966).

FIG. 20A is a heatmap showing the top 12 protein markers found in control and tumor plasma derived extracellular vesicles and particles. The identified 12 protein markers are utilized as described herein in a method to detect the presence of cancer in a subject. FIG. 20B shows the sensitivity and specificity of these markers in detecting cancer in a sample (AUC 0.936).

FIG. 21A is a heatmap showing the top 43 protein markers found in control and pancreatic (PDAC) tumor tissue derived extracellular vesicles and particles. The identified 43 protein markers are utilized as described herein in a method to detect the presence of pancreatic cancer in a subject. FIG. 21B shows the sensitivity and specificity of these markers in detecting pancreatic cancer in a sample (AUC 0.961).

FIG. 22A is a heatmap showing the top 21 protein markers found in control and pancreatic (PDAC) tumor plasma derived extracellular vesicles and particles. The identified 21 protein markers are utilized as described herein in a method to detect the presence of pancreatic cancer in a subject. FIG. 22B shows the sensitivity and specificity of these markers in detecting pancreatic cancer in a sample (AUC 1.000).

FIG. 23A is a heatmap showing the top 22 protein markers found in control and lung tumor tissue derived extracellular vesicles and particles. The identified 22 protein markers are utilized as described herein in a method to detect the presence of lung cancer in a subject.

FIG. 23B shows the sensitivity and specificity of these markers in detecting lung cancer in a sample (AUC 0.967).

FIG. 24A is a heatmap showing the top 15 protein markers found in control and lung tumor plasma derived extracellular vesicles and particles. The identified 15 protein markers are utilized as described herein in a method to detect the presence of lung cancer in a subject.

FIG. 24B shows the sensitivity and specificity of these markers in detecting lung cancer in a sample (AUC 0.986).

FIG. 25 contains Table 3 listing the proteins expressed in young age non-neuroblastoma plasma control samples. Proteins in Table 3 are listed by their gene name.

FIG. 26 contains Table 4 listing the proteins expressed in young non-osteosarcoma plasma control samples. Proteins in Table 4 are listed by their gene name.

FIG. 27 contains Table 8 listing proteins expressed in healthy lung tissue exosomes, but not lung tumor tissue derived exosomes. Proteins in Table 8 are listed by their gene name.

FIG. 28 contains Table 9 listing proteins expressed in healthy pancreatic tissue exosomes, but not pancreatic tumor tissue derived exosomes. Proteins in Table 9 are listed by their gene name.

FIG. 29 contains Table 10 listing proteins expressed in healthy breast tissue exosomes, but not breast tumor tissue derived exosomes. Proteins in Table 10 are listed by their gene name.

FIG. 30 contains Table 11 listing proteins expressed in healthy colon tissue exosomes, but not colon tumor tissue derived exosomes. Proteins in Table 11 are listed by their gene name.

### DETAILED DESCRIPTION

The present disclosure is directed to methods of diagnosing and characterizing cancer conditions, or lack thereof, in a subject based on plasma derived and tissue derived exosomal protein signatures. These methods involve obtaining a liquid biopsy sample and/or a tissue sample from a subject, separating from these samples extracellular vesicles and particles, isolating protein from the separated extracellular vesicles and particles, and detecting the presence and/or absence of the proteins as described here. Proteins of the protein signatures described herein are referred to interchangeably by their protein name and gene name.

### Diagnostic Methods Based on Plasma Derived Extracellular Vesicles and Particles

A first aspect of the present disclosure is directed to a method for screening a subject for the presence of cancer that involves obtaining a liquid biopsy sample from a subject. Extracellular vesicles and particles are separated from the sample, and protein from the separated extracellular vesicles and particles is isolated to form an extracellular vesicle and particle protein sample. The extracellular vesicle and particle protein sample is subjected to a detection assay suitable for detecting: (i) a protein selected from the group consisting of ferritin light chain, von Willebrand factor, immunoglobulin lambda constant 2, keratin 17, immunoglobulin heavy constant gamma 1, keratin 6B, radixin, cofilin 1, protease, serine 1, tubulin alpha 1c, ADAM metallopeptidase with thrombospondin type 1 motif 13, immunoglobulin kappa variable 6D-21, tyrosine 3-monooxygenase/tryptophan 5-monooxygenase activation protein theta, POTE ankyrin domain family member I, POTE ankyrin domain family member F, and combinations thereof, and (ii) a protein selected from the group consisting of actin gamma 1, immunoglobulin lambda variable 3-27, immunoglobulin kappa variable 1D-12, coagulation factor XI, complement C1r subcomponent like, attractin, butyrylcholinesterase, immunoglobulin heavy variable 3-35, immunoglobulin kappa variable 1-17, C1q and TNF related 3, immunoglobulin heavy variable 3-20, immunoglobulin heavy variable 3/OR15-7, collectin subfamily member 11, immunoglobulin heavy constant delta, immunoglobulin kappa variable 3D-11, immunoglobulin heavy variable 3/OR16-10, immunoglobulin kappa variable 2D-24, immunoglobulin kappa variable 2-40, immunoglobulin kappa variable 1-27, immunoglobulin heavy variable 3/OR16-9, immunoglobulin lambda variable 5-45, immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6, and combinations thereof, thereby detecting the presence or absence of the protein of (i) and the protein of (ii) in the extracellular vesicle and particle protein sample. In accordance with this method, detecting the presence of one or more proteins from (i) is indicative of the presence of cancer in the subject and detecting the presence of one or more proteins from (ii) is indicative of the absence of cancer in the subject.

In one embodiment, the protein of (i) is immunoglobulin lambda constant 2. Detection of this protein in a sample is indicative of the presence of cancer in the subject.

In another embodiment, the protein of (ii) is immunoglobulin kappa variable 2D-30. Detection of this protein in a sample is indicative of the absence of cancer in the subject.

In certain embodiments, at least two proteins of (i) are detected. In one embodiment, the at least two proteins of (i) are immunoglobulin lambda constant 2 and keratin 17. In another embodiment, the at least two proteins of (i) are ferritin light chain and von Willebrand factor.

In certain embodiments, at least two proteins of (ii) are detected. In one embodiment, the at least two proteins of (ii) are immunoglobulin kappa variable 2D-30 and immunoglobulin lambda constant 6.

In other embodiments, at least two proteins of (i) and at least two proteins of (ii) are detected. In one embodiment, the at least two proteins of (i) are ferritin light chain and von Willebrand factor, and the at least two proteins of (ii) are immunoglobulin kappa variable 2D-30 and immunoglobulin lambda constant 6.

Another aspect of the present disclosure is directed to a method for screening a subject for the presence of cancer that involves obtaining a liquid biopsy sample from a subject. Extracellular vesicles and particles are separated from the sample, and protein from the separated extracellular vesicles and particles is isolated to form an extracellular vesicle and particle protein sample. The extracellular vesicle and particle protein sample is subjected to a detection assay suitable for detecting: (i) one or more proteins selected from the group consisting of Ferritin light chain (FTL), ABC-type oligopeptide transporter ABCB9 (ABCB9), Protein Z-dependent protease inhibitor (SERPINA10), Coagulation factor VIII (F8), Lactotransferrin (LTF), Basement membrane-specific heparan sulfate proteoglycan core protein (HSPG2), Protein disulfide-isomerase (P4HB), Trypsin-1 (PRSS1), Keratin, type II cytoskeletal 1b (KRT77), Endoplasmic reticulum chaperone BiP (HSPA5); and (ii) one or both proteins selected from the group consisting of Complement C1q tumor necrosis factor-related protein 3 (C1QTNF3) and Immunoglobulin heavy constant delta (IGHD), thereby detecting the presence or absence of the protein of (i) and the protein of (ii) in the extracellular vesicle and particle protein sample. In some embodiments, the method involves detecting at least the presence of one or more of LTF, HSPG2, P4HB, and PRSS1. In accordance with this method, detecting the presence of one or more proteins from (i) is indicative of the presence of cancer in the subject and detecting the presence of one or more proteins from (ii) is indicative of the absence of cancer in the subject.

In one embodiment, the protein of group (i) that is detected is LTF. In one embodiment, the protein of group (i) that is detected is HSPG2. In one embodiment, the protein of group (i) that is detected is P4HB. In one embodiment, the protein of group (i) that is detected is PRSS1. In one embodiment, at least four proteins of (i) are detected. In one embodiment, the four proteins of (i) that are detected are LTF, HSPG2, P4HB, and PRSS1

In accordance with this aspect of the present disclosure, these methods are employed to screen a subject for the general presence of cancer based on the presence and/or absence of the described proteins in the extracellular vesicle and particle protein sample. For example, these methods can be employed during a regularly scheduled physical examination to achieve early detection of cancer in the subject. Alternatively, these methods may be employed in a subject possessing a tumor or abnormal tissue mass, where it is unknown if the tumor or tissue mass is benign or malignant. Accordingly, when either method is employed to detect the general presence of cancer in a subject, the presence of one or more proteins from (i) is indicative of the presence of cancer in the subject and detecting the presence of one or more proteins from (ii) is indicative of the absence of cancer in the subject.

In some embodiments, at least one, at least two, at least three, at least four, at least 5, at least 6, at least 7, at least 8, at least 9, at least 10 or great than 10 proteins from the proteins of group (i) are subject to detection, and the detection of any one or more of these proteins in the sample indicates the presence of cancer in the subject.

In some embodiments, at least one, at least two, at least three, at least four, at least 5, at least 6, at least 7, at least 8, at least 9, at least 10 or great than 10 proteins from the proteins of group (ii) are subject to detection, and the presence of any one or more in these proteins in the sample indicates the absence of cancer in the subject.

When utilized together, the detection of one or more proteins of group (i) and the absence of one or more proteins in group (ii) is indicative of the presence of cancer in the subject or the presence of a malignant tumor in the subject. Alternatively, detecting the absence of one or more proteins of group (i) and the presence of one or more proteins of group (ii) is indicative that the subject does not have cancer or that any tumor or tissue mass is a benign tumor or tissue mass. Detecting both the presence and/or absence of tumor-associated and non-tumor associated exosomal proteins significantly improves the diagnostic integrity of the methods described herein.

The method described herein can be used as a diagnostic approach before more invasive testing (e.g., liquid biopsy prior to tissue biopsy) or it may follow another diagnostic approach (e.g., liquid biopsy to detect biomarkers after mammogram, ultrasound, MRI, tissue biopsy, PSA blood test, or genetic testing) to provide additional information or clarify unclear results. Alternatively, the method may be used as a standard test during a yearly doctor's visit to generally detect the presence or absence of cancer in a subject. Accordingly, the subject tested using the method disclosed herein may have one or more risk factors for a cancer and be asymptomatic. The subject may be asymptomatic of a cancer. The subject may have one or more risk factors for a cancer. The subject may be symptomatic for a cancer and have one or more risk factors of the cancer. The subject may have or be suspected of having a cancer or a tumor. The subject may have a tumor, and the status of the tumor, e.g., benign or malignant, is unknown. The subject may be a patient being treated for a cancer. The subject may be predisposed to a risk of developing a cancer or a tumor. The subject may be in remission from a cancer or a tumor. The subject may not have a cancer, may not have a tumor, or may not have a cancer or a tumor. The subject may be healthy.

In other embodiments, at least two proteins of (i) are detected. In one embodiment, the at least two proteins of (i) are selected from LTF, HSPG2, P41113, and PRSS1

Another aspect of the present disclosure is directed to a method for screening a subject for the presence of cancer that involves obtaining a liquid biopsy sample from a subject. Extracellular vesicles and particles are separated from the sample, and protein from the separated extracellular vesicles and particles is isolated to form an extracellular vesicle and particle protein sample. The extracellular vesicle and particle protein sample is subjected to a detection assay suitable for detecting any one or more of the proteins of Table 1 (identified by their gene name) and any one or more of the proteins of Table 2 (identified by their gene name) below.

TABLE 1
Top 50 highly expressed proteins on tumors based on tumor
plasma EVPs (n = 77) versus healthy control plasma-derived
EVPs (n = 43) comparison (>5-fold higher in tumor plasmas)

| | | | FOLD | TUMOR | NORMAL |
|---|---|---|---|---|---|
| No. | Protein | FDR | CHANGE | (%) | (%) |
| 1 | KRT2 | 0.002 | 379.3 | 83% | 53% |
| 2 | ABCB9 | 0.002 | 199.5 | 39% | 9% |
| 3 | FTH1 | 0.005 | 190.5 | 40% | 12% |
| 4 | KRT4 | 0.007 | 177.0 | 55% | 28% |
| 5 | HSPA5 | 0.002 | 149.0 | 51% | 23% |
| 6 | YWHAB | 0.010 | 147.8 | 47% | 19% |
| 7 | KRT77 | 0.014 | 143.7 | 64% | 40% |
| 8 | FTL | 0.007 | 139.8 | 70% | 49% |
| 9 | KRT7 | 0.010 | 131.9 | 51% | 26% |
| 10 | GAPDH | 0.005 | 102.6 | 49% | 26% |
| 11 | CFL1 | 0.014 | 93.0 | 51% | 26% |
| 12 | YWHAH | 0.014 | 87.7 | 43% | 19% |
| 13 | F8 | 0.002 | 86.0 | 39% | 14% |
| 14 | PRSS1 | 0.002 | 81.3 | 23% | 0% |
| 15 | KRT8 | 0.031 | 81.1 | 55% | 33% |
| 16 | KRT73 | 0.012 | 77.1 | 42% | 19% |
| 17 | KRT74 | 0.012 | 77.0 | 42% | 19% |
| 18 | ADAMTS13 | 0.007 | 77.0 | 36% | 12% |
| 19 | P4HB | 0.002 | 72.4 | 26% | 0% |
| 20 | SFN | 0.014 | 70.4 | 42% | 19% |
| 21 | KRT5 | 0.017 | 69.8 | 61% | 40% |
| 22 | HSPG2 | 0.002 | 69.6 | 27% | 0% |
| 23 | YWHAQ | 0.014 | 69.2 | 42% | 19% |
| 24 | YWHAG | 0.014 | 69.2 | 47% | 23% |
| 25 | PPIA | 0.019 | 65.0 | 39% | 16% |
| 26 | YWHAE | 0.010 | 58.3 | 43% | 21% |
| 27 | KRT72 | 0.012 | 55.1 | 38% | 16% |
| 28 | GNAI3 | 0.007 | 54.0 | 34% | 12% |
| 29 | KRT10 | 0.007 | 51.2 | 92% | 74% |
| 30 | AZGP1 | 0.010 | 51.0 | 79% | 58% |
| 31 | HSPA1L | 0.017 | 47.9 | 45% | 26% |
| 32 | GNAI1 | 0.017 | 45.2 | 35% | 14% |
| 33 | KRT3 | 0.026 | 43.8 | 58% | 40% |
| 34 | EHD1 | 0.010 | 41.9 | 29% | 7% |
| 35 | PGK1 | 0.002 | 40.3 | 23% | 2% |
| 36 | LTF | 0.002 | 40.3 | 21% | 0% |
| 37 | GFAP | 0.041 | 40.2 | 38% | 19% |
| 38 | KRT76 | 0.031 | 37.8 | 60% | 42% |
| 39 | CA1 | 0.017 | 37.1 | 35% | 16% |
| 40 | SERPINA10 | 0.010 | 34.5 | 42% | 21% |
| 41 | KRT6A | 0.034 | 34.2 | 61% | 44% |
| 42 | KRT6C | 0.036 | 33.9 | 61% | 44% |
| 43 | UGT8 | 0.007 | 32.5 | 99% | 84% |
| 44 | LYZ | 0.045 | 32.3 | 53% | 35% |
| 45 | CFHR4 | 0.012 | 32.3 | 25% | 5% |
| 46 | TMSB10 | 0.041 | 32.3 | 34% | 16% |
| 47 | KRT6B | 0.038 | 31.9 | 62% | 47% |
| 48 | S100A9 | 0.034 | 31.9 | 77% | 60% |
| 49 | BASP1 | 0.017 | 30.4 | 31% | 9% |
| 50 | EEF1A1 | 0.019 | 28.1 | 27% | 7% |

TABLE 2
Tod 50 highly expressed proteins in non-tumors based on tumor
plasma EVPs (n = 77) versus healthy control plasma-derived
EVPs (n = 43) comparison (>5-fold higher in tumor plasmas)

| | | | FOLD | TUMOR | NORMAL |
|---|---|---|---|---|---|
| No. | Protein | FDR | CHANGE | (%) | (%) |
| 1 | IGHD | 0.002 | −3571.0 | 34% | 77% |
| 2 | COLEC11 | 0.002 | −2285.8 | 42% | 84% |
| 3 | IGLV4-69 | 0.002 | −1881.1 | 62% | 98% |
| 4 | THBS2 | 0.002 | −1875.7 | 32% | 74% |
| 5 | IGKV1-27 | 0.002 | −1291.6 | 53% | 93% |
| 6 | IGLV4-60 | 0.002 | −1176.3 | 27% | 65% |
| 7 | C1QTNF3 | 0.002 | −977.6 | 25% | 63% |
| 8 | IGHV3-35 | 0.002 | −906.0 | 57% | 88% |
| 9 | IGLV2-18 | 0.002 | −697.9 | 48% | 81% |
| 10 | IGKV3D-15 | 0.002 | −616.6 | 55% | 88% |
| 11 | IGKV3D-11 | 0.002 | −513.4 | 73% | 100% |
| 12 | IGKV1-6 | 0.002 | −482.1 | 65% | 98% |
| 13 | IGKV1-17 | 0.002 | −480.6 | 65% | 98% |
| 14 | ATRN | 0.002 | −247.3 | 53% | 84% |
| 15 | IGKV3OR2-268 | 0.007 | −246.0 | 57% | 81% |
| 16 | IGLV3-27 | 0.002 | −177.8 | 68% | 98% |
| 17 | BCHE | 0.005 | −157.6 | 58% | 86% |
| 18 | IGHV3OR15-7 | 0.005 | −142.1 | 60% | 86% |
| 19 | THBS1 | 0.007 | −127.3 | 64% | 88% |
| 20 | IGKV1-8 | 0.010 | −121.8 | 60% | 86% |
| 21 | MMRN1 | 0.012 | −119.2 | 29% | 56% |
| 22 | IGKV3-7 | 0.036 | −100.5 | 60% | 79% |
| 23 | IGLV3-16 | 0.014 | −97.4 | 60% | 86% |
| 24 | IGLV9-49 | 0.010 | −95.2 | 26% | 51% |
| 25 | APOM | 0.002 | −93.5 | 75% | 100% |
| 26 | IGKV2-29 | 0.002 | −89.2 | 74% | 95% |
| 27 | IGLV1-44 | 0.002 | −88.4 | 68% | 88% |
| 28 | SVEP1 | 0.005 | −72.9 | 19% | 47% |
| 29 | COLEC10 | 0.005 | −69.4 | 12% | 35% |
| 30 | ITGA2B | 0.019 | −62.3 | 52% | 77% |
| 31 | C1RL | 0.007 | −60.7 | 78% | 93% |
| 32 | IGKV1-39 | 0.012 | −55.1 | 71% | 91% |
| 33 | IGLV5-45 | 0.024 | −54.3 | 21% | 42% |
| 34 | IGFALS | 0.012 | −49.9 | 30% | 51% |
| 35 | HYI | 0.041 | −49.7 | 58% | 77% |
| 36 | MBL2 | 0.019 | −47.6 | 61% | 81% |
| 37 | PF4 | 0.022 | −46.4 | 71% | 91% |
| 38 | F11 | 0.026 | −46.0 | 56% | 79% |
| 39 | TGFBI | 0.012 | −45.0 | 17% | 40% |
| 40 | IGKV2D-24 | 0.002 | −38.9 | 81% | 98% |
| 41 | IGKV2-24 | 0.002 | −37.6 | 84% | 100% |
| 42 | IGKV2D-29 | 0.005 | −37.0 | 81% | 98% |
| 43 | MAN1C1 | 0.014 | −35.6 | 6% | 26% |
| 44 | CHMP4A | 0.002 | −35.2 | 3% | 21% |
| 45 | SERPINA4 | 0.038 | −33.5 | 52% | 74% |
| 46 | CLEC3B | 0.026 | −31.2 | 47% | 65% |
| 47 | PF4V1 | 0.045 | −29.6 | 69% | 86% |
| 48 | IGKV1-16 | 0.045 | −29.3 | 75% | 91% |
| 49 | IGKV1-12 | 0.024 | −28.8 | 69% | 86% |
| 50 | IGHV3OR16-12 | 0.012 | −26.1 | 83% | 98% |

Detecting the presence of one or more proteins from Table 1 is indicative of the presence of cancer in the subject and detecting the presence of one or more proteins from Table 2 is indicative of the absence of cancer in the subject.

In some embodiments, at least one, at least two, at least three, at least four, at least 5, at least 6, at least 7, at least 8, at least 9, at least 10 or great than 10 proteins from the proteins listed in Table 1 are subject to detection, and the detection of any one or more in the sample indicates the presence of cancer in the subject.

In some embodiments, at least one, at least two, at least three, at least four, at least 5, at least 6, at least 7, at least 8, at least 9, at least 10 or great than 10 proteins from the proteins listed in Table 2 are subject to detection, and the absence of any one or more in the sample indicates the absence of cancer in the subject.

When utilized together, the detection of one or more proteins of Table 1 and the absence of one or more proteins in Table 2 is indicative of the presence of cancer in the subject. Alternatively, detecting the absence of one or more proteins of Table 1 and the presence of one or more proteins in Table 2 is indicative that the subject does not have cancer. Detecting both the presence and/or absence of tumor-associated and non-tumor associated exosomal proteins significantly improves the diagnostic integrity of the methods described herein.

In accordance with all aspects of the present disclosure, a “subject” as referred to herein encompasses any animal, but preferably a mammal, e.g., human, non-human primate, a dog, a cat, a horse, a cow, or a rodent. More preferably, the subject is a human. In any embodiment of the present disclosure, the subject has a tumor or tissue mass, where the status of the tumor or mass (i.e., benign or malignant) is unknown. In any embodiment of the present disclosure, the subject has cancer, for example and without limitation, lung cancer, pancreatic cancer, neuroblastoma, osteosarcoma, breast cancer, colorectal cancer, and mesothelioma. In any embodiment, the cancer is a primary tumor, while in other embodiments, the cancer is a secondary or metastatic tumor. In any embodiment, the cancer involves of a tumor of unknown origin.

“Extracellular vesicles and particles” refers to any one or more of the subpopulations of exosomes (i.e., Exo-S and Exo-L) and exomeres. Generally, exosomes are microvesicles released from a variety of different cells, including cancer cells (i.e., “cancer-derived exosomes”). These small vesicles derive from large multivesicular endosomes and are secreted into the extracellular milieu. The precise mechanisms of exosome release/shedding remain unclear; however, this release is an energy-requiring phenomenon, modulated by extracellular signals. They appear to form by invagination and budding from the limiting membrane of late endosomes, resulting in vesicles that contain cytosol and that expose the extracellular domain of membrane-bound cellular proteins on their surface. Using electron microscopy, studies have shown fusion profiles of multivesicular endosomes with the plasma membrane, leading to the secretion of the internal vesicles into the extracellular environment. The rate of exosome release is significantly increased in most neoplastic cells and occurs continuously. Increased release of exosomes and their accumulation appear to be important in the malignant transformation process.

As described in WO2019/109077 to Lyden et al, which is hereby incorporated by reference in its entirety, two exosome subpopulations (i.e., Exo-S and Exo-L) have been identified. Exo-S refers to a population of small exosomes having a diameter of 60 to 80 nm, an average surface charge of −9.0 mV to −12.3 mV, and a particle stiffness of 70 to 420 mPa. Exo-S are also enriched in genes involved in membrane vesicle biogenesis and transport, protein secretion and receptor signaling. Exo-L refers to a population of large exosomes having a diameter of 90 to 120 nm, an average surface charge of −12.3 to −16.0 mV, and a particle stiffness of 26 to 73 mPa. Exo-L are also enriched in genes involved in the mitotic spindle, TL-2/Stat5 signaling, multi-organism organelle organization, and G-protein signaling.

As described above, “extracellular vesicles and particles” also encompasses exomeres. Exomeres are non-membranous nanoparticles having a diameter of less than 50 nm, often approximately 35 nm, an average surface charge of −2.7 mV to −9.7 mV, and a particle stiffness of 145 to 816 mPa. Exomeres are enriched in metabolic enzymes and hypoxia, microtubule and coagulation proteins as well as proteins involved in glycolysis and mTOR signaling

In accordance with the methods of the present disclosure, for the purposes of a liquid biopsy, extracellular vesicles and particles can be isolated or obtained from most biological fluids including, without limitation, whole blood, blood serum, blood plasma, ascites fluid, cyst fluid, pleural fluid, peritoneal fluid, cerebrospinal fluid, tears, urine, saliva, sputum, nipple aspirates, lymph fluid, synovial fluid, amniotic fluid, semen, follicular fluid, fluid of the respiratory, intestinal, and genitourinary trances, breast milk, intra-organ system fluid, conditioned media from tissue explant culture, or combinations thereof.

The extracellular vesicles and particles, i.e., exomeres, small exosomes, or large exosomes, can be isolated from the aforementioned biological fluid sample using methods described in more detail herein or otherwise known in the art.

Another aspect of the present disclosure relates to a method of determining the presence of lung cancer in a subject. In one embodiment, this method involves obtaining a liquid biopsy sample from the subject, separating extracellular vesicles and particles from the liquid biopsy sample, and isolating protein from the separated extracellular vesicles and particles to form an extracellular vesicle and particle protein sample. The extracellular vesicle and particle protein sample are subjected to a detection assay suitable for detecting: (i) a protein selected from the group consisting of selenoprotein P (SELENOP), rho-related GTP binding protein RhoV (RHOV), roquin-2 (RC3H2), claudin-5 (CLDN5), dematin (DMTN), serine/threonine-protein kinase/endoribonuclease IRE1 (ERN1), IGCL2, radixin (RDX), complement factor B (CFB), trypsin-1, EC 3.4.21.4 (PRSS1), leukocyte surface antigen CD53 (CD53), charged multivesicular body protein 4b (CHMP4B), proteasome subunit beta type-1 (PSMB1), actin aortic smooth muscle (ACTA2), guanine nucleotide-binding protein (GNG5), histone H2A.Z (H2AFZ), histone H2A type 1-C (HIST1H2AC), POTE ankyrin domain family member E (POTEE), POTE ankyrin domain family member I (POTEI) and combinations thereof, and (ii) a protein selected from immunoglobulin heavy constant delta (IGHD), collectin-11 (COLEC11), immunoglobulin lambda variable 4-69 (IGLV4-69), thrombospondin-2 (THBS2), immunoglobulin kappa variable 1-27 (IGKV1-27), immunoglobulin lambda variable 4-60 (IGLV4-60), complement C1q tumor necrosis factor-related protein 3 (C1QTNF3), probable non-functional immunoglobulin heavy variable 3-35 (IGHV3-35), Immunoglobulin lambda variable 2-18 (IGLV2-18), immunoglobulin kappa variable 3D-15 (IGKV3D-15), immunoglobulin kappa variable 3D-11 (IGKV3D-11), immunoglobulin kappa variable 1-6 (IGKV1-6), immunoglobulin kappa variable 1-17 (IGKV1-17), Attractin (ATRN), immunoglobulin kappa variable 3/OR2-268 (non-functional) (IGKV30R2-268), immunoglobulin lambda variable 3-27 (IGLV3-27), cholinesterase (BCHE), immunoglobulin heavy variable 3/OR15-7 (IGHV3OR15-7), thrombospondin-1 (Glycoprotein G) (THBS1), immunoglobulin kappa variable 1-8 (IGKV1-8), multimerin-1 (MMRN1), probable non-functional immunoglobulin kappa variable 3-7 (IGKV3-7), immunoglobulin lambda variable 3-16 (IGLV3-16), immunoglobulin lambda variable 9-49 (IGLV9-49), apolipoprotein M (APOM), immunoglobulin kappa variable 2-29 (IGKV2-29), immunoglobulin lambda variable 1-44 (IGLV1-44), sushi, von Willebrand factor type A (SVEP1), collectin-10 (COLEC10), integrin alpha-IIb (ITGA2B), complement C1r subcomponent-like protein (C1RL), immunoglobulin kappa variable 1-39 (IGKV1-39), immunoglobulin lambda variable 5-45 (IGLV5-45), insulin-like growth factor-binding protein complex acid labile subunit (IGFALS), HY1, Mannose-binding protein C (MBL2), Platelet factor 4, PF-4 (PF4), Coagulation factor XI, FXI, EC 3.4.21.27 (F11), Transforming growth factor beta-1 proprotein (TGFB1), Probable non-functional immunoglobulin kappa variable 2D-24 (IGKV2D-24), Immunoglobulin kappa variable 2-24 (IGKV2-24), Immunoglobulin kappa variable 2D-29 (IGKV2D-29), Mannosyl-oligosaccharide 1,2-alpha-mannosidase IC (MAN1C1), Charged multivesicular body protein 4a (CHMP4A), SERPIN4A, C-type lectin domain family 3 member B (CLEC3B), Platelet factor 4 variant (PF4V1), Immunoglobulin kappa variable 1-16 (IGKV1-16), Immunoglobulin kappa variable 1-12 (IGKV1-12), Immunoglobulin heavy variable 3/OR16-12 (non-functional) (IGHV3OR16-12), and any combination thereof; thereby detecting the presence or absence of the protein of (i) and the protein of (ii) in the extracellular vesicle and particle protein sample. In accordance with this embodiment of the present disclosure, the method is employed to screen a subject for lung cancer based on the presence and/or absence of the described proteins in the extracellular vesicle and particle protein sample. Accordingly, detecting the presence of one or more proteins from (i) is indicative of the presence of lung cancer in the subject and detecting the presence of one or more proteins from (ii) is indicative of the absence of lung cancer in the subject.

In another embodiment of this aspect of the disclosure, this method involves obtaining a liquid biopsy sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting: (i) a protein selected from the group consisting of Putative alpha-1-antitrypsin-related protein (SERPINA2), Immunoglobulin kappa joining 1 (IGKJ1), Protein 4.2 (EPB42), Histone H2A type 1-D (H2AC7), Proteasome subunit alpha type-2 (PSMA2), Nebulette (NEBL), Tripeptidyl-peptidase 2 (TPP2), Monocyte differentiation antigen CD14 (CD14), Fc receptor-like protein 3 (FCRL3), Charged multivesicular body protein 4b (CHMP4B), Rho-related GTP-binding protein RhoV (RHOV), Leukocyte surface antigen CD53 (CD53), Basement membrane-specific heparan sulfate proteoglycan core protein (HSPG2), Trypsin-1 (PRSS1), and combinations therefore, and (ii) transforming growth factor-beta-induced protein ig-h3 (TGFBI), thereby detecting the presence or absence of the protein of (i) and the protein of (ii) in the extracellular vesicle and particle protein sample. In accordance with this embodiment of the present disclosure, the method is employed to screen a subject for lung cancer based on the presence and/or absence of the described proteins in the extracellular vesicle and particle protein sample. Accordingly, detecting the presence of one or more proteins from (i) is indicative of the presence of lung cancer in the subject and detecting the presence of the protein from (ii) is indicative of the absence of lung cancer in the subject. In one embodiment, the method involves detecting at least one or more proteins selected from CHMP4B, RHOV, CD53, HSPG2, and PRSS1.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of lung cancer in the subject.

In some embodiments, at least one, at least two, at least three, at least four, at least 5, at least 6, at least 7, at least 8, at least 9, at least 10, or greater than 10 proteins from the proteins of group (ii) are subject to detection, and the presence of any one or more of these proteins in the sample indicates the absence of lung cancer in the subject.

When utilized together, the detection of one or more proteins of (i) and the absence of one or more proteins in (ii) is indicative of the presence of lung cancer in the subject. Alternatively, detecting the absence of one or more proteins of (i) and the presence of one or more proteins in (ii) is indicative that the subject does not have lung cancer.

As used herein, the term “lung cancer” generally refers to a cancer or tumor of a lung or lung-associated tissue. For example, a lung cancer may comprise a non-small cell lung cancer, a small cell lung cancer, a lung carcinoid tumor, or any combination thereof. A non-small cell lung cancer may comprise an adenocarcinoma, a squamous cell carcinoma, a large cell carcinoma, or any combination thereof. A lung carcinoid tumor may comprise a bronchial carcinoid. A lung cancer may comprise a cancer of a lung tissue, such as a bronchiole, an epithelial cell, a smooth muscle cell, an alveolus, or any combination thereof. A lung cancer may comprise a cancer of a trachea, a bronchus, a bronchiole, a terminal bronchiole, or any combination thereof. A lung cancer may comprise a cancer of a basal cell, a goblet cell, a ciliated cell, a neuroendocrine cell, a fibroblast cell, a macrophage cell, a Clara cell, or any combination thereof.

Accordingly, the methods of this aspect the present disclosure may permit a subject to be screened or monitored for a progression or regression of lung cancer, using a sample non-invasively obtained from the subject. This may advantageously be used to screen for subjects that are asymptomatic for lung cancer, but who may otherwise be at risk of developing lung cancer (e.g., subjects exposed to cigarette smoke or air pollution), or to monitor subjects that have or are suspected of having lung cancer. These methods can also be advantageously used to detect re-current lung cancer in patients in remission, particularly complete remission.

A further aspect of the present disclosure relates to a method for determining presence of pancreatic cancer in a subject. In one embodiment, this method involves obtaining a liquid biopsy sample from the subject, separating extracellular vesicles and particles from the liquid biopsy sample, and isolating protein from the separated extracellular vesicles and particles to form an extracellular vesicle and particle protein sample. The extracellular vesicle and particle protein sample are subjected to a detection assay suitable for detecting: (i) a protein selected from the group consisting of Heat shock-related 70 kDa protein 2 (HSPA2), Carbonic anhydrase 2 (CA2), Ras-related protein Rab-1A (RAB1A), Ras-related protein Rab-8B (RAB8B), Ras-related protein Rap-1A (RAP1A), Brain-specific angiogenesis inhibitor 1-associated protein 2-like protein 1 (BAIAP2L1), Complement decay-accelerating factor (CD55), Golgi-associated plant pathogenesis-related protein 1 (GLIPR2), GTPase KRas (KRAS), Leucine-rich repeat-containing protein 26 (LRRC26), Lactotransferrin (LTF), Protein disulfide-isomerase (P4HB), Phosphatidylethanolamine-binding protein 1 (PEBP1), Radixin (RDX), ATP-dependent translocase ABCB1 (ABCB1), ATP-binding cassette sub-family B member 11 (ABCB11), ATP-binding cassette sub-family B member 4 (ABCB4), Adhesion G-protein coupled receptor G6 (ADGRG6), Alcohol dehydrogenase 1A (ADH1A), Alkaline phosphatase, tissue-nonspecific isozyme (ALPL), Integrin alpha-1 (ITGA1), Protein kinase C and casein kinase substrate in neurons protein 2 (PACSIN2), Receptor-type tyrosine-protein phosphatase eta (PTPRJ), Ras-related protein Rap-2b (RAP2B), Sorcin (SRI), Xaa-Pro aminopeptidase 2 (XPNPEP2), Alcohol dehydrogenase 1C (ADH1C), All-trans-retinol dehydrogenase [NAD(+)]ADH4 (ADH4), Annexin A11 (ANXA11), T-complex protein 1 subunit zeta, TCP-1-zeta (CCT6A), Copine-1 (CPNE1), Desmocollin-1 (DSC1), Desmoglein-1 (DSG1), DSP, Glutamyl aminopeptidase (ENPEP), Fatty acid-binding protein, liver (FABP1), High affinity immunoglobulin epsilon receptor subunit gamma (FCER1G), Filamin-B (FLNB), Guanine nucleotide-binding protein G(I)/G(S)/G(O) subunit gamma-5 (GNG5), Keratin, type II cytoskeletal 8 (KRT8), Keratin, type II cuticular Hb1 (KRT81), Keratin, type II cuticular Hb5 (KRT85), 55 kDa erythrocyte membrane protein, p55 (MPP1), PLGLB1, PRDX6, PSMA4, PSMA5, SFN, SNX18, TGM2, cell surface hyaluronidase (TMEM2), and combinations thereof, and (ii) a protein selected from IGHD, collectin-11 (COLEC11), IGLV4-69, Thrombospondin-2 (THBS2), IGKV1-27, IGLV4-60, C1QTNF3, IGHV3-35, IGLV2-18, IGKV3D-15, IGKV3D-11, IGKV1-6, IGKV1-17, ATRN, IGKV3OR2-268, IGLV3-27, BCHE, IGHV3OR15-7, THBS1, IGKV1-8, MMRN1, IGKV3-7, IGLV3-16, IGLV9-49, APOM, IGKV2-29, IGLV1-44, SVEP1, COLEC10, ITGA2B, C1RL, IGKV1-39, IGLV5-45, IGFALS, HY1, MBL2, PF4, F11, TGFB1, IGKV2D-24, IGKV2-24, IGKV2D-29, MAN1C1, CHMP4A, SERPIN4A, CLEC3B, PF4V1, IGKV1-16, IGKV1-12, IGHV3OR16-12, and any combination thereof, thereby detecting the presence or absence of the protein of (i) and the protein of (ii) in the extracellular vesicle and particle protein sample. In accordance with this aspect of the disclosure, the method is employed to screen a subject for pancreatic cancer based on the presence and/or absence of the described proteins in the extracellular vesicle and particle protein sample. Detecting the presence of one or more proteins from (i) and the absence of one or more proteins from (ii) identifies pancreatic cancer in the subject. Alternatively, detecting the absence of one or more proteins from (i) and the presence of one or more proteins from (ii) identifies the absence of pancreatic cancer in the subject.

In another embodiment, this method of determining presence of pancreatic cancer in a subject involves obtaining a liquid biopsy sample from a subject. Extracellular vesicles and particles are separated from the tissue sample, and protein is isolated from the separated extracellular vesicles and particles to form an extracellular vesicle and particle protein sample. The extracellular vesicle and particle protein sample is subjected to a detection assay suitable for detecting: (i) a protein selected from the group consisting of Calmodulin-like protein 5 (CALML5), Carboxypeptidase N subunit 2 (CPN2), Carbonic anhydrase 2 (CA2), Heat shock-related 70 kDa protein 2 (HSPA2), Lactotransferrin (LTF), GTPase KRas (KRAS), Complement decay-accelerating factor (CD55), Brain-specific angiogenesis inhibitor 1-associated protein 2-like protein 1 (BAIAP2L1), Phosphatidylethanolamine-binding protein 1 (PEBP1), Ras-related protein Rab-1A (RAB1A), Ras-related protein Rab-8B (RAB8B), Desmoplakin (DSP), Leucine-rich repeat-containing protein 26 (LRRC26), and combinations therefore, and (ii) a protein selected from the group consisting of Thrombospondin-1 (THBS1), Complement C1r subcomponent-like protein (C1RL), Immunoglobulin kappa variable 1-6 (IGKV1.6), Immunoglobulin kappa variable 1-17 (IGKV1.17), Immunoglobulin kappa variable 1-39 (IGKV1.39), Immunoglobulin kappa variable 1-27 (IGKV1.27), Immunoglobulin kappa variable 1-12 (IGKV1.12), and Immunoglobulin kappa variable 1D-33 (IGKV1D.33), and combinations thereof, thereby detecting the presence or absence of the protein of (i) and the protein of (ii) in the extracellular vesicle and particle protein sample. In accordance with this aspect of the disclosure, the method is employed to screen a subject for pancreatic cancer based on the presence and/or absence of the described proteins in the extracellular vesicle and particle protein sample. Detecting the presence of one or more proteins from (i) and the absence of one or more proteins from (ii) identifies pancreatic cancer in the subject. Alternatively, detecting the absence of one or more proteins from (i) and the presence of one or more proteins from (ii) identifies the absence of pancreatic cancer in the subject. In one embodiment, the method involves detecting at least one or more proteins selected from LTF, KRAS, CD55, BAIAP2L1, PEBP1, DSP, and LRRC26.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of pancreatic cancer in the subject.

In some embodiments, at least one, at least two, at least three, at least four, at least 5, at least 6, at least 7, at least 8, at least 9, at least 10, or greater than 10 proteins from the proteins of group (ii) are subject to detection, and the presence of any one or more in the sample indicates the absence of pancreatic cancer in the subject.

When utilized together, the detection of one or more proteins of (i) and the absence of one or more proteins in (ii) is indicative of the presence of pancreatic cancer in the subject. Alternatively, detecting the absence of one or more proteins of (i) and the presence of one or more proteins in (ii) is indicative that the subject does not have pancreatic cancer.

As used herein, “pancreatic cancer” refers to all malignant tumors formed in the pancreas. Specific examples include, without limitation, serous cystadenocarcinoma, mucinous cystadenocarcinoma, intraductal papillary mucinous adenocarcinoma, invasive pancreatic duct cancer, acinar cell carcinoma, and neuroendocrine cancer.

Accordingly, the methods of this aspect the present disclosure may permit a subject to be screened or monitored for a progression or regression of pancreatic cancer, using a liquid biopsy sample non-invasively obtained from the subject. These methods may advantageously be used to screen for subjects that are asymptomatic for pancreatic cancer, but who may otherwise be at risk of developing pancreatic cancer (e.g., subjects with a genetic predisposition), or to monitor subjects that have or are suspected of having pancreatic cancer. These methods may also be advantageously used to identify re-current pancreatic cancer in a subject that is in remission, e.g., complete remission.

Another aspect of the present disclosure relates to a method of identifying a pancreatic lesion in a subject. This method involves obtaining a liquid biopsy sample from a subject, separating extracellular vesicles and particles from the biopsy sample, and isolating protein from the separated extracellular vesicles and particles to form an extracellular vesicle and particle protein sample. The extracellular vesicle and particle protein sample is subjected to a detection assay suitable for detecting one or more proteins selected from PSMA2, CA2, EPB41, CD59, CNP, RAB8A, TPI1, GGT1, GGT3P, GGT2, PEBP1, IGLV8-61, C6, PON1, CPN2, ECM1, Ig kappa chain V-I region AG, IGKV4-1, IG lambda chain V-1 region, CFP, TUBB, TUBB4B, TUBB2B, TUBB2A, VCL, RSU1, FERMT3, and ADAMTS13.

In one embodiment, the presence of a cancerous pancreatic lesion is identified in the subject when expression of one or more proteins selected from IGLV8-61, CD59, CA2, CNP, EPB41, C6, CGT1, PON1, TPI1, RAB8A, ECM1, PSMA2, CPN2, and PEBP1 are detected in the extracellular vesicle and particle protein sample.

In another embodiment, the presence of a cancerous pancreatic lesion in the subject is identified when expression of one or more proteins selected from PSMA2, CPN2, and PEBP1 are detected in the extracellular vesicle and particle protein sample.

In yet another embodiment, the presence of a pre-cancerous pancreatic lesion is identified in the subject when expression of one or more proteins selected from VCL, CFP, and FERMT3 are detected in the extracellular vesicle and particle protein sample during said subjecting.

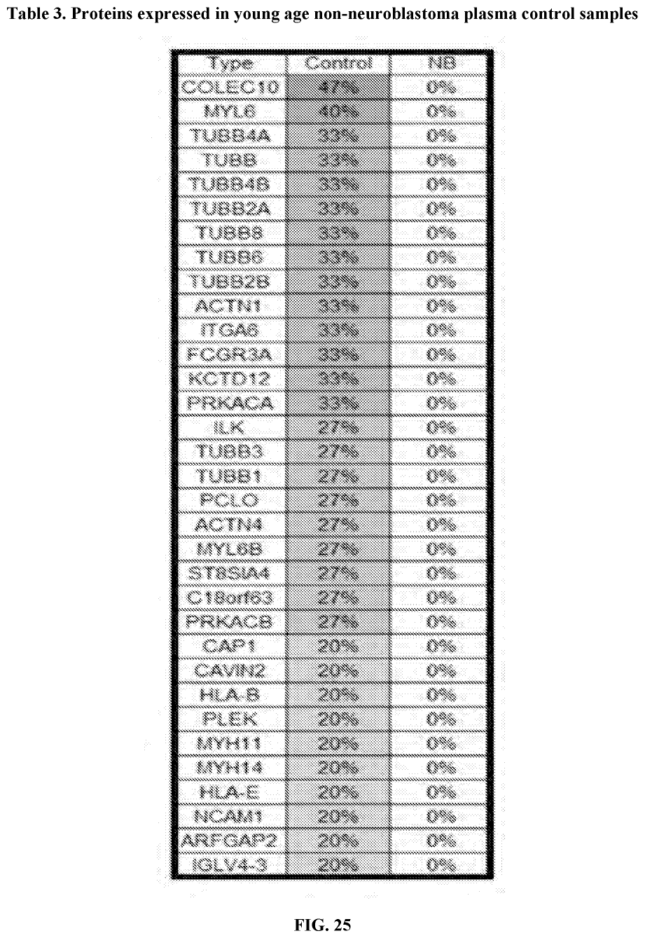

In yet another aspect, the present disclosure relates to a method of determining the presence of neuroblastoma in a subject. The method involves obtaining a liquid biopsy sample from the subject, separating extracellular vesicles and particles from the liquid biopsy sample, and isolating protein from the separated extracellular vesicles and particles to form an extracellular vesicle and particle protein sample. The extracellular vesicle and particle protein sample are subjected to a detection assay suitable for detecting (i) a protein selected from the group consisting of ferritin heavy chain (FTH1), keratin, type I cytoskeletal 17 (KRT17), histone H3.3 (H3F3A), ATP-binding cassette sub-family B member 9 (ABCB9), a disintegrin and metalloproteinase with thrombospondin motifs 13 (ADAMTS13), CD14, erythrocyte membrane protein band 4.2 (EPB42), hepatocyte growth factor activator (HGFAC), keratin, type I cytoskeletal 13 (KRT13), and Keratin, type II cytoskeletal 8 (KRT8), and combinations thereof and (ii) a protein selected from the list of proteins provided in Table 3 (FIG. 25) or any combination of proteins thereof, thereby detecting the presence or absence of the protein of (i) and the protein of (ii) in the extracellular vesicle and particle protein sample. In accordance with this aspect of disclosure, this method is employed to screen a subject for neuroblastoma based on the presence and/or absence of the described proteins in the extracellular vesicle and particle protein sample. Accordingly, detecting the presence of a protein from group (i) is indicative of the presence of neuroblastoma in the subject, and detecting the presence of one or more proteins from group (ii) identifies the absence of neuroblastoma in the subject.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of neuroblastoma in the subject.

In some embodiments, at least one, at least two, at least three, at least four, at least 5, at least 6, at least 7, at least 8, at least 9, at least 10, or greater than 10 proteins from the proteins of group (ii) are subject to detection, and the presence of any one or more of these proteins in the sample indicates the absence of neuroblastoma in the subject.

When utilized together, the detection of one or more proteins of (i) and the absence of one or more proteins in (ii) is indicative of the presence of neuroblastoma in the subject. Alternatively, detecting the absence of one or more proteins of (i) and the presence of one or more proteins in (ii) is indicative that the subject does not neuroblastoma.

As used herein, “neuroblastoma” refers to a tumor that develops from the sympathetic nervous system, such as the adrenal gland or sympathetic ganglia (Brodeur, Nat. Rev. Cancer 3:203-216 (2003), which is hereby incorporated by reference in its entirety). It is one of the most frequent solid tumors in children. It is the most common malignancy diagnosed in the first year of life and shows a wide range of clinical phenotypes with some patients having tumors that regress spontaneously, whereas the majority of patients have aggressive metastatic disease (Maris et al., Lancet 369:2106-20 (2007), which is hereby incorporated by reference in its entirety). These latter neuroblastoma cases have survival probabilities of less than 40% despite intensive chemoradiotherapy, and the disease continues to account for 15% of childhood cancer mortality (Maris et al. Lancet 369:2106-20 (2007); Matthay et al. N. Eng. J. Med. 341:1165-73 (1999), which are hereby incorporated by reference in their entirety). The cancer can start in neuroblasts (e.g., early nerve cells) of the sympathetic nervous system. The term neuroblastoma includes any stage of the cancer as determined according to, for example, the International Neuroblastoma Staging System (INSS) or the International Neuroblastoma Risk Group Staging System (INRGSS).

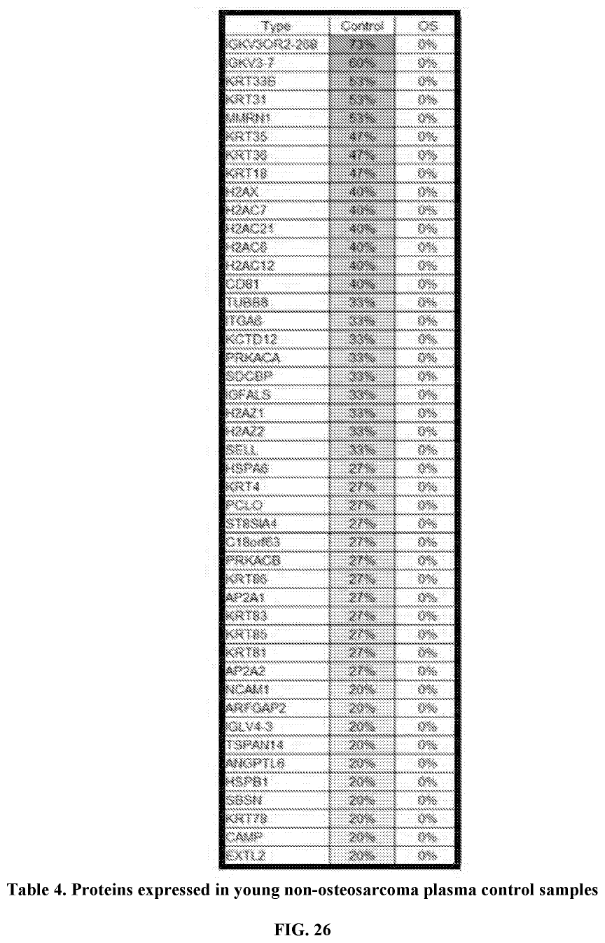

In another aspect, the present disclosure relates to a method of determining the presence of osteosarcoma in a subject. The method involves obtaining a liquid biopsy sample from a subject, separating extracellular vesicles and particles from the liquid biopsy sample, and isolating protein from the separated extracellular vesicles and particles to form an extracellular vesicle and particle protein sample. The extracellular vesicle and particle protein sample are subjected to a detection assay suitable for detecting (i) a protein selected from the group consisting of actin, alpha skeletal muscle (ACTA1), actin, gamma-enteric smooth muscle (ACTG2), A disintegrin and metalloproteinase with thrombospondin motifs 13 (ADAMTS1), Hepatocyte growth factor activator (HGFAC), neprilysin (MME), and Tenascin (TNC), and combinations thereof and (ii) a protein selected from the proteins listed in Table 4 (FIG. 26) or any combination of proteins thereof. In accordance with this aspect of the present disclosure, the method is employed to screen a subject for osteosarcoma based on the presence and/or absence of the described proteins in the extracellular vesicle and particle protein sample. Accordingly, detecting the presence of a protein from group (i) and the absence of a protein in group (ii) (Table 4) shown in FIG. 26 identifies osteosarcoma in the subject. When utilized together, the detection of one or more proteins of (i) and the absence of one or more proteins in (ii) is indicative of the presence of osteosarcoma in the subject. Alternatively, detecting the absence of one or more proteins of (i) and the presence of one or more proteins in (ii) is indicative that the subject does not osteosarcoma.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of osteosarcoma in the subject.

In some embodiments, at least one, at least two, at least three, at least four, at least 5, at least 6, at least 7, at least 8, at least 9, at least 10, or greater than 10 proteins from the proteins of group (ii) are subject to detection, and the presence of any one or more of these proteins in the sample indicates the absence of osteosarcoma in the subject.

As used herein “osteosarcoma” refers to abnormal and/or malignant bone growth. Osteosarcoma (osteogenic sarcoma) is the second most common primary bone tumor and is highly malignant. It is most common in people aged 10 to 20, although it can occur at any age. Osteosarcoma usually develops around the knee or in other long bones, particularly the metaphyses. It can metastasize, usually to lung or bone.

In a further aspect, the present disclosure relates to a method of cancer sub-type identification. This method involves obtaining a liquid biopsy sample from a subject, separating extracellular vesicles and particles from the sample, and isolating protein from the separated extracellular vesicles and particles to form an extracellular vesicle and particle protein sample. The extracellular vesicle and particle protein sample are subjected to a detection assay suitable for detecting levels of at least three proteins selected from the group consisting of fibrinogen beta chain (FGB), fibrinogen alpha chain (FGA), fibrinogen gamma chain (FGG), complement factor H (CFH), plasminogen (PLG), immunoglobulin heavy variable 3-53 (IGHV3-53), serum amyloid P-component, SAP (APCS), complement factor H-related protein 1 (CFHR1), immunoglobulin heavy variable 3-48 (IGHV3-48), immunoglobulin heavy variable 3-74 (IGHV3-74), immunoglobulin heavy variable 3-72 (IGHV3-72), immunoglobulin heavy variable 3-43 (IGHV3-43), immunoglobulin heavy variable 5-10-1 (IGHV5-10-1), immunoglobulin lambda variable 7-46 (IGLV7-46), immunoglobulin kappa variable 3D-20 (IGKV3D-20), immunoglobulin kappa variable 2-24 (IGKV2-24), complement factor H-related protein 2 (CFHR2), immunoglobulin heavy variable 4-59 (IGHV4-59), immunoglobulin heavy variable 3-20 (IGHV3-20), Immunoglobulin heavy variable 3-64 (IGHV3-64), probable non-functional immunoglobulin heavy variable 3-16 (IGHV3-16), immunoglobulin heavy variable 3-11 (IGHV3-11), immunoglobulin heavy variable 3/OR16-9 (IGHV3OR16-9), probable non-functional immunoglobulin kappa variable 2D-24 (IGKV2D-24), immunoglobulin lambda constant 3 (IGLC3), Immunoglobulin heavy variable 3/OR16-13 (IGHV3OR16-13), complement factor H-related protein 3 (CFHR3), immunoglobulin heavy constant gamma 3 (IGHG3), immunoglobulin lambda constant 2 (IGLC2), immunoglobulin kappa variable 1-8 (IGKV1-8) and Ficolin-3.

In accordance with this aspect of the methods described herein, detecting the presence of at least three proteins described above identifies a tumor of unknown origin in the subject. The tumor of unknown origin may include a primary tumor, a metastasis, or a putative metastasis.

In any embodiment, at least three of the aforementioned proteins shown herein to be useful for identifying a cancer type are detected. Alternatively, more than three of these proteins are detected. In any embodiment, the presence or absence of at least 4, at least 5, at least 6, at least 7, at least 8, at least 9, at least 10, or greater than 10 proteins of the proteins shown herein to be useful for identifying a cancer type are detected. In one embodiment, the presence or absence of all of the proteins are detected as a result of said subjecting.

In one embodiment, this method is utilized to identify the origin of a primary tumor in a subject. The origin of the primary tumor is identified by subjecting the liquid biopsy derived extracellular vesicle and particle protein sample to one or more detection assays suitable to detect the presence or absence of at least three proteins selected from the group consisting of fibrinogen beta chain (FGB), fibrinogen alpha chain (FGA), fibrinogen gamma chain (FGG), complement factor H (CFH), plasminogen (PLG), immunoglobulin heavy variable 3-53 (IGHV3-53), serum amyloid P-component, SAP (APCS), complement factor H-related protein 1 (CFHR1), immunoglobulin heavy variable 3-48 (IGHV3-48), immunoglobulin heavy variable 3-74 (IGHV3-74), immunoglobulin heavy variable 3-72 (IGHV3-72), immunoglobulin heavy variable 3-43 (IGHV3-43), immunoglobulin heavy variable 5-10-1 (IGHV5-10-1), immunoglobulin lambda variable 7-46 (IGLV7-46), immunoglobulin kappa variable 3D-20 (IGKV3D-20), immunoglobulin kappa variable 2-24 (IGKV2-24), complement factor H-related protein 2 (CFHR2), immunoglobulin heavy variable 4-59 (IGHV4-59), immunoglobulin heavy variable 3-20 (IGHV3-20), Immunoglobulin heavy variable 3-64 (IGHV3-64), probable non-functional immunoglobulin heavy variable 3-16 (IGHV3-16), immunoglobulin heavy variable 3-11 (IGHV3-11), immunoglobulin heavy variable 3/OR16-9 (IGHV3OR16-9), probable non-functional immunoglobulin kappa variable 2D-24 (IGKV2D-24), immunoglobulin lambda constant 3 (IGLC3), Immunoglobulin heavy variable 3/OR16-13 (IGHV3OR16-13), complement factor H-related protein 3 (CFHR3), immunoglobulin heavy constant gamma 3 (IGHG3), immunoglobulin lambda constant 2 (IGLC2), and immunoglobulin kappa variable 1-8 (IGKV1-8).

In a further embodiment, once the origin of a primary tumor in a subject is identified, an appropriate therapeutic drug known to treat that primary tumor is administered to the subject.

In one embodiment, the at least three proteins that are detected to identify the type of cancer present in the subject include immunoglobulin kappa variable 1-8 (IGKV1-8), immunoglobulin lambda constant 3 (IGLC3), and immunoglobulin heavy variable 3/OR16-13 (IGHV3OR16-13). In accordance with this embodiment, lung cancer is detected in the subject when the expression of IGKV1-8 is detected and expression of IGLC3 and IGHV3OR16-13 are not detected in the extracellular vesicle and particle protein sample. If lung cancer is detected and identified as the cancer type present in the subject, the subject can be administered one or more therapies suitable for treating the identified lung cancer. Suitable therapies for treating lung cancer are known in the art, and include, for example and without limitation surgery (e.g., pneumonectomy, lobectomy, segmentectomy or wedge resection, sleeve resection); radiation therapy (e.g., external beam radiation therapy and brachytherapy); chemotherapy, including, without limitation, Cisplatin, Carboplatin, Paclitaxel (Taxol), Albumin-bound paclitaxel (nab-paclitaxel, Abraxane), Docetaxel (Taxotere), Gemcitabine (Gemzar), Vinorelbine (Navelbine), Etoposide (VP-16), Pemetrexed (Alimta); targeted therapeutics, including, without limitation, angiogenesis inhibitors (e.g., Bevacizumab (Avastin) and Ramucirumab (Cyramza)), KRAS inhibitors (e.g., Sotorasib (Lumakras)), EGFR inhibitors (e.g., Erlotinib (Tarceva), Afatinib (Gilotrif), Gefitinib (Iressa), Osimertinib (Tagrisso), Dacomitinib (Vizimpro), Amivantamab (Rybrevant), and Necitumumab (Portrazza)), ALK inhibitors (e.g., Crizotinib (Xalkori), Ceritinib (Zykadia), Alectinib (Alecensa), Brigatinib (Alunbrig), and Lorlatinib) (Lorbrena)), ROS1 inhibitors (e.g., Crizotinib (Xalkori), Ceritinib (Zykadia), Lorlatinib (Lorbrena), Entrectinib (Rozlytrek)), BRAF inhibitors (e.g., Dabrafenib (Tafinlar) and Trametinib (Mekinist)), RET inhibitors (e.g., Selpercatinib (Retevmo) and pralsetinib (Gavreto)), MET inhibitors (e.g., Capmatinib (Tabrecta) and tepotinib (Tepmetko)), NTRK inhibitors (e.g., Larotrectinib (Vitrakvi) and entrectinib (Rozlytrek)); and immunotherapeutics, including, without limitation, immune checkpoint inhibitors, e.g., PD-1 inhibitors (Pembrolizumab (Keytruda), nivolumab (Opdivo), cemiplimab (Libtayo)), PD-L1 inhibitor (e.g., Atezolizumab (Tecentriq) and Durvalumab (Imfinzi)) and CTLA-4 inhibitor (e.g., Ipilimumab (Yervoy)).

In another embodiment, the at least three proteins detected to identify the type of cancer present in the subject include are selected from immunoglobulin lambda constant 3 (IGLC3), immunoglobulin heavy variable 4-59 (IGHV4-59), immunoglobulin heavy variable 3-20 (IGHV3-20), immunoglobulin heavy variable 3-64 (IGHV3-64), immunoglobulin heavy variable 3-16 (IGHV3-16), immunoglobulin heavy variable 3-11 (IGHV3-11), complement factor H-related protein 3 (CFHR3), and immunoglobulin heavy variable 3 or 16-9 (IGHV3OR16-9). In accordance with this embodiment, pancreatic cancer is detected in the subject when the expression of IGLC3 is detected and the expression of CFHR3, IGHG3, IGHV4-59, IGHV3-20, IGHV3-64, IGLV3-16, IGHV3-11, IGHV3OR16-9 or any combination thereof is not detected in the extracellular vesicle and particle protein sample. If pancreatic cancer is detected and identified as the type of cancer in the subject, the subject can be administered one or more therapies suitable for treating the identified pancreatic cancer. Suitable therapies for treating pancreatic cancer are known in the art, and include, for example and without limitation surgery (e.g., pancreaticoduodenectomy, distal pancreatectomy, total pancreatectomy); ablation therapy (e.g., radiofrequency ablation, microwave thermotherapy, ethanol ablation, cryosurgery); embolization therapy (e.g., arterial embolization, chemoembolization, radioembolization); radiation therapy; chemotherapy, including, but not limited to Gemcitabine (Gemzar), 5-fluorouracil (5-FU), Oxaliplatin (Eloxatin), Albumin-bound paclitaxel (Abraxane), Capecitabine (Xeloda), Cisplatin, Irinotecan (Camptosar), Platinum agents (Cisplatin and Oxaliplatin), and Taxanes (Paclitaxel (Taxol), Docetaxel (Taxotere), and Albumin-bound paclitaxel (Abraxane); targeted therapies, including, but not limited to, EGFR inhibitors (e.g., Erlotinib (Rarceva)), PARP inhibitors (e.g., Olaparib (Lynparza)), NTRK inhibitors (e.g., larotrectinib (Vitrakvi) and entrectinib (Rozlytrek); and immunotherapies, including, without limitation, immune checkpoint inhibitors, e.g., PD-1 inhibitor (Pembrolizumab).

In another embodiment, the at least three proteins detected in accordance with this method to identify the type of cancer present in the subject are selected from IGHG3, IGHV3-74, IGHV3-72, IGHV3-43, IGHV5-10-1, IGLV7-46, IGKV3D-20, IGKV2-24, and Ficolin-3. In accordance with this embodiment, breast cancer is detected in the subject when the expression of IGHG3 is detected and the expression of IGHV3-74, IGHV3-72, IGHV3-43, IGHV5-10-1, IGLV7-46, IGKV3D-20, IGKV2-24, Ficolin-3, or any combination thereof is not detected in the extracellular vesicle and particle protein sample. If breast cancer is detected and identified as the cancer type in the subject, the subject can be administered one or more therapies suitable for treating the identified breast cancer. Suitable therapies for treating breast cancer are known in the art, and include, for example and without limitation chemotherapy, including, without limitation, anthracyclines, such as doxorubicin (Adriamycin) and epirubicin (Ellence), taxanes, such as paclitaxel (Taxol) and docetaxel (Taxotere), 5-fluorouracil (5-FU), capecitabine, cyclophosphamide (Cytoxan), carboplatin (Paraplatin), albumin-bound paclitaxel (Abraxane), anthracyclines (Doxorubicin, pegylated liposomal doxorubicin, and Epirubicin), platinum agents (cisplatin, carboplatin), vinorelbine (Navelbine), capecitabine (Xeloda), gemcitabine (Gemzar), ixabepilone (Ixempra), eribulin (Halaven); hormone therapy, including, without limitation, tamoxifen, toremifene (Fareston), fulvestrant (faslodex); aromatase inhibitors (e.g., Letrozole (Femara), Anastrozole (Arimidex), and Exemestane (Aromasin)); targeted therapeutics, including, without limitation, monoclonal antibody therapeutics (e.g., HER2 antibody (Trastuzumab (Herceptin)), Pertuzumab (Perjeta), Margetuximab (Margenza), antibody-drug conjugates (e.g., Ado-trastuzumab emtansine (Kadcyla or TDM-1), Fam-trastuzumab deruxtecan (Enhertu), Sacituzumab govitecan (Trodelvy)), kinase inhibitors (e.g., Lapatinib (Tykerb), Neratinib (Nerlynx), Tucatinib (Tukysa)), CDK4/6 inhibitors (e.g., Palbociclib (Ibrance), ribociclib (Kisqali), and abemaciclib (Verzenio)), mTOR inhibitors (e.g., Everolimus (Afinitor)), PI3K inhibitor (e.g., Alpelisib (Piqray)), PARP inhibitors (e.g., Olaparib (Lynparza) and talazoparib (Talzenna)); and immunotherapy, including, without limitation, immune checkpoint inhibitors, e.g., PD-1 inhibitors (Pembrolizumab and Atezolizumab (Tecentriq)).

In another embodiment, the at least three proteins detected in accordance with this method to identify the type of cancer present in the subject are selected from IGHV5-10-1, IGLV7-46, IGHG3 and IGLC2. In accordance with this embodiment, colorectal cancer is identified in the subject when expression of IGLC2 is detected and expression of IGHV5-10-1, IGLV7-46, IGHG3, or any combination thereof is not detected in the extracellular vesicle and particle protein sample. If colorectal cancer is detected and identified as the cancer type in the subject, the subject can be administered one or more therapies suitable for treating the identified colorectal cancer. Suitable therapies for treating colorectal cancer are known in the art, and include, for example and without limitation surgery (polypectomy, local excision, partial or total colectomy); ablation therapy (radiofrequency ablation, microwave ablation, ethanol ablation, cryosurgery); embolization (arterial embolization, chemoembolization, radioembolization); radiation therapy; chemotherapeutics, including, but not limited to, 5-Fluorouracil (5-FU), Capecitabine (Xeloda) (a 5-FU prodrug), Irinotecan (Camptosar), Oxaliplatin (Eloxatin), and Trifluridine and tipiracil (Lonsurf) (a combination drug); targeted therapeutics, including, but not limited to VEGF inhibitors (e.g., Bevacizumab (Avastin), Ramucirumab (Cyramza), Ziv-aflibercept (Zaltrap)), EGFR inhibitors (e.g., Cetuximab (Erbitux) and Panitumumab (Vectibix)), BRAF inhibitors (e.g., Encorafenib (Braftovi)), kinase inhibitors (e.g., regorafenib (stivarga)); and immunotherapeutics, including, without limitation, immune checkpoint inhibitors, e.g., PD-1 inhibitors (Pembrolizumab and nivolumab), and CTLA-4 inhibitor (e.g., Ipilimumab (Yervoy)).

In another embodiment, the at least three proteins detected in accordance with this method to identify the type of cancer present in the subject are IGLC2, IFKV1, and CFHR3. In accordance with this embodiment, mesothelioma is identified in the subject when expression of CFHR3 is detected and expression of IGLC2 and/or IFKV1 are not detected in the extracellular vesicle and particle protein sample. If mesothelioma is detected and identified as the cancer type in the subject, the subject can be administered one or more therapies suitable for treating the identified mesothelioma. Suitable therapies for treating mesothelioma are known in the art, and include, for example and without limitation surgery (wide local excision, pleurectomy and decortication, extrapleural pneumonectomy, pleurodesis); radiation therapy; chemotherapeutics, including, but not limited to, Alimta (Pemetrexed Disodium), Ipilimumab, Nivolumab, Opdivo (Nivolumab), Pemetrexed Disodium, Gemcitabine-Cisplatin combination; immunotherapeutics, including, without limitation, immune checkpoint inhibitors, e.g., PD-1 inhibitors (Pembrolizumab and nivolumab), and CTLA-4 inhibitor (e.g., Ipilimumab (Yervoy)); and targeted therapeutics, including, but not limited to VEGF inhibitors (e.g., Bevacizumab (Avastin), and kinase inhibitors (e.g., regorafenib (stivarga)).

In accordance with all aspects and embodiments described herein where extracellular vesicles and particles are separated or isolated from a biological tissue or fluid sample, this separation and/or isolation can be performed using a method that involves contacting the biological tissue or fluid sample with one or more binding molecules specific for the thirteen exosomal protein markers identified herein. As disclosed herein, thirteen universal protein exosomal markers have been identified to improve the isolation of human exosomes from biological samples. The thirteen identified exosomal markers include alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin. The biological sample is thus contacted with the one or more binding molecules specific for the aforementioned exosomal markers under conditions suitable for the one or more binding molecules to bind its respective exosomal marker protein in the sample to form one or more binding molecule-target protein complexes. The one or more binding molecule-target protein complexes are selected for, thereby separating the extracellular vesicle and particles from the biological sample. Binding molecules capable of binding an exosomal marker proteins can be used alone or in combination to isolate or separate exosome from a sample or to enrich the purity of a previously fractionated biological sample.

In certain embodiment, the sample is contacted with at least two different binding molecules or with at least three different binding molecules to enhance the isolation of extracellular vesicles and particles from a biological liquid or tissue sample.

An enriched population of extracellular vesicles and particles can also be obtained from a biological sample using other methods known in the art (see e.g., WO2019/109077 to Lyden et al., which is hereby incorporated by reference in its entirety). For example, exosomes may be concentrated or isolated from a biological sample using size exclusion chromatography, density gradient centrifugation, differential centrifugation (Raposo et al. “B lymphocytes Secrete Antigen-presenting Vesicles,” J Exp Med 183(3): 1161-72 (1996), which is hereby incorporated by reference in its entirety), anion exchange and/or gel permeation chromatography (for example, as described in U.S. Pat. No. 6,899,863 to Dhellin et al., and 6,812,023 to Lamparski et al., which are hereby incorporated by reference in their entirety), sucrose density gradients or organelle electrophoresis (for example, as described in U.S. Pat. No. 7,198,923), magnetic activated cell sorting (MACS) (Taylor et al., “MicroRNA Signatures of Tumor-derived Exosomes as Diagnostic Biomarkers of Ovarian Cancer,” Gynecol Oncol 110(1): 13-21 (2008), which is hereby incorporated by reference in its entirety), nanomembrane ultrafiltration (Cheruvanky et al., “Rapid Isolation of Urinary Exosomal Biomarkers using a Nanomembrane Ultrafiltration Concentrator,” Am J Physiol Renal Physiol 292(5): F1657-61 (2007), which is hereby incorporated by reference in its entirety), immunoabsorbent capture, affinity purification, microfluidic separation, asymmetric flow field-flow fractionation (AF4) (Fraunhofer et al., “The Use of Asymmetrical Flow Field-Flow Fractionation in Pharmaceutics and Biopharmaceutics,” European Journal of Pharmaceutics and Biopharmaceutics 58:369-383 (2004); Yohannes et al., “Asymmetrical Flow Field-Flow Fractionation Technique for Separation and Characterization of Biopolymers and Bioparticles,” Journal of Chromatography. A 1218:4104-4116 (2011), which are hereby incorporated by reference in their entirety), or combinations thereof.

In one embodiment, the extracellular vesicles and particles are separated using a method that involves subjecting the sample to at least three sequential centrifugations. By way of example, and as described in the Examples infra, first, cell contamination may be removed from 3-4 day cell culture supernatant, bodily fluids or resected tissue culture supernatant by centrifugation at 500×g for 10 minutes. Apoptotic bodies and large cell debris may then be removed by centrifuging the supernatants at 3,000×g for 20 minutes, followed by centrifugation at 12,000×g for 20 minutes to remove large microvesicles. Finally, exosomes are collected by spinning at 100,000×g for 70 minutes.

In one embodiment, the extracellular vesicles and particles are separated from a sample using a method that involves contacting the sample with one or more binding molecules capable of binding to alpha-2-macroglobulin, moesin, and galectin-3-binding protein. The complex of the binding molecule bound to extracellular vesicles and particles is separated from the sample. In one embodiment, the sample is contacted with one or more antibodies capable of binding to alpha-2-macroglobulin, moesin, and galectin-3-binding protein.

In another embodiment, the sample is contacted with a binding molecule capable of binding alpha-2-macroglobulin, a binding molecule capable of binding moesin, and a binding molecule capable of binding galectin-3-binding protein. In one embodiment, the binding molecules capable of binding alpha-2-macroglobulin, moesin, and galectin-3-binding protein are antibodies.

As used herein, a “binding molecule” may include an antibody or binding fragment thereof, or an antibody derivative that binds specifically to the protein of interest.

An antibody of the present disclosure is an intact immunoglobulin as well as a molecule having an epitope-binding fragment thereof that binds to a portion of the amino acid sequence of protein of interest. As used herein, the terms “fragment”, “region”, and “domain” are generally intended to be synonymous, unless the context of their use indicates otherwise. Full antibodies typically comprise a tetramer which is usually composed of at least two heavy (H) chains and at least two light (L) chains. Each heavy chain is comprised of a heavy chain variable (VH) region and a heavy chain constant (CH) region, usually comprised of three domains (CH1, CH2 and CH3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable (VL) region and a light chain constant (CL) region.

Antibody fragments (including Fab and (Fab)2 fragments) that exhibit epitope-binding ability can be utilized and are obtained, for example, by protease cleavage of intact antibodies. Examples of the epitope-binding fragments suitable for use in the methods described herein include (i) Fab′ or Fab fragments, which are monovalent fragments containing the VL, VH, CL and CH1 domains; (ii) F(ab′)2 fragments, which are bivalent fragments comprising two Fab fragments linked by a disulfide bridge at the hinge region; (iii) Fd fragments consisting essentially of the VH and CH1 domains; (iv) Fv fragments consisting essentially of a VL and VH domain, (v) dAb fragments (Ward et al. “Binding Activities Of A Repertoire Of Single Immunoglobulin Variable Domains Secreted From Escherichia coli,” Nature 341:544-546 (1989) which is hereby incorporated by reference in its entirety), which consist essentially of a VH or VL domain and also called domain antibodies (Holt et al. “Domain Antibodies: Proteins For Therapy,” Trends Biotechnol. 21(11):484-490 (2003), which is hereby incorporated by reference in its entirety); (vi) camelid or nanobodies (Revets et al. “Nanobodies As Novel Agents For Cancer Therapy,” Expert Opin. Biol. Ther. 5(1):111-124 (2005), which is hereby incorporated by reference in its entirety) and (vii) isolated complementarity determining regions (CDR). An epitope-binding fragment may contain 1, 2, 3, 4, 5 or all 6 of the CDR domains of such antibody.

Antibody derivatives suitable for use in the methods disclosed herein include those molecules that contain at least one epitope-binding domain of an antibody, and are typically formed using recombinant techniques. One exemplary antibody derivative includes a single chain Fv (scFv). A scFv is formed from the two domains of the Fv fragment, the VL region and the VH region, which are encoded by separate gene.

Once extracellular vesicles and particles are isolated from the biological sample using the methods described supra, protein from the vesicles and particles is isolated and obtained. The resulting extracellular vesicle and particle protein sample is subjected to a detection assay suitable for detecting the various protein biomarkers as described supra.

In accordance with all aspects of the disclosure relating to subjecting the extracellular vesicle and particle protein sample to a detection assay, suitable detection assays include, but are not limited to, those that measure protein expression levels. Methods for detecting and measuring protein expression levels generally involve an immunoassay, where the exosomal protein sample is contacted with one or more detectable binding reagents that is suitable for measuring protein expression, e.g., a labeled antibody that binds to the protein of interest, i.e., a biomarker as described herein, or a primary antibody that binds to a biomarker used in conjunction with a secondary antibody. The one or more binding reagents bound to the biomarker (i.e., a binding reagent-biomarker complex) in the sample is detected, and the amount of labeled binding reagent that is detected and normalized to total protein in the sample, serves as an indicator of the amount or expression level of the biomarker present in the sample.

Suitable immunoassays for detecting protein expression level in an exosome sample that are commonly employed in the art include, for example and without limitation, western blot, immunoprecipitation, enzyme-linked immunosorbent assay (ELISA), radioimmunoassay (RIA), fluorescent activated cell sorting (FACS), immunoradiometric assay, gel diffusion precipitation reaction, immunodiffusion assay, in situ immunoassay, imaging mass cytometry, complement fixation assay, and immunoelectrophoresis assay.

In another embodiment, biomarker expression levels are measured using one-dimensional and two-dimensional electrophoretic gel analysis, high performance liquid chromatography (HPLC), reverse phase HPLC, Fast protein liquid chromatograph (FPLC), mass spectrometry (MS), tandem mass spectrometry, liquid crystal-MS (LC-MS), surface enhanced laser desorption/ionization (SELDI), MALDI, and/or protein sequencing.

In accordance with all aspects of the disclosure, protein biomarker expression levels, can also or alternatively be measured by detecting and quantifying biomarker nucleic acid levels using a nucleic acid detection assay. In one embodiment, RNA, e.g., mRNA, levels are measured. RNA is preferably reverse-transcribed to synthesize complementary DNA (cDNA), which is then amplified and detected or directly detected. The detected cDNA is measured and the levels of cDNA serve as an indicator of the RNA or mRNA levels present in a sample. Reverse transcription may be performed alone or in combination with an amplification step, e.g., reverse transcription polymerase chain reaction (RT-PCR), which may be further modified to be quantitative, e.g., quantitative RT-PCR as described in U.S. Pat. No. 5,639,606, which is hereby incorporated by reference in its entirety.

It may be beneficial or otherwise desirable to extract RNA from the primary tumor cells prior to or for analysis. RNA molecules can be isolated from cells and the concentration (i.e., total RNA) quantified using any number of procedures, which are well-known in the art, the particular extraction procedure chosen based on the particular biological sample. In some instances, with some techniques, it may also be possible to analyze the nucleic acid without extraction from the cells.

In one embodiment, mRNA is analyzed directly without an amplification step. Direct analysis may be performed with different methods including, but not limited to, nanostring technology (Geiss et al. “Direct Multiplexed Measurement of Gene Expression with Color-Coded Probe Pairs,” Nat Biotechnol 26(3): 317-25 (2008), which is hereby incorporated by reference in its entirety). Nanostring technology enables identification and quantification of individual target molecules in a biological sample by attaching a color coded fluorescent reporter to each target molecule. This approach is similar to the concept of measuring inventory by scanning barcodes. Reporters can be made with hundreds or even thousands of different codes allowing for highly multiplexed analysis. In another embodiment, direct analysis can be performed using immunohistochemical techniques.

In another embodiment, it may be beneficial or otherwise desirable to reverse transcribe and amplify the RNA prior to detection/analysis. Methods of nucleic acid amplification, including quantitative amplification, are commonly used and generally known in the art. Quantitative amplification will allow quantitative determination of relative amounts of RNA in the cells.

Nucleic acid amplification methods include, without limitation, polymerase chain reaction (PCR) (U.S. Pat. No. 5,219,727, which is hereby incorporated by reference in its entirety) and its variants such as in situ polymerase chain reaction (U.S. Pat. No. 5,538,871, which is hereby incorporated by reference in its entirety), quantitative polymerase chain reaction (U.S. Pat. No. 5,219,727, which is hereby incorporated by reference in its entirety), nested polymerase chain reaction (U.S. Pat. No. 5,556,773), self sustained sequence replication and its variants (Guatelli et al. “Isothermal, In vitro Amplification of Nucleic Acids by a Multienzyme Reaction Modeled after Retroviral Replication,” Proc Natl Acad Sci USA 87(5): 1874-8 (1990), which is hereby incorporated by reference in its entirety), transcriptional amplification and its variants (Kwoh et al. “Transcription-based Amplification System and Detection of Amplified Human Immunodeficiency Virus type 1 with a Bead-Based Sandwich Hybridization Format,” Proc Natl Acad Sci USA 86(4): 1173-7 (1989), which is hereby incorporated by reference in its entirety), Qb Replicase and its variants (Miele et al. “Autocatalytic Replication of a Recombinant RNA.” J Mol Biol 171(3): 281-95 (1983), which is hereby incorporated by reference in its entirety), cold-PCR (Li et al. “Replacing PCR with COLD-PCR Enriches Variant DNA Sequences and Redefines the Sensitivity of Genetic Testing.” Nat Med 14(5): 579-84 (2008), which is hereby incorporated by reference in its entirety) or any other nucleic acid amplification method known in the art. Depending on the amplification technique that is employed, the amplified molecules are detected during amplification (e.g., real-time PCR) or subsequent to amplification using detection techniques known to those of skill in the art. Suitable nucleic acid detection assays include, for example and without limitation, northern blot, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, and mass spectrometry (MS) methods (e.g., MassARRAY® System).

### Diagnostic Methods Based on Tissue Derived Extracellular Vesicles and Particles

Another aspect of the present disclosure is directed to a method of detecting cancer in a subject that involves obtaining and analyzing a tissue sample from a subject. Extracellular vesicles and particles are separated from the tissue sample, and protein from the separated extracellular vesicles and particles is isolated to form an extracellular vesicle and particle protein sample.

Extracellular vesicles and particles, suitable subjects, and methods of separating extracellular vesicles and particles from a biological sample are described supra.

In one aspect, the present disclosure is directed to a method for screening a subject for the presence of cancer that involves obtaining a tissue sample from a subject. In one embodiment, this method involves separating extracellular vesicles and particles from a tissue sample, and isolating protein from the separated extracellular vesicles and particles to form an extracellular vesicle and particle protein sample. The extracellular vesicle and particle protein sample is subjected to a detection assay suitable for detecting: (i) a protein selected from the group consisting of thrombospondin 2, versican, serrate, RNA effector molecule, tenascin C, dihydropyrimidinase like 2, adenosylhomocysteinase, DnaJ heat shock protein family (Hsp40) member A1, phosphoglycerate kinase 1, EH domain containing 2, and combinations thereof, and (ii) a protein selected from the group consisting of alcohol dehydrogenase 1B (class I), beta polypeptide, caveolae associated protein 1, FGGY carbohydrate kinase domain containing, ATP binding cassette subfamily A member 3, syntaxin 11, caveolae associated protein 2, CD36 molecule, and combinations thereof, thereby detecting the presence or absence of the protein of (i) and the protein of (ii) in the extracellular vesicle and particle protein sample. Detecting the presence of one or more proteins from group (i) is indicative of the presence of cancer in the subject and detecting the presence of one or more proteins from (ii) is indicative of the absence of cancer in the subject.

In any embodiment, the protein of group (i) is thrombospondin 2, and in another embodiment the protein of group (i) is versican. In any embodiment, the protein of (ii) is CD36 molecule, and in another embodiment the protein of (ii) is caveloae associated protein 2.

In any embodiment, at least two proteins of (i) are detected in the method. In any embodiment, at least two proteins of (ii) are detected in the method.

In any embodiment, at least two proteins of (i) and at least two proteins of (ii) are detected in the method. In one embodiment, the at least two proteins of (i) are thrombospondin 2 and versican, and the at least two proteins of (ii) are caveolae associated protein 2 and CD36 molecule.

In another aspect, this method involves separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from the group consisting of tenacin (TNC), Periostin (POSTN), Versican core protein (VCAN), signal recognition particle 9 kDa protein (SRP9), Nucleophosmin (NPM1), Serrate RNA effector molecule homolog (SRRT), ELAV-like protein 1 (ELAVL1), Cytosolic acyl coenzyme A thioester hydrolase (ACOT7), 5′-3′ exoribonuclease 2 (XRN2), Flap endonuclease 1 (FEN1), ADP-ribosylation factor-like protein 1 (ARL1), Heat shock protein 105 kDa (HSPH1), Nucleolar RNA helicase 2 (DDX21), Src-associated in mitosis 68 kDa protein (KHDRBS1), Importin subunit alpha-1 (KPNA2), SLIT-ROBO Rho GTPase-activating protein 1 (SRGAP1), WD repeat-containing protein 3 (WDR3), and combinations thereof, and (ii) a protein selected from the group consisting of Voltage-dependent calcium channel subunit alpha-2/delta-2 (CACNA2D2), Specifically androgen-regulated gene protein (C1orf116), Caveolin-2 (CAV2), Syntaxin-11 (STX11), Caveolae-associated protein 2 (CAVIN2), and combinations thereof, thereby detecting the presence or absence of the protein of (i) and the protein of (ii) in the extracellular vesicle and particle protein sample. Detecting the presence of one or more proteins from group (i) is indicative of the presence of cancer in the subject and detecting the presence of one or more proteins from (ii) is indicative of the absence of cancer in the subject.

In any embodiment, the protein of group (i) is KPNA2. In another embodiment the protein of group (i) is SRGAP1. In another embodiment, the protein of group (i) is WDR3. In another embodiment each of KPNA2, SRGAP1 and WDR3 are detected.

In accordance with this aspect of the present disclosure, and similar to the methods described above using a liquid biopsy, these methods are employed to screen a subject for the general presence of cancer based on the presence and/or absence of the described proteins in the extracellular vesicle and particle protein sample.

In accordance with this aspect of the present disclosure, these methods are employed to detect the general presence of cancer in the subject based on the presence and/or absence of the described proteins in the extracellular vesicle and particle protein sample. Accordingly, detecting the presence of one or more proteins from group (i) is indicative of the presence of cancer in the subject and detecting the presence of one or more proteins from (ii) is indicative of the absence of cancer in the subject.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of cancer in the subject.

In some embodiments, at least one, at least two, at least three, at least four, at least 5, at least 6, at least 7, at least 8, at least 9, at least 10 or greater than 10 proteins from the proteins of group (ii) are subject to detection, and the presence of any one or more of these proteins in the sample indicates the absence of cancer in the subject.

When utilized together, the detection of one or more proteins of (i) and the absence of one or more proteins in (ii) is indicative of the presence of cancer in the subject. Alternatively, detecting the absence of one or more proteins of (i) and the presence of one or more proteins in (ii) is indicative that the subject does not have cancer. Detecting both the presence and/or absence of tumor-associated and non-tumor associated exosomal proteins significantly improves the diagnostic integrity of the methods described herein.

Suitable subjects are described above. For example, this method can be employed during a regularly scheduled physical examination to achieve early detection of cancer in the subject. Alternatively, the method may be employed in a subject possessing a tumor or abnormal tissue mass, where it is unknown if the tumor or tissue mass is benign or malignant. Accordingly, when the method is employed to detect the general presence of cancer in a subject, the presence of one or more proteins from (i) is indicative of the presence of cancer in the subject and detecting the presence of one or more proteins from (ii) is indicative of the absence of cancer in the subject.

In another aspect, the present disclosure is directed to a method that involves obtaining a tissue sample from a subject. Extracellular vesicles and particles are separated from the tissue sample, and protein from the separated extracellular vesicles and particles is isolated to form an extracellular vesicle and particle protein sample. The extracellular vesicle and particle protein sample is subjected to a detection assay suitable for detecting one or more proteins from Table 5 and/or detecting one or more proteins from Table 6 below.

TABLE 5
Top 50 highly expressed EVP proteins in tumors based on tumor
tissue-derived EVPs (n = 85) versus non-tumor tissue-
derived EVP (n = 66) comparison (>100-fold higher in tumors).

| | | | FOLD | TUMOR | NORMAL |
|---|---|---|---|---|---|
| No. | Gene/Protein Name | FDR | CHANGE | (%) | (%) |
| 1 | VCAN/ | 0.005 | 133048.8 | 78% | 18% |
| | Versican core protein | | | | |
| 2 | KHDRBS1/ | 0.005 | 21721.9 | 73% | 21% |
| | KH domain-containing, RNA- | | | | |
| | binding, signal transduction- | | | | |
| | associated protein 1 | | | | |
| 3 | POSTN/ | 0.005 | 14766.4 | 75% | 23% |
| | Periostin | | | | |
| 4 | ACOT7/ | 0.005 | 13551.0 | 65% | 14% |
| | Cytosolic acyl coenzyme A | | | | |
| | thioester hydrolase | | | | |
| 5 | HNRNPK/ | 0.005 | 10226.8 | 98% | 52% |
| | Heterogeneous nuclear | | | | |
| | ribonucleoprotein K | | | | |
| 6 | ARL1/ | 0.005 | 10146.0 | 71% | 18% |
| | ADP-ribosylation factor-like | | | | |
| | protein 1 | | | | |
| 7 | SRRT/ | 0.005 | 8604.1 | 64% | 12% |
| | Serrate RNA effector molecule | | | | |
| | homolog | | | | |
| 8 | HSPH1/ | 0.005 | 8033.1 | 60% | 11% |
| | Heat shock protein 105 kDa | | | | |
| 9 | FKBP4/ | 0.005 | 6100.1 | 84% | 36% |
| | Peptidyl-prolyl cis-trans | | | | |
| | isomerase FKBP4 | | | | |
| 10 | HNRNPA3/ | 0.005 | 6001.7 | 86% | 42% |
| | Heterogeneous nuclear | | | | |
| | ribonucleoprotein A3 | | | | |
| 11 | NPM1/ | 0.005 | 5704.4 | 78% | 35% |
| | Nucleophosmin | | | | |
| 12 | HDLBP/ | 0.005 | 5088.9 | 72% | 24% |
| | Vigilin | | | | |
| 13 | BZW2/ | 0.005 | 4867.7 | 85% | 38% |
| | Basic leucine zipper and W2 | | | | |
| | domain-containing protein 2 | | | | |
| 14 | NCL/Nucleolin | 0.005 | 4491.8 | 73% | 27% |
| 15 | PAPSS1/ | 0.005 | 4447.5 | 67% | 20% |
| | Bifunctional 3′- | | | | |
| | phosphoadenosine 5′- | | | | |
| | phosphosulfate synthase 1 | | | | |
| 16 | ELAVL1/ | 0.005 | 4422.7 | 55% | 9% |
| | ELAV-like protein 1 | | | | |
| 17 | LYPLA1/ | 0.005 | 4395.8 | 87% | 42% |
| | Acyl-protein thioesterase 1 | | | | |
| 18 | IMPDH2/ | 0.005 | 4371.3 | 82% | 36% |
| | Inosine-5′-monophosphate | | | | |
| | dehydrogenase 2 | | | | |
| 19 | TNC/ | 0.005 | 4209.1 | 56% | 9% |
| | Tenascin | | | | |
| 20 | GMPS/ | 0.005 | 4074.2 | 73% | 29% |
| | GMP synthase [glutamine- | | | | |
| | hydrolyzing] | | | | |
| 21 | SRM/ | 0.005 | 4054.6 | 76% | 32% |
| | Spermidine synthase | | | | |
| 22 | HSP90AA4P/ | 0.005 | 4021.4 | 92% | 52% |
| | Putative heat shock protein | | | | |
| | HSP 90-alpha A4 | | | | |
| 23 | SRP9/ | 0.005 | 4000.5 | 56% | 11% |
| | Signal recognition particle 9 | | | | |
| | kDa protein | | | | |
| 24 | DPYSL3/ | 0.005 | 3930.2 | 94% | 52% |
| | Dihydropyrimidinase-related | | | | |
| | protein 3 | | | | |
| 25 | TOP1MT/ | 0.005 | 3919.1 | 71% | 26% |
| | DNA topoisomerase I, | | | | |
| | mitochondrial | | | | |
| 26 | XRN2/ | 0.005 | 3683.1 | 55% | 8% |
| | 5′-3′ exoribonuclease 2 | | | | |
| 27 | COPZ1/ | 0.005 | 3628.2 | 65% | 20% |
| | Coatomer subunit zeta-1 | | | | |
| 28 | KIF5B/ | 0.005 | 3554.2 | 92% | 47% |
| | Kinesin-1 heavy chain | | | | |
| 29 | NSUN2/ | 0.005 | 3549.6 | 65% | 20% |
| | RNA cytosine C(5)- | | | | |
| | methyltransferase NSUN2 | | | | |
| 30 | NANS/ | 0.005 | 3461.0 | 86% | 44% |
| | Sialic acid synthase | | | | |
| 31 | DNAJA2/ | 0.005 | 3419.4 | 84% | 39% |
| | DnaJ homolog subfamily A | | | | |
| | member 2 | | | | |
| 32 | PRPF4/ | 0.005 | 3393.8 | 62% | 17% |
| | U4/U6 small nuclear | | | | |
| | ribonucleoprotein Prp4 | | | | |
| 33 | THBS2/ | 0.005 | 3373.2 | 80% | 38% |
| | Thrombospondin-2 | | | | |
| 34 | FEN1/ | 0.005 | 3361.8 | 48% | 5% |
| | Flap endonuclease 1 | | | | |
| 35 | DDX21/ | 0.005 | 3328.5 | 54% | 9% |
| | Nucleolar RNA helicase 2 | | | | |
| 36 | ARPC1B/ | 0.005 | 3289.2 | 96% | 53% |
| | Actin-related protein 2/3 | | | | |
| | complex subunit 1B | | | | |
| 37 | ATIC/ | 0.005 | 3118.8 | 95% | 55% |
| | Bifunctional purine biosynthesis | | | | |
| | protein ATIC | | | | |
| 38 | CPSF7/ | 0.005 | 3040.0 | 64% | 17% |
| | Cleavage and polyadenylation | | | | |
| | specificity factor subunit 7 | | | | |
| 39 | SF3A3/ | 0.005 | 2937.3 | 54% | 8% |
| | Splicing factor 3A subunit 3 | | | | |
| 40 | MARCKSL1/ | 0.005 | 2907.3 | 62% | 17% |
| | MARCKS-related protein | | | | |
| 41 | RPL12/ | 0.005 | 2887.9 | 93% | 53% |
| | 60S ribosomal protein L12 | | | | |
| 42 | SSRP1/ | 0.005 | 2816.3 | 65% | 21% |
| | FACT complex subunit SSRP1 | | | | |
| 43 | PLEC/ | 0.005 | 2751.3 | 93% | 50% |
| | Plectin | | | | |
| 44 | GARS/ | 0.005 | 2683.4 | 93% | 52% |
| | Glycine--tRNA ligase | | | | |
| 45 | AARS/ | 0.005 | 2521.0 | 92% | 50% |
| | Alanine--tRNA ligase, | | | | |
| | cytoplasmic | | | | |
| 46 | EIF4G3/ | 0.005 | 2493.9 | 67% | 24% |
| | Eukaryotic translation initiation | | | | |
| | factor 4 gamma 3 | | | | |
| 47 | CBR1/ | 0.005 | 2457.8 | 95% | 53% |
| | Carbonyl reductase [NADPH] 1 | | | | |
| 48 | AKR1B1/ | 0.005 | 2435.2 | 93% | 52% |
| | Aldo-keto reductase family 1 | | | | |
| | member B1 | | | | |
| 49 | CRMP1/ | 0.005 | 2414.9 | 93% | 52% |
| | Dihydropyrimidinase-related | | | | |
| | protein 1 | | | | |
| 50 | U2AF2/ | 0.005 | 2393.1 | 72% | 30% |
| | Splicing factor U2AF 65 kDa | | | | |
| | subunit | | | | |

TABLE 6
Top 50 highly expressed EVP proteins in non-tumors based on tumor
tissue-derived EVPs (n = 85) versus non-tumor tissue-derived
EVP (n = 66) comparison (>100-fold higher in non-tumors).

| | | | FOLD | TUMOR | NORMAL |
|---|---|---|---|---|---|
| No. | Protein | FDR | CHANGE | (%) | (%) |
| 1 | CD36/ | 0.005 | −11027.5 | 28% | 76% |
| | Platelet glycoprotein 4 | | | | |
| 2 | CAVIN2/Caveolae- | 0.005 | −7042.6 | 8% | 55% |
| | associated protein 2 | | | | |
| 3 | PF4/ | 0.005 | −1553.3 | 6% | 42% |
| | Platelet factor 4 | | | | |
| 4 | STX11/ | 0.005 | −823.3 | 1% | 39% |
| | Syntaxin-11 | | | | |
| 5 | IGHV3OR16-12/ | 0.005 | −780.7 | 14% | 47% |
| | Immunoglobulin heavy | | | | |
| | variable 3/OR16-12 (non- | | | | |
| | functional) | | | | |
| 6 | AOC3/ | 0.005 | −616.8 | 42% | 74% |
| | Membrane primary amine | | | | |
| | oxidase | | | | |
| 7 | APOC4-APOC2/ | 0.005 | −545.7 | 11% | 42% |
| | Apolipoprotein C-II | | | | |
| 8 | PIGR/ | 0.005 | −525.1 | 41% | 77% |
| | Polymeric immunoglobulin | | | | |
| | receptor | | | | |
| 9 | FABP4/ | 0.005 | −519.6 | 6% | 39% |
| | Fatty acid-binding protein, | | | | |
| | adipocyte | | | | |
| 10 | APCS/ | 0.005 | −505.2 | 54% | 85% |
| | Serum amyloid P- | | | | |
| | component | | | | |
| 11 | PF4V1/ | 0.005 | −461.8 | 5% | 35% |
| | Platelet factor 4 variant | | | | |
| 12 | IGKV2D-24/ | 0.005 | −351.0 | 4% | 33% |
| | Probable non-functional | | | | |
| | immunoglobulin kappa | | | | |
| | variable 2D-24 | | | | |
| 13 | IGKV2-24/ | 0.005 | −348.4 | 4% | 33% |
| | Immunoglobulin kappa | | | | |
| | variable 2-24 | | | | |
| 14 | MASP2/ | 0.005 | −346.8 | 2% | 33% |
| | Mannan-binding lectin | | | | |
| | serine protease 2 | | | | |
| 15 | SELENOP/ | 0.005 | −339.8 | 19% | 48% |
| | Selenoprotein P | | | | |
| 16 | IGHV3-43/ | 0.005 | −309.1 | 26% | 53% |
| | Immunoglobulin heavy | | | | |
| | variable 3-43 | | | | |
| 17 | IGHV3-13/ | 0.010 | −272.3 | 22% | 50% |
| | Immunoglobulin heavy | | | | |
| | variable 3-13 | | | | |
| 18 | IGHV1-69D/ | 0.005 | −258.7 | 6% | 36% |
| | Immunoglobulin heavy | | | | |
| | variable 1-69D | | | | |
| 19 | IGKV3D-11/ | 0.005 | −257.4 | 27% | 53% |
| | Immunoglobulin kappa | | | | |
| | variable 3D-11 | | | | |
| 20 | IGHV1-69/ | 0.005 | −256.8 | 6% | 36% |
| | Immunoglobulin heavy | | | | |
| | variable 1-69 | | | | |
| 21 | IGHV1-46/ | 0.005 | −250.7 | 6% | 36% |
| | Immunoglobulin heavy | | | | |
| | variable 1-46 | | | | |
| 22 | CFP/ | 0.005 | −242.6 | 0% | 29% |
| | Properdin | | | | |
| 23 | IGLV3-27/ | 0.005 | −241.3 | 9% | 36% |
| | Immunoglobulin lambda | | | | |
| | variable 3-27 | | | | |
| 24 | IGHV1-3/ | 0.005 | −237.3 | 6% | 36% |
| | Immunoglobulin heavy | | | | |
| | variable 1-3 | | | | |
| 25 | IGLV1-47/ | 0.005 | −218.0 | 4% | 32% |
| | Immunoglobulin lambda | | | | |
| | variable 1-47 | | | | |
| 26 | IGHV3-33/ | 0.005 | −217.5 | 40% | 65% |
| | Immunoglobulin heavy | | | | |
| | variable 3-33 | | | | |
| 27 | IGHV3OR16-13/ | 0.005 | −214.9 | 18% | 42% |
| | Immunoglobulin heavy | | | | |
| | variable 3/OR16-13 (non- | | | | |
| | functional) | | | | |
| 28 | IGHV5-51/ | 0.005 | −205.7 | 9% | 36% |
| | Immunoglobulin heavy | | | | |
| | variable 5-51 | | | | |
| 29 | PPBP/ | 0.005 | −203.7 | 2% | 29% |
| | Platelet basic protein | | | | |
| 30 | IGKV3D-20/ | 0.005 | −200.3 | 12% | 39% |
| | Immunoglobulin kappa | | | | |
| | variable 3D-20 | | | | |
| 31 | IGHV3-53/ | 0.005 | −193.1 | 39% | 64% |
| | Immunoglobulin heavy | | | | |
| | variable 3-53 | | | | |
| 32 | COLEC11/ | 0.005 | −189.1 | 1% | 30% |
| | Collectin-11 | | | | |
| 33 | CES1/ | 0.005 | −182.0 | 20% | 47% |
| | Liver carboxylesterase 1 | | | | |
| 34 | CA4/ | 0.005 | −169.3 | 12% | 41% |
| | Carbonic anhydrase 4 | | | | |
| 35 | SERPIND1/ | 0.005 | −167.0 | 42% | 70% |
| | Heparin cofactor 2 | | | | |
| 36 | IGKV1-17/ | 0.005 | −162.3 | 1% | 29% |
| | Immunoglobulin kappa | | | | |
| | variable 1-17 | | | | |
| 37 | SPN/ | 0.005 | −159.9 | 7% | 35% |
| | Leukosialin | | | | |
| 38 | CFI/ | 0.005 | −158.7 | 5% | 30% |
| | Complement factor I | | | | |
| 39 | IGKV1-27/ | 0.005 | −158.4 | 2% | 30% |
| | Immunoglobulin kappa | | | | |
| | variable 1-27 | | | | |
| 40 | IGLV3-16/ | 0.005 | −153.9 | 6% | 32% |
| | Immunoglobulin lambda | | | | |
| | variable 3-16 | | | | |
| 41 | IGHV4-4/ | 0.005 | −153.4 | 11% | 38% |
| | Immunoglobulin heavy | | | | |
| | variable 4-4 | | | | |
| 42 | IGHV3-20/ | 0.014 | −150.8 | 28% | 52% |
| | Immunoglobulin heavy | | | | |
| | variable 3-20 | | | | |
| 43 | IGHV3OR16-10/ | 0.014 | −149.9 | 28% | 52% |
| | Immunoglobulin heavy | | | | |
| | variable 3/OR16-10 (non- | | | | |
| | functional) | | | | |
| 44 | SFTPA2/ | 0.005 | −146.3 | 14% | 39% |
| | Pulmonary surfactant- | | | | |
| | associated protein A2 | | | | |
| 45 | IGHV4-28/ | 0.005 | −145.9 | 7% | 35% |
| | Immunoglobulin heavy | | | | |
| | variable 4-28 | | | | |
| 46 | IGLV3-25/ | 0.005 | −144.2 | 15% | 39% |
| | Immunoglobulin lambda | | | | |
| | variable 3-25 | | | | |
| 47 | CAV2/ | 0.005 | −139.0 | 4% | 30% |
| | Caveolin-2 | | | | |
| 48 | IGKV1D-33/ | 0.005 | −137.3 | 9% | 36% |
| | Immunoglobulin kappa | | | | |
| | variable 1D-33 | | | | |
| 49 | IGLV2-11/ | 0.010 | −137.2 | 15% | 39% |
| | Immunoglobulin lambda | | | | |
| | variable 2-11 | | | | |
| 50 | IGHV3-11/ | 0.010 | −135.0 | 41% | 64% |
| | Immunoglobulin heavy | | | | |
| | variable 3-11 | | | | |

Detecting the presence of one or more proteins from Table 5 is indicative of the presence of cancer in the subject and detecting the presence of one or more proteins from Table 6 is indicative of the absence of cancer in the subject.

In some embodiments, at least one, at least two, at least three, at least four, at least 5, at least 6, at least 7, at least 8, at least 9, at least 10, or greater than 10 proteins from the proteins listed in Table 5 are subject to detection, and the detection of any one or more of these protein in the tissue derived exosomal sample indicates the presence of cancer in the subject.

In some embodiments, at least one, at least two, at least three, at least four, at least 5, at least 6, at least 7, at least 8, at least 9, at least 10, or greater than 10 proteins from the proteins listing in Table 6 are subject to detection, and the presence of any one or more of these proteins in the sample indicates the absence of cancer in the subject.

When utilized together, the detection of one or more proteins of Table 5 and the absence of one or more proteins in Table 6 is indicative of the presence of cancer in the subject. Alternatively, detecting the absence of one or more proteins of Table 5 and the presence of one or more proteins in Table 6 is indicative that the subject does not have cancer. Detecting both the presence and/or absence of tumor-associated and non-tumor associated exosomal proteins significantly improves the diagnostic integrity of the methods described herein.

In another embodiment, the presence or absence of one or more proteins from Table 7 can be detected as an alternative to the proteins identified in Table 6 above. In this embodiment, the detection of one or more proteins of Table 5 and the absence of one or more proteins in Table 7 is indicative of the presence of cancer in the subject. Alternatively, detecting the absence of one or more proteins of Table 5 and the presence of one or more proteins in Table 7 is indicative that the subject does not have cancer.

TABLE 7
Proteins highly expressed non-tumor tissue-derived
EVPs, but not tumor tissue-derived EVPs

| | Type | | | | |
|---|---|---|---|---|---|
| | normal | cancer | cancer | cancer | cancer |
| | Origin | | | | |
| | normal | pancreas | lung | breast | colon |
| | N | | | | |
| | n = 66 | n = 21 | n = 18 | n = 6 | n = 3 |
| STX11 | 39% | 0% | 0% | 0% | 0% |
| COLEC11 | 30% | 0% | 0% | 0% | 0% |
| CFP | 29% | 0% | 0% | 0% | 0% |
| CD34 | 23% | 0% | 0% | 0% | 0% |
| IGHV4OR15-8 | 23% | 0% | 0% | 0% | 0% |
| GP1BB | 23% | 0% | 0% | 0% | 0% |
| SMIM1 | 21% | 0% | 0% | 0% | 0% |
| IGHV3OR15-7 | 21% | 0% | 0% | 0% | 0% |
| GIMAP1 | 20% | 0% | 0% | 0% | 0% |
| LRG1 | 20% | 0% | 0% | 0% | 0% |
| IGLV5-45 | 20% | 0% | 0% | 0% | 0% |
| IGLV1-44 | 20% | 0% | 0% | 0% | 0% |
| GP1BA | 20% | 0% | 0% | 0% | 0% |
| IGHV2-26 | 18% | 0% | 0% | 0% | 0% |
| F8 | 18% | 0% | 0% | 0% | 0% |
| GP5 | 18% | 0% | 0% | 0% | 0% |
| GP9 | 18% | 0% | 0% | 0% | 0% |
| MPIG6B | 17% | 0% | 0% | 0% | 0% |
| IGLV4-69 | 15% | 0% | 0% | 0% | 0% |
| IGFBP3 | 15% | 0% | 0% | 0% | 0% |
| VCAM1 | 15% | 0% | 0% | 0% | 0% |
| HSD17B6 | 14% | 0% | 0% | 0% | 0% |
| RTKN2 | 14% | 0% | 0% | 0% | 0% |
| ANGPTL6 | 14% | 0% | 0% | 0% | 0% |
| LPCAT2 | 14% | 0% | 0% | 0% | 0% |
| HPN | 12% | 0% | 0% | 0% | 0% |
| DMTN | 12% | 0% | 0% | 0% | 0% |
| APOC4 | 12% | 0% | 0% | 0% | 0% |
| CMTM5 | 12% | 0% | 0% | 0% | 0% |
| PRDM1 | 12% | 0% | 0% | 0% | 0% |
| ASPA | 11% | 0% | 0% | 0% | 0% |
| LIMD1 | 11% | 0% | 0% | 0% | 0% |
| IGLV2-23 | 11% | 0% | 0% | 0% | 0% |
| AHSP | 11% | 0% | 0% | 0% | 0% |
| TMBIM6 | 11% | 0% | 0% | 0% | 0% |
| RPL39 | 11% | 0% | 0% | 0% | 0% |

In another aspect, the present disclosure is directed to a method for screening a subject for the presence of cancer that involves obtaining a tissue sample from a subject. Extracellular vesicles and particles are separated from the tissue sample, and protein from the separated extracellular vesicles and particles is isolated to form an extracellular vesicle and particle protein sample. The extracellular vesicle and particle protein sample is subjected to a detection assay suitable for detecting one or more proteins listed in Table 7.

The detection of one or more proteins listed in Table 7 is indicative that the subject does not have cancer. In some embodiments, the detection of one or more proteins listed in Table 7 of the tissue derived extracellular vesicle and particle protein sample indicates that the subject does not have pancreatic cancer, lung cancer, breast cancer, or colon cancer.

In some embodiments, at least one, at least two, at least three, at least four, at least 5, at least 6, at least 7, at least 8, at least 9, at least 10 or greater than 10 proteins from the proteins listed in Table 7 are subject to detection, and the detection of any one or more in the sample indicates the absence of cancer in the subject.

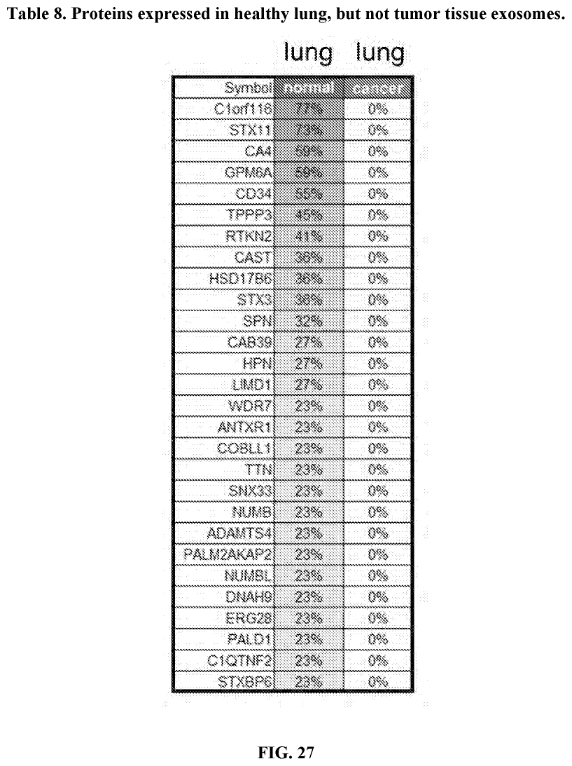

In another aspect, the present disclosure relates to methods of determining the presence of lung cancer in a subject. In one embodiment, this method involves obtaining a tissue sample from the subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting (i) a protein selected from the group consisting of Four and a half LIM domains protein 2 (FHL2), 5′-3′ exoribonuclease 2, EC 3.1.13. (XRN2), Glutaredoxin-3 (GLRX), Vigilin (High density lipoprotein-binding protein, HDL-binding protein) (HDLBP), Serrate RNA effector molecule homolog (SRRT), Regulator of chromosome condensation (RCC1), AP-3 complex subunit sigma-1 (AP3S1), Small nuclear ribonucleoprotein Sm D3, Sm-D3 (SNRPD3), NOP2, 60S ribosomal protein L22 (RPL22), DnaJ homolog subfamily C member 7 (DNAJC7), STE20/SPS1-related proline-alanine-rich protein kinase, Ste-20-related kinase (STK39), Signal recognition particle 54 kDa protein (SRP54), ATP-dependent DNA/RNA helicase DHX36 (DHX36), ELAV-like protein 1 (ELAVL1), Thrombospondin-2 (THBS2), Aconitate hydratase, mitochondrial, Aconitase (ACO2), Acyl-CoA-binding domain-containing protein 3 (ACBD3), Signal recognition particle 9 kDa protein (SRP9), THO complex subunit 2 (THOC2), Heterogeneous nuclear ribonucleoproteins C1/C2 (HNRNPC), Eukaryotic translation initiation factor 5B (EIF5B), RNA-binding protein Raly (RALY), Ubiquitin carboxyl-terminal hydrolase isozyme L5 (UCHL5), KH domain-containing, RNA-binding, signal transduction-associated protein 1 (KHDRBS1), Splicing factor 3B subunit 6 (SF3B6), WD repeat-containing protein 44 (WDR44), BRISC and BRCA1-A complex member 2 (BABAM2), Cleavage stimulation factor subunit 3 (CSTF3), HIV-1 Tat interactive protein 2 (HTATIP2), methyltransferase like 1 (METTL1), and any combination thereof; and (ii) a protein selected from the proteins listed in Table 8 (FIG. 27), and any combination of proteins listed in Table 8 (FIG. 27); thereby detecting the presence or absence of the protein of (i) and the protein of (ii) in the extracellular vesicle and particle protein sample. In accordance with this methods of the present disclosure, detecting the presence of one or more proteins from (i) and the absence of a one or more proteins from (ii) (i.e., Table 8 as shown in FIG. 27) in the extracellular vesicle and particle protein sample identifies lung cancer in the subject. Alternatively, detecting the absence of one or more proteins from (i) and the presence of a one or more proteins from (ii) (Table 8; FIG. 27) in the extracellular vesicle and particle protein sample indicates the subject does not have lung cancer.

In another embodiment, this method of detecting the presence of lung cancer in a subject involves obtaining a tissue sample from the subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from the group consisting of Small nuclear ribonucleoprotein Sm D3 (SNRPD3), Four and a half LIM domains protein 2 (FHL2), 60S ribosomal protein L26 (RPL26), 60S ribosomal protein L22 (RPL22), ELAV-like protein 1 (ELAVL1), 5′-3′ exoribonuclease 2 (XRN2), ATP-dependent DNA/RNA helicase DHX36 (DHX36), DnaJ homolog subfamily C member 7 (DNAJC7), Oxidoreductase HTATIP2 (HTATIP2), Amidophosphoribosyltransferase (PPAT), and combinations thereof, and (ii) a proteins selected from the group consisting of Caveolae-associated protein 2 (CAVIN2), Na(+)/H(+) exchange regulatory cofactor NHE-RF2 (SLC9A3R2), Protein mab-21-like 4 (MAB21L4), Fructose-1,6-bisphosphatase 1 (FBP1), Heat shock 70 kDa protein 12B (HSPA12B), Sciellin (SCEL), Pulmonary surfactant-associated protein C (SFTPC), Caveolin-2 (CAV2), F-actin-uncapping protein LRRC16A (CARMIL1), Advanced glycosylation end product-specific receptor (AGER), Protein XRP2 (RP2), Specifically androgen-regulated gene protein (C1orf116), and combinations thereof, thereby detecting the presence or absence of the protein of (i) and the protein of (ii) in the extracellular vesicle and particle protein sample. In accordance with this method of the present disclosure, detecting the presence of one or more proteins from (i) and the absence of a one or more proteins from (ii) in the extracellular vesicle and particle protein sample identifies lung cancer in the subject. Alternatively, detecting the absence of one or more proteins from (i) and the presence of a one or more proteins from (ii) in the extracellular vesicle and particle protein sample indicates the subject does not have lung cancer. In some embodiments, this method involves detecting at least the presence or absence of HTATIP and PPAT.

Subjects suitable for screening in accordance with this method of the present disclosure are described supra. In any embodiment, the subject is one having a lung tumor, where the status of the tumor, i.e., benign or malignant, is unknown, and the method is utilized to identify the status of the tumor.

In accordance with this method of the present disclosure, the tissue sample obtained from the subject is a lung tissue sample. In some embodiment, the lung tissue sample is a lung tumor tissue sample.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of lung cancer in the subject.

In some embodiments, at least one, at least two, at least three, at least four, at least 5, at least 6, at least 7, at least 8, at least 9, at least 10, or greater than 10 proteins from the proteins of group (ii) (i.e., Table 8) are subject to detection, and the presence of any one or more of these proteins in the sample indicates the absence of lung cancer in the subject.

When utilized together, the detection of one or more proteins of (i) and the absence of one or more proteins in (ii) is indicative of the presence of lung cancer in the subject. Alternatively, detecting the absence of one or more proteins of (i) and the presence of one or more proteins in (ii) is indicative that the subject does not have lung cancer.

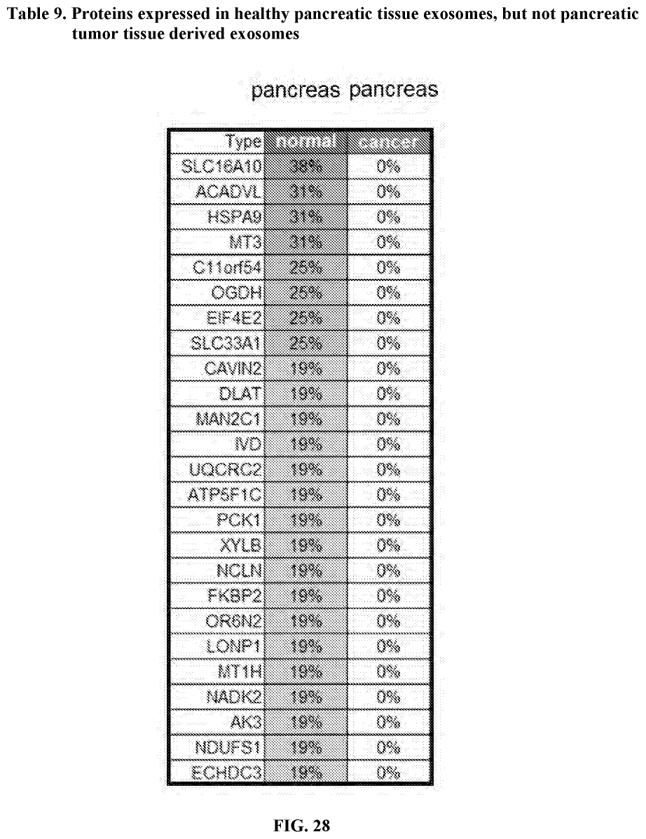

In another aspect, the present disclosure relates to a method of determining the presence of pancreatic cancer in a subject. In one embodiment, the method involves obtaining a tissue sample from a subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from, Myosin light polypeptide 6 (MYL6), EH domain-containing protein 1 (EHD1), Myosin-10 (MYH10), Fibronectin (FN1), Tropomyosin alpha-4 chain (TPM4), Flotillin-2 (FLOT2), Apolipoprotein A-I (APOA1), Thrombospondin-1 (THBS1), Tropomyosin alpha-3 chain (TPM3), Versican (VCAN), Dihydropyrimidinase-related protein 3 (DPYSL3), Actin-related protein 2/3 complex subunit 3 (ARPC3), Cathepsin B (CTSB), Thrombospondin-2 (THBS2), Coagulation factor XIII A chain (F13A1), Rho-related GTP-binding protein (RHOG), Myosin-9 (MYH9), Actin-related protein 2 (ACTR2), F-actin-capping protein subunit alpha-1 (CAPZA1), Actin-related protein 3 (ACTR3), Annexin A3 (ANXA3), Vimentin (VIM), Transitional endoplasmic reticulum ATPase (VCP), AP-2 complex subunit beta (AP2B1), Cytoplasmic dynein 1 heavy chain 1 (DYNC1H1), Vacuolar protein sorting-associated protein 35 (VPS35), High affinity immunoglobulin epsilon receptor subunit gamma (FCER1G), TB/POZ domain-containing protein KCTD12 (KCTD12), Guanine nucleotide-binding protein G(q) subunit alpha (GNAQ), Serpin H1 (SERPINH1), Ras-related protein Rab-31 (RAB31), Cytochrome b-245 heavy chain (CYBB), Protein S100-A13 (S100A13), Tropomyosin beta chain (TPM2), Milk fat globule-EGF factor 8 (MFGE8), Periostin (POSTN), Platelet-derived growth factor receptor beta, PDGF-R-beta (PDGFRB), Histidine-rich glycoprotein (HRG), Interferon-induced GTP-binding protein Mx1 (MX1), LIM and senescent cell antigen-like-containing domain protein 1 (LIMS1), Acyl-protein thioesterase 2 (LYPLA2), Inactive tyrosine-protein kinase 7 (PTK7), Ras-related protein Rab-22A (RAB22A), IST1 homolog (IST1), Raftlin (RFTN1), Plexin-B2 (PLXNB2), Vacuolar protein sorting-associated protein 28 homolog (VPS28), C-type mannose receptor 2 (MRC2), Neutrophil elastase (ELANE), Formin-like protein 1 (FMNL1), Cyclin-dependent kinase 4 (CDK4), Cyclin-dependent kinase 2 (CDK2), AP-2 complex subunit sigma (AP2S1), Prolyl endopeptidase FAP (FAP), Basigin (BSG), NADH-cytochrome b5 reductase 3 (CYB5R3), Fibulin-2 (FBLN2), Beta-hexosaminidase subunit beta (HEXB), Cyclin-dependent kinase 17 (CDK17), Tyrosine-protein kinase Lck (LCK), Retinoid-inducible serine carboxypeptidase (SCPEP1), Integrin alpha-X (ITGAX), Complement C1q subcomponent subunit B (C1QB), Macrophage-capping protein (CAPG), Osteoclast-stimulating factor 1 (OSTF1), Syntaxin-7 (STX7), Ectonucleoside triphosphate diphosphohydrolase 1 (ENTPD1), Neutrophil cytosol factor 2 (NCF2), Intercellular adhesion molecule 1 (ICAM1), Kinesin light chain 1 (KLC1), S-phase kinase-associated protein 1 (SKP1), Polyunsaturated fatty acid 5-lipoxygenase (ALOX5), Anoctamin-6 (ANO6), Metalloproteinase inhibitor 1 (TIMP1), 5′-AMP-activated protein kinase subunit gamma-1 (PRKAG1), Unconventional myosin-If (MYO1F), Mucin-5B (MUC5B), Alpha-1-antitrypsin (SERPINA1), and any combination thereof, and (ii) a protein selected from the list in Table 9 (FIG. 28) and any combination of proteins listed in Table 9 (FIG. 28); thereby detecting the presence or absence of the protein of (i) and the protein of (ii) in the extracellular vesicle and particle protein sample. In accordance with this method of the disclosure, detecting the presence of one or more proteins from (i) and the absence of a one or more proteins from (ii) identifies pancreatic cancer in the subject. Alternatively, detecting the absence of one or more proteins from (i) and the presence of a one or more proteins from (ii) indicates the subject does not have pancreatic cancer.

In another embodiment, the method of detecting the presence of pancreatic cancer in a subject involves obtaining a tissue sample from a subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting: (i) a protein selected from the group consisting of Protein S100-A9 (S100A9), Protein S100-A11 (S100A11), Protein S100-A13 (S100A13), Integrin alpha-6 (ITGA6), Integrin alpha-V (ITGAV), Versican (VCAN), Fibronectin (FN1), Annexin A1 (ANXA1), Annexin A3 (ANXA3), Cathepsin B (CTSB), Protein-glutamine gamma-glutamyltransferase 2 (TGM2), Complement decay-accelerating factor (CD55), Thymosin beta-10 (TMSB10), Syntenin-2 (SDCBP2), Fermitin family homolog 3 (FERMT3), Myosin-10 (MYH10), Myosin-14 (MYH14), Dihydropyrimidinase-related protein 3 (DPYSL3), Lactadherin (MFGE8), Inactive tyrosine-protein kinase 7 (PTK7), Dipeptidyl peptidase 1 (CTSC), Serpin B5 (SERPINB5), Epidermal growth factor receptor kinase substrate 8-like protein 1 (EPS8L1), Neutrophil cytosol factor 2 (NCF2), Metalloproteinase inhibitor 1 (TIMP1), Cathepsin S (CTSS), Glutamine synthetase (GLUL), Integrin alpha-L (ITGAL), Formin-like protein 1 (FMNL1), Intercellular adhesion molecule 1 (ICAM1), Vascular endothelial growth factor receptor 3 (FLT4), Platelet-derived growth factor receptor alpha (PDGFRA), Integrin alpha-X (ITGAX), Sequestosome-1 (SQSTM1), Retinoic acid-induced protein 3 (GPRC5A), Disintegrin and metalloproteinase domain-containing protein 9 (ADAM9), and combinations thereof, and (ii) one or more proteins selected from the group consisting of Syncollin (SYCN), Pancreatic lipase-related protein 2 (PNLIPRP2), Inactive pancreatic lipase-related protein 1 (PNLIPRP1), Phospholipase A2 (PLA2G1B), Chymotrypsin-like elastase family member 2B (CELA2B), Stress-70 protein, mitochondrial (HSPA9), Very long-chain specific acyl-CoA dehydrogenase, mitochondrial (ACADVL), and combinations thereof, thereby detecting the presence or absence of the protein of (i) and the protein of (ii) in the extracellular vesicle and particle protein sample. In accordance with this method of the disclosure, detecting the presence of one or more proteins from (i) and the absence of a one or more proteins from (ii) identifies pancreatic cancer in the subject. Alternatively, detecting the absence of one or more proteins from (i) and the presence of a one or more proteins from (ii) indicates the subject does not have pancreatic cancer. In some embodiments, this method involves detecting at least the presence or absence of one or more of CTSC, SERPINB5, EPS8L1, NCF2, TIMP1, CTSS, GLUL, ITGAL, FMNL1, ICAM1, FLT4, PDGFRA, ITGAX, SQSTM1, GPRC5A, ADAM9 (as indicators of the presence of pancreatic cancer), and HSPA9 and ACADVL (as indicators of the absence of pancreatic cancer).

Subjects suitable for screening in accordance with this method of the present disclosure are described supra. In any embodiment, the subject is one having a pancreatic lesion or tumor, where the status of the lesion or tumor, i.e., benign or malignant, is unknown, and the method is utilized to identify the status of the tumor.

In accordance with this method of the present disclosure, the tissue sample obtained from the subject is a pancreatic tissue sample. In some embodiments, the pancreatic tissue sample is a pancreatic tumor tissue sample.

In some embodiments, at least one, at least two, at least three, at least four, at least 5, at least 6, at least 7, at least 8, at least 9, at least 10, or greater than 10 proteins from the proteins of group (i) are subject to detection, and the detection of any one or more of these proteins in the sample indicates the presence of pancreatic cancer in the subject.

In some embodiments, at least one, at least two, at least three, at least four, at least 5, at least 6, at least 7, at least 8, at least 9, at least 10, or greater than 10 proteins from the proteins of group (ii) (i.e., Table 8) are subject to detection, and the presence of any one or more of these proteins in the sample indicates the absence of pancreatic cancer in the subject.

Another aspect of the present disclosure relates to a method of cancer sub-type identification. The method involves obtaining a tissue sample from a subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting at least three proteins selected from the group consisting of Apolipoprotein D (APOD), Polyubiquitin-C (UBC), Transaldolase (TALDO1), Thymidine phosphorylase (TYMP), Aminopeptidase B (RNPEP), Transgelin (TAGLN), Septin (SEPT7), Histone H2A type 2-B (HIST2H2AB), Gamma-enolase (ENO2), NADH-cytochrome b5 reductase 3 (CYB5R3), Actin-related protein 2/3 complex subunit 4 (ARPC4), Interleukin enhancer-binding factor 2 (ILF2), Protein transport protein Sec23B (SEC23B), COMM domain-containing protein 3 (COMMD3), Ankyrin-3 (ANK3), Glycogen phosphorylase, muscle form (PYGM), Putative histone H2B type 2-D (HIST2H2BD), Keratin, type I cytoskeletal 19 (KRT19), Sulfotransferase 1A2 (SULT1A2), Desmin (DES), Histone H2B (HIST1H2BD), Histone H2B type 1-A (HIST1H2BA), Histone H3.1t (HIST3H3), Tubulin beta-1 chain (TUBB1), Retinal dehydrogenase 2 (ALDH1A2), HLA class II histocompatibility antigen, DP beta 1 chain (HLA-DPB1), Bifunctional epoxide hydrolase 2 (EPHX2), Mitochondrial-processing peptidase subunit alpha (PMPCA), and Xylulose kinase (XYLB).

In accordance with this method as described herein, detecting the presence of at least three proteins described above identifies a tumor of unknown origin in the subject. The tumor of unknown origin may include a primary tumor, a metastasis, or a putative metastasis.

In any embodiment, at least three of the aforementioned proteins shown herein to be useful for identifying a cancer type from tissue-derived exosomes are detected. Alternatively, more than three of these proteins are detected. In any embodiment, the presence or absence of at least 4, at least 5, at least 6, at least 7, at least 8, at least 9, at least 10, or greater than 10 proteins of the proteins shown herein to be useful for identifying a cancer type from tissue-derived exosomal protein sample are detected. In one embodiment, the presence or absence of all of the proteins are detected as a result of said subjecting.

In one embodiment, this method is utilized to identify the origin of a primary tumor in a subject. Accordingly, the tissue sample is obtained from a metastatic cancer site. The origin of the primary tumor is identified by subjecting the tissue-derived extracellular vesicle and particle protein sample to one or more detection assays suitable to detect the presence or absence of at least three proteins selected from the group consisting of APOD, UBC, TALDO1, TYMP, RNPEP, TAGLN, SEPT7, HIST2H2AB, ENO2, CYB5R3, ARPC4, ILF2, SEC23B, COMMD3, ANK3, PYGM, HIST2H2BD, KRT19, SULT1A2, DES, HIST1H2BD, HIST1H2BA, HIST3H3, TUBB1, ALDH1A2, HLA-DPB1, EPHX2, PMPCA, and XYLB.

In a further embodiment, once the origin of a primary tumor in a subject is identified, an appropriate therapeutic drug known to treat that primary tumor is administered to the subject.

In one embodiment, the at least three proteins that are detected to identify the type of cancer present in the subject include immunoglobulin kappa variable 1-8 (IGKV1-8), immunoglobulin lambda constant 3 (IGLC3), and immunoglobulin heavy variable 3/OR16-13 (IGHV3OR16-13). In accordance with this embodiment, lung cancer is detected in the subject when the expression of IGKV1-8 is detected and expression of IGLC3 and IGHV3OR16-13 are not detected in the extracellular vesicle and particle protein sample. If lung cancer is detected and identified as the cancer type present in the subject, the subject can be administered one or more therapies suitable for treating the identified lung cancer. Suitable therapies for treating lung cancer are known in the art and described supra.

In one embodiment, the at least three proteins that are detected are selected from histone H2B type 1-D (HIST1H2BD), histone H2B type 1-A (HIST1H2BA), histone H3.1t (HIST3H3), tubulin beta-1 chain (TUBB1), retinal dehydrogenase 2 (ALDH1A2), HLA-DPB1, and polyubiquitin-C (UBC). In accordance with this embodiment, lung cancer is identified in the subject when expression of HIST1H2BD, HIST1H2BA, HIST3H3, TUBB1, ALDH1A2, HLA-DPB1 or any combination thereof is detected and expression of UBC is not detected in the extracellular vesicle and particle protein sample. If lung cancer is detected and identified as the cancer type present in the subject, the subject can be administered one or more therapies suitable for treating the identified lung cancer. Suitable therapies for treating lung cancer are known in the art and described supra.

In another embodiment, the at least three proteins that are detected are selected from apolipoprotein D (APOD), polyubiquitin-C (UBC), bifunctional epoxide hydrolase 2 (EPHX2), mitochondrial-processing peptidase subunit alpha (PMPCA), and xylulose kinase (XYLB). In accordance with this embodiment, pancreatic cancer is identified in the subject when expression of UBC, APOD, or any combination thereof are detected and expression of EPHX2, PMPCA, XYLB, or any combination thereof is not detected in the extracellular vesicle and particle protein sample. If pancreatic cancer is detected and identified as the cancer type present in the subject, the subject can be administered one or more therapies suitable for treating the identified pancreatic cancer. Suitable therapies for treating pancreatic cancer are known in the art and described supra.

In another embodiment, the at least three proteins that are detected are selected from SULT1A2, KRT19, HIST2H2BD, COMMD3, and ANK3. In accordance with this embodiment, melanoma is identified in the subject when expression of XYLB is detected and expression of SEPT7, COMMD3, ANK3, PYGM, or any combination thereof is not detected in the extracellular vesicle and particle protein sample. If melanoma is detected and identified as the cancer type present in the subject, the subject can be administered one or more therapies suitable for treating the identified melanoma. Suitable therapies for treating melanoma are known in the art and include, for example and without surgery (e.g., Mohs surgery); chemotherapeutics, including, but not limited to, Alimta (Pemetrexed Disodium), Ipilimumab Nivolumab, Opdivo (Nivolumab), Pemetrexed Disodium, Gemcitabine-Cisplatin combination; immunotherapeutics, including, without limitation, immune checkpoint inhibitors, e.g., PD-1 inhibitors (Pembrolizumab and nivolumab), PD-L1 inhibitor (e.g., Atezolizumab), and CTLA-4 inhibitor (e.g., Ipilimumab (Yervoy)); IL-2, oncolytic viruses (e.g. Talimogene laherparepvec (Imlygic)), Bacille Calmette-Guerin vaccine; targeted therapeutics, including, but not limited to BRAF inhibitors (e.g., Vemurafenib (Zelboraf), dabrafenib (Tafinlar), and encorafenib (Braftovi)), MEK inhibitors (e.g., trametinib (Mekinist), cobimetinib (Cotellic), and binimetinib (Mektovi)), C-Kit modulators (e.g., matinib (Gleevec) and nilotinib (Tasigna)).

In another embodiment, the at least three proteins that are detected are selected from COMMD3, ANK3, SULT1A2, KRT19, HIST2H2BD. In accordance with this embodiment, colorectal cancer is identified in the subject when expression of COMMD3 and/or ANK3 are detected and expression of SULT1A2, KRT19, HIST2H2BD, or any combination thereof is not detected. If colorectal cancer is detected and identified as the cancer type present in the subject, the subject can be administered one or more therapies suitable for treating the identified colorectal cancer. Suitable therapies for treating colorectal cancer are known in the art and described supra.

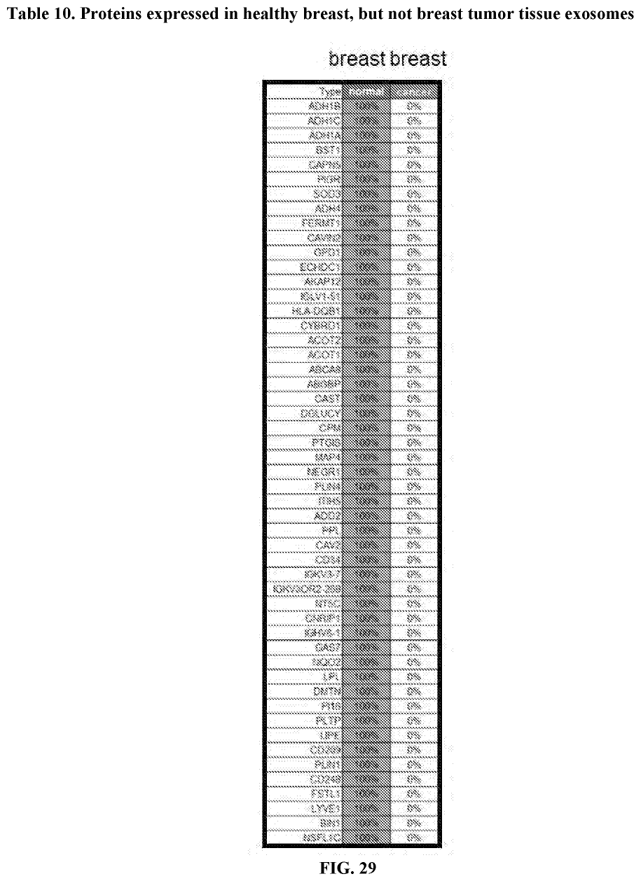

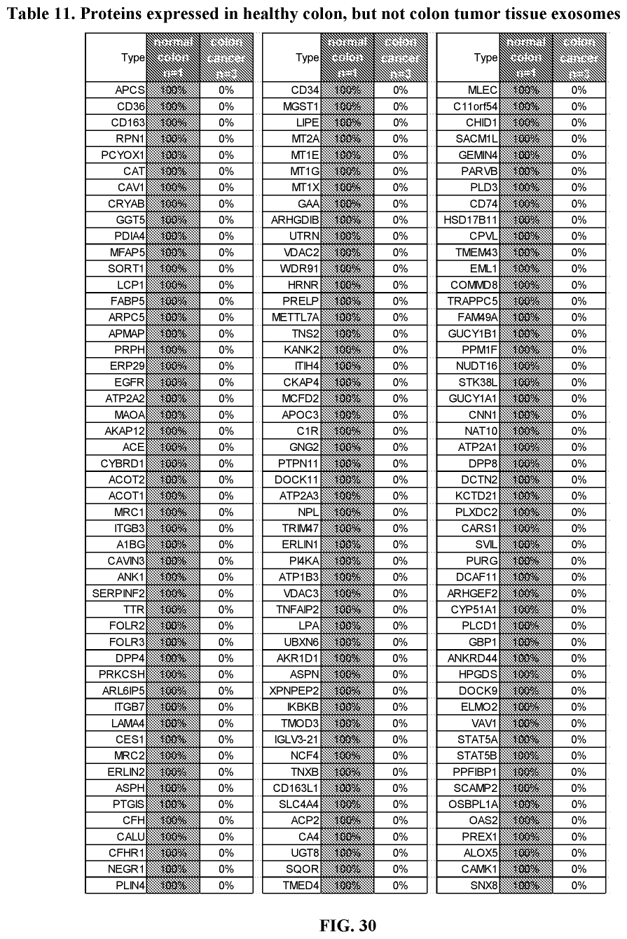

In another aspect, a breast tissue sample is obtained from a subject, extracellular vesicles and particles are separated from the tissue sample, and protein from the separated extracellular vesicles and particles is isolated to form an extracellular vesicle and particle protein sample. The extracellular vesicle and particle protein sample is subjected to a detection assay suitable for detecting one or more of the proteins listed in Table 10 (shown in FIG. 29).

In accordance with this method of the disclosure, detecting the presence or expression of one or more of the proteins listed in Table 10 (FIG. 29) is the tissue-derived extracellular vesicle and particle protein sample is indicative that the subject does not have breast cancer.

In some embodiments, at least one, at least two, at least three, at least four, at least 5, at least 6, at least 7, at least 8, at least 9, at least 10, or greater than 10 proteins from the proteins listed in Table 10 are subject to detection, and the detection of any one or more of these proteins in the sample indicates subject does not have breast cancer.

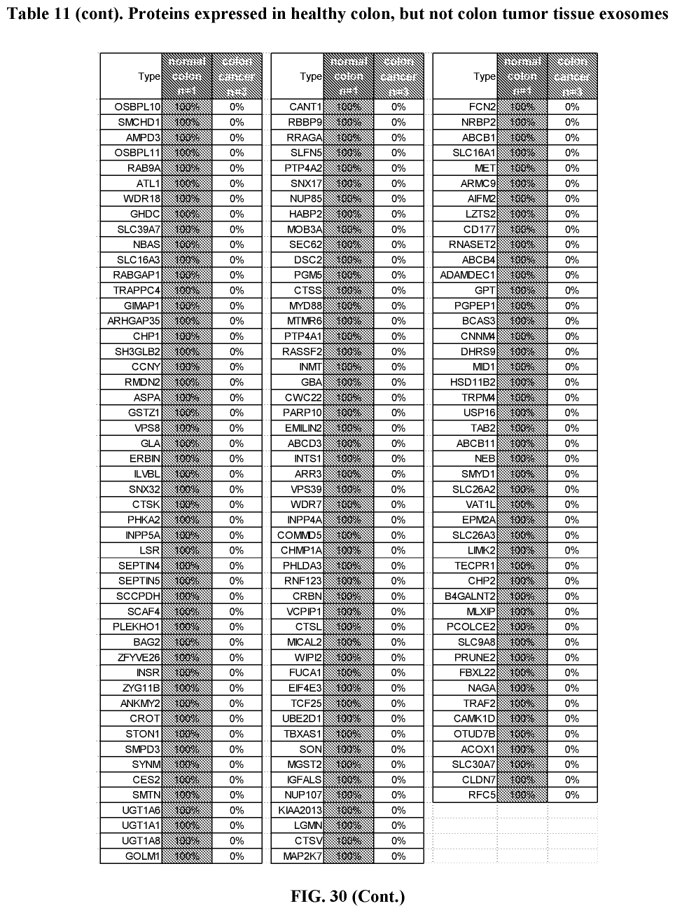

In yet another method of the present disclosure, a colon tissue sample is obtained from a subject, extracellular vesicles and particles are separated from the tissue sample, and protein from the separated extracellular vesicles and particles is isolated to form an extracellular vesicle and particle protein sample. The extracellular vesicle and particle protein sample is subjected to a detection assay suitable for detecting one or more of the protein listed in Table 11 (shown in FIG. 30).

In accordance with this aspect of the methods described herein, detecting the presence or expression of one or more of the proteins listed in Table 11 (FIG. 30) is indicative that the subject does not have colon cancer.

In some embodiments, at least one, at least two, at least three, at least four, at least 5, at least 6, at least 7, at least 8, at least 9, at least 10, or greater than 10 proteins from the proteins listed in Table 10 are subject to detection, and the detection of any one or more of these proteins in the sample indicates subject does not have colon cancer.

As discussed supra, once the origin of a primary tumor in a subject is identified, a therapeutic drug suitable for treating the primary tumor is administered to the subject.

In practicing the methods of the present disclosure, the administering step is carried out to achieve treatment of the identified tumor. Such administration can be carried out systemically or via direct or local administration to the primary tumor site. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterially, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. By way of example, intra-ommaya and intrathecal administration are suitable modes for direct administration into the brain for existing metastases. The mode of affecting delivery of agent will vary depending on the type of prophylactic agent (e.g., an antibody or small molecule).

The therapeutic drug may be orally administered, for example, with an inert diluent, or with an assimilable edible carrier, or it may be enclosed in hard or soft shell capsules, or it may be compressed into tablets, or they may be incorporated directly with the food of the diet. Therapeutic drugs may also be administered in a time release manner incorporated within such devices as time-release capsules or nanotubes. Such devices afford flexibility relative to time and dosage. For oral therapeutic administration, the agents may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit.

When the treatment is administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations of the therapeutic drug suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In addition to the formulations described previously, the therapeutic drug may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the therapeutic drug vary depending upon many different factors, including type and stage of the primary cancer, means of administration, target site, physiological state of the patient, other medications or therapies administered, and physical state of the patient relative to other medical complications. Treatment dosages need to be titrated to optimize safety and efficacy.

In yet another aspect, the present disclosure relates to a method of identifying a primary tumor of unknown origin. The method involves obtaining a tissue sample from a subject, separating extracellular vesicles and particles from the tissue sample, and isolating protein from the separated extracellular vesicles and particles to form an extracellular vesicle and particle protein sample. The extracellular vesicle and particle protein sample is subjected to a detection assay suitable for detecting one or more proteins independently selected from the proteins of Table 12, 13, 14, and 15. In accordance with this aspect, the tissue sample is obtained from a metastatic tumor or metastatic cancer site and analyzed to identify the origin of the primary tumor.

In one embodiment, the primary tumor of unknown origin is identified as a pancreatic tumor when one or more proteins from Table 12 is detected in the extracellular vesicle and particle protein sample. In accordance with this embodiment, the tissue sample from the subject is obtained from a metastatic cancer site, i.e., a non-pancreatic tumor tissue sample.

TABLE 12
Proteins expressed exclusively in pancreatic tissue and pancreatic cancer (19 proteins)

| | Type | | | | | | | |
|---|---|---|---|---|---|---|---|---|
| | normal | cancer | normal | cancer | normal | cancer | normal | cancer |
| | Origin | | | | | | | |
| | pancreas | pancreas | lung | lung | breast | breast | colon | colon |
| | N | | | | | | | |
| | n = 16 | n = 21 | n = 22 | n = 18 | n = 2 | n = 6 | n = 1 | n = 3 |
| PNLIP/ | 81% | 57% | 0% | 0% | 0% | 0% | 0% | 0% |
| Pancreatic | | | | | | | | |
| triacylglycerol lipase | | | | | | | | |
| CLPS/ | 81% | 43% | 0% | 0% | 0% | 0% | 0% | 0% |
| Colipase | | | | | | | | |
| CPB1/ | 81% | 38% | 0% | 6% | 0% | 0% | 0% | 0% |
| Carboxypeptidase B | | | | | | | | |
| CELA2B/ | 81% | 33% | 0% | 0% | 0% | 0% | 0% | 0% |
| Chymotrypsin-like | | | | | | | | |
| elastase family member | | | | | | | | |
| 2B | | | | | | | | |
| PNLIPRP1/ | 81% | 33% | 0% | 0% | 0% | 0% | 0% | 0% |
| Inactive pancreatic | | | | | | | | |
| lipase-related protein 1 | | | | | | | | |
| PNLIPRP2/ | 81% | 33% | 0% | 0% | 0% | 0% | 0% | 0% |
| Pancreatic lipase-related | | | | | | | | |
| protein 2 | | | | | | | | |
| SYCN/ | 81% | 24% | 0% | 0% | 0% | 0% | 0% | 0% |
| Syncollin | | | | | | | | |
| CEL/ | 75% | 48% | 0% | 0% | 0% | 0% | 0% | 0% |
| Bile salt-activated lipase | | | | | | | | |
| PLA2G1B/ | 75% | 33% | 0% | 0% | 0% | 0% | 0% | 0% |
| Phospholipase A2 | | | | | | | | |
| CTRB2/ | 63% | 57% | 0% | 0% | 0% | 0% | 0% | 0% |
| Chymotrypsinogen B2 | | | | | | | | |
| CTRB1/ | 56% | 52% | 0% | 0% | 0% | 0% | 0% | 0% |
| Chymotrypsinogen B | | | | | | | | |
| CPA2/ | 56% | 33% | 0% | 0% | 0% | 0% | 0% | 0% |
| Carboxypeptidase A2 | | | | | | | | |
| GPLD1/ | 38% | 33% | 0% | 0% | 0% | 0% | 0% | 0% |
| Phosphatidylinositol- | | | | | | | | |
| glycan-specific | | | | | | | | |
| phospholipase D | | | | | | | | |
| TFF2/ | 38% | 24% | 5% | 6% | 0% | 0% | 0% | 0% |
| Trefoil factor 2 | | | | | | | | |
| MUC6/ | 31% | 52% | 5% | 6% | 0% | 0% | 0% | 0% |
| Mucin-6 | | | | | | | | |
| GATM/ | 31% | 33% | 0% | 0% | 0% | 0% | 0% | 0% |
| Glycine | | | | | | | | |
| amidinotransferase, | | | | | | | | |
| mitochondrial | | | | | | | | |
| ERP27/ | 31% | 24% | 0% | 0% | 0% | 0% | 0% | 0% |
| Endoplasmic reticulum | | | | | | | | |
| resident protein 27 | | | | | | | | |
| GNMT/ | 25% | 33% | 0% | 0% | 0% | 0% | 0% | 0% |
| Glycine N- | | | | | | | | |
| methyltransferase | | | | | | | | |
| GCG/ | 25% | 33% | 0% | 0% | 0% | 0% | 0% | 0% |
| Pro-glucagon | | | | | | | | |

In another embodiment, the primary tumor of unknown origin is identified as a lung tumor when one or more proteins from Table 13 is detected in the extracellular vesicle and particle protein sample during said subjecting. In accordance with this embodiment, the tissue sample from the subject is obtained from a metastatic cancer site, i.e., a non-lung tumor tissue sample.

TABLE 13
Proteins expressed exclusively in lung tissue and lung cancer (21 proteins)

| | Type | | | | | | | |
|---|---|---|---|---|---|---|---|---|
| | normal | cancer | normal | cancer | normal | cancer | normal | cancer |
| | Origin | | | | | | | |
| | pancreas | pancreas | lung | lung | breast | breast | colon | colon |
| | N | | | | | | | |
| | n = 16 | n = 21 | n = 22 | n = 18 | n = 2 | n = 6 | n = 1 | n = 3 |
| SLC34A2/ | 0% | 0% | 100% | 78% | 0% | 0% | 0% | 0% |
| Sodium-dependent | | | | | | | | |
| phosphate transport | | | | | | | | |
| protein 2B | | | | | | | | |
| NAPSA/ | 0% | 0% | 100% | 78% | 0% | 0% | 0% | 0% |
| Napsin-A | | | | | | | | |
| SFTPB/ | 0% | 0% | 100% | 67% | 0% | 0% | 0% | 0% |
| Pulmonary surfactant- | | | | | | | | |
| associated protein B | | | | | | | | |
| SFTPA2/ | 0% | 0% | 100% | 61% | 0% | 0% | 0% | 0% |
| Pulmonary surfactant- | | | | | | | | |
| associated protein A2 | | | | | | | | |
| ABCA3/ | 0% | 0% | 95% | 56% | 0% | 0% | 0% | 0% |
| Phospholipid- | | | | | | | | |
| transporting ATPase | | | | | | | | |
| ABCA3 | | | | | | | | |
| TGM5/ | 0% | 0% | 68% | 50% | 0% | 0% | 0% | 0% |
| Protein-glutamine | | | | | | | | |
| gamma- | | | | | | | | |
| glutamyltransferase 5 | | | | | | | | |
| KYNU/ | 0% | 0% | 32% | 50% | 0% | 0% | 0% | 0% |
| Kynureninase | | | | | | | | |
| PKD1/ | 0% | 0% | 27% | 44% | 0% | 0% | 0% | 0% |
| Polycystin-1 | | | | | | | | |
| SCYL2/ | 0% | 0% | 27% | 39% | 0% | 0% | 0% | 0% |
| SCY1-like protein 2 | | | | | | | | |
| LRRK2/ | 0% | 0% | 91% | 33% | 0% | 0% | 0% | 0% |
| Leucine-rich repeat | | | | | | | | |
| serine/threonine- | | | | | | | | |
| protein kinase 2 | | | | | | | | |
| CACNA2D2/ | 0% | 0% | 86% | 33% | 0% | 0% | 0% | 0% |
| Voltage-dependent | | | | | | | | |
| calcium channel | | | | | | | | |
| subunit alpha-2/delta-2 | | | | | | | | |
| NUDT3/ | 0% | 0% | 32% | 33% | 0% | 0% | 0% | 0% |
| Diphosphoinositol | | | | | | | | |
| polyphosphate | | | | | | | | |
| phosphohydrolase 1 | | | | | | | | |
| AGER/ | 0% | 0% | 82% | 28% | 0% | 0% | 0% | 0% |
| Advanced | | | | | | | | |
| glycosylation end | | | | | | | | |
| product-specific | | | | | | | | |
| receptor | | | | | | | | |
| LIMCH1/ | 0% | 0% | 73% | 28% | 0% | 0% | 0% | 0% |
| LIM and calponin | | | | | | | | |
| homology domains- | | | | | | | | |
| containing protein 1 | | | | | | | | |
| DOK2/ | 0% | 0% | 41% | 28% | 0% | 0% | 0% | 0% |
| Docking protein 2 | | | | | | | | |
| SFTPC/ | 0% | 0% | 86% | 22% | 0% | 0% | 0% | 0% |
| Pulmonary surfactant- | | | | | | | | |
| associated protein C | | | | | | | | |
| SFTPD/ | 0% | 0% | 64% | 22% | 0% | 0% | 0% | 0% |
| Pulmonary surfactant- | | | | | | | | |
| associated protein D | | | | | | | | |
| PDE5A/ | 0% | 0% | 55% | 22% | 0% | 0% | 0% | 0% |
| cGMP-specific 3′,5′- | | | | | | | | |
| cyclic | | | | | | | | |
| phosphodiesterase | | | | | | | | |
| PREPL/ | 0% | 0% | 32% | 22% | 0% | 0% | 0% | 0% |
| Prolyl endopeptidase- | | | | | | | | |
| like | | | | | | | | |
| NMRK1/ | 0% | 0% | 27% | 22% | 0% | 0% | 0% | 0% |
| Nicotinamide riboside | | | | | | | | |
| kinase 1 | | | | | | | | |
| CPTP/ | 0% | 0% | 23% | 22% | 0% | 0% | 0% | 0% |
| Ceramide-1-phosphate | | | | | | | | |
| transfer protein | | | | | | | | |

In another embodiment, the primary tumor of unknown origin is identified as a breast tumor when one or more proteins from Table 14 is detected in the extracellular vesicle and particle protein sample during said subjecting. In accordance with this embodiment, the tissue sample from the subject is obtained from a metastatic cancer site, i.e., a non-breast tumor tissue sample.

TABLE 14
Proteins expressed exclusively in breast
tissue and breast cancer (5 proteins)

[symbol]

[symbol]

indicates data missing or illegible when filed

In another embodiment, the primary tumor of unknown origin is identified as a colon tumor when one or more proteins from Table 15 is detected in the extracellular vesicle and particle protein sample during said subjecting. In accordance with this embodiment, the tissue sample from the subject is obtained from a metastatic cancer site, i.e., a non-colon tumor tissue sample.

TABLE 15
Proteins expressed exclusively in colon tissue and colon cancer (4 proteins)

| | Type | | | | | | | |
|---|---|---|---|---|---|---|---|---|
| | normal | cancer | normal | cancer | normal | cancer | normal | cancer |
| | Origin | | | | | | | |
| | pancreas | pancreas | lung | lung | breast | breast | colon | colon |
| | N | | | | | | | |
| | n = 16 | n = 21 | n = 22 | n = 18 | n = 2 | n = 6 | n = 1 | n = 3 |
| FUT3/ | 0% | 0% | 0% | 0% | 0% | 0% | 100% | 33% |
| 3-galactosyl-N- | | | | | | | | |
| acetylglucosaminide | | | | | | | | |
| 4-alpha-L-fucosyltransferase | | | | | | | | |
| FUT3 | | | | | | | | |
| ST6GALNAC1 | 0% | 0% | 0% | 0% | 0% | 0% | 100% | 33% |
| Alpha-N-acetylgalactosaminide | | | | | | | | |
| alpha-2,6-sialyltransferase 1 | | | | | | | | |
| MUC12/ | 0% | 0% | 0% | 0% | 0% | 0% | 100% | 33% |
| Mucin-12 | | | | | | | | |
| LYPD8/ | 0% | 0% | 0% | 0% | 0% | 0% | 100% | 33% |
| Ly6/PLAUR domain- | | | | | | | | |
| containing protein 8 | | | | | | | | |

Another aspect of the present disclosure is directed to a method of isolating extracellular vesicles and particles from a biological sample. This method involves obtaining a biological sample from a subject and contacting the sample with one or more binding molecules, wherein each binding molecule is capable of binding to a target extracellular vesicle and particle protein. As disclosed herein, proteins that are selective for extracellular vesicles and particles include alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin. Thus, suitable binding molecules for carrying out this method including binding molecules, e.g., antibodies, that bind one of these aforementioned extracellular vesicle and particle proteins. The sample, after contacting with one or more binding molecules, is subjected to conditions effective for the one or more binding molecules to bind to its respective target extracellular vesicle and particle protein in the sample to form one or more binding molecule-target protein complexes. The one or more binding molecule-target protein complexes are separated from the sample, thereby isolating extracellular vesicles and particles from the sample.

Suitable binding molecules, e.g., antibodies and antibody based molecules, are described above.

In certain embodiments, the sample is contacted with at least two different binding molecules or with at least three different binding molecules.

In one embodiment, the sample is contacted with one or more binding molecules capable of binding to alpha-2-macroglobulin, moesin, and galectin-3-binding protein. In another embodiment, the sample is contacted with a binding molecule capable of binding alpha-2-macroglobulin, a binding molecule capable of binding moesin, and a binding molecule capable of binding galectin-3-binding protein.

In various related aspects, the present disclosure also relates to kits for performing the methods described herein. Such kits contain reagents and procedures that can be utilized in a clinical or research setting or adapted for either the field laboratory or on-site use. In particular, kits comprising the disclosed reagents used in practicing the methods described herein include any of a number of means for detecting the proteins of interest and measuring the presence or absence of such proteins, along with appropriate instructions, are contemplated. Suitable kits comprise reagents sufficient for performing an assay to detect a protein of interest including, without limitation, antibodies and fragments thereof.

It is to be understood that such a kit is useful for any of the methods of the present disclosure. The choice of particular components is dependent upon the particular method the kit is designed to carry out. Additional components can be provided for detection of the analytical output.

As described above, the kit optionally further comprises instructions for detecting the proteins of interest by the methods described herein. The instructions present in such a kit instruct the user on how to use the components of the kit to perform the various methods of the present disclosure. These instructions can include a description of the detection methods of the present disclosure.

Another aspect of the present disclosure is directed to a kit suitable for detecting, in a liquid biopsy sample from a subject, the presence of cancer in subject. The kit includes reagents, e.g., detectable binding molecules, suitable for detecting: (i) a protein selected from the group consisting of ferritin light chain, von Willebrand factor, immunoglobulin lambda constant 2, keratin 17, immunoglobulin heavy constant gamma 1, keratin 6B, radixin, cofilin 1, protease, serine 1, tubulin alpha 1c, ADAM metallopeptidase with thrombospondin type 1 motif 13, immunoglobulin kappa variable 6D-21, tyrosine 3-monooxygenase/tryptophan 5-monooxygenase activation protein theta, POTE ankyrin domain family member I, POTE ankyrin domain family member F, and immunoglobulin kappa variable 2D-30, and combinations thereof, and reagents suitable for detecting (ii) a protein selected from the group consisting of actin gamma 1, immunoglobulin lambda variable 3-27, immunoglobulin kappa variable 1D-12, coagulation factor XI, complement C1r subcomponent like, attractin, butyrylcholinesterase, immunoglobulin heavy variable 3-35, immunoglobulin kappa variable 1-17, C1q and TNF related 3, immunoglobulin heavy variable 3-20, immunoglobulin heavy variable 3/OR15-7, collectin subfamily member 11, immunoglobulin heavy constant delta, immunoglobulin kappa variable 3D-11, immunoglobulin heavy variable 3/OR16-10, immunoglobulin kappa variable 2D-24, immunoglobulin kappa variable 2-40, immunoglobulin kappa variable 1-27, immunoglobulin heavy variable 3/OR16-9, immunoglobulin lambda variable 5-45, immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6 and combinations thereof.

Another aspect of the present disclosure is directed to a kit suitable for detecting, in a liquid biopsy sample from a subject, the presence of cancer in the subject. The kit includes reagents suitable for detecting: (i) one or more proteins selected from the group consisting of Ferritin light chain (FTL), ABC-type oligopeptide transporter ABCB9 (ABCB9), Protein Z-dependent protease inhibitor (SERPINA10), Coagulation factor VIII (F8), Lactotransferrin (LTF), Basement membrane-specific heparan sulfate proteoglycan core protein (HSPG2), Protein disulfide-isomerase (P4HB), Trypsin-1 (PRSS1), Keratin, type II cytoskeletal 1b (KRT77), Endoplasmic reticulum chaperone BiP (HSPA5); and (ii) one or both proteins selected from the group consisting of Complement C1q tumor necrosis factor-related protein 3 (C1QTNF3) and Immunoglobulin heavy constant delta (IGHD). In some embodiments, the kit includes at least reagent for detecting the presence of one or more of LTF, HSPG2, P4HB, and PRSS1.

Another aspect of the present disclosure is directed to a kit suitable for detecting, in a tissue derived exosomal sample from a subject, the presence of cancer in a subject. The kit includes reagents suitable for detecting (i) a protein selected from the group consisting thrombospondin 2, versican, serrate, RNA effector molecule, tenascin C, dihydropyrimidinase like 2, adenosylhomocysteinase, DnaJ heat shock protein family (Hsp40) member A1, phosphoglycerate kinase 1, EH domain containing 2, and combinations thereof, and reagents suitable for detecting (ii) a protein selected from the group consisting of alcohol dehydrogenase 1B (class I), beta polypeptide, caveolae associated protein 1, FGGY carbohydrate kinase domain containing, ATP binding cassette subfamily A member 3, syntaxin 11, caveolae associated protein 2, CD36 molecule, and combinations thereof.

Another aspect of the present disclosure is directed to a kit suitable for detecting, in a tissue derived exosomal sample from a subject, the presence of cancer in the subject. The kit includes reagents suitable for detecting: (i) one or more proteins selected from the group consisting of tenacin (TNC), Periostin (POSTN), Versican core protein (VCAN), signal recognition particle 9 kDa protein (SRP9), Nucleophosmin (NPM1), Serrate RNA effector molecule homolog (SRRT), ELAV-like protein 1 (ELAVL1), Cytosolic acyl coenzyme A thioester hydrolase (ACOT7), 5′-3′ exoribonuclease 2 (XRN2), Flap endonuclease 1 (FEN1), ADP-ribosylation factor-like protein 1 (ARL1), Heat shock protein 105 kDa (HSPH1), Nucleolar RNA helicase 2 (DDX21), Src-associated in mitosis 68 kDa protein (KHDRBS1), Importin subunit alpha-1 (KPNA2), SLIT-ROBO Rho GTPase-activating protein 1 (SRGAP1), WD repeat-containing protein 3 (WDR3), and (ii) one or more proteins selected from the group consisting of Voltage-dependent calcium channel subunit alpha-2/delta-2 (CACNA2D2), Specifically androgen-regulated gene protein (C1orf116), Caveolin-2 (CAV2), Syntaxin-11 (STX11), Caveolae-associated protein 2 (CAVIN2). In some embodiments, the kit includes at least reagent for detecting the presence of one or more of KPNA2, SRGAP1, WDR3.

Another aspect of the present disclosure is directed to a kit suitable for detecting, in a liquid biopsy sample from a subject, the presence of pancreatic cancer in the subject. The kit includes reagents suitable for detecting: (i) one or more proteins selected from the group consisting of Calmodulin-like protein 5 (CALML5), Carboxypeptidase N subunit 2 (CPN2), Carbonic anhydrase 2 (CA2), Heat shock-related 70 kDa protein 2 (HSPA2), Lactotransferrin (LTF), GTPase KRas (KRAS), Complement decay-accelerating factor (CD55), Brain-specific angiogenesis inhibitor 1-associated protein 2-like protein 1 (BAIAP2L1), Phosphatidylethanolamine-binding protein 1 (PEBP1), Ras-related protein Rab-1A (RAB1A), Ras-related protein Rab-8B (RAB8B), Desmoplakin (DSP), Leucine-rich repeat-containing protein 26 (LRRC26), and (ii) one or more proteins selected from the group consisting of Thrombospondin-1 (THBS1), Complement C1r subcomponent-like protein (C1RL), Immunoglobulin kappa variable 1-6 (IGKV1.6), Immunoglobulin kappa variable 1-17 (IGKV1.17), Immunoglobulin kappa variable 1-39 (IGKV1.39), Immunoglobulin kappa variable 1-27 (IGKV1.27), Immunoglobulin kappa variable 1-12 (IGKV1.12), and Immunoglobulin kappa variable 1D-33 (IGKV1D.33). In some embodiments, the kit includes at least reagents for detecting the presence of one or more proteins selected from LTF, KRAS, CD55, BAIAP2L1, PEBP1, DSP, and LRRC26.

Another aspect of the present disclosure is directed to a kit suitable for detecting, in a tissue derived exosomal sample from a subject, the presence of pancreatic cancer in the subject. The kit includes reagents suitable for detecting: (i) one or more proteins selected from the group consisting of Protein S100-A9 (S100A9), Protein S100-A11 (S100A11), Protein S100-A13 (S100A13), Integrin alpha-6 (ITGA6), Integrin alpha-V (ITGAV), Versican (VCAN), Fibronectin (FN1), Annexin A1 (ANXA1), Annexin A3 (ANXA3), Cathepsin B (CTSB), Protein-glutamine gamma-glutamyltransferase 2 (TGM2), Complement decay-accelerating factor (CD55), Thymosin beta-10 (TMSB10), Syntenin-2 (SDCBP2), Fermitin family homolog 3 (FERMT3), Myosin-10 (MYH10), Myosin-14 (MYH14), Dihydropyrimidinase-related protein 3 (DPYSL3), Lactadherin (MFGE8), Inactive tyrosine-protein kinase 7 (PTK7), Dipeptidyl peptidase 1 (CTSC), Serpin B5 (SERPINB5), Epidermal growth factor receptor kinase substrate 8-like protein 1 (EPS8L1), Neutrophil cytosol factor 2 (NCF2), Metalloproteinase inhibitor 1 (TIMP1), Cathepsin S (CTSS), Glutamine synthetase (GLUL), Integrin alpha-L (ITGAL), Formin-like protein 1 (FMNL1), Intercellular adhesion molecule 1 (ICAM1), Vascular endothelial growth factor receptor 3 (FLT4), Platelet-derived growth factor receptor alpha (PDGFRA), Integrin alpha-X (ITGAX), Sequestosome-1 (SQSTM1), Retinoic acid-induced protein 3 (GPRC5A), Disintegrin and metalloproteinase domain-containing protein 9 (ADAM9), and (ii) one or more proteins selected from the group consisting of Syncollin (SYCN), Pancreatic lipase-related protein 2 (PNLIPRP2), Inactive pancreatic lipase-related protein 1 (PNLIPRP1), Phospholipase A2 (PLA2G1B), Chymotrypsin-like elastase family member 2B (CELA2B), Stress-70 protein, mitochondrial (HSPA9), Very long-chain specific acyl-CoA dehydrogenase, mitochondrial (ACADVL). In some embodiments, the kit includes at least reagents for detecting the presence of one or more proteins selected from CTSC, SERPINB5, EPS8L1, NCF2, TIMP1, CTSS, GLUL, ITGAL, FMNL1, ICAM1, FLT4, PDGFRA, ITGAX, SQSTM1, GPRC5A, ADAM9, HSPA9, and ACADVL.

Another aspect of the present disclosure is directed to a kit suitable for detecting, in a liquid biopsy sample from a subject, the presence of lung cancer in the subject. The kit includes reagents suitable for detecting: (i) one or more proteins selected from the group consisting of Putative alpha-1-antitrypsin-related protein (SERPINA2), Immunoglobulin kappa joining 1 (IGKJ1), Protein 4.2 (EPB42), Histone H2A type 1-D (H2AC7), Proteasome subunit alpha type-2 (PSMA2), Nebulette (NEBL), Tripeptidyl-peptidase 2 (TPP2), Monocyte differentiation antigen CD14 (CD14), Fc receptor-like protein 3 (FCRL3), Charged multivesicular body protein 4b (CHMP4B), Rho-related GTP-binding protein RhoV (RHOV), Leukocyte surface antigen CD53 (CD53), Basement membrane-specific heparan sulfate proteoglycan core protein (HSPG2), Trypsin-1 (PRSS1), and (ii) Transforming growth factor-beta-induced protein ig-h3 (TGFBI). In some embodiments, the kit includes at least reagents for detecting the presence of one or more proteins selected from CHMP4B, RHOV, CD53, HSPG2, and PRSS1.

Another aspect of the present disclosure is directed to a kit suitable for detecting, in a tissue derived exosomal sample from a subject, the presence of lung cancer in the subject. The kit includes reagents suitable for detecting: (i) one or more proteins selected from the group consisting of Small nuclear ribonucleoprotein Sm D3 (SNRPD3), Four and a half LIM domains protein 2 (FHL2), 60S ribosomal protein L26 (RPL26), 60S ribosomal protein L22 (RPL22), ELAV-like protein 1 (ELAVL1), 5′-3′ exoribonuclease 2 (XRN2), ATP-dependent DNA/RNA helicase DHX36 (DHX36), DnaJ homolog subfamily C member 7 (DNAJC7), Oxidoreductase HTATIP2 (HTATIP2), Amidophosphoribosyltransferase (PPAT), and (ii) one or more proteins selected from the group consisting of Caveolae-associated protein 2 (CAVIN2), Na(+)/H(+) exchange regulatory cofactor NHE-RF2 (SLC9A3R2), Protein mab-21-like 4 (MAB21L4), Fructose-1,6-bisphosphatase 1 (FBP1), Heat shock 70 kDa protein 12B (HSPA12B), Sciellin (SCEL), Pulmonary surfactant-associated protein C (SFTPC), Caveolin-2 (CAV2), F-actin-uncapping protein LRRC16A (CARMIL1), Advanced glycosylation end product-specific receptor (AGER), Protein XRP2 (RP2), Specifically androgen-regulated gene protein (C1orf116). In some embodiments, the kit includes at least reagents for detecting the presence of one or more proteins selected from HTATIP2 and PPAT.

Another aspect of the present disclosure is directed to a kit suitable for identifying the origin of a tumor from a liquid biopsy. The kit includes reagents, i.e., binding molecules, suitable for detecting at least three proteins selected from the group consisting of Fibrinogen beta chain (FGB), FGA (Fibrinogen alpha chain), Fibrinogen gamma chain (FGG), Complement factor H (CFH), Plasminogen (PLG), Immunoglobulin heavy variable 3-53 (IGHV3-53), Serum amyloid P-component, SAP (APCS), Complement factor H-related protein 1 (CFHR1), Immunoglobulin heavy variable 3-48 (IGHV3-48), Immunoglobulin heavy variable 3-74 (IGHV3-74), Immunoglobulin heavy variable 3-72 (IGHV3-72), Immunoglobulin heavy variable 3-43 (IGHV3-43), Immunoglobulin heavy variable 5-10-1 (IGHV5-10-1), Immunoglobulin lambda variable 7-46 (IGLV7-46), Immunoglobulin kappa variable 3D-20 (IGKV3D-20), Immunoglobulin kappa variable 2-24 (IGKV2-24), Complement factor H-related protein 2 (CFHR2), Immunoglobulin heavy variable 4-59 (IGHV4-59), Immunoglobulin heavy variable 3-20 (IGHV3-20), Immunoglobulin heavy variable 3-64 (IGHV3-64), Probable non-functional immunoglobulin heavy variable 3-16 (IGHV3-16), Immunoglobulin heavy variable 3-11 (IGHV3-11), Immunoglobulin heavy variable 3/OR16-9 (IGHV3OR16-9), Probable non-functional immunoglobulin kappa variable 2D-24 (IGKV2D-24), Immunoglobulin lambda constant 3 (IGLC3), Immunoglobulin heavy variable 3/OR16-13 (IGHV3OR16-13), Complement factor H-related protein 3 (CFHR3), Immunoglobulin heavy constant gamma 3 (IGHG3), Immunoglobulin lambda constant 2 (IGLC2), and Immunoglobulin kappa variable 1-8 (IGKV1-8). In a preferred embodiment, the binding molecules of the kit are antibodies that have binding specificity for the aforementioned proteins. Suitable antibodies are known in the art.

Another aspect of the present disclosure is directed to a kit suitable for identifying the origin of a tumor from a tissue biopsy. The kit includes reagents, i.e., binding molecules, suitable for detecting at least three proteins selected from the group consisting of APOD, UBC, TALDO1, TYMP, RNPEP, TAGLN, SEPT7, HIST2H2AB, ENO2, CYB5R3, ARPC4, ILF2, SEC23B, COMMD3, ANK3, PYGM, HIST2H2BD, KRT19, SULT1A2, DES, HIST1H2BD, HIST1H2BA, HIST3H3, TUBB1, ALDH1A2, HLA-DPB1, EPHX2, PMPCA, and XYLB. In a preferred embodiment, the binding molecules of the kit are antibodies that having binding specificity for the aforementioned proteins. Suitable antibodies are known in the art.

Another aspect of the present disclosure is directed to a kit suitable for identifying the origin of a metastatic tumor from a tissue biopsy. The kit includes reagents, i.e., binding molecules, suitable for detecting at least one or more proteins selected from the proteins listed in Tables 12, 13, 14, and 15. In a preferred embodiment, the binding molecules of the kit are antibodies that having binding specificity for the proteins listed in Tables 12, 13, 14, and 15. Suitable antibodies are known in the art.

Another aspect of the present disclosure is directed to a kit suitable for isolating exosomes from a human sample. The kit includes at least one binding molecule capable of binding a protein selected from the group of proteins consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin. In a preferred embodiment, the binding molecules of the kit are antibodies that having binding specificity for the aforementioned proteins. Suitable antibodies are known in the art.

In one embodiment, the kit comprises at least three different binding molecules, each binding molecule capable of binding a different protein in the group of proteins consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin. In a preferred embodiment, the binding molecules of the kit are antibodies that having binding specificity for the aforementioned proteins. Suitable antibodies are known in the art.

In accordance with this embodiment, the at least three different binding molecules comprise a binding molecule capable of binding to alpha-2-macroglobulin, a binding molecule capable of binding moesin, and a binding molecule capable of binding galectin-3-binding protein.

Another aspect of the present disclosure is directed to a method of determining a treatment regimen for a subject having a tumor. The method involves obtaining, from the subject having the tumor, a biopsy of tumor tissue and a biopsy of tissue adjacent to the tumor, and separating extracellular vesicles and particles from the obtained samples. Protein from the separated extracellular vesicle and particles is isolated to form extracellular vesicle and particle protein samples, and the extracellular vesicle and particle protein samples are subjected to a detection assay suitable for detecting proteins differentially expressed in the tumor tissue versus adjacent, non-tumor tissue. A treatment regimen for the subject is identified based on said subjecting, i.e., based on the differential protein expression between tumor tissue and tissue adjacent the tumor.

The term “treatment” refers to administration of a therapy to a patient having a tumor, where the therapy is administered in manner effective to inhibit growth of the tumor or inhibit or prevent metastasis from occurring. Treatment as used herein also encompasses treatment that is effective to delay, slow, or lessen the severity of a primary tumor or metastasis.

In one embodiment, the extracellular vesicle and particle protein sample is subjected to a detection assay suitable for detecting one or more proteins selected from the group consisting of versican (VCAN), cathepsin B (CTSB), thrombospondin 2 (THBS2), septin 9 (SEPTIN9), basigin (BSG), fibulin 2 (FBLN2), four and a half LIM domains 2 (FHL2), inosine triphosphatase (ITPA), galectin-9 (LGALS9), splicing factor 3b subunit 1 3 (SF3B3) and calcium/calmodulin dependent serine protein kinase (CASK).

In another embodiment, the extracellular vesicle and particle protein sample is subjected to a detection assay suitable for detecting one or more proteins selected from the group consisting of HIV-1 Tat interactive protein 2 (HTATIP2) and methyltransferase like 1 (METTL1).

In a further embodiment, the subject has pancreatic cancer and the extracellular vesicle and particle protein sample is subjected to a detection assay suitable for detecting one or more proteins selected from the group consisting of FLOT2, TPM3, FCER1G, GNAQ, RAB31, CYBB, S100A13, TPM2, MFGE8, POSTN, PDGFRB, HRG, MX1, LIMS1, LYPLA2, PTK7, RAB22A, IST1, RFTN1, PLXNB2, VPS28, MRC2, ELANE, FMNL1, CDK4, CDK2, AP2S1, FAP, BSG, CYB5R3, FBLN2, HEXB, CDK17, LCK, SCPEP1, ITGAX, C1QB, CAPG, OSTF1, STX7, ENTPD1, NCF2, ICAM1, KLC1, SKP1, ALOX5, ANO6, TIMP1, PRKAG1, and MYO1F.

Another aspect of the present disclosure is directed to a method of identifying drug targets for cancer therapy. The method involves obtaining, from each of a plurality of subjects having a particular tumor, a biopsy of tumor tissue and a biopsy of tissue adjacent to said tumor, and separating extracellular vesicles and particles from the obtained samples. Protein from the separated extracellular vesicle and particles is isolated to form extracellular vesicle and particle protein samples, and the extracellular vesicle and particle protein samples is subjected to proteomic analysis to identify proteins differentially expressed in the tumor tissue versus tissue adjacent said tumor. Drug targets for cancer therapy are identified based on said subjecting.

In accordance with this aspect, to make an impact on drug development strategies, the extensive dataset described herein is able to identify tumor-specific EVP proteins that could be targeted with minimal side effects for normal tissues. Thus, it is important to identify tumor tissue extracellular vesicle and particle proteins that were not present in adjacent tissue and distant tissue extracellular vesicle and particles in the organ of interest.

Another aspect of the present disclosure is directed to a method of treating a subject having a tumor. The method involves selecting a subjecting having a tumor, wherein exosomes from the tumor tissue express nucleolin (NCL), and administering to said subject a nucleolin inhibitor.

Methods and modes of administration are described above.

In accordance with this embodiment, any suitable nucleolin inhibitor known in the art can be administered to the subject. In one embodiment, the nucleolin inhibitor is AGR100 (AS1411).

Another aspect of the present disclosure is directed to a method of treating a subject having a tumor. The method involves administering a tenacin inhibitor to a subject having a tumor, wherein exosomes from the tumor tissue of the subject express tenacin (TNC).

Methods and modes of administration are described above.

In accordance with this embodiment, any suitable tenacin inhibitor known in the art can be administered to the subject. In one embodiment, the tenacin inhibitor is an F16-IL2 fusion protein.

Another aspect of the present disclosure is directed to a method of treating a subject having a tumor. The method involves administering an inosine-5′-monophosphate dehydrogenase 2 inhibitor to a subject having a tumor, wherein exosomes from the tumor tissue of the subject express inosine-5′-monophosphate dehydrogenase 2 (IMPDH2).

Methods and modes of administration are described above.

In accordance with this embodiment, any suitable inosine-5′-monophosphate dehydrogenase 2 inhibitor known in the art can be administered to the subject. In one embodiment, the inosine-5′-monophosphate dehydrogenase 2 inhibitor is selected from the group consisting of mycophenolic acid, thioguanine, mycophenolate mofetil, imatinib/thioguanine, VX-944, pegintron/ribavirin, mycophenolate mofetil/prednisone, methylprednisolone/mycophenolate mofetil, interferon alfacon-1/ribavirin, 6-mercaptopurine/prednisone/thioguanine, cytarabine/daunorubicin/thioguanine, cytarabine/thioguanine, IFNA2B/ribavirin, and ribavirin.

Another aspect of the present disclosure is directed to a method of treating a subject having a tumor. The method involves administering a glutamine amidotransferase inhibitor to a subject having a tumor, wherein exosomes from the tumor tissue of the subject express GMP synthase (GMPS).

Methods and modes of administration are described above.

In accordance with this embodiment, any suitable glutamine admidotransferase inhibitor known in the art can be administered to the subject. In one embodiment, the glutamine admidotransferase inhibitor is azaserine.

Another aspect of the present disclosure is directed to a method of treating a subject having a tumor. The method involves administering a DNA topoisomerase I inhibitor to a subject having a tumor, wherein exosomes from the tumor tissue of the subject express DNA topoisomerase I (TOP1MT).

Methods and modes of administration are described above.

In accordance with this embodiment, any suitable DNA topoisomerase I inhibitor known in the art can be administered to the subject. In one embodiment, the is selected from the group consisting of capecitabine/cetuximab/irinotecan, irinotecan/leucovorin, cetuximab/irinotecan, gemcitabine/irinotecan, aflibercept/irinotecan, capecitabine/irinotecan, cetuximab/irinotecan/vemurafenib, and irinotecan.

Another aspect of the present disclosure is directed to a method of treating a subject having a tumor. The method involves administering an ATIC inhibitor to a subject having a tumor, wherein exosomes from the tumor tissue of the subject express bifunctional purine biosynthesis protein ATIC (ATIC).

Methods and modes of administration are described above.

In accordance with this embodiment, any suitable ATIC inhibitor known in the art can be administered to the subject. In one embodiment, the ATIC inhibitor is selected from the group consisting of pemetrexed, pembrolizumab/pemetrexed, and gemcitabine/pemetrexed.

Another aspect of the present disclosure is directed to a method of treating a subject having a tumor. The method involves selecting a subjecting having a tumor, wherein exosomes from the tumor tissue express aldo-keto reductase family 1 member B1 (AKR1B1), and administering to said subject an aldo-keto reductase family 1 member B1 inhibitor.

Methods and modes of administration are described above.

In accordance with this embodiment, any suitable aldo-keto reductase family 1 member B1 inhibitor known in the art can be administered to the subject. In one embodiment, the aldo-keto reductase family 1 member B1 inhibitor is selected from the group consisting of pemetrexed, pembrolizumab/pemetrexed, and gemcitabine/pemetrexed.

Another aspect of the present disclosure is directed to a method of treating a subject having a tumor. The method involves administering to a subject having a tumor a cytokeratin-2e inhibitor, wherein plasma tumor derived exosomes of the subject express cytokeratin-2e (KRT2).

Methods and modes of administration are described above.

In accordance with this embodiment, any suitable cytokeratin-2e inhibitor known in the art can be administered to the subject. In one embodiment, the cytokeratin-2e inhibitor is selected from the group consisting of CIGB-300 and silmitasertib.

Another aspect of the present disclosure is directed to a method of treating a subject having a tumor. The method involves administering a coagulation factor VIII inhibitor to a subject having a tumor, wherein plasma tumor derived exosomes of the subject express coagulation factor VIII (F8).

Methods and modes of administration are described above.

In accordance with this embodiment, any suitable coagulation factor VIII inhibitor known in the art can be administered to the subject. In one embodiment, the coagulation factor VIII inhibitor is drotrecogin alfa (recombinant human activated protein C) or recombinant coagulation factor IX.

Another aspect of the present disclosure is directed to a method of treating a subject having a tumor. The method involves administering a peptidyl-prolyl cis-trans isomerase A inhibitor to a subject having a tumor, wherein plasma tumor derived exosomes of the subject express peptidyl-prolyl cis-trans isomerase A (PPIA).

Methods and modes of administration are described above.

In accordance with this embodiment, any suitable peptidyl-prolyl cis-trans isomerase A inhibitor known in the art can be administered to the subject. In one embodiment, the peptidyl-prolyl cis-trans isomerase A inhibitor is selected from the group consisting of cyclosporine A/sirolimus/tacrolimus, N-methyl-4-Ile-cyclosporin, alemtuzumab/cyclosporin A, cyclosporin A, cyclosporine A/tacrolimus, and cyclosporin A/methotrexate.

Another aspect of the present disclosure is directed to a method of treating a subject having a tumor. The method involves administering a carbonic anhydrase I inhibitor to a subject having a tumor, wherein plasma tumor derived exosomes of the subject express carbonic anhydrase I (CA1).

Methods and modes of administration are described above.

In accordance with this embodiment, any suitable carbonic anhydrase I inhibitor known in the art can be administered to the subject. In one embodiment, the carbonic anhydrase I inhibitor is selected from the group consisting of benzthiazide, ethoxyzolamide, brimonidine/brinzolamide, dorzolamide, diazoxide, dichlorphenamide, methazolamide, hydrochlorothiazide, sulfacetamide, dorzolamide/timolol, brinzolamide, topiramate, chlorothiazide/reserpine, chlorothiazide, chlorthalidone, acetazolamide, quinethazone, and trichloromethiazide.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

### EXAMPLES

The following examples are provided to illustrate embodiments of the present disclosure but they are by no means intended to limit its scope.

### Materials and Methods for Examples 1-10

Cell lines and cell culture. B16-F10, B16-F1, 4T1, MDA-MB-231 series (parental, -1833, -4175 and -831; -4173 and -4180; 231BR), SW620, HCT116 (Horizon Discovery), uveal melanoma, 131/4-5B2 and 131/8-2L, CCG9911 and CLS1, MCF10A, MDA-MB-468, VCAP, HLIEC, HT29, MiaPaca2, Kasumi, SNU1, SNU16, CLS1, LNCaP, human rhabdomyosarcoma CT10 and RD, human osteosarcoma Saos-2 and U2OS and human Ewing sarcoma SK-NP-DW, PaCa cell lines PANC-1, AsPC-1, Pan02 (purchased from the National Cancer Institute Tumor Repository), and NIH3T3 cell were cultured in DMEM, supplemented with penicillin (100 U/ml), streptomycin (100 μg/ml) and 10% FBS. Human melanoma cells (SK-Mel03, A375M and A375P were obtained from MSKCC), human prostatic carcinoma cell lines PC3 and DU145, as well as human PaCa cell lines BXPC-3, HPAF-II, human LuCa cell lines LLC, PC-9, H1650, H1975, H292, H358, H2228, A549, 1118A and ET2B, human leukemia cell line Nalm6, K-562 (DSMZ) and NB-4 (DSMZ) cells and murine breast cancer cell line E0771 were cultured in RPMI, supplemented with penicillin (100 U/ml), streptomycin (100 g/ml) and 10% FBS. Human breast cancer cell line SK-1 BR-3 was cultured in McCoy's 5a Medium Modified, supplemented with penicillin (100 U/ml), streptomycin (100 μg/ml) and 10% FBS. WI-38 cells were cultured in MEM alpha, supplemented with penicillin (100 U/ml), streptomycin (100 μg/ml) and 10% FBS. Primary HMEC strains were generated and maintained as described (Labarge et al., “Processing of Human Reduction Mammoplasty and Mastectomy Tissues for Cell Culture,” J Vis Exp. 71:50011 (2013), which is hereby incorporated by reference in its entirety). Human mammary epithelia were derived from discarded reduction mammoplasty tissue in accordance with applicable legal and ethical standards per the internal review board at City of Hope; IRB #15418. Human mammary epithelial cells and fibroblasts cell line N253_LEP, N253_MEP, N255_MEP, N274_fibroblast and N274_MEP were cultured in DMEM/F12, supplemented with penicillin (100 U/ml), streptomycin (100 μg/ml) and 10% FBS. Human osteosarcoma cell line 143B, human Ewing sarcoma cell line SKES1, human neuroblastoma SK-N-BE(2) and IMR5 were cultured in RPMI, supplemented with penicillin (100 U/ml), streptomycin (100 μg/ml), non-essential amino acids, sodium pyruvate, Hepes, and 10% FBS. S1 and T4-2 cells were grown in H14 medium on collagen-coated tissue culture flasks. HepG were cultured in collagen-coated plates in DMEM, supplemented with 10% FBS. Cell lines not otherwise mentioned were obtained from American Type Culture Collection. For human cell lines, authentication using STR profiling by commercial providers were done. Mycoplasma testing by ATCC test kit were performed prior to exosome isolation for all of the cell lines. All cells were maintained in a humidified incubator with 5% CO₂ at 37° C. and routinely tested and confirmed to be free of mycoplasma contamination. When collecting conditioned media for exosome isolation, FBS (Gibco, Thermo Fisher Scientific) was first depleted of exosomes by ultracentrifugation at 100,000×g for 90 minutes. Cells were cultured for 3-4 days before supernatant collection.

Human specimens and processing. Fresh human tumor tissues were obtained at Memorial Sloan Kettering Cancer Center (MSKCC). All individuals provided informed consent for tissue donation according to a protocol approved by the institutional review board of MSKCC (IRB 11-033A, MSKCC; IRB 0604008488, WCM). The study is compliant with all relevant ethical regulations regarding research involving human participants.

Tissue samples. Fresh tumor and peritumoral adjacent tissue were collected from patients with localized PaCa undergoing resection with curative intent (either pancreaticoduodenectomy or distal pancreatectomy) at MSKCC. The tissue was placed in ice-cold PBS within minutes of collection and submitted for downstream processing and analysis. The pancreatic tissue collection was conducted through the Tumor Procurement Service (TBS), Department of Pathology, MSKCC. TPS separated a biopsy of tumor tissue and procured a separate biopsy of peritumoral non-involved pancreas (AT) wherever there was a sufficient resection margin. Tissues were cut into small pieces and cultured for 24 h in serum-free RPMI, supplemented with penicillin (100 U/ml) and streptomycin (100 μg/ml). Conditioned media was processed for exosome isolation with final step using sucrose cushion. LuCa, breast cancer, colorectal cancer, DSRCT, epithelioid sarcoma, fibrolamellar sarcoma, fibromeller HCC, hepatoblastoma, immature teratoma, renal cell carcinoma, melanoma, MPNST, neuroblastoma, osteosarcoma, rhabdomyosarcoma, synovial sarcoma and Wilms' tumor were collected from patients undergoing resection at MSKCC. Tissues were cut into small pieces and cultured for 24 h in serum-free RPMI, supplemented with penicillin (100 U/ml) and streptomycin (100 g/ml). Conditioned media was processed for exosome isolation.

Human melanoma lymphatic fluid. Lymph fluid was collected after radical lymphadenectomy from routinely used sucking drainage. To ensure that the sample of lymph fluid did not contain any surgical debris, only the fluid between 24 and 48 hours was collected (the first 24 hour batch was discarded). Samples were centrifuged (500×g, 10 minutes), and the supernatant was collected and stored at −80° C. for exosome isolation.

Human bile duct fluid. With the approval of the MSKCC IRB, a bile bank was established in 2010 and prospectively maintained for patients undergoing resection of hepatopancreatobiliary cancer. Bile was collected for the bank by needle cannulation of the common bile duct at the time of operation. Patients had pathologically confirmed extra-hepatic cholangiocarcinoma when the bile was collected. Bile was snap-frozen in liquid nitrogen and stored at −80° C. until analysis. One milliliter of bile from each patient was used for exosome isolation and analysis. One milliliter of ice-cold PBS was added to each thawed bile fluid, and the mixture was homogenized with repeated pipetting followed by exosome isolation.

EVP purification, characterization and analyses. Exosomes were purified by sequential centrifugation, as previously described (Hoshino et al., “Tumour Exosome Integrins Determine Organotropic Metastasis,” Nature 527:329-335 (2015), which is hereby incorporated by reference in its entiretyjhn). In brief, cell contamination was removed from 3-4 day cell culture supernatant, bodily fluids or resected tissue culture supernatant by centrifugation at 500×g for 10 minutes. To remove apoptotic bodies and large cell debris, the supernatants were then spun at 3,000×g for 20 minutes, followed by centrifugation at 12,000×g for 20 minutes to remove large microvesicles. Finally, exosomes were collected by spinning at 100,000×g for 70 minutes. Exosomes were washed in PBS and pelleted again by ultracentrifugation in a Beckman Coulter Optima XE or XPE ultracentrifuge. The final exosome pellet was resuspended in PBS, and protein concentration was measured by BCA (Pierce, Thermo Fisher Scientific). Exosome size and particle number were analyzed using the LM10 or DS500 nanoparticle characterization system (NanoSight, Malvern Instruments) equipped with a violet laser (405 nm).

LC-MS/MS and Proteomic Data Analysis. Samples were denatured in 8 M urea in 100 mM ammonium bicarbonate buffer (pH 8), reduced using 10 mM DTT and alkylated using 100 mM iodoacetamide. This was followed by proteolytic digestion with endoproteinase LysC (Wako Chemicals) overnight at room temperature after diluting urea to <4 M. The samples were trypsinized (Trypsin Gold, Promega) for 5 hours after further urea dilution to <2 M. The digestion was quenched with formic acid, and resulting peptide mixtures were desalted using in-house manufactured C18 Empore (3M) StAGE tips (Rappsilber et al., “Stop and go Extraction Tips for Matrix16 Assisted Laser Desorption/Ionization, Nanoelectrospray, and LC/MS Sample Pretreatment in Proteomics,” Anal Chem 75:663-670 (2003), which is hereby incorporated by reference in its entirety). Samples were dried and resolubilized in 2% acetonitrile and 2% formic acid. Approximately 3 μg of each sample was injected for analysis by reverse phase nano-LC-MS/MS (Ultimate 3000 coupled to a Q-Exactive, Thermo Scientific). After loading on a C18 PepMap trap column (5 μm particles, 100 m×2 cm, Thermo Scientific) at a flow rate of 3 μl/min, peptides were separated using a 12 cm×75 μm C18 column (3 m particles, Nikkyo Technos Co., Ltd. Japan) at a flow rate of 200 nL/min, with a gradient increasing from 5% Buffer B (0.1% formic acid in acetonitrile)/95% Buffer A (0.1% formic acid) to 40% Buffer B/60% Buffer A, over 140 minutes. All LC-MS/MS experiments were performed in data dependent mode with lockmass of 445.12003. Precursor mass spectra were recorded for 300-1400 m/z at 70,000 resolution and 17,500 resolution for fragment ions (lowest mass: m/z 100) in profile mode. Up to twenty precursors per cycle were selected for fragmentation, and dynamic exclusion was set to 45 seconds. Normalized collision energy was set to 27. MS/MS spectra were extracted and searched against Uniprot complete Human or Murine proteome databases concatenated with common contaminants (Bunkenborg et al., “The minotaurproteome: Avoiding Cross-Species Identifications Deriving from Bovine Serum in Cell Culture Models,” Proteomics 10:3040-3044 (2010), which is hereby incorporated by reference in its entirety), using Proteome Discoverer 1.4 (Thermo Scientific) and Mascot 2.4 (Matrix Science). All cysteines were considered as alkylated. N-terminal glutamate to pyroglutamate conversion, oxidation of methionine and protein N-terminal acetylation were allowed as variable modifications. Data were searched using fully tryptic constraints (Trypsin/P). Matched peptides were filtered using a Percolator (Kall et al., “Semi-Supervised Learning for Peptide Identification from Shotgun Proteomics Datasets,” Nat Methods 4:923-925 (2007), which is hereby incorporated by reference in its entirety) based 1% false discovery rate. Proteins were sorted out according to highest area. The average area of the three most abundant peptides for a matched protein (Silva et al., “Absolute Quantification of Proteins by LCMSE: a Virtue of Parallel MS Acquisition,” Mol Cell Proteomics 5:144-156 (2006), which is hereby incorporated by reference in its entirety) was used to gauge protein amounts within and between samples. Proteins not detected or present in low amounts were assigned an area equal to zero.

Proteomic Data Processing. Software tools used for this study are available as open source R packages (https://www.r-project.org, v3.2.5). For key analyses these include: ‘limma’ for QC, analysis and exploration of proteomic expression data; ‘fgsea’ for gene set enrichment analysis and gene-gene correlations; ‘randomForest’, ‘PAM’ and ‘caret’ for training and plotting classification and regression models. Additional data exploration results were generated using custom functions in ‘skitools’.

Tandem MS data were queried against a database using Proteome Discoverer v1.4/MASCOT software. The relative abundance of a given protein was calculated from the average area of the three most intense peptide signals. For this software, this abundance measure ranges approximately 4 orders of magnitude, resulting in a lower signal range of 0.8-1.2×10⁶ that can be integrated for proteins of low abundance. Proteins for which area intensities were below the minimum range or were not detected were assigned an area of zero. For the proteins that were identified by multiple UniProt ID, the probe (based on UniProt ID) values were collapsed at the protein level using the probe with the maximum intensity.

For EVP protein frequency analysis based on presence and absence of the proteins, protein abundance was not considered; proteins were classified as detected or not detected across all samples. For pairwise comparison of PaCa and LuCa, proteins were considered as tumor exclusive markers if they were detected in at least two of the TT samples and not detected in any of the AT/DT samples. The same criteria were applied for identifying exclusive markers across plasma samples. To identify enriched proteins, a fold change cut-off of >10 was applied to select tumor-specific markers (FDR<0.01). This list was further filtered for those proteins detected in at least half of TT samples (i.e. at least 2 out of 4 samples). For plasma analysis in PaCa and LuCa samples, EVP proteins that were never found in healthy control plasma but found in at least two of the patient samples were chosen. For supervised random forest, the entire proteomic expression data set was used.

For Gene Set Enrichment Analysis (GSEA), the entire proteomic expression data set (Subramanian et al., “Gene set Enrichment Analysis: A Knowledge-Based Approach for Interpreting Genome-Wide Expression Profiles,” Proc Natl Acad Sci USA 102:15545-15550 (2005), which is hereby incorporated by reference in its entirety) was used. Gene sets from Molecular signatures database (MSigDB, http://www.broadinstitute.org/gsea/msigdb/index.jsp) v5.1 were used for GSEA (H: 50 hallmark gene sets; CS:KEGG: 186 canonical pathways from Kyoto Encyclopedia of Genes and Genomes [KEGG] pathway database; C5: 825 gene sets based on Gene Ontology [GO] term) (Liberzon et al., “Molecular Signatures Database (MSigDB) 3.0,” Bioinformatics 27:1739-1740 (2011), which is hereby incorporated by reference in its entirety). The default parameters were used to identify significantly enriched gene sets.

Random Forest is a machine learning method that combines the output of an ensemble of regression trees to predict the value of a response variable. The use of this method reduces the risk of over-fitting and makes the method robust to outliers and noise in the input data. Recursive Feature Elimination (RFE) provided by the caret R package was used for feature selection using default options and the minimal number of top features with the best accuracy according to the variable importance measure was determined. The data was divided into training set and independent test set. Heatmap based on random forest algorithm was performed to find highest predictive values. To identify enriched proteins, a fold change cut-off of >100 or <1/100 was applied to select tumor- or non-tumor specific markers (FDR<0.05). Next, Random Forest algorithm (RFE algorithm) was applied to identify biomarker differentiating tumor from non-tumor samples. Analyses were performed using R statistical software version 3.5.0.

Transmission electron microscopy (TEM). For negative staining TEM analysis, 5 μl of exosomes in PBS were placed on a formvar/carbon coated grid and allowed to settle for 1 minute. The sample was blotted and negatively stained with 4 successive drops of 1.5% (aqu) uranyl acetate, blotting between each drop. Following the last drop of stain, the grid was blotted and air-dried. Grids were imaged with a JEOL JSM 1400 (JEOL, USA, Ltd, Peabody, Mass.) transmission electron microscope operating at 100 Kv. Images were captured on a Veleta 2K×2K CCD camera (Olympus-SIS, Munich, Germany).

Asymmetric-flow field-flow fractionation (AF4) fractionation. Exosome subpopulations (exomeres, <50 nm with an average of 35 nm in diameter; Exo-S, 60-80 nm in diameter; Exo-L, 90-120 nm in diameter and small exosome vesicles) were separated using AF4 as previously described (Zhang et al., “Identification of Distinct Nanoparticles and Subsets of Extracellular Vesicles by Asymmetric Flow Field-Flow Fractionation,” Nature Cell Biology 20:332-343 (2018); Zhang et al., “Asymmetric-Flow Field-Flow Fractionation Technology for Exomere and Small Extracellular Vesicle Separation and Characterization,” Nat Protoc 14:1027-1053 (2019), which are hereby incorporated by reference in their entirety). Briefly, samples were separated in a short channel (144 mm length, Wyatt Technology, Santa Barbara) with a 10 kDa molecular weight cutoff (MWCO) Regenerated Cellulose membrane (Millipore) on the accumulation bottom wall and a 490 μm spacer (channel thickness). The fractionation was operated by the Eclipse AF4 system (Wyatt Technology). Lastly, the system was eluted twice. Chemstation software (Agilent Technologies) with integrated Eclipse module (Wyatt Technology) was used to operate the AF4 flow and Astra 6 (Wyatt Technology) was used for data acquisition and analysis. 100 μg of proteins per sample (at 1 μg/μl, i.e. 100 μl) isolated using the sequential ultracentrifugation method was spun at 12,000×g for 5 minutes right before loading onto the AF4 system (to remove aggregates) and then injected using the autosampler.

### Example 1—Proteomic Characterization of Human EVPs

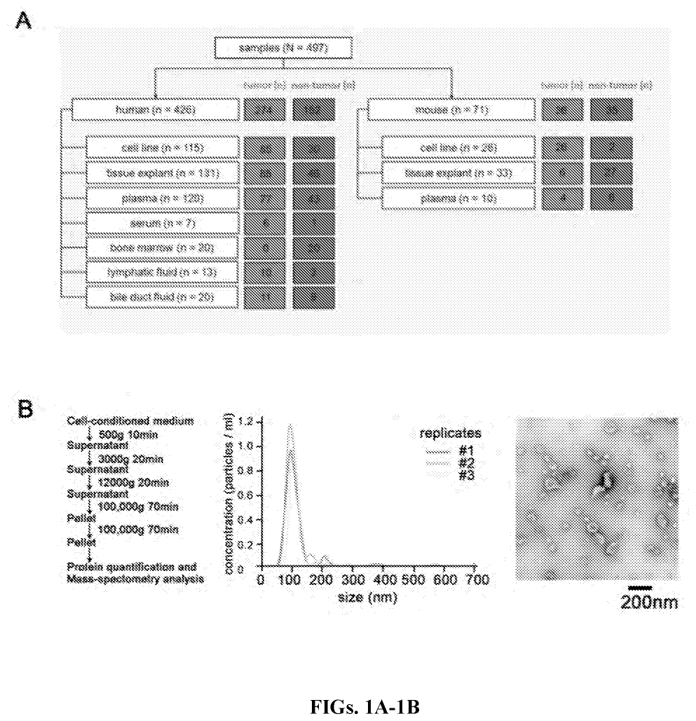

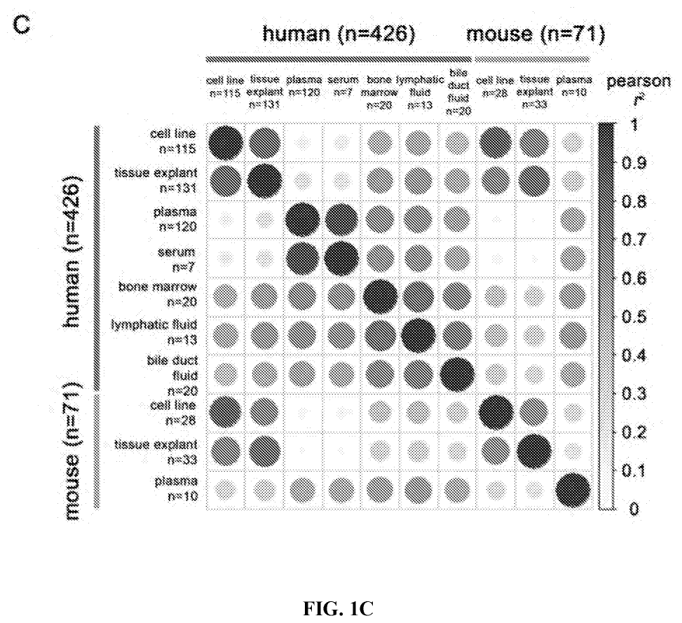

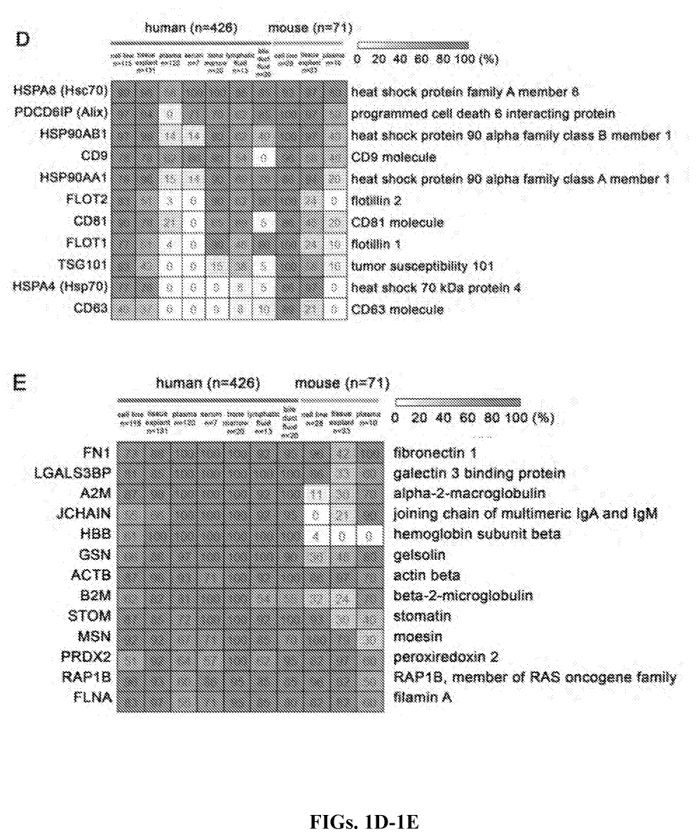

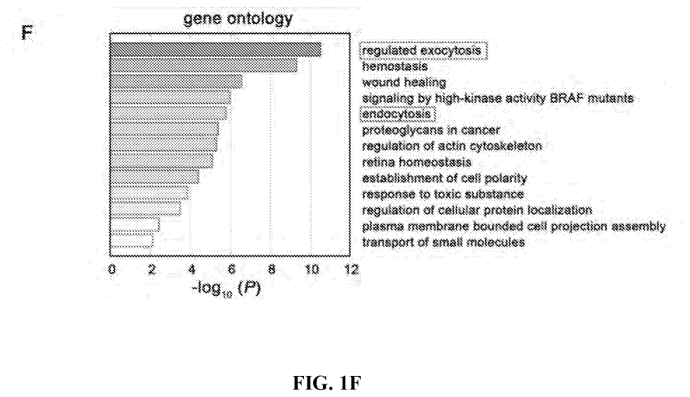

To characterize the proteomic composition of EVPs, high-speed and ultra high-speed centrifugation was used to isolate EVPs from a total of 497 normal and cancer-associated human and murine-derived samples from cell lines, tissues, plasma and other bodily fluids (FIG. 1A; Tables 16 and 17).

TABLE 16
Patient sample information (tissue)

| | sample size | | average | |
|---|---|---|---|---|
| Cancer type | (% male) | | age (yr) | stage (%) |
| Lung adenocarcinoma | 18 | (33.3) | 63 | I (38.9); II (27.8); |
| | | | | III (11.1); IV (22.2) |
| Pancreatic ductal | 21 | (66.7) | 68.9 | I (4.8); II (71.4); |
| adenocarcinoma | | | | III (23.8) |
| Neuroblastoma | 9 | (36.4) | 8.1 | IV (100) |
| Osteosarcoma | 7 | (57.1) | 19 | IV (100) |
| Breast cancer | 6 | (0) | 66.7 | IV (100) |
| Melanoma | 5 | (NA) | NA | III (100) |
| Colorectal cancer | 3 | (33.3) | 63.7 | 0 (33.3); II (66.7) |
| Hepatoblastoma | 3 | (100) | 2.3 | IV (100) |
| MPNST (malignant | 1 | (100) | 16 | IV (100) |
| peripheral nerve | | | | |
| sheath tumor) | | | | |
| Fibrolabellar | 2 | (100) | 16 | IV (100) |
| hepatocellular | | | | |
| carcinoma | | | | |
| Wilms tumor | 2 | (100) | 3 | IV (100) |
| Immature teratoma | 1 | (0) | 14 | IV (100) |
| Desmoplastic small- | 1 | (100) | 14 | IV (100) |
| round-cell tumor | | | | |
| Embryonal | 2 | (0) | 5.5 | IV (100) |
| rhabdomyosarcoma | | | | |
| Epithelioid sarcoma | 1 | (0) | 16 | IV (100) |
| Synovial sarcoma | 1 | (100) | 54 | IV (100) |
| Renal cell carcinoma | 1 | (100) | 60 | IV (100) |
| Anasplastic ependymoma | 1 | (0) | 10 | IV (100) |

| | |
|---|---|
| Total | 85 |

TABLE 17
Patient sample information (plasma).

| | sample size | | average | |
|---|---|---|---|---|
| Cancer type | (% male) | | age (yr) | stage (%) |
| Lung adenocarcinoma | 12 | (41.7) | 61 | I (50.0); II (41.7); |
| | | | | III (8.3) |
| Pancreatic ductal | 9 | (66.7) | 69.9 | II (77.8); III (22.2) |
| adenocarcinoma | | | | |
| Neuroblastoma | 15 | (66.7) | 5.3 | III (6.7); IV (93.3) |
| Osteosarcoma | 5 | (80) | 21.6 | IV (100) |
| Breast cancer | 8 | (0) | 52.1 | II (25); III (12.5); |
| | | | | IV (62.5) |
| Melanoma | 1 | (NA) | NA | NA |
| Colorectal cancer | 3 | (33.3) | 63.7 | 0 (33.3); II (66.7) |
| Hepatoblastoma | 1 | (100) | 2 | IV (100) |
| Germinoma | 1 | (NA) | NA | NA |
| MPNST (malignant | 1 | (100) | 16 | IV (100) |
| peripheral nerve | | | | |
| sheath tumor) | | | | |
| Fibrolabellar | 2 | (100) | 15.5 | IV (100) |
| hepatocellular | | | | |
| carcinoma | | | | |
| Wilms tumor | 1 | (100) | 1 | IV (100) |
| Mesothelioma | 15 | (73.3) | 64.3 | I (13.3); III (6.7); |
| | | | | IV (6.7); NA (73.3) |
| DSRCT (desmoplastic | 1 | (100) | 14 | IV (100) |
| small-round-cell tumor) | | | | |
| Hodgkin's lymphoma | 1 | (0) | 19 | IV (100) |
| Anasplastic ependymoma | 1 | (0) | 10 | IV (100) |
| Total | 77 | | | |
| Adult control | 28 | (46.4%) | 46.4 | NA |
| Pediatric control | 15 | (80%) | 7.47 | NA |

| | |
|---|---|
| Total | 43 |

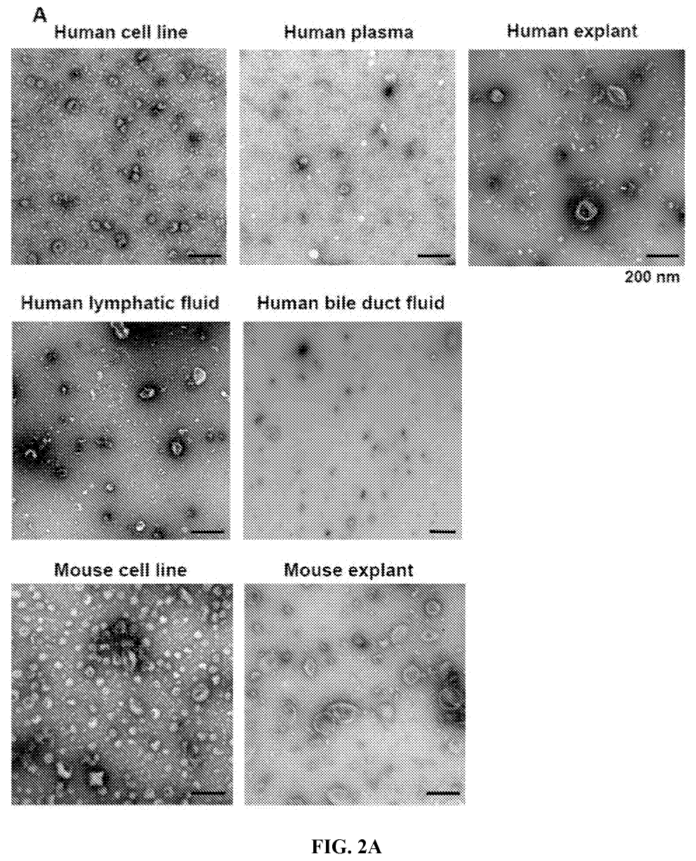

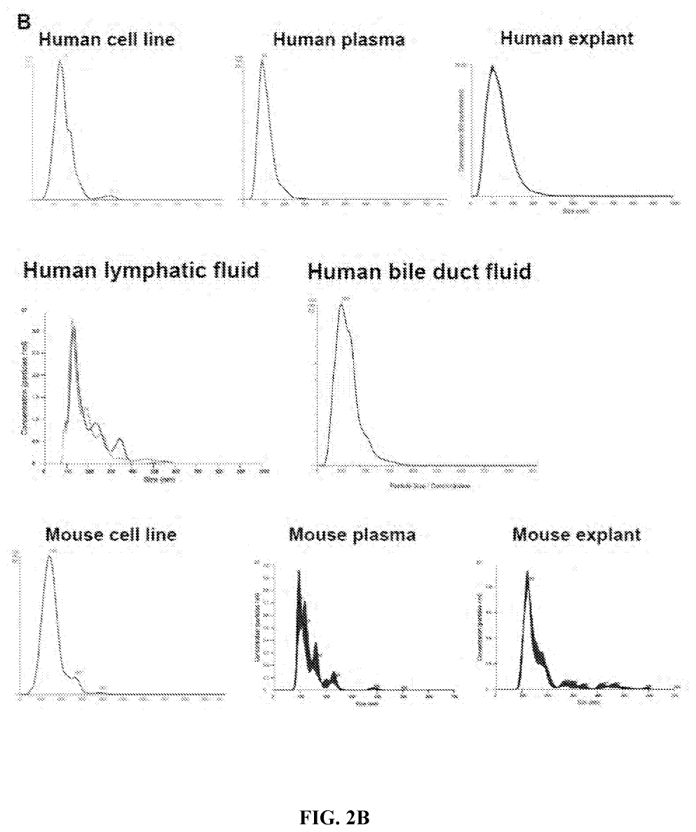

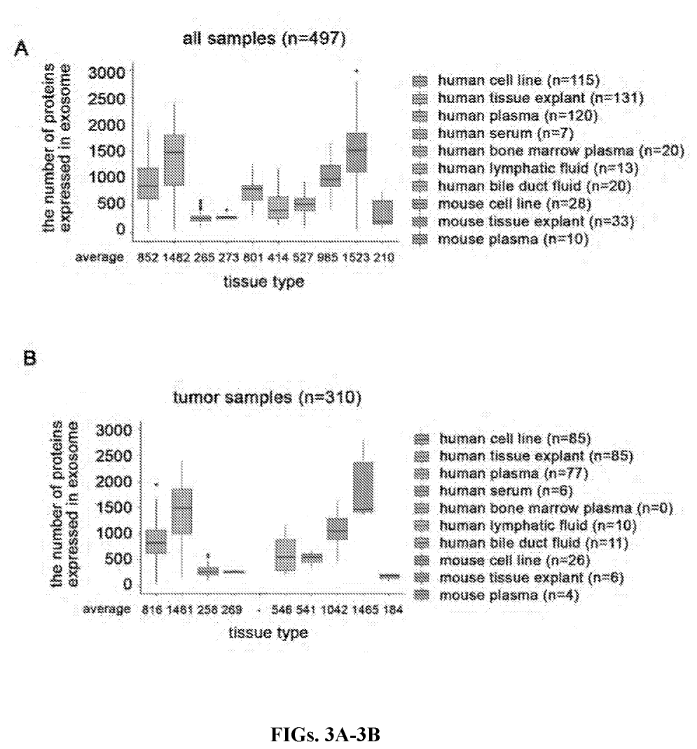

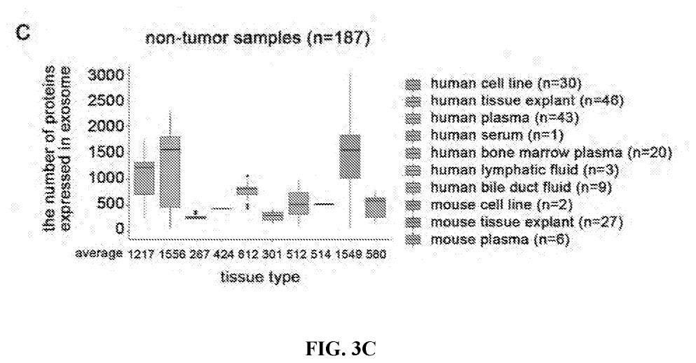

All EVP samples isolated by sequential ultracentrifugation (SUC) represent a heterogeneous population categorized into three prominent sub-populations that include exomeres (non-vesicular particles <50 nm) and two exosome subpopulations (exosome small 50-70 nm; exosome large 90-120 nm) (Zhang et al., “Identification of Distinct Nanoparticles and Subsets of Extracellular Vesicles by Asymmetric Flow Field-Flow Fractionation,” Nature Cell Biology 20:332-343 (2018); Zhang et al., “Asymmetric-Flow Field-Flow Fractionation Technology for Exomere and Small Extracellular Vesicle Separation and Characterization,” Nat Protoc 14:1027-1053 (2019), which are hereby incorporated by reference in their entirety) (FIG. 1). Heterogeneous EVP populations isolated in the present study were characterized in terms of size range (30-150 nm) and morphology via nanoparticle tracking analysis (NTA) and transmission electron microscopy (TEM), respectively (FIG. 1B and FIG. 2). A database composed of EVPs isolated from 426 human samples was constructed, which included resected normal and malignant tissues (n=131), blood plasma (n=120), cell lines (n=115), blood serum (n=7), bone marrow (n=20), lymphatic fluid (n=13) and bile duct fluid specimens (n=20) from 152 control and 274 cancer samples (FIG. 1A). The cancer patient-resected tissue and plasma samples analyzed included both adult cancers (such as pancreatic, lung, breast and colorectal carcinomas and melanoma) and pediatric cancers (such as neuroblastoma and osteosarcoma). The average number of unique proteins detected in the EVP preparations was 862 (25% to 75% percentile, 310 to 1,282 proteins), with the lowest numbers detected in plasma and serum (an average of 265 and 273 proteins in human plasma and serum, respectively and 210 proteins in murine plasma) and the highest numbers of proteins found in explant tissues (average of 1,482 and 1,523 proteins in human and murine explant tissues, respectively) (FIG. 3A). While for some specific EVP proteins, concentrations have been found to increase with cancer stage (Peinado et al., “Melanoma Exosomes Educate Bone Marrow Progenitor Cells Toward a Pro-Metastatic Phenotype through MET,” Nat Med 18:883-891 (2012), which is hereby incorporated by reference in its entirety), differences between non-tumor and tumor samples was not observed in the number of distinct EVP proteins detected, consisting of either common or unique proteins (FIG. 3B and FIG. 3C).

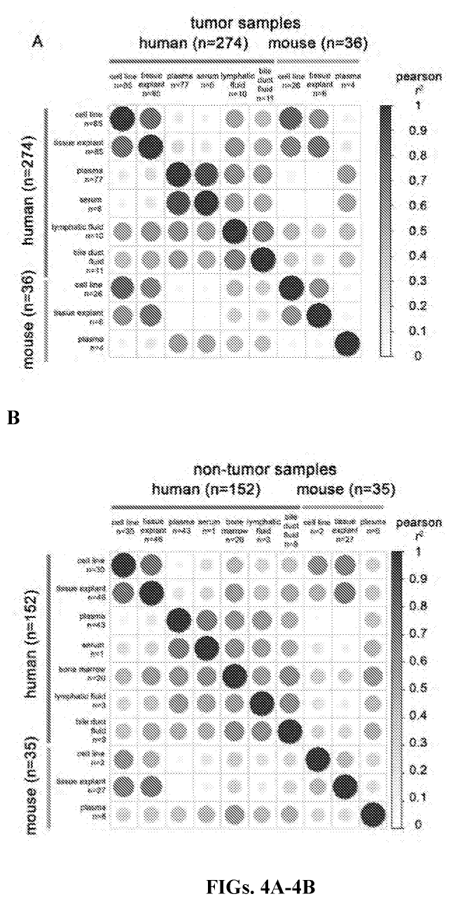

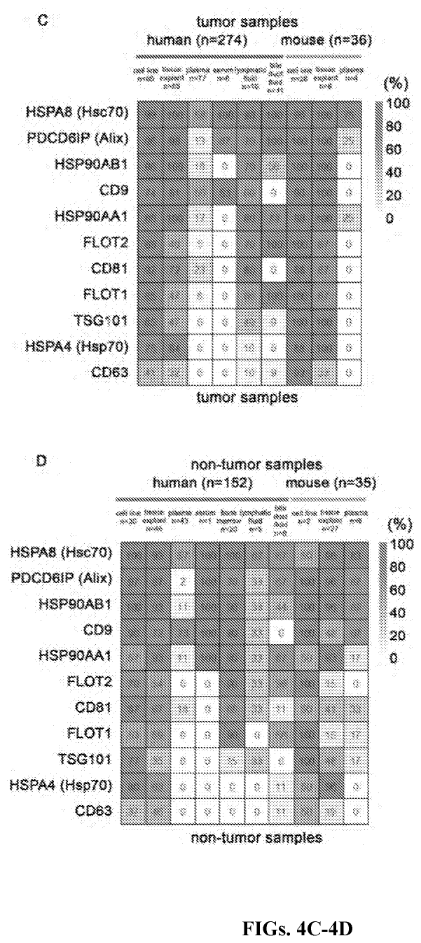

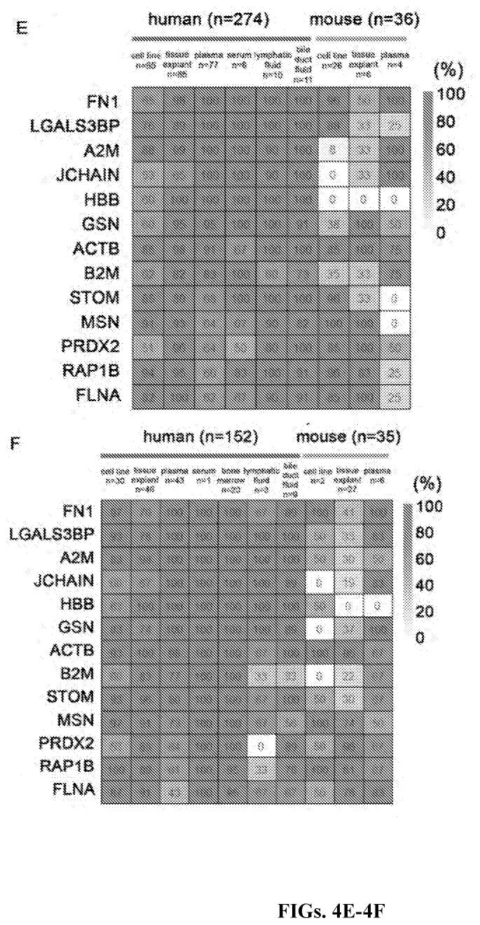

To evaluate the overall correlation between EVP proteomes derived from different sources, a Pearson correlation analysis was performed comparing specimen types (plasma versus tissue explants) and species (human versus murine) for all tumor and non-tumor exosome samples. The sample source was the strongest determinant of EVP protein signatures (FIG. 1C). EVP proteins from human plasma overlapped best with human serum-derived EVPs (r²=0.92), followed by human bone marrow (r²=0.65) and lymphatic fluid EVPs (r²=0.64), and correlated least with human cell line (r²=0.15) and tissue explant-derived EVPs (r²=0.24), suggesting the complexity of plasma and lymph EVP proteomes may drive the divergence from tissue EVP proteomes (FIG. 1C). In terms of inter-species differences, the proteomes of human and murine cell line- and tissue explant-derived EVPs were similar (r²=0.85 and 0.78, respectively), while the proteomes of plasma-derived EVPs largely differed between mice and humans (r²=0.52) (FIG. 1C). These observations held true whether tumor samples or non-tumor samples were analyzed together or separately (FIG. 1C, FIG. 4A, and FIG. 4B). These data suggest that, in general, EVP profiles differ significantly depending on the tissue source and species and that murine plasma-derived EVP proteomes cannot be used to guide liquid biopsy studies in patients.

### Example 2—Unbiased EVP Proteome Analysis Identifies 13 Additional Common Exosome Biomarkers

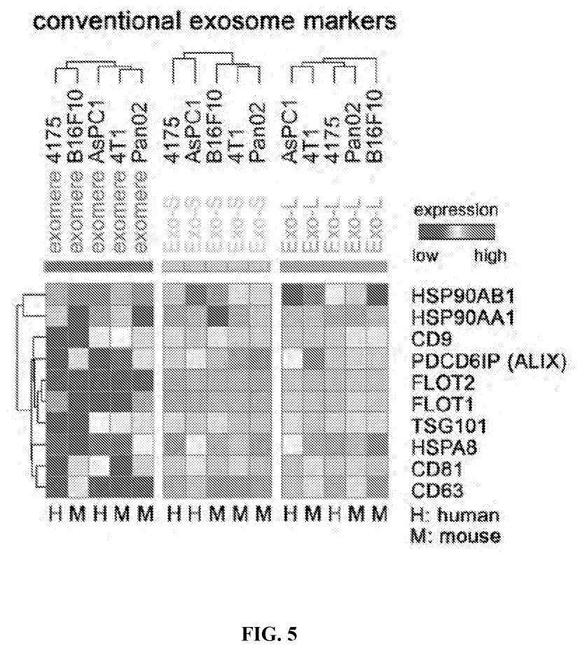

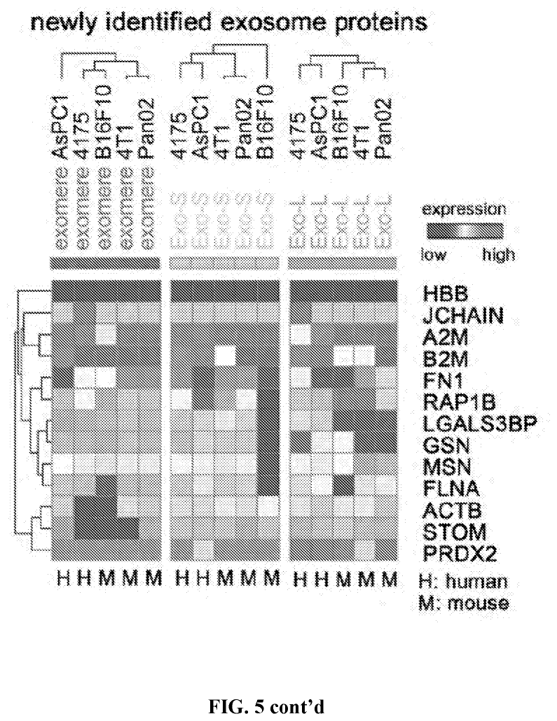

In order to better define ubiquitous pan-EVP markers for improved isolation from various human and murine sources, the frequency of specific proteins found in EVPs from different sources was investigated. Traditional exosomal markers (i.e., tetraspanins, heat shock proteins) were investigated first and, of 11 conventional exosomal markers examined (Thery et al., “Isolation and Characterization of Exosomes from Cell Culture Supernatants and Biological Fluids,” Curr Protoc Cell Biol Chapter 3, Unit 3 22 (2006), which is hereby incorporated by reference in its entirety), HSPA8 was the only protein found in >50% of EVP samples from all sources (FIG. 1D). Remarkably, CD63 was present in 89% of the examined murine cell line-derived EVPs, but was rarely detected in EVPs isolated from ex vivo tissues or biofluids of either human or mouse origin (FIG. 1D). Among the human cell line-derived EVP proteins, all of the established exosome markers, except CD63, were present in >77% of 115 human cell line-derived samples (FIG. 1D), supporting the idea that the SUC approach specifically enriches the preparations in exosomes. Importantly, interrogation of proteomics data for the extracellular nanoparticle sub-populations confirmed that of the traditional exosome markers, CD63 was rarely found in human exosome samples, in either Exo-S/Exo-L or exomeres, while CD9, HSPA8, ALIX, HSP90AB1 were commonly detected, and represent pan exosome/exomere markers (FIG. 5). For mouse cell line-derived EVPs, all 11 markers were highly represented (≥86%) (FIG. 1D). However, for human plasma or serum, CD9 and HSPA8 were the only proteins found at ≥50% frequency (FIG. 1D), suggesting that pan-exosome markers currently used to verify exosomal origin in in vitro studies may not be directly translated to patient-derived biofluids. These findings were similar regardless of whether analyses were performed with tumor- or non tumor-derived specimens (FIG. 4C and FIG. 4D) and highlight the need to identify novel pan EVP markers.

To identify proteins found at high frequency in all human-derived EVPs, irrespective of source, proteins that met a threshold of ≥50% representation across specimens were examined. Of 11,000 human EVP proteins, only 13 matched this criterion (FIG. 1E, FIG. 4E, and FIG. 4F). Gene ontology analysis demonstrated that the vast majority of these proteins, including alpha-2-macroglobulin (A2M), beta-2-Microglobulin (B2M), stomatin (STOM), filamin A (FLNA), fibronectin 1 (FN1), gelsolin (GSN), hemoglobin subunit Beta (HBB), galectin-3-binding protein (LGALS3BP), ras-related protein 1b (RAP1B), actin beta (ACTB) and joining chain of multimeric IgA and IgM (JCHAIN), were involved in the regulation of exocytosis and endocytosis (FIG. 1F). Of these 13 molecules, actin beta, moesin (MSN), and RAP1B represent pan exosome/exomere markers that can be identified in human Exo-S/Exo-L as well as exomeres, while stomatin is a specific exosome marker as it is only found in Exo-S/Exo-L and thus can distinguish exosomes from exomeres (FIG. 5). Given their relationship to endosomal and exosomal pathways, these newly identified EVP proteins may represent additional bona fide exosomal markers that could potentially be used to improve EVP isolation across any type of human source (FIG. 1F; Table 18).

TABLE 18
Gene ontology analysis using Metascape with 13 common exosome proteins expressed more than 50% in human samples.

| Category | Term | Description | Proteins | Log P | Log (q-value) |
|---|---|---|---|---|---|
| Reactome Gene | R-HSA-109582 | Hemostasis | A2M, ACTB, FLNA, FN1, HBB, JCHAIN, | −10.9 | −6.6 |
| Sets | | | LGALS3BP, RAP1B, Y [symbol] | | |
| GO Biological | GO:0045055 | regulated exocytosis | A2M, B2M, STOM, FLNA, FN1, GSN, | −10.2 | −6.2 |
| Processes | | | HBB, LGALS3BP, RAP1B [symbol] | | |
| GO Biological | GO:0042060 | wound healing | A2M, ACTB, FLNA, FN1, GSN, HBB, | −6.0 | −4.2 |
| Processes | | | YWHAZ, MSN | | |
| Reactome Gene | R-HSA-6802948 | Signaling by high-kinase | ACTB, FN1, RAP1B, FLNA | −5.9 | −2.7 |
| Sets | | activity BRAF mutants | | | |
| GO Biological | GO:1905475 | regulation of protein | ACTB, STOM, GSN, YWHAZ, MSN, | −5.7 | −2.6 |
| Processes | | localization to membrane | RAP1B, FLNA, FN1 | | |
| GO Biological | GO:0006897 | endocytosis | ACTB, B2M, GSN, HBB, JCHAIN, LGALS3BP | −5.5 | −2.5 |
| Processes | | | | | |
| KEGG Pathway | hsa05205 | Proteoglycans in cancer | ACTB, FLNA, FN1, MSN, GSN, B2M | −5.3 | −2.3 |
| KEGG Pathway | hsa04810 | Regulation of actin cytoskeleton | ACTB, FN1, GSN, MSN, HBB, FLNA, | −5.2 | −2.3 |
| | | | JCHAIN, YWHAZ, B2M | | |
| Reactome Gene | R-HSA-1280215 | Cytokine Signaling in Immune | B2M, FN1, MSN, RAP1B, YWHAZ, ACTB, | −4.4 | −1.8 |
| Sets | | system | GSN, FLNA, STOM | | |
| GO Biological | GO:0003014 | renal system process | GSN, HBB, JCHAIN, ACTB, YWHAZ, STOM | −4.4 | −1.7 |
| Processes | | | | | |
| GO Biological | GO:0009636 | response to toxic substance | ACTB, GSN, HBB, PRDX2, B2M | −3.7 | −1.1 |
| Processes | | | | | |
| GO Biological | GO:0034762 | regulation of transmembrane | ACTB, STOM, FLNA, GSN, RAP1B | −2.4 | −0.1 |
| Processes | | transport | | | |
| Reactome Gene | R-HSA-382551 | Transport of small molecules | A2M, STOM, HBB | −2.1 | 0.0 |
| Sets | | | | | |

[symbol]

indicates data missing or illegible when filed

These markers were present at a similar frequency regardless of whether the samples were of tumor or non-tumor origin. Thus, common exosome markers have been identified for a variety of potential sources of liquid biopsy for improved EVP detection and isolation methodologies (FIGS. 4C-4F).

### Example 3—Identification of Tissue-Specific Tumor-Derived EVP Proteins in Patients

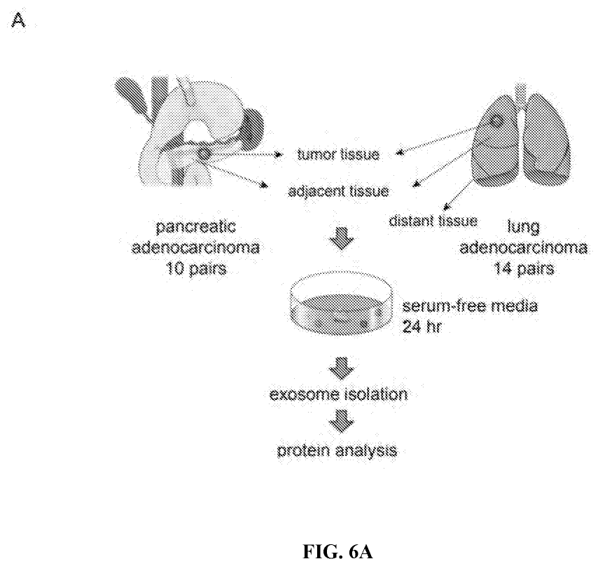

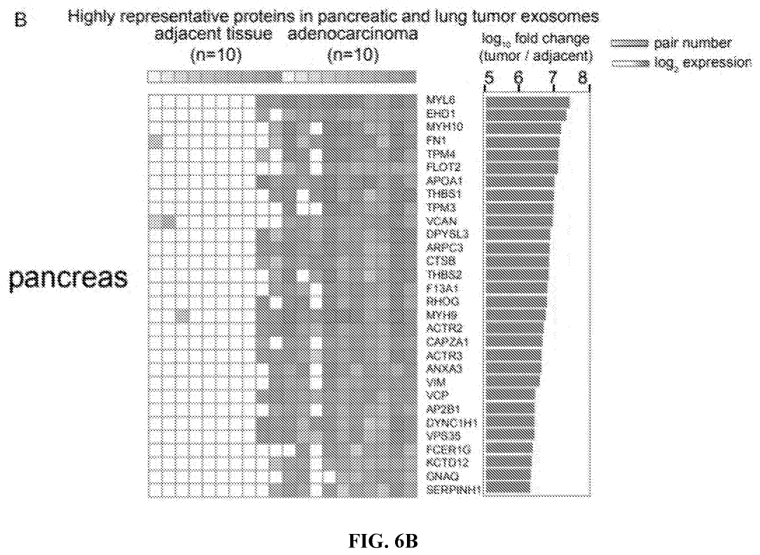

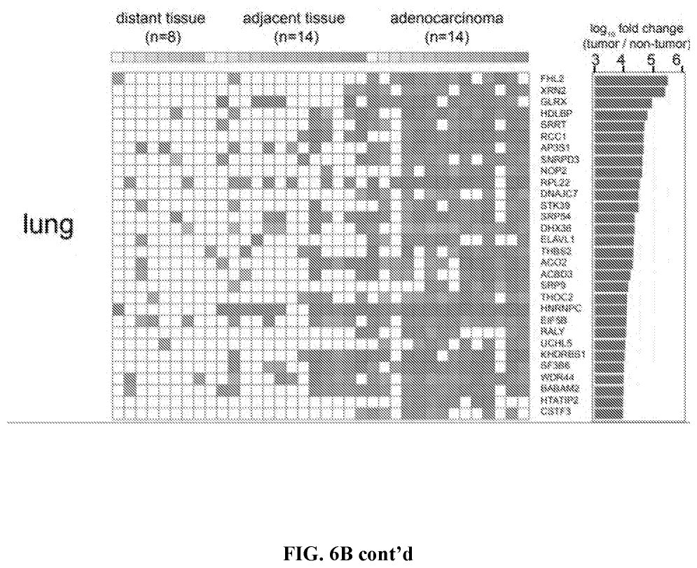

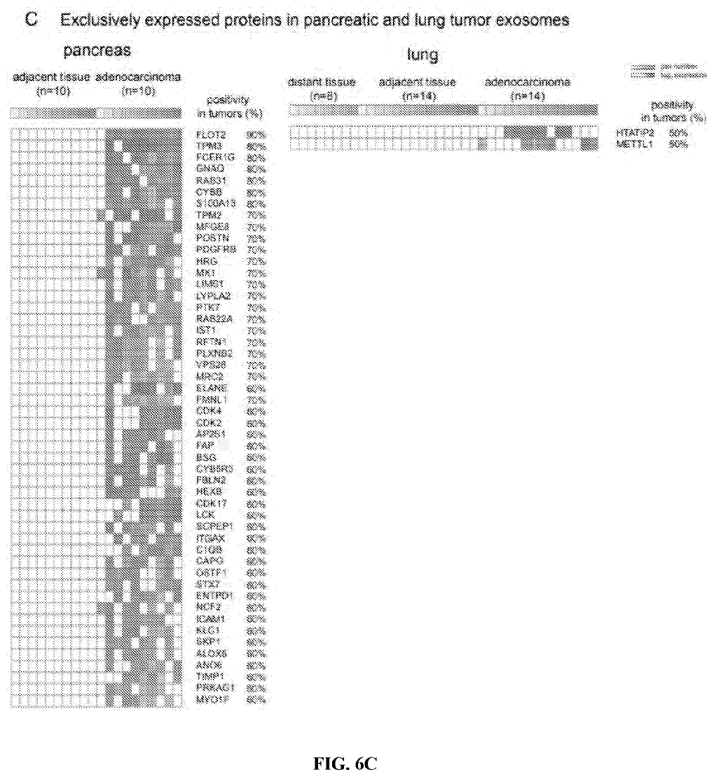

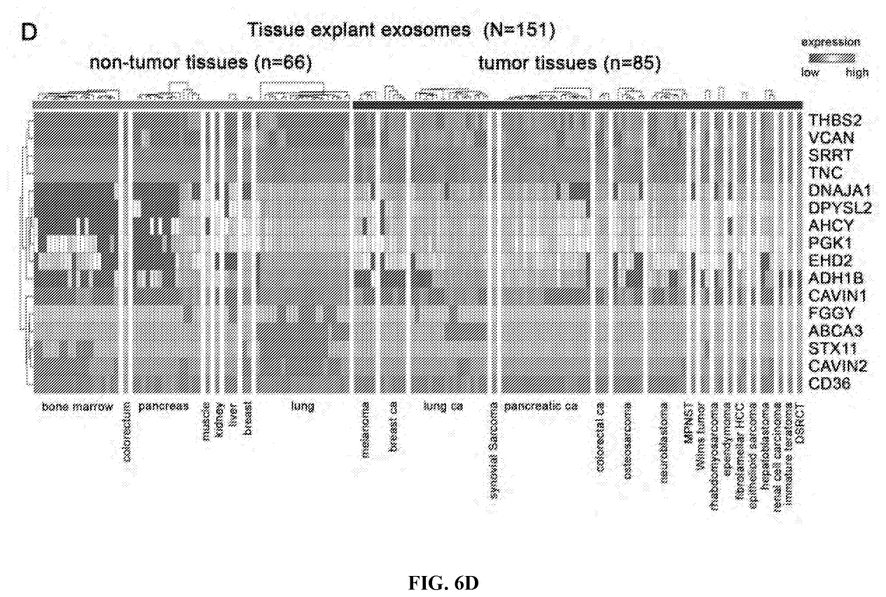

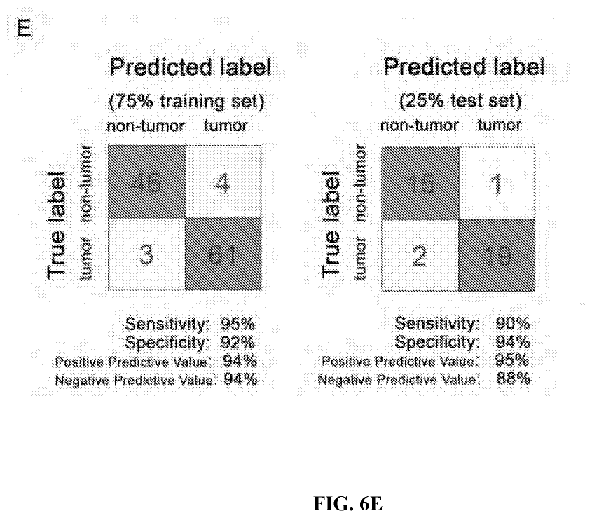

To identify EVP proteins that could be used as diagnostic biomarkers for cancer patients, it was first sought to identify shared and non-shared tumor-specific EVP proteins by performing a pairwise comparison between tumor tissues (TT) EVP proteomes, as tumor exosome-enriched sources, and non-tumor adjacent tissues (AT) EVP proteomes from the same cancer patients. TT and AT were resected from 10 patients with pancreatic adenocarcinoma (PaCa) and 14 patients with lung adenocarcinoma (LuCa), and EVPs were isolated for pairwise comparison (FIG. 6A). In addition, eight non-tumor distant tissues (DT) resected from LuCa patients were also obtained, as non-malignant tissues collected distally from tumor sites are less likely to be affected by tumor-secreted factors, and EVPs isolated from these DT were included as a third group in the comparison studies (FIG. 6A).

Distinct EVP proteins with potential biomarker value and biological relevance in PaCa and LuCa were identified by analyzing EVP proteins most enriched in TT as compared to AT and DT. EVP proteins were searched that were present in ≥50% of the samples and, of those proteins, the ones showing a 10-fold or larger increase compared to AT or AT/DT with a false discovery rate (FDR) of <0.1 were selected. Based on these criteria, 530 and 176 EVP proteins were identified as TT-enriched proteins in PaCa and LuCa, respectively (top proteins shown in FIG. 6B; complete list in Tables 19 and 20).

TABLE 19
530 Tumor-enriched EVP proteins in PaCa (10 pairs; >10-fold,
false discovery rate [FDR] <0.1, and ≥50% found in tumor-
derived EVP from tumor tissue (TT). Proteins in bold
are exclusively expressed in TT.

| | | log₁₀ | | |
|---|---|---|---|---|
| | false | fold | positivity | positivity |
| | discovery | change | in tumor | in adjacent |
| | rate | (tumor/ | tissue | tissue |
| protein | (FDR) | adjacent) | (n = 10) | (n = 10) |
| MYL6 | 0.005 | 7.5 | 100% | 20% |
| EHD1 | 0.005 | 7.4 | 100% | 10% |
| MYH10 | 0.005 | 7.2 | 90% | 10% |
| FN1 | 0.005 | 7.2 | 100% | 20% |
| TPM4 | 0.005 | 7.1 | 90% | 10% |
| FLOT2 | 0.005 | 7.1 | 90% | 0% |
| APOA1 | 0.005 | 7.0 | 100% | 20% |
| THBS1 | 0.010 | 7.0 | 90% | 10% |
| TPM3 | 0.005 | 7.0 | 80% | 0% |
| VCAN | 0.005 | 6.9 | 100% | 20% |
| DPYSL3 | 0.005 | 6.9 | 100% | 20% |
| ARPC3 | 0.005 | 6.9 | 100% | 20% |
| CTSB | 0.005 | 6.9 | 100% | 20% |
| THBS2 | 0.005 | 6.8 | 90% | 10% |
| F13A1 | 0.010 | 6.8 | 90% | 10% |
| RHOG | 0.005 | 6.8 | 90% | 10% |
| MYH9 | 0.005 | 6.8 | 100% | 30% |
| ACTR2 | 0.005 | 6.7 | 100% | 20% |
| CAPZA1 | 0.005 | 6.7 | 90% | 10% |
| ACTR3 | 0.005 | 6.6 | 100% | 20% |
| ANXA3 | 0.005 | 6.6 | 90% | 10% |
| VIM | 0.010 | 6.6 | 90% | 10% |
| VCP | 0.005 | 6.4 | 100% | 20% |
| AP2B1 | 0.010 | 6.4 | 90% | 10% |
| DYNC1H1 | 0.005 | 6.4 | 100% | 20% |
| VPS35 | 0.005 | 6.4 | 100% | 20% |
| FCER1G | 0.005 | 6.4 | 80% | 0% |
| KCTD12 | 0.005 | 6.3 | 90% | 10% |
| GNAQ | 0.005 | 6.3 | 80% | 0% |
| SERPINH1 | 0.005 | 6.3 | 90% | 10% |
| RAB31 | 0.005 | 6.3 | 80% | 0% |
| GSTO1 | 0.005 | 6.3 | 100% | 20% |
| TPM1 | 0.010 | 6.2 | 80% | 10% |
| TGM2 | 0.005 | 6.2 | 90% | 20% |
| CYBB | 0.005 | 6.2 | 80% | 0% |
| ITGB2 | 0.005 | 6.2 | 100% | 30% |
| S100A13 | 0.005 | 6.2 | 80% | 0% |
| SLC3A2 | 0.005 | 6.1 | 90% | 10% |
| APFC1B | 0.015 | 6.1 | 80% | 10% |
| RAB5B | 0.010 | 6.1 | 90% | 20% |
| RAB5C | 0.010 | 6.1 | 90% | 20% |
| SLC44A1 | 0.010 | 6.0 | 80% | 10% |
| ALDOA | 0.005 | 6.0 | 90% | 20% |
| RPLP0 | 0.024 | 5.9 | 90% | 20% |
| RPLP2 | 0.024 | 5.9 | 90% | 20% |
| PPP1CA | 0.015 | 5.9 | 90% | 20% |
| PPP1CB | 0.015 | 5.9 | 90% | 20% |
| EIF4A2 | 0.024 | 5.9 | 90% | 20% |
| EPB41L2 | 0.010 | 5.9 | 90% | 20% |
| TPM2 | 0.015 | 5.9 | 70% | 0% |
| S100A4 | 0.015 | 5.9 | 80% | 10% |
| FBLN1 | 0.005 | 5.8 | 80% | 10% |
| SEPT11 | 0.005 | 5.8 | 90% | 20% |
| CPA1 | 0.024 | 5.8 | 90% | 20% |
| PTPRC | 0.005 | 5.8 | 90% | 20% |
| ESD | 0.024 | 5.7 | 90% | 20% |
| PLS3 | 0.005 | 5.7 | 90% | 20% |
| CBR1 | 0.015 | 5.7 | 90% | 20% |
| DDAH2 | 0.005 | 5.7 | 80% | 10% |
| DYNLL2 | 0.010 | 5.7 | 80% | 10% |
| PGK1 | 0.010 | 5.7 | 100% | 40% |
| AKR1B1 | 0.015 | 5.7 | 90% | 20% |
| MFGE8 | 0.010 | 5.7 | 70% | 0% |
| SEPT2 | 0.015 | 5.7 | 90% | 20% |
| POSTN | 0.020 | 5.7 | 70% | 0% |
| PLEC | 0.024 | 5.7 | 90% | 20% |
| TAGLN | 0.010 | 5.7 | 90% | 20% |
| MYO1D | 0.010 | 5.7 | 90% | 20% |
| PSME1 | 0.010 | 5.7 | 90% | 20% |
| FGB | 0.005 | 5.7 | 100% | 40% |
| PDGFRB | 0.005 | 5.6 | 70% | 0% |
| DDK5 | 0.010 | 5.6 | 80% | 10% |
| ITGB5 | 0.005 | 5.6 | 90% | 20% |
| GLIPR2 | 0.010 | 5.6 | 80% | 10% |
| FERMT3 | 0.015 | 5.6 | 80% | 10% |
| AP1B1 | 0.015 | 5.6 | 80% | 10% |
| GSN | 0.005 | 5.6 | 90% | 30% |
| ITGAM | 0.015 | 5.6 | 80% | 10% |
| COL12A1 | 0.020 | 5.6 | 80% | 10% |
| HRG | 0.020 | 5.6 | 70% | 0% |
| MX1 | 0.020 | 5.5 | 70% | 0% |
| GSTP1 | 0.015 | 5.5 | 90% | 30% |
| ARL8A | 0.010 | 5.5 | 80% | 10% |
| LIMS1 | 0.015 | 5.5 | 70% | 0% |
| MYH14 | 0.015 | 5.5 | 80% | 20% |
| AP2M1 | 0.010 | 5.5 | 90% | 20% |
| HNRNPDL | 0.015 | 5.5 | 80% | 10% |
| ATL3 | 0.020 | 5.5 | 80% | 10% |
| LYPLA2 | 0.020 | 5.5 | 70% | 0% |
| PTK7 | 0.005 | 5.5 | 70% | 0% |
| MYH11 | 0.015 | 5.5 | 80% | 20% |
| GNA11 | 0.005 | 5.5 | 90% | 20% |
| CD9 | 0.010 | 5.5 | 80% | 20% |
| RAB22A | 0.005 | 5.5 | 70% | 0% |
| A1BG | 0.010 | 5.5 | 80% | 10% |
| FARP1 | 0.015 | 5.5 | 90% | 20% |
| TCP1 | 0.024 | 5.4 | 90% | 20% |
| UGP2 | 0.015 | 5.4 | 80% | 10% |
| AZU1 | 0.010 | 5.4 | 80% | 20% |
| SEPTS | 0.015 | 5.4 | 90% | 20% |
| IST1 | 0.015 | 5.4 | 70% | 0% |
| HIST1H2BL | 0.020 | 5.4 | 70% | 20% |
| CLIC1 | 0.033 | 5.4 | 90% | 30% |
| EEF2 | 0.010 | 5.4 | 100% | 40% |
| PSMD6 | 0.028 | 5.4 | 90% | 20% |
| SPTAN1 | 0.015 | 5.4 | 90% | 20% |
| RFTN1 | 0.010 | 5.4 | 70% | 0% |
| NSF | 0.015 | 5.4 | 90% | 20% |
| NNMT | 0.024 | 5.4 | 70% | 10% |
| WDR1 | 0.020 | 5.4 | 90% | 30% |
| ITG84 | 0.015 | 5.3 | 80% | 10% |
| KPNB1 | 0.015 | 5.3 | 90% | 20% |
| RAB7A | 0.015 | 5.3 | 90% | 30% |
| PLXNB2 | 0.010 | 5.3 | 70% | 0% |
| LGALS3 | 0.005 | 5.3 | 80% | 20% |
| RAN | 0.020 | 5.3 | 100% | 40% |
| ST13 | 0.015 | 5.3 | 80% | 10% |
| ACTN1 | 0.005 | 5.3 | 90% | 30% |
| VPS28 | 0.010 | 5.2 | 70% | 0% |
| CAPZB | 0.005 | 5.2 | 90% | 30% |
| GCA | 0.010 | 5.2 | 90% | 30% |
| ACTN4 | 0.010 | 5.2 | 90% | 30% |
| MRC2 | 0.015 | 5.2 | 70% | 0% |
| HIST2H3PS2 | 0.033 | 5.2 | 80% | 30% |
| ELANE | 0.028 | 5.2 | 60% | 0% |
| ATP1B1 | 0.015 | 5.2 | 90% | 30% |
| F2 | 0.020 | 5.2 | 88% | 20% |
| ITIH1 | 0.010 | 5.2 | 70% | 10% |
| DPYSL2 | 0.015 | 5.2 | 90% | 30% |
| PLG | 0.028 | 5.1 | 80% | 20% |
| ARPC2 | 0.041 | 5.1 | 80% | 20% |
| MYOF | 0.010 | 5.1 | 100% | 40% |
| ANKFY1 | 0.020 | 5.1 | 80% | 10% |
| FMNL1 | 0.015 | 5.1 | 70% | 0% |
| EIF4A1 | 0.045 | 5.1 | 80% | 20% |
| FLNB | 0.010 | 5.1 | 100% | 40% |
| MVP | 0.005 | 5.1 | 100% | 50% |
| ADH5 | 0.037 | 5.1 | 90% | 30% |
| APOB | 0.045 | 5.1 | 70% | 10% |
| XRCC5 | 0.045 | 5.1 | 80% | 20% |
| TAGLN2 | 0.037 | 5.1 | 80% | 20% |
| MNDA | 0.024 | 5.0 | 70% | 10% |
| CD44 | 0.028 | 5.0 | 80% | 20% |
| CAPN2 | 0.010 | 5.0 | 100% | 40% |
| TYMP | 0.024 | 5.0 | 70% | 10% |
| ANXA1 | 0.005 | 5.0 | 100% | 50% |
| GC | 0.020 | 5.0 | 80% | 20% |
| EHD2 | 0.020 | 5.0 | 90% | 30% |
| CLIC4 | 0.024 | 5.0 | 90% | 30% |
| ILK | 0.020 | 5.0 | 80% | 20% |
| UBA1 | 0.041 | 5.0 | 90% | 30% |
| IGHV3-72 | 0.041 | 5.0 | 70% | 10% |
| CYFIP1 | 0.010 | 5.0 | 90% | 30% |
| ADAM10 | 0.024 | 5.0 | 80% | 20% |
| ITGB6 | 0.020 | 4.9 | 70% | 10% |
| CDK4 | 0.033 | 4.9 | 60% | 0% |
| GPX1 | 0.057 | 4.9 | 80% | 20% |
| CTSZ | 0.028 | 4.9 | 70% | 10% |
| FLOT1 | 0.005 | 4.9 | 80% | 20% |
| CALR | 0.024 | 4.9 | 100% | 40% |
| CDK2 | 0.033 | 4.9 | 60% | 0% |
| MAPK3 | 0.041 | 4.9 | 70% | 10% |
| TALDO1 | 0.033 | 4.9 | 80% | 20% |
| C4A | 0.020 | 4.9 | 80% | 20% |
| MAPK1 | 0.033 | 4.9 | 80% | 20% |
| DYNLL1 | 0.045 | 4.9 | 80% | 20% |
| ALDH2 | 0.041 | 4.9 | 80% | 20% |
| AP2S1 | 0.028 | 4.9 | 60% | 0% |
| SDCBP2 | 0.020 | 4.9 | 70% | 10% |
| ARL8B | 0.010 | 4.9 | 80% | 20% |
| LCP1 | 0.015 | 4.9 | 80% | 20% |
| CAPNS1 | 0.024 | 4.9 | 80% | 20% |
| GNAS | 0.010 | 4.9 | 90% | 40% |
| FAP | 0.024 | 4.9 | 60% | 0% |
| CD5L | 0.015 | 4.9 | 80% | 20% |
| LRP1 | 0.010 | 4.8 | 90% | 30% |
| HSPB6 | 0.049 | 4.8 | 80% | 20% |
| SEPT7 | 0.041 | 4.8 | 80% | 20% |
| BSG | 0.033 | 4.8 | 60% | 0% |
| ALDOC | 0.041 | 4.8 | 70% | 10% |
| CAND1 | 0.045 | 4.8 | 90% | 30% |
| ATIC | 0.069 | 4.8 | 80% | 20% |
| PARP4 | 0.049 | 4.8 | 70% | 10% |
| GPX4 | 0.049 | 4.8 | 70% | 10% |
| ARHGAP1 | 0.057 | 4.8 | 80% | 20% |
| GPI | 0.020 | 4.8 | 80% | 20% |
| COPG1 | 0.041 | 4.8 | 80% | 20% |
| CYB5R3 | 0.053 | 4.8 | 60% | 0% |
| VOL | 0.010 | 4.8 | 70% | 10% |
| FBLN2 | 0.033 | 4.8 | 60% | 0% |
| FGG | 0.020 | 4.8 | 90% | 40% |
| ATP2B4 | 0.005 | 4.8 | 70% | 10% |
| COPA | 0.049 | 4.8 | 80% | 20% |
| GNA13 | 0.015 | 4.8 | 90% | 40% |
| CAPZA2 | 0.028 | 4.8 | 80% | 20% |
| ORM1 | 0.020 | 4.8 | 80% | 20% |
| PDXK | 0.041 | 4.8 | 80% | 20% |
| CPNE1 | 0.020 | 4.8 | 90% | 30% |
| TWF2 | 0.024 | 4.8 | 80% | 20% |
| HEXB | 0.028 | 4.8 | 60% | 0% |
| CDK17 | 0.015 | 4.8 | 60% | 0% |
| CTNNB1 | 0.010 | 4.8 | 80% | 20% |
| PEBP1 | 0.024 | 4.8 | 90% | 30% |
| PSME2 | 0.065 | 4.8 | 80% | 20% |
| DERA | 0.065 | 4.8 | 80% | 20% |
| RAP2B | 0.037 | 4.8 | 90% | 30% |
| LCK | 0.037 | 4.8 | 60% | 0% |
| JUP | 0.041 | 4.8 | 80% | 20% |
| CTSC | 0.041 | 4.7 | 70% | 10% |
| CCT3 | 0.053 | 4.7 | 80% | 20% |
| PSMD2 | 0.049 | 4.7 | 80% | 20% |
| SUSD2 | 0.049 | 4.7 | 70% | 10% |
| UGDH | 0.041 | 4.7 | 80% | 20% |
| TWF1 | 0.028 | 4.7 | 80% | 20% |
| SCPEP1 | 0.037 | 4.7 | 60% | 0% |
| ITGAX | 0.024 | 4.7 | 60% | 0% |
| RPS12 | 0.045 | 4.7 | 80% | 20% |
| CCT8 | 0.045 | 4.7 | 80% | 20% |
| PSMC2 | 0.049 | 4.7 | 80% | 20% |
| SNAP23 | 0.041 | 4.7 | 80% | 20% |
| SARS | 0.049 | 4.7 | 80% | 20% |
| HNRNPR | 0.045 | 4.7 | 80% | 20% |
| FAM129B | 0.024 | 4.7 | 90% | 30% |
| C1QB | 0.015 | 4.7 | 60% | 0% |
| KIF5B | 0.041 | 4.7 | 80% | 20% |
| CCT5 | 0.041 | 4.7 | 60% | 20% |
| CAPG | 0.024 | 4.7 | 60% | 0% |
| DNAJA2 | 0.041 | 4.7 | 70% | 10% |
| ATP6V0D1 | 0.020 | 4.7 | 70% | 10% |
| EPB41L1 | 0.028 | 4.7 | 70% | 10% |
| VAT1 | 0.015 | 4.6 | 90% | 40% |
| RP2 | 0.005 | 4.6 | 70% | 70% |
| S100A16 | 0.045 | 4.6 | 70% | 70% |
| ITIH2 | 0.041 | 4.6 | 70% | 20% |
| OSTF1 | 0.020 | 4.6 | 60% | 0% |
| GLO1 | 0.041 | 4.6 | 70% | 10% |
| EML4 | 0.045 | 4.6 | 80% | 20% |
| STX7 | 0.010 | 4.6 | 60% | 0% |
| CD81 | 0.037 | 4.6 | 70% | 20% |
| ITGAL | 0.024 | 4.6 | 70% | 10% |
| EHD4 | 0.010 | 4.6 | 70% | 10% |
| ENTPD1 | 0.033 | 4.6 | 60% | 0% |
| NCF2 | 0.037 | 4.6 | 60% | 0% |
| BANF1 | 0.033 | 4.6 | 90% | 40% |
| STXBP3 | 0.024 | 4.6 | 70% | 10% |
| ICAM1 | 0.041 | 4.6 | 60% | 0% |
| KLC1 | 0.033 | 4.6 | 60% | 0% |
| LUM | 0.041 | 4.6 | 70% | 10% |
| FGA | 0.024 | 4.6 | 100% | 50% |
| SKP1 | 0.045 | 4.6 | 60% | 0% |
| ALOX5 | 0.037 | 4.6 | 60% | 0% |
| ANO6 | 0.037 | 4.5 | 60% | 0% |
| ATP6V1E1 | 0.041 | 4.5 | 70% | 10% |
| TIMP1 | 0.024 | 4.5 | 60% | 0% |
| NCKAP1L | 0.041 | 4.5 | 70% | 10% |
| CD47 | 0.024 | 4.4 | 70% | 20% |
| AHCY | 0.015 | 4.4 | 100% | 50% |
| H2AFY2 | 0.083 | 4.4 | 70% | 20% |
| EEF1G | 0.041 | 4.4 | 90% | 40% |
| PRKAG1 | 0.049 | 4.4 | 60% | 0% |
| PDIA3 | 0.053 | 4.4 | 90% | 30% |
| ITGA3 | 0.010 | 4.4 | 90% | 40% |
| H3F3A | 0.069 | 4.4 | 80% | 40% |
| PRKAA1 | 0.041 | 4.4 | 70% | 10% |
| YES1 | 0.020 | 4.4 | 90% | 40% |
| YWHAG | 0.015 | 4.4 | 90% | 40% |
| MYO1F | 0.037 | 4.4 | 60% | 0% |
| MIF | 0.079 | 4.4 | 80% | 30% |
| TPP2 | 0.049 | 4.4 | 80% | 30% |
| RAB34 | 0.033 | 4.4 | 60% | 0% |
| APOH | 0.015 | 4.4 | 90% | 40% |
| ENDOD1 | 0.033 | 4.3 | 70% | 10% |
| IDH1 | 0.005 | 4.3 | 100% | 50% |
| S100A11 | 0.020 | 4.3 | 90% | 50% |
| MYL9 | 0.076 | 4.3 | 50% | 0% |
| IGHG4 | 0.061 | 4.3 | 60% | 10% |
| DSTN | 0.049 | 4.3 | 90% | 40% |
| S100A9 | 0.020 | 4.3 | 70% | 20% |
| CNP | 0.010 | 4.3 | 90% | 40% |
| TMSB10 | 0.028 | 4.3 | 60% | 10% |
| C7 | 0.020 | 4.3 | 90% | 40% |
| HNRNPU | 0.076 | 4.3 | 80% | 30% |
| COPB1 | 0.049 | 4.2 | 90% | 40% |
| PDCD6 | 0.015 | 4.2 | 70% | 20% |
| PFKP | 0.057 | 4.2 | 70% | 20% |
| XRCC6 | 0.073 | 4.2 | 80% | 30% |
| CORO1A | 0.079 | 4.2 | 70% | 20% |
| M6PR | 0.020 | 4.2 | 80% | 30% |
| TACSTD2 | 0.024 | 4.2 | 80% | 30% |
| CLU | 0.057 | 4.2 | 70% | 20% |
| THBS3 | 0.028 | 4.2 | 60% | 10% |
| FERMT2 | 0.024 | 4.2 | 90% | 40% |
| NAMPT | 0.037 | 4.2 | 70% | 20% |
| PYGB | 0.091 | 4.2 | 70% | 20% |
| CSE1L | 0.083 | 4.2 | 70% | 20% |
| ARPC4 | 0.094 | 4.2 | 60% | 10% |
| SRC | 0.028 | 4.2 | 80% | 30% |
| CYFIP2 | 0.024 | 4.2 | 70% | 20% |
| PPP2R1A | 0.083 | 4.2 | 70% | 20% |
| CTNNA1 | 0.010 | 4.2 | 90% | 40% |
| STAT1 | 0.049 | 4.2 | 70% | 20% |
| PPP1CC | 0.073 | 4.2 | 70% | 20% |
| OLFM4 | 0.020 | 4.2 | 90% | 50% |
| HNRNPK | 0.098 | 4.2 | 80% | 30% |
| PRMT1 | 0.065 | 4.2 | 70% | 20% |
| H2AFZ | 0.091 | 4.1 | 80% | 40% |
| CAMP | 0.065 | 4.1 | 50% | 0% |
| TUBB3 | 0.041 | 4.1 | 90% | 50% |
| ASS1 | 0.069 | 4.1 | 70% | 20% |
| MTPN | 0.076 | 4.1 | 70% | 20% |
| C6 | 0.024 | 4.1 | 100% | 50% |
| DHX9 | 0.091 | 4.1 | 70% | 20% |
| SQSTM1 | 0.053 | 4.1 | 80% | 0% |
| ACTG1 | 0.005 | 4.1 | 100% | 70% |
| COPG2 | 0.073 | 4.1 | 70% | 20% |
| PALLD | 0.069 | 4.1 | 60% | 10% |
| AKR1A1 | 0.061 | 4.1 | 90% | 40% |
| PDLIM5 | 0.024 | 4.1 | 70% | 20% |
| THY1 | 0.037 | 4.1 | 80% | 40% |
| FGFR4 | 0.005 | 4.1 | 50% | 0% |
| PSMD13 | 0.087 | 4.1 | 70% | 20% |
| APOE | 0.083 | 4.1 | 70% | 20% |
| ARHGDIB | 0.069 | 4.1 | 90% | 40% |
| MYO1C | 0.033 | 4.1 | 90% | 40% |
| RHOC | 0.005 | 4.1 | 100% | 60% |
| HNRNPM | 0.053 | 4.1 | 60% | 10% |
| VPS29 | 0.079 | 4.1 | 70% | 20% |
| EPB41L3 | 0.053 | 4.1 | 70% | 20% |
| BASP1 | 0.005 | 4.1 | 100% | 60% |
| SEPT8 | 0.061 | 4.1 | 60% | 10% |
| C1QC | 0.010 | 4.1 | 50% | 0% |
| PSMD12 | 0.091 | 4.1 | 70% | 20% |
| CNN2 | 0.079 | 4.1 | 50% | 0% |
| TUBB6 | 0.037 | 4.0 | 90% | 50% |
| CDK1 | 0.069 | 4.0 | 50% | 0% |
| CDK15 | 0.069 | 4.0 | 50% | 0% |
| FGFR3 | 0.069 | 4.0 | 50% | 0% |
| TUBA4A | 0.045 | 4.0 | 90% | 50% |
| PAFAH1B2 | 0.079 | 4.0 | 70% | 20% |
| CLIC2 | 0.073 | 4.0 | 80% | 30% |
| CD151 | 0.010 | 4.0 | 50% | 0% |
| IGKV3-20 | 0.076 | 4.0 | 50% | 0% |
| CTSD | 0.045 | 4.0 | 90% | 50% |
| C8B | 0.057 | 4.0 | 90% | 40% |
| EIF3F | 0.049 | 4.0 | 80% | 30% |
| AP2A1 | 0.073 | 4.0 | 80% | 30% |
| FERMT1 | 0.033 | 4.0 | 70% | 20% |
| HLA-C | 0.045 | 4.0 | 80% | 40% |
| RNH1 | 0.091 | 4.0 | 70% | 20% |
| HNRNPL | 0.091 | 4.0 | 70% | 20% |
| EHD3 | 0.028 | 4.0 | 50% | 0% |
| ATP6V1A | 0.098 | 4.0 | 70% | 20% |
| BGN | 0.083 | 4.0 | 50% | 0% |
| HSPB1 | 0.033 | 4.0 | 90% | 50% |
| DHAJA1 | 0.076 | 4.0 | 60% | 10% |
| AP2A2 | 0.083 | 4.0 | 80% | 30% |
| PRKDC | 0.083 | 4.0 | 70% | 20% |
| FHL2 | 0.079 | 4.0 | 50% | 0% |
| HSPG2 | 0.057 | 4.0 | 70% | 20% |
| PRDX5 | 0.079 | 4.0 | 80% | 30% |
| IFI16 | 0.073 | 4.0 | 60% | 10% |
| PSMD7 | 0.091 | 4.0 | 70% | 20% |
| ITPA | 0.098 | 4.0 | 60% | 10% |
| PSMC1 | 0.094 | 4.0 | 70% | 20% |
| PSMD11 | 0.087 | 4.0 | 70% | 20% |
| RAB21 | 0.079 | 4.0 | 70% | 20% |
| SNRNP200 | 0.073 | 4.0 | 70% | 20% |
| EPRS | 0.098 | 4.0 | 70% | 20% |
| MAPRE2 | 0.079 | 4.0 | 50% | 0% |
| APOA4 | 0.098 | 3.9 | 60% | 10% |
| CCT6A | 0.098 | 3.9 | 80% | 30% |
| PLAA | 0.037 | 3.9 | 60% | 10% |
| RAP1A | 0.005 | 3.9 | 100% | 60% |
| C1S | 0.079 | 3.9 | 50% | 0% |
| C1QA | 0.087 | 3.9 | 50% | 0% |
| PLS1 | 0.045 | 3.9 | 80% | 30% |
| DDX3X | 0.094 | 3.9 | 70% | 20% |
| TSG101 | 0.020 | 3.9 | 60% | 10% |
| VWF | 0.076 | 3.9 | 70% | 20% |
| USP14 | 0.076 | 3.9 | 80% | 30% |
| LIN7C | 0.061 | 3.9 | 50% | 0% |
| PSMC6 | 0.098 | 3.9 | 70% | 20% |
| GRN | 0.061 | 3.9 | 60% | 10% |
| RDX | 0.049 | 3.9 | 80% | 40% |
| NAP1L4 | 0.065 | 3.9 | 60% | 10% |
| CLTC | 0.005 | 3.9 | 100% | 60% |
| AEBP1 | 0.053 | 3.9 | 50% | 0% |
| SNX6 | 0.094 | 3.9 | 70% | 20% |
| LGALS9 | 0.079 | 3.9 | 50% | 0% |
| PTGIS | 0.053 | 3.9 | 50% | 0% |
| SF3B3 | 0.076 | 3.9 | 70% | 20% |
| GALE | 0.028 | 3.9 | 50% | 0% |
| YWHAH | 0.024 | 3.9 | 90% | 50% |
| PROM1 | 0.049 | 3.9 | 50% | 0% |
| FCGRT | 0.079 | 3.9 | 70% | 20% |
| TTLL12 | 0.073 | 3.9 | 60% | 10% |
| EIF2AK2 | 0.069 | 3.9 | 50% | 0% |
| AKAP12 | 0.083 | 3.9 | 50% | 0% |
| IPO7 | 0.079 | 3.9 | 70% | 20% |
| VAMP8 | 0.061 | 3.9 | 50% | 0% |
| LGALS3BP | 0.020 | 3.9 | 100% | 60% |
| CFH | 0.083 | 3.9 | 50% | 0% |
| TMED9 | 0.041 | 3.9 | 60% | 10% |
| GNG2 | 0.065 | 3.8 | 60% | 10% |
| RAC1 | 0.053 | 3.8 | 80% | 40% |
| PRTN3 | 0.053 | 3.8 | 60% | 10% |
| VTA1 | 0.079 | 3.8 | 50% | 0% |
| ATP1B3 | 0.083 | 3.8 | 60% | 10% |
| SWAP70 | 0.079 | 3.8 | 50% | 0% |
| BROX | 0.076 | 3.8 | 80% | 30% |
| PRKAR2A | 0.091 | 3.8 | 80% | 30% |
| MYO1E | 0.076 | 3.8 | 50% | 0% |
| TTYH3 | 0.087 | 3.8 | 60% | 10% |
| RABGGTA | 0.079 | 3.8 | 50% | 0% |
| CASK | 0.098 | 3.8 | 60% | 10% |
| PPP2R2A | 0.098 | 3.8 | 70% | 20% |
| ITGA5 | 0.076 | 3.8 | 50% | 0% |
| NCF1 | 0.079 | 3.8 | 50% | 0% |
| MYL12A | 0.076 | 3.8 | 50% | 10% |
| GLUL | 0.083 | 3.8 | 50% | 0% |
| CAMK2D | 0.079 | 3.8 | 70% | 20% |
| ARHGEF1 | 0.061 | 3.8 | 70% | 20% |
| HSP90AB4P | 0.041 | 3.8 | 90% | 50% |
| PDCD6IP | 0.041 | 3.8 | 90% | 50% |
| UCHL1 | 0.079 | 3.8 | 50% | 0% |
| ABR | 0.061 | 3.8 | 60% | 10% |
| SEPT10 | 0.091 | 3.8 | 60% | 10% |
| NAPA | 0.098 | 3.8 | 70% | 20% |
| FHL1 | 0.091 | 3.8 | 90% | 40% |
| STX4 | 0.028 | 3.8 | 50% | 0% |
| DNAJC13 | 0.057 | 3.8 | 60% | 10% |
| IFIT3 | 0.079 | 3.8 | 50% | 0% |
| CD55 | 0.049 | 3.8 | 50% | 0% |
| C4BPA | 0.073 | 3.8 | 60% | 20% |
| TLN1 | 0.024 | 3.8 | 90% | 50% |
| KCNAB2 | 0.083 | 3.8 | 50% | 0% |
| PRKCB | 0.087 | 3.7 | 60% | 10% |
| AKR1B10 | 0.094 | 3.7 | 60% | 10% |
| RRAGC | 0.079 | 3.7 | 50% | 0% |
| YWHAE | 0.024 | 3.7 | 90% | 50% |
| PARVG | 0.079 | 3.7 | 50% | 0% |
| SLC44A2 | 0.061 | 3.7 | 60% | 10% |
| PEF1 | 0.005 | 3.7 | 50% | 0% |
| CYBA | 0.015 | 3.7 | 50% | 0% |
| LDHA | 0.005 | 3.7 | 100% | 60% |
| YWHAQ | 0.033 | 3.7 | 90% | 50% |
| ITGAV | 0.024 | 3.7 | 90% | 50% |
| CD59 | 0.057 | 3.7 | 90% | 50% |
| SERPINA1 | 0.037 | 3.7 | 90% | 50% |
| ENO1 | 0.005 | 3.7 | 100% | 70% |
| IMPDH2 | 0.083 | 3.7 | 60% | 10% |
| PARP9 | 0.079 | 3.6 | 50% | 0% |
| LSR | 0.015 | 3.6 | 50% | 0% |
| JCHAIN | 0.053 | 3.6 | 80% | 40% |
| RALA | 0.045 | 3.6 | 80% | 40% |
| RASA1 | 0.076 | 3.6 | 50% | 0% |
| GAPDH | 0.015 | 3.6 | 100% | 70% |
| PTGFRN | 0.061 | 3.6 | 80% | 40% |
| STK4 | 0.076 | 3.6 | 50% | 0% |
| STK10 | 0.069 | 3.6 | 50% | 0% |
| PARK7 | 0.098 | 3.6 | 70% | 20% |
| ADGRE5 | 0.065 | 3.6 | 50% | 0% |
| CACNA2D1 | 0.079 | 3.6 | 50% | 0% |
| RAB5A | 0.083 | 3.5 | 50% | 10% |
| LYN | 0.061 | 3.5 | 80% | 40% |
| LAP3 | 0.010 | 3.5 | 100% | 60% |
| PGAM1 | 0.041 | 3.5 | 80% | 40% |
| MUC5B | 0.076 | 3.5 | 60% | 20% |
| ARF5 | 0.061 | 3.5 | 80% | 40% |
| ITGA2 | 0.033 | 3.5 | 90% | 50% |
| RALB | 0.024 | 3.5 | 90% | 50% |
| NQO1 | 0.065 | 3.5 | 50% | 10% |
| KRT8 | 0.037 | 3.5 | 90% | 50% |
| XPO1 | 0.091 | 3.5 | 70% | 30% |
| SFN | 0.094 | 3.4 | 70% | 30% |
| CTSG | 0.049 | 3.4 | 80% | 40% |
| PRDX6 | 0.087 | 3.4 | 90% | 50% |
| ITGA1 | 0.079 | 3.4 | 80% | 40% |
| LXN | 0.076 | 3.4 | 50% | 10% |
| S100A8 | 0.041 | 3.4 | 60% | 20% |
| HLA-B | 0.053 | 3.3 | 90% | 60% |
| HSP90AA1 | 0.005 | 3.3 | 100% | 70% |
| IGKV4-1 | 0.065 | 3.3 | 50% | 10% |
| GGT5 | 0.076 | 3.3 | 60% | 20% |
| AMBP | 0.065 | 3.3 | 50% | 10% |
| GNG12 | 0.073 | 3.3 | 60% | 20% |
| CSTB | 0.094 | 3.2 | 60% | 20% |
| PPIA | 0.073 | 3.2 | 90% | 60% |
| HPX | 0.079 | 3.2 | 50% | 10% |
| CSRP1 | 0.098 | 3.2 | 60% | 20% |
| EZR | 0.005 | 3.2 | 100% | 70% |
| LBP | 0.061 | 3.2 | 50% | 10% |
| AHNAK | 0.061 | 3.2 | 90% | 60% |
| CAP1 | 0.033 | 3.2 | 100% | 70% |
| ARL3 | 0.087 | 3.1 | 50% | 10% |
| HDAC1 | 0.098 | 3.1 | 50% | 10% |
| GOLPH3 | 0.079 | 3.1 | 50% | 10% |
| ARF3 | 0.020 | 3.1 | 100% | 70% |
| GNB4 | 0.061 | 3.1 | 90% | 60% |
| CD99 | 0.069 | 3.1 | 50% | 10% |
| TES | 0.033 | 3.1 | 50% | 10% |
| INF2 | 0.087 | 3.1 | 50% | 10% |
| ADH1B | 0.091 | 3.1 | 90% | 60% |
| RAC2 | 0.076 | 3.1 | 90% | 60% |
| ENPP4 | 0.061 | 3.1 | 50% | 10% |
| ANXA11 | 0.028 | 3.0 | 100% | 70% |
| VAC14 | 0.079 | 3.0 | 50% | 10% |
| CP | 0.024 | 3.0 | 100% | 70% |
| YWHAB | 0.076 | 3.0 | 90% | 60% |
| ANXA7 | 0.073 | 2.9 | 90% | 60% |
| FLNA | 0.033 | 2.9 | 100% | 70% |
| PKM | 0.010 | 2.9 | 100% | 80% |
| MPO | 0.010 | 2.7 | 100% | 80% |
| CTNND1 | 0.057 | 2.7 | 90% | 60% |
| PFN1 | 0.024 | 2.6 | 100% | 80% |
| GNAI2 | 0.005 | 2.6 | 100% | 80% |
| ITGB1 | 0.005 | 2.5 | 100% | 80% |
| HSP90AB1 | 0.015 | 2.5 | 100% | 80% |
| ANXA5 | 0.024 | 2.4 | 100% | 80% |
| EEF1A1 | 0.057 | 2.4 | 100% | 80% |
| HSPA8 | 0.015 | 2.4 | 100% | 80% |
| SDCBP | 0.020 | 2.4 | 100% | 80% |
| CDC42 | 0.024 | 2.2 | 100% | 80% |
| LTF | 0.061 | 2.2 | 100% | 80% |
| YWHAZ | 0.037 | 2.2 | 100% | 80% |
| RAP1B | 0.015 | 2.2 | 100% | 80% |
| RAB10 | 0.069 | 2.0 | 100% | 80% |
| ANXA2 | 0.005 | 1.8 | 100% | 90% |
| ACTB | 0.010 | 1.2 | 100% | 100% |
| ANXA6 | 0.037 | 1.0 | 100% | 109% |

TABLE 20
176 tumor-enriched EVP proteins in LuCa (14 pairs; >10-fold, false
discovery rate [FDR] <0.1, and ≥50% found in tumor-derived
EVPs from tumor tissues (TT). Proteins in bold are exclusively expressed in TT.

| | false | | posivitiy | posivitiy | Positivity | Positivity |
|---|---|---|---|---|---|---|
| | discovery | log₁₀ | in tumor | in normal | in adjacent | in distant |
| | rate | fold change | tissue | tissue | tissue | tissue |
| protein | (FDR) | (tumor/normal) | (n = 14) | (n = 22) | (n = 14) | (n = 8) |
| FHL2 | 0.012 | 5.4 | 79% | 9% | 7% | 13% |
| XRN2 | 0.012 | 5.4 | 79% | 9% | 14% | 0% |
| GLRX | 0.012 | 4.9 | 86% | 23% | 36% | 0% |
| HDLBP | 0.012 | 4.8 | 86% | 27% | 36% | 13% |
| SRRT | 0.032 | 4.7 | 79% | 18% | 29% | 0% |
| RCC1 | 0.012 | 4.6 | 79% | 23% | 36% | 0% |
| AP3S1 | 0.012 | 4.6 | 86% | 27% | 29% | 25% |
| SNRPD3 | 0.022 | 4.6 | 79% | 18% | 21% | 13% |
| NOP2 | 0.012 | 4.6 | 64% | 5% | 7% | 0% |
| RPL22 | 0.022 | 4.5 | 86% | 36% | 43% | 25% |
| DNAJC7 | 0.012 | 4.5 | 64% | 5% | 7% | 0% |
| STK39 | 0.012 | 4.5 | 71% | 14% | 14% | 13% |
| SRP54 | 0.012 | 4.3 | 86% | 32% | 43% | 13% |
| DHX36 | 0.012 | 4.3 | 86% | 27% | 36% | 13% |
| ELAVL1 | 0.022 | 4.3 | 64% | 9% | 7% | 13% |
| THBS2 | 0.012 | 4.3 | 71% | 18% | 29% | 0% |
| ACO2 | 0.040 | 4.3 | 86% | 32% | 43% | 13% |
| ACBD3 | 0.022 | 4.2 | 71% | 16% | 21% | 13% |
| SRP9 | 0.012 | 4.1 | 57% | 5% | 7% | 0% |
| THOC2 | 0.012 | 4.1 | 79% | 23% | 29% | 13% |
| HNRNPC | 0.012 | 4.1 | 100% | 55% | 79% | 13% |
| EIF5B | 0.012 | 4.1 | 93% | 41% | 43% | 38% |
| RALY | 0.012 | 4.0 | 57% | 5% | 7% | 0% |
| UCHL5 | 0.022 | 4.0 | 57% | 5% | 0% | 13% |
| KHDRBS1 | 0.040 | 4.0 | 79% | 32% | 50% | 0% |
| SF3B6 | 0.040 | 4.0 | 86% | 36% | 57% | 0% |
| WDR44 | 0.012 | 4.0 | 93% | 41% | 50% | 25% |
| BABAM2 | 0.022 | 4.0 | 86% | 36% | 50% | 13% |
| HTATIP2 | 0.012 | 3.9 | 50% | 0% | 0% | 0% |
| CSTF3 | 0.012 | 3.9 | 57% | 5% | 7% | 0% |
| RPL38 | 0.012 | 3.9 | 57% | 9% | 7% | 13% |
| RPL35A | 0.040 | 3.9 | 86% | 41% | 50% | 25% |
| PPP6R3 | 0.056 | 3.8 | 79% | 27% | 43% | 0% |
| POLR2L | 0.012 | 3.8 | 64% | 14% | 14% | 13% |
| NUP43 | 0.012 | 3.8 | 64% | 14% | 14% | 13% |
| CKAP5 | 0.022 | 3.8 | 64% | 14% | 21% | 0% |
| FBLIM1 | 0.012 | 3.8 | 71% | 23% | 21% | 25% |
| GLA | 0.022 | 3.8 | 57% | 9% | 14% | 0% |
| DCPS | 0.040 | 3.8 | 57% | 9% | 14% | 0% |
| TPRKB | 0.022 | 3.8 | 71% | 23% | 29% | 13% |
| ESRP1 | 0.040 | 3.8 | 64% | 14% | 14% | 13% |
| PABPC4 | 0.022 | 3.8 | 100% | 55% | 71% | 25% |
| GORASP2 | 0.022 | 3.8 | 71% | 27% | 36% | 13% |
| VCAN | 0.048 | 3.7 | 71% | 27% | 21% | 38% |
| RPS17 | 0.032 | 3.7 | 64% | 18% | 21% | 13% |
| LSM6 | 0.064 | 3.7 | 57% | 9% | 14% | 0% |
| PGM2L1 | 0.022 | 3.7 | 79% | 32% | 14% | 63% |
| RANGAP1 | 0.022 | 3.7 | 86% | 41% | 43% | 38% |
| COG5 | 0.012 | 3.7 | 57% | 9% | 14% | 0% |
| LSM4 | 0.012 | 3.7 | 71% | 27% | 36% | 13% |
| CTNNBL1 | 0.040 | 3.6 | 71% | 23% | 29% | 13% |
| FEN1 | 0.040 | 3.6 | 50% | 5% | 7% | 0% |
| CPSF7 | 0.032 | 3.6 | 79% | 32% | 43% | 13% |
| TEP1 | 0.012 | 3.6 | 93% | 45% | 36% | 63% |
| CPSF6 | 0.056 | 3.6 | 79% | 32% | 43% | 13% |
| DHX16 | 0.040 | 3.6 | 57% | 14% | 14% | 13% |
| METTL1 | 0.032 | 3.6 | 50% | 0% | 0% | 0% |
| TBC1D8B | 0.048 | 3.5 | 57% | 14% | 21% | 0% |
| POLR2C | 0.022 | 5.5 | 64% | 18% | 21% | 13% |
| PSPC1 | 0.022 | 3.5 | 64% | 23% | 29% | 13% |
| TRA2B | 0.040 | 3.5 | 64% | 18% | 29% | 0% |
| SF3A3 | 0.032 | 3.5 | 50% | 5% | 7% | 0% |
| RPF2 | 0.022 | 3.5 | 50% | 5% | 7% | 0% |
| NAA50 | 0.032 | 3.5 | 71% | 27% | 29% | 25% |
| TP53RK | 0.032 | 3.5 | 64% | 23% | 29% | 13% |
| USP39 | 0.032 | 3.4 | 79% | 36% | 36% | 38% |
| RPP30 | 0.012 | 3.4 | 50% | 5% | 7% | 0% |
| METAP1 | 0.040 | 3.4 | 64% | 23% | 29% | 13% |
| NCL | 0.040 | 3.4 | 64% | 23% | 21% | 25% |
| NSUN2 | 0.048 | 3.4 | 86% | 45% | 50% | 38% |
| RRP12 | 0.040 | 3.4 | 50% | 5% | 7% | 0% |
| PSMD4 | 0.040 | 3.4 | 79% | 36% | 29% | 50% |
| FAM91A1 | 0.032 | 3.4 | 93% | 50% | 50% | 50% |
| HERC4 | 0.048 | 3.4 | 79% | 36% | 50% | 13% |
| KDM1A | 0.056 | 3.4 | 71% | 27% | 29% | 25% |
| ASCC2 | 0.048 | 3.4 | 50% | 5% | 7% | 0% |
| UBE2Z | 0.056 | 3.4 | 79% | 36% | 50% | 13% |
| RAE1 | 0.048 | 3.4 | 79% | 36% | 50% | 13% |
| FLAD1 | 0.032 | 3.4 | 50% | 5% | 7% | 0% |
| KHSRP | 0.048 | 3.3 | 79% | 36% | 43% | 25% |
| HEATR5B | 0.022 | 3.3 | 50% | 5% | 7% | 0% |
| G3BP1 | 0.064 | 3.3 | 71% | 32% | 43% | 13% |
| PAICS | 0.032 | 3.3 | 79% | 38% | 36% | 38% |
| DBR1 | 0.048 | 3.3 | 57% | 14% | 21% | 0% |
| SEC23IP | 0.040 | 3.3 | 93% | 55% | 64% | 38% |
| RPS13 | 0.056 | 3.3 | 64% | 27% | 43% | 0% |
| FBXO22 | 0.078 | 3.3 | 64% | 23% | 21% | 25% |
| RPS25 | 0.071 | 3.2 | 50% | 14% | 14% | 13% |
| DTX3L | 0.022 | 3.2 | 86% | 45% | 50% | 38% |
| NPM1 | 0.040 | 3.2 | 86% | 50% | 57% | 38% |
| PSMG1 | 0.098 | 3.2 | 64% | 23% | 29% | 13% |
| NELFB | 0.098 | 3.2 | 64% | 23% | 29% | 13% |
| MRE11 | 0.022 | 3.2 | 50% | 9% | 14% | 0% |
| NAA10 | 0.085 | 3.2 | 64% | 23% | 21% | 25% |
| NT5C3A | 0.012 | 3.1 | 50% | 9% | 7% | 13% |
| CSTF1 | 0.048 | 3.1 | 50% | 9% | 0% | 25% |
| SUB1 | 0.040 | 3.1 | 93% | 59% | 57% | 63% |
| BRIX1 | 0.078 | 3.1 | 79% | 41% | 50% | 25% |
| PABPC1 | 0.022 | 3.1 | 100% | 64% | 79% | 38% |
| NXF1 | 0.091 | 3.1 | 64% | 23% | 29% | 13% |
| LUC7L2 | 0.022 | 3.1 | 50% | 9% | 7% | 13% |
| DDX23 | 0.022 | 3.1 | 50% | 9% | 7% | 13% |
| PYCR3 | 0.048 | 3.1 | 50% | 14% | 14% | 13% |
| CASP8 | 0.012 | 3.0 | 50% | 9% | 7% | 13% |
| RPL27 | 0.085 | 3.0 | 71% | 36% | 50% | 13% |
| FAM98B | 0.085 | 3.0 | 71% | 32% | 36% | 25% |
| SRM | 0.064 | 3.0 | 93% | 59% | 64% | 50% |
| CLUH | 0.071 | 3.0 | 50% | 9% | 14% | 0% |
| ZFPL1 | 0.032 | 3.0 | 50% | 14% | 14% | 13% |
| BSG | 0.040 | 3.0 | 50% | 14% | 21% | 0% |
| PHKB | 0.078 | 3.0 | 71% | 32% | 29% | 38% |
| TNC | 0.022 | 3.0 | 64% | 27% | 36% | 13% |
| TBK1 | 0.078 | 3.0 | 57% | 16% | 21% | 13% |
| DDX21 | 0.071 | 3.0 | 50% | 14% | 21% | 0% |
| CASP7 | 0.091 | 2.9 | 57% | 18% | 14% | 25% |
| RPS24 | 0.078 | 2.9 | 86% | 55% | 64% | 38% |
| SCYL1 | 0.048 | 2.9 | 86% | 50% | 50% | 50% |
| CASK | 0.091 | 2.9 | 57% | 18% | 29% | 0% |
| ITPA | 0.048 | 2.9 | 93% | 59% | 57% | 63% |
| UAP1 | 0.085 | 2.9 | 86% | 50% | 50% | 50% |
| UBE48 | 0.085 | 2.9 | 57% | 18% | 21% | 13% |
| DPP8 | 0.048 | 2.9 | 50% | 9% | 14% | 0% |
| RANBP1 | 0.032 | 2.9 | 50% | 14% | 21% | 0% |
| PHF5A | 0.091 | 2.9 | 71% | 36% | 36% | 38% |
| CPSF1 | 0.085 | 2.9 | 79% | 41% | 57% | 13% |
| SAMD9 | 0.098 | 2.8 | 64% | 27% | 43% | 0% |
| PMM2 | 0.078 | 2.8 | 86% | 55% | 57% | 50% |
| SSRP1 | 0.078 | 2.8 | 79% | 45% | 50% | 38% |
| WDR33 | 0.085 | 2.8 | 64% | 27% | 29% | 25% |
| SMG1 | 0.091 | 2.8 | 57% | 18% | 14% | 25% |
| NT5C2 | 0.064 | 2.8 | 79% | 45% | 36% | 63% |
| TBC1D9B | 0.085 | 2.8 | 57% | 23% | 21% | 25% |
| PACS1 | 0.032 | 2.7 | 50% | 14% | 7% | 25% |
| LGALS9 | 0.091 | 2.7 | 86% | 55% | 50% | 63% |
| SRPRA | 0.085 | 2.7 | 79% | 45% | 64% | 13% |
| TARS | 0.012 | 2.6 | 100% | 73% | 79% | 63% |
| ILKAP | 0.071 | 2.6 | 93% | 59% | 64% | 50% |
| PSMB7 | 0.078 | 2.6 | 93% | 64% | 64% | 63% |
| PBLD | 0.085 | 2.6 | 50% | 18% | 21% | 13% |
| UTP3 | 0.091 | 2.6 | 64% | 32% | 29% | 38% |
| ISG15 | 0.091 | 2.5 | 57% | 27% | 36% | 13% |
| RRP9 | 0.098 | 2.5 | 50% | 18% | 29% | 0% |
| SEC24D | 0.012 | 2.3 | 100% | 77% | 79% | 75% |
| RPL9 | 0.012 | 2.2 | 100% | 77% | 86% | 63% |
| SBDS | 0.098 | 2.2 | 93% | 68% | 64% | 75% |
| SYNCRIP | 0.032 | 2.1 | 100% | 77% | 79% | 75% |
| CUL4B | 0.032 | 2.1 | 100% | 77% | 86% | 63% |
| NANS | 0.012 | 2.0 | 100% | 82% | 86% | 75% |
| SRP68 | 0.012 | 1.9 | 100% | 82% | 86% | 75% |
| RCC2 | 0.022 | 1.8 | 100% | 82% | 86% | 75% |
| GIT2 | 0.012 | 1.6 | 100% | 82% | 86% | 75% |
| RPS7 | 0.022 | 1.6 | 100% | 86% | 86% | 88% |
| GMDS | 0.022 | 1.6 | 100% | 86% | 79% | 100% |
| ELOB | 0.032 | 1.5 | 100% | 86% | 93% | 75% |
| MCTS1 | 0.022 | 1.5 | 100% | 86% | 93% | 75% |
| HNRNPUL1 | 0.078 | 1.4 | 100% | 86% | 93% | 75% |
| SF3B3 | 0.056 | 1.4 | 100% | 86% | 79% | 100% |
| SEC23B | 0.056 | 1.4 | 100% | 86% | 79% | 100% |
| EIF4E | 0.078 | 1.4 | 100% | 86% | 93% | 75% |
| CTSB | 0.012 | 1.3 | 100% | 91% | 93% | 86% |
| NAA15 | 0.091 | 1.3 | 100% | 86% | 86% | 88% |
| CMPK1 | 0.096 | 1.3 | 100% | 86% | 86% | 86% |
| CCAR2 | 0.056 | 1.3 | 100% | 86% | 93% | 75% |
| RPS11 | 0.022 | 1.3 | 100% | 91% | 93% | 88% |
| ARFGEF1 | 0.022 | 1.2 | 100% | 91% | 93% | 88% |
| RPL3 | 0.022 | 1.2 | 100% | 91% | 93% | 88% |
| FBLN2 | 0.022 | 1.2 | 100% | 91% | 86% | 100% |
| GARS | 0.022 | 1.2 | 100% | 91% | 93% | 86% |
| RPL7 | 0.040 | 1.2 | 100% | 91% | 93% | 88% |
| RPS14 | 0.040 | 1.1 | 100% | 91% | 93% | 88% |
| AARS | 0.032 | 1.1 | 100% | 91% | 93% | 86% |
| CUL4A | 0.064 | 1.1 | 100% | 91% | 93% | 88% |
| GLB1 | 0.012 | 1.1 | 100% | 91% | 93% | 86% |
| SEPT9 | 0.056 | 1.1 | 100% | 91% | 100% | 75% |
| RPS18 | 0.022 | 1.0 | 100% | 95% | 100% | 86% |
| SUPT16H | 0.022 | 1.0 | 100% | 91% | 86% | 100% |

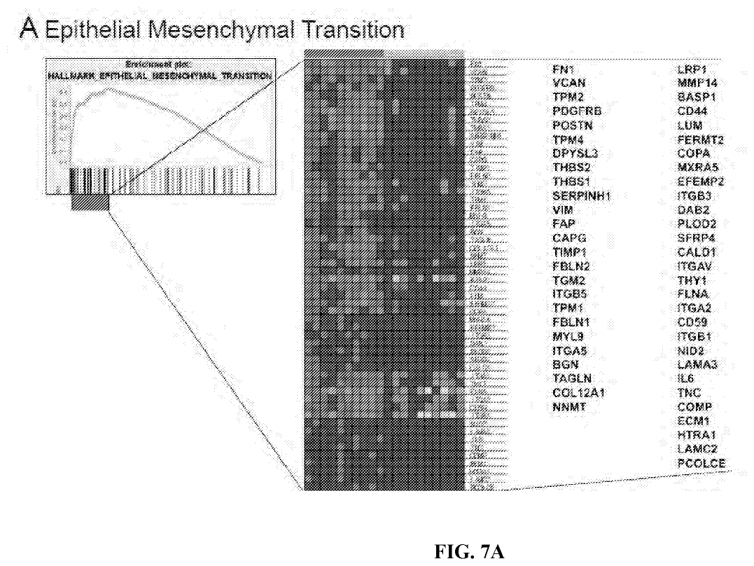

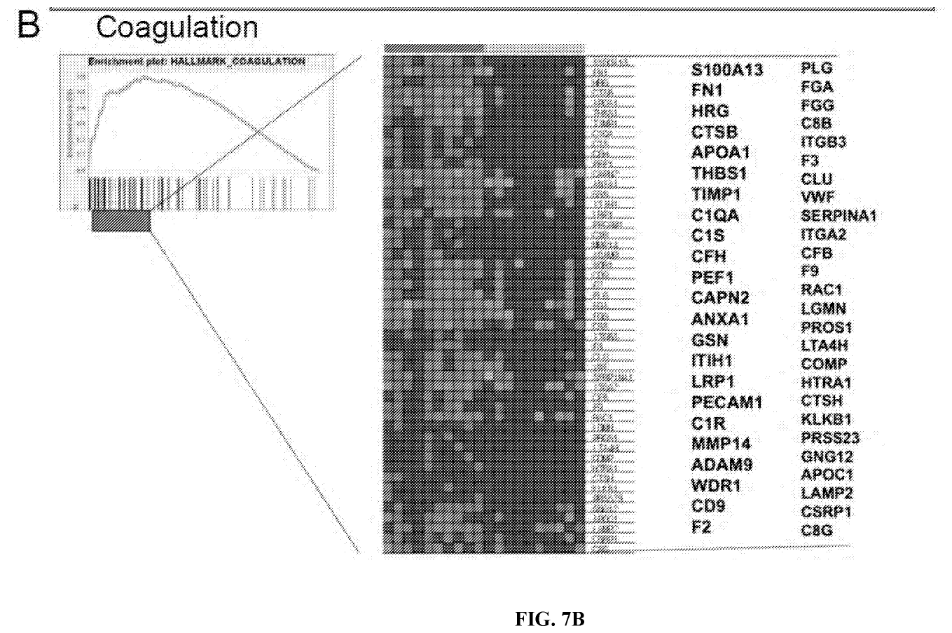

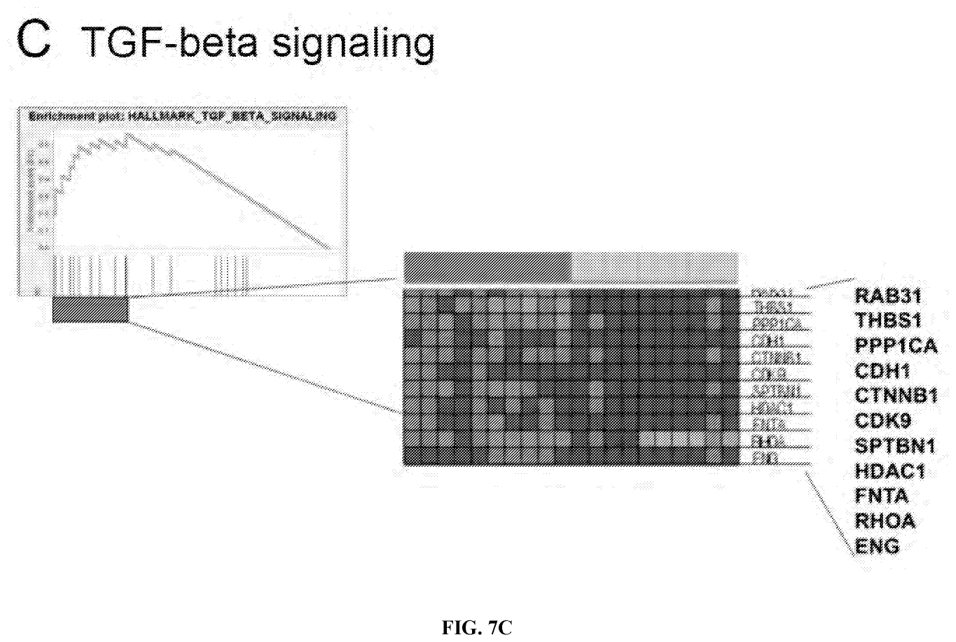

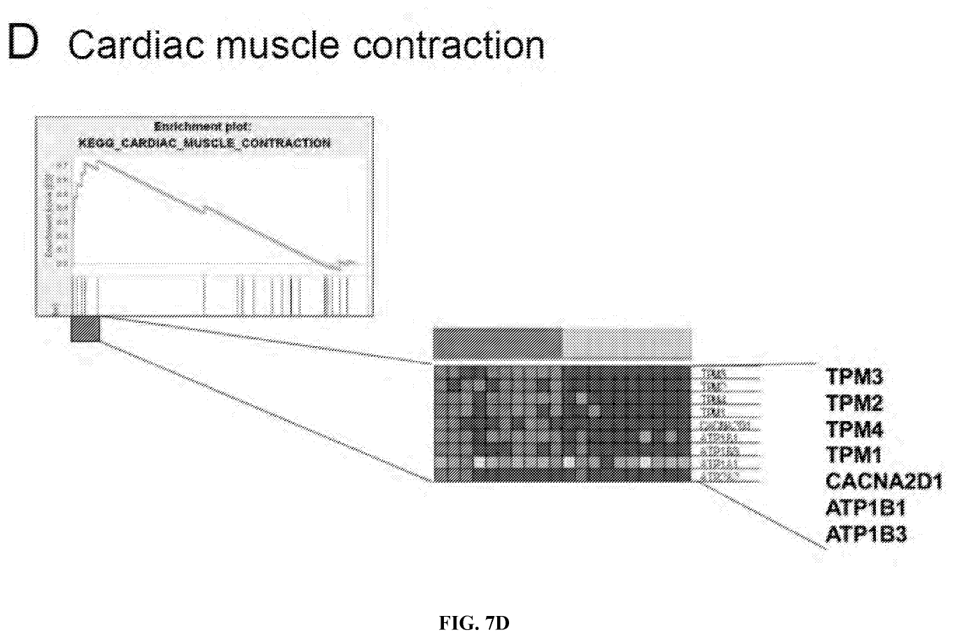

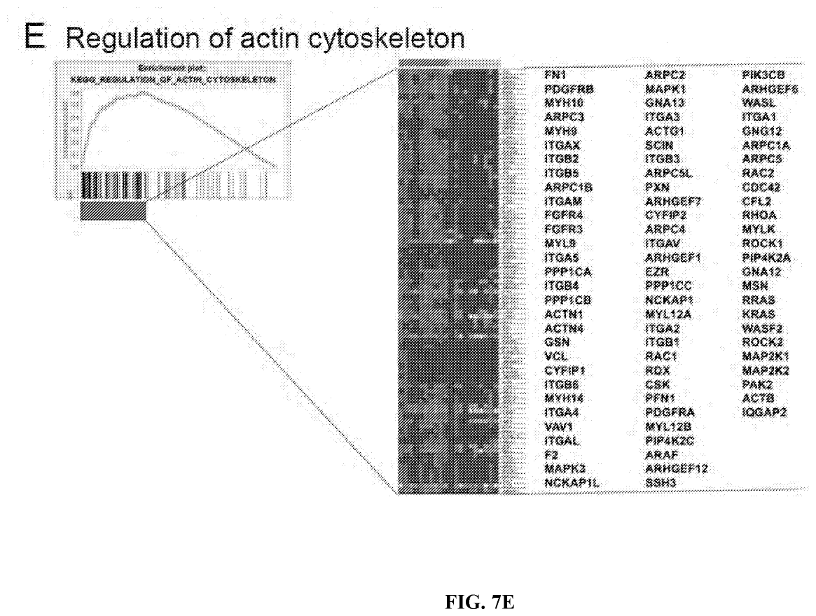

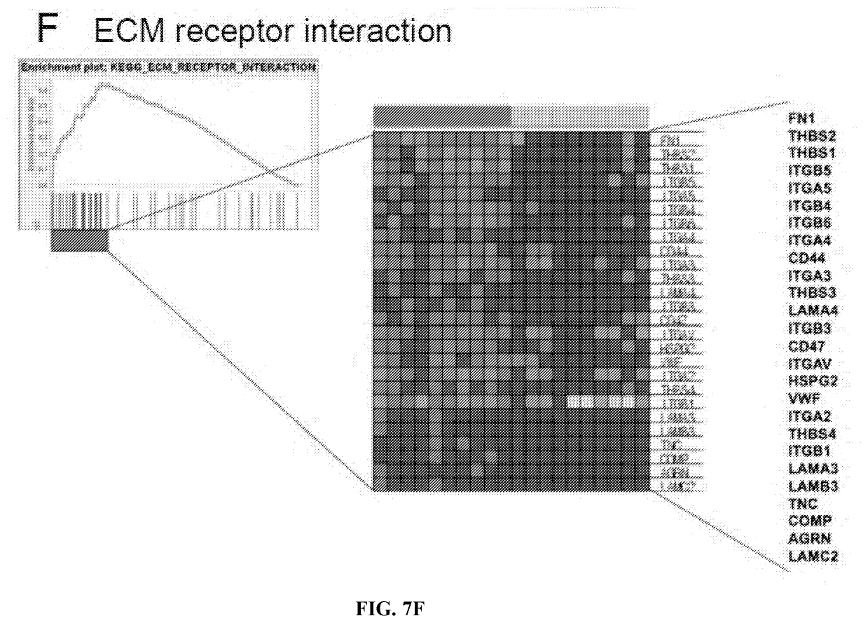

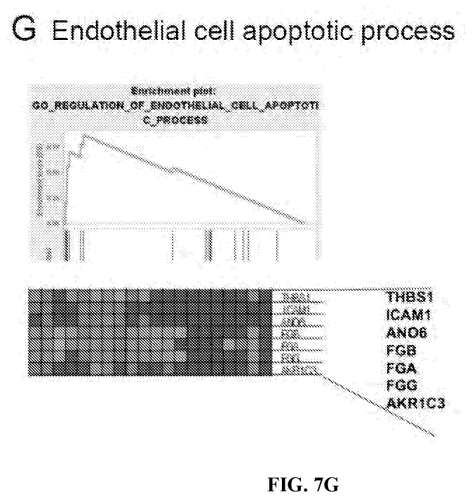

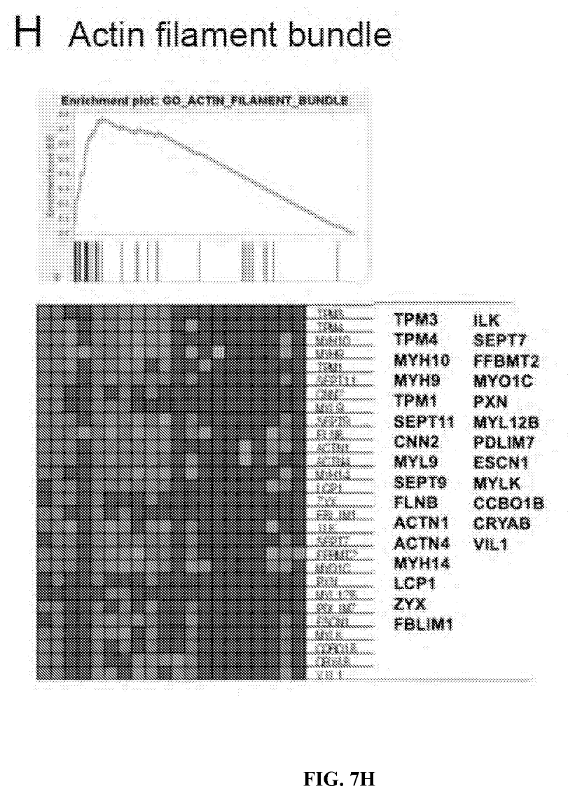

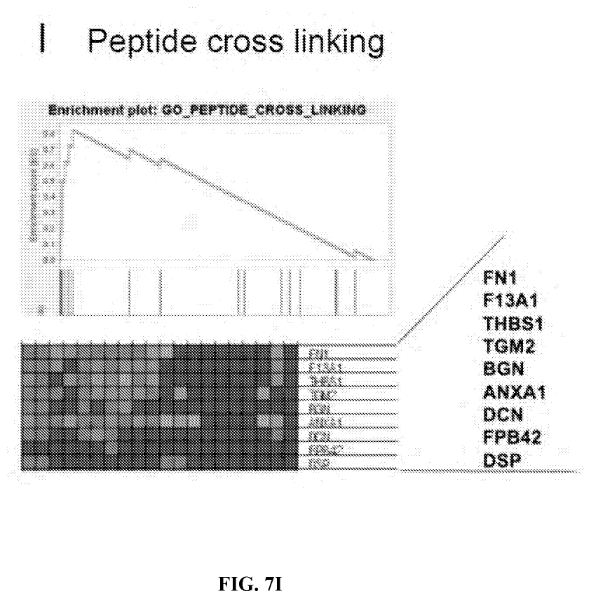

Of the 695 EVP proteins highly expressed in both PaCa and LuCa TT, 11 shared EVP proteins were identified: versican (VCAN), cathepsin B (CTSB), thrombospondin 2 (THBS2), septin 9 (SEPTIN9), basigin (BSG), fibulin 2 (FBLN2), four and a half LIM domains 2 (FHL2), inosine triphosphatase (ITPA), galectin-9 (LGALS9), splicing factor 3b subunit 1 3 (SF3B3) and calcium/calmodulin dependent serine protein kinase (CASK) (Tables 8 and 9). Classification of the pathways related to the top 30 enriched proteins from PaCa TT-derived EVPs using the GO Term Finder revealed that PaCa EVP-packaged proteins were involved in epithelial mesenchymal transition (EMT) [i.e., FN1, VCAN, tropomyosin alpha-4 chain (TPM4), dihydropyriminase-related protein 3 (DPYSL3), THBS2, thrombospondin 1 (THBS1), serpine H1 (SERPINH1), and vimentin (VIM)] and associated with cytoskeleton, filament assembly and the extracellular matrix (ECM) [i.e., FN1, myosin-10 (MYH10), actin-related protein 2/3 complex suunit 3 (ARPC3), myosin-9 (MYH9), THBS1, THBS2, tropomyosin alpha-3 chain (TPM3), and tropomyosin alpha-4 chain (TPM4)] consistent with many studies reporting changes in stiffness and ECM deposition in pancreatic cancer (Costa-Silva et al., “Pancreatic Cancer Exosomes Initiate Pre-Metastatic Niche Formation in the Liver,” Nature Cell Biology 17:816-826 (2015); Nielsen et al., “Key Players in Pancreatic Cancer4 Stroma Interaction: Cancer-Associated Fibroblasts, Endothelial and Inflammatory Cells,” World J Gastroenterol 22:2678-2700 (2016); Procacci et al., “Tumor(−)Stroma Cross-Talk in Human Pancreatic Ductal Adenocarcinoma: A Focus on the Effect of the Extracellular Matrix on Tumor Cell Phenotype and Invasive Potential,” Cells 7(10):158 (2018), which are hereby incorporated by reference in their entirety) (Table 21; FIG. 7).

TABLE 21
Gene Set Enrichment Analysis (GSEA) of enriched proteins in pancreatic cancer EVPs.

| | | | | | FDR q |
|---|---|---|---|---|---|
| | | | | | value |
| | | | | | (cut- |
| | | | | P | off < |
| Name | Size | ES | NES | value | 0.2) |
| HALLMARK_EPITHELIAL_MESENCHYMAL_TRANSITION | 114 | 0.64 | 1.56 | <0.001 | 0.008 |
| HALLMARK_COAGULATION | 92 | 0.60 | 1.45 | <0.001 | 0.020 |
| HALLMARK_TGF_BETA_SIGNALING | 19 | 0.67 | 1.41 | 0.018 | 0.024 |
| HALLMARK_COMPLEMENT | 115 | 0.58 | 1.40 | <0.001 | 0.022 |
| HALLMARK_MITOTIC_SPINDLE | 94 | 0.58 | 1.40 | <0.001 | 0.019 |
| HALLMARK_APICAL_JUNCTION | 108 | 0.58 | 1.39 | <0.001 | 0.018 |
| HALLMARK_PI3K_AKT_MTOR_SIGNALING | 65 | 0.58 | 1.37 | 0.002 | 0.022 |
| HALLMARK_HYPOXIA | 81 | 0.53 | 1.28 | 0.007 | 0.088 |
| HALLMARK_INTERFERON_GAMMA_RESPONSE | 104 | 0.52 | 1.27 | 0.003 | 0.096 |
| HALLMARK_INFLAMMATORY_RESPONSE | 49 | 0.54 | 1.27 | 0.033 | 0.090 |
| HALLMARK_ESTROGEN_RESPONSE_EARLY | 68 | 0.53 | 1.25 | 0.017 | 0.100 |
| HALLMARK_ESTROGEN_RESPONSE_LATE | 85 | 0.52 | 1.25 | 0.013 | 0.093 |
| HALLMARK_ANGIOGENESIS | 19 | 0.58 | 1.24 | 0.102 | 0.100 |
| HALLMARK_BILE_ACID_METABOLISM | 37 | 0.54 | 1.23 | 0.061 | 0.039 |
| HALLMARK_INTERFERON_ALPHA_RESPONSE | 57 | 0.53 | 1.22 | 0.042 | 0.110 |
| HALLMARK_MYOGENESIS | 70 | 0.51 | 1.22 | 0.032 | 0.110 |
| HALLMARK_IL6_JAK_STAT3_SIGNALING | 26 | 0.55 | 1.21 | 0.110 | 0.121 |
| HALLMARK_APOPTOSIS | 76 | 0.51 | 1.21 | 0.044 | 0.119 |
| HALLMARK_PROTEIN_SECRETION | 78 | 0.50 | 1.20 | 0.047 | 0.124 |
| HALLMARK_ALLOGRAFT_REJECTION | 83 | 0.50 | 1.19 | 0.037 | 0.146 |
| HALLMARK_IL2_STAT5_SIGNALING | 71 | 0.49 | 1.18 | 0.064 | 0.160 |
| KEGG_CARDIAC_MUSCLE_CONTRACTION | 25 | 0.74 | 1.64 | <0.001 | 0.016 |
| KEGG_REGULATION_OF_ACTIN_CYTOSKELETON | 120 | 0.61 | 1.49 | <0.001 | 0.103 |
| KEGG_ECM_RECEPTOR_INTERACTION | 50 | 0.65 | 1.49 | <0.001 | 0.069 |
| KEGG_COMPLEMENT_AND_COAGULATION_CASCADES | 56 | 0.63 | 1.47 | 0.001 | 0.079 |
| KEGG_SNARE_INTERACTIONS_IN_VESICULAR_TRANSPORT | 16 | 0.71 | 1.47 | 0.006 | 0.065 |
| KEGG_LEUKOCYTE_TRANSENDOTHELIAL_MIGRATION | 67 | 0.62 | 1.47 | <0.901 | 0.054 |
| KEGG_HYPERTROPHIC_CARDIOMYOPATHY_HCM | 43 | 0.63 | 1.46 | 0.001 | 0.055 |
| KEGG_DILATED_CARDIOMYOPATHY | 41 | 0.63 | 1.45 | 0.002 | 0.055 |
| KEGG_P53_SIGNALING_PATHWAY | 21 | 0.66 | 1.41 | 0.008 | 0.088 |
| KEGG_ARRHYTHMOGENIC_RIGHT_VENTRICULAR_CARDIOMYOPATHY_ARVC | 40 | 0.61 | 1.41 | 0.003 | 0.083 |
| KEGG_TIGHT_JUNCTION | 70 | 0.59 | 1.41 | <0.001 | 0.081 |
| KEGG_CELL_ADHESION_MOLECULES_CAMS | 50 | 0.60 | 1.41 | 0.002 | 0.076 |
| KEGG_PANCREATIC_CANCER | 34 | 0.62 | 1.40 | 0.006 | 0.074 |
| KEGG_FOCAL_ADHESION | 118 | 0.57 | 1.39 | <0.001 | 0.076 |
| KEGG_NATURAL_KILLER_CELL_MEDIATED_CYTOTOXICITY | 46 | 0.59 | 1.38 | 0.003 | 0.087 |
| KEGG_BLADDER_CANCER | 22 | 0.64 | 1.38 | 0.017 | 0.085 |
| KEGG_VASOPRESSIN_REGULATED_WATER_REABSORPTION | 25 | 0.62 | 1.38 | 0.017 | 0.098 |
| KEGG_TYPE_II_DIABETES_MELLITUS | 16 | 0.64 | 1.34 | 0.042 | 0.137 |
| KEGG_CHRONIC_MYELOID_LEUKEMIA | 32 | 0.60 | 1.33 | 0.023 | 0.137 |
| KEGG_PROSTATE_CANCER | 36 | 0.58 | 1.32 | 0.028 | 0.155 |
| KEGG_B_CELL_RECEPTOR_SIGNALING_PATHWAY | 39 | 0.57 | 1.31 | 0.023 | 0.155 |
| KEGG_VASCULAR_SMOOTH_MUSCLE_CONTRACTION | 44 | 0.56 | 1.31 | 0.018 | 0.164 |
| KEGG_ACUTE_MYELOID_LEUKEMIA | 27 | 0.59 | 1.30 | 0.044 | 0.177 |
| KEGG_ALDOSTERONE_REGULATED_SODIUM_REABSORPTION | 18 | 0.61 | 1.29 | 0.068 | 0.180 |
| KEGG_TGF_BETA_SIGNALING_PATHWAY | 27 | 0.58 | 1.29 | 0.045 | 0.180 |
| KEGG_PATHWAYS_IN_CANCER | 115 | 0.53 | 1.29 | 0.001 | 0.175 |
| KEGG_FC_GAMMA_R_MEDIATED_PHAGOCYTOSIS | 56 | 0.55 | 1.28 | 0.016 | 0.174 |
| KEGG_CELL_CYCLE | 46 | 0.55 | 1.28 | 0.019 | 0.168 |
| KEGG_PATHOGENIC_ESCHERICHIA_COLI_INFECTION | 43 | 0.56 | 1.28 | 0.024 | 0.162 |
| KEGG_LEISHMANIA_INFECTION | 39 | 0.57 | 1.28 | 0.030 | 0.159 |
| KEGG_AMINO_SUGAR_AND_NUCLEOTIDE_SUGAR_METABOLISM | 36 | 0.56 | 1.27 | 0.032 | 0.175 |
| KEGG_OOCYTE_MEIOSIS | 55 | 0.54 | 1.27 | 0.027 | 0.174 |
| KEGG_NUCLEOTIDE_EXCISION_REPAIR | 18 | 0.60 | 1.26 | 0.092 | 0.181 |
| KEGG_AMYOTROPHIC_LATERAL_SCLEROSIS_ALS | 15 | 0.61 | 1.26 | 0.112 | 0.195 |
| KEGG_VIRAL_MYOCARDITIS | 41 | 0.55 | 1.25 | 0.037 | 0.191 |
| (TOP 40 GO PATHWAY) | | | | | |
| GO_REGULATION_OF_ENDOTHELIAL_CELL_APOPTOTIC_PROCESS | 15 | 0.87 | 1.79 | <0.001 | 0.014 |
| GO_ACTIN_FILAMENT_BUNDLE | 37 | 0.77 | 1.75 | <0.001 | 0.014 |
| GO_PEPTIDE_CROSS_LINKING | 16 | 0.82 | 1.72 | <0.001 | 0.021 |
| GO_REGULATION_OF_ANTIGEN_RECEPTOR_MEDIATED_SIGNALING_PATHWAY | 21 | 0.79 | 1.70 | <0.001 | 0.022 |
| GO_ACTOMYOSIN | 41 | 0.74 | 1.69 | <0.001 | 0.021 |
| GO_CYCLIN_DEPENDENT_PROTEIN_KINASE_ACTIVITY | 15 | 0.83 | 1.69 | 0.001 | 0.020 |
| GO_GLIAL_CELL_MIGRATION | 15 | 0.81 | 1.66 | <0.001 | 0.032 |
| GO_STRUCTURAL_CONSTITUENT_OF_MUSCLE | 16 | 0.80 | 1.65 | <0.001 | 0.032 |
| GO_NEGATIVE_REGULATION_OF_ACTIN_FILAMENT_DEPOLYMERIZATION | 26 | 0.74 | 1.63 | 0.001 | 0.044 |
| GO_CELL_MATRIX_ADHESION | 61 | 0.69 | 1.63 | <0.001 | 0.041 |
| GO_NEGATIVE_REGULATION_OF_CELL_SUBSTRATE_ADHESION | 26 | 0.74 | 1.63 | <0.001 | 0.039 |
| GO_PLATELET_ALPHA_GRANULE_LUMEN | 31 | 0.73 | 1.62 | <0.001 | 0.039 |
| GO_FIBRONECTIN_BINDING | 15 | 0.77 | 1.60 | 0.002 | 0.051 |
| GO_CORTICAL_ACTIN_CYTOSKELETON | 38 | 0.70 | 1.60 | <0.001 | 0.049 |
| GO_ACTIN_FILAMENT_BASED_MOVEMENT | 41 | 0.70 | 1.60 | <0.001 | 0.046 |
| GO_PLASMA_MEMBRANE_RAFT | 36 | 0.71 | 1.60 | 0.001 | 0.044 |
| GO_COLLAGEN_BINDING | 35 | 0.70 | 1.60 | <0.001 | 0.042 |
| GO_REGULATION_OF_T_CELL_RECEPTOR_SIGNALING_PATHWAY | 15 | 0.77 | 1.59 | 0.002 | 0.046 |
| GO_CELL_CELL_ADHERENS_JUNCTION | 22 | 0.74 | 1.59 | 0.001 | 0.047 |
| GO_PROTEIN_COMPLEX_INVOLVED_IN_CELL_ADHESION | 23 | 0.72 | 1.59 | <0.001 | 0.045 |
| GO_INTEGRIN_MEDIATED_SIGNALING_PATHWAY | 52 | 0.67 | 1.58 | <0.001 | 0.049 |
| GO_NEGATIVE_REGULATION_OF_CELL_MATRIX_ADHESION | 16 | 0.75 | 1.58 | 0.003 | 0.047 |
| GO_REGULATION_OF_ACTIN_FILAMENT_DEPOLYMERIZATION | 33 | 0.70 | 1.57 | <0.001 | 0.050 |
| GO_POSITIVE_REGULATION_OF_COAGULATION | 16 | 0.76 | 1.57 | 0.002 | 0.050 |
| GO_VOLTAGE_GATED_ION_CHANNEL_ACTIVITY | 19 | 0.74 | 1.57 | 0.001 | 0.049 |
| GO_REGULATION_OF_SUBSTRATE_ADHESION_DEPENDENT_CELL_SPREADING | 31 | 0.70 | 1.57 | <0.001 | 0.050 |
| GO_PROTEOGLYCAN_BINDING | 15 | 0.76 | 1.56 | 0.003 | 0.051 |
| GO_ACTIN_MEDIATED_CELL_CONTRACTION | 28 | 0.71 | 1.56 | <0.001 | 0.051 |
| GO_MOTOR_ACTIVITY | 49 | 0.67 | 1.56 | <0.001 | 0.050 |
| GO_POSITIVE_REGULATION_OF_PHAGOCYTOSIS | 30 | 0.69 | 1.56 | 0.001 | 0.049 |
| GO_PLATELET_AGGREGATION | 30 | 0.70 | 1.56 | <0.001 | 0.049 |
| GO_CARDIAC_MUSCLE_CELL_DIFFERENTIATION | 24 | 0.70 | 1.56 | <0.001 | 0.047 |
| GO_BLOOD_COAGULATION_FIBRIN_CLOT_FORMATION | 18 | 0.74 | 1.56 | 0.001 | 0.048 |
| GO_PLATELET_DEGRANULATION | 66 | 0.66 | 1.56 | <0.001 | 0.047 |
| GO_REGULATION_OF_ENDOCYTOSIS | 106 | 0.64 | 1.55 | <0.001 | 0.047 |
| GO_PLATELET_ALPHA_GRANULE | 45 | 0.67 | 1.55 | <0.001 | 0.046 |
| GO_CORTICAL_CYTOSKELETON | 52 | 0.66 | 1.55 | <0.001 | 0.045 |
| GO_LAMININ_BINDING | 24 | 0.71 | 1.55 | <0.001 | 0.044 |
| GO_POSITIVE_REGULATION_OF_SUBSTRATE_ADHESION_DEPENDENT_CELL_SPREADING | 21 | 0.72 | 1.55 | 0.003 | 0.044 |
| GO_ACTIN_BINDING | 199 | 0.62 | 1.55 | <0.001 | 0.048 |

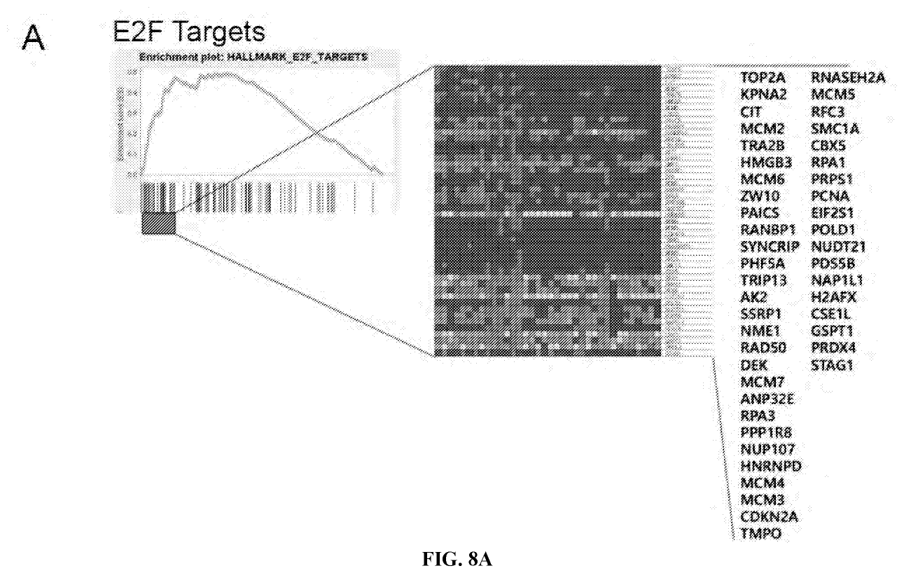

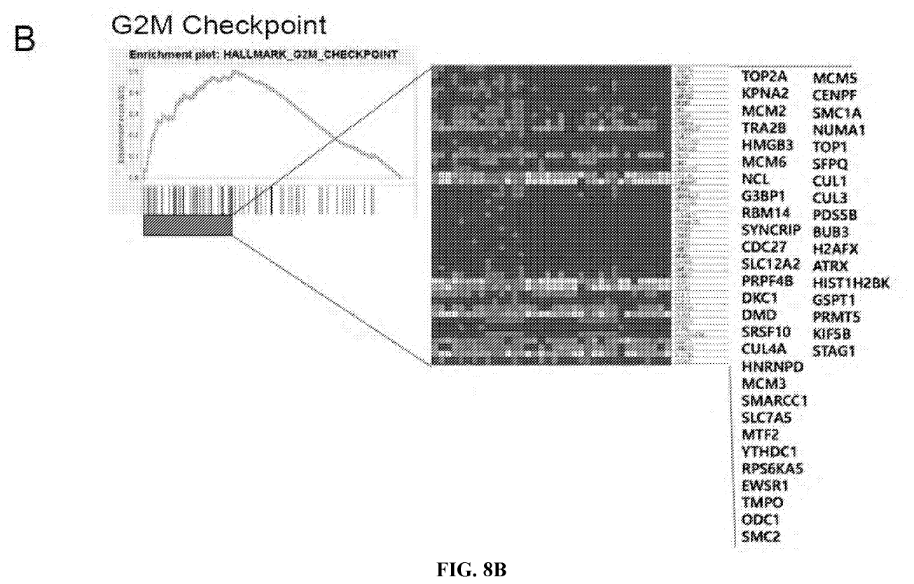

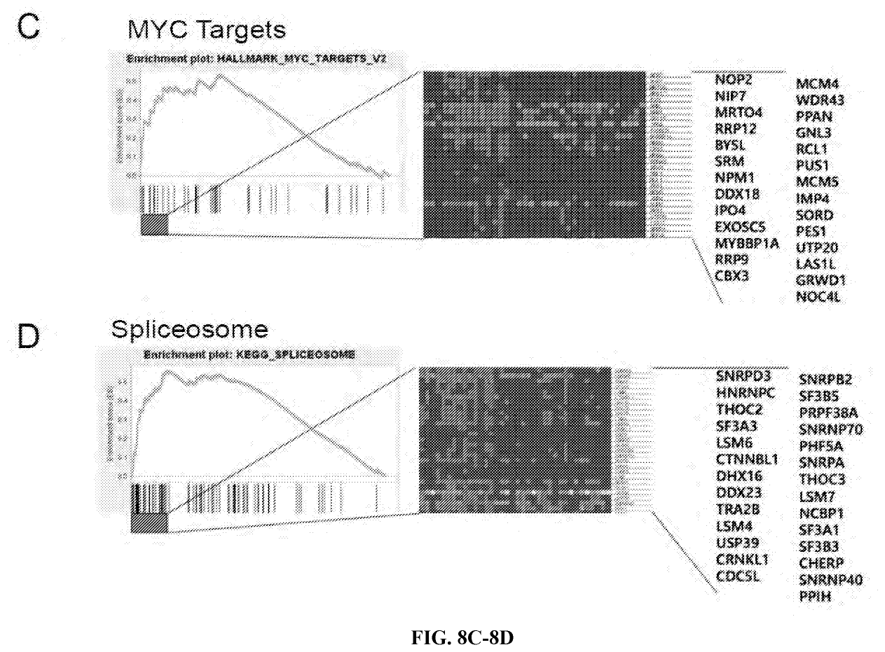

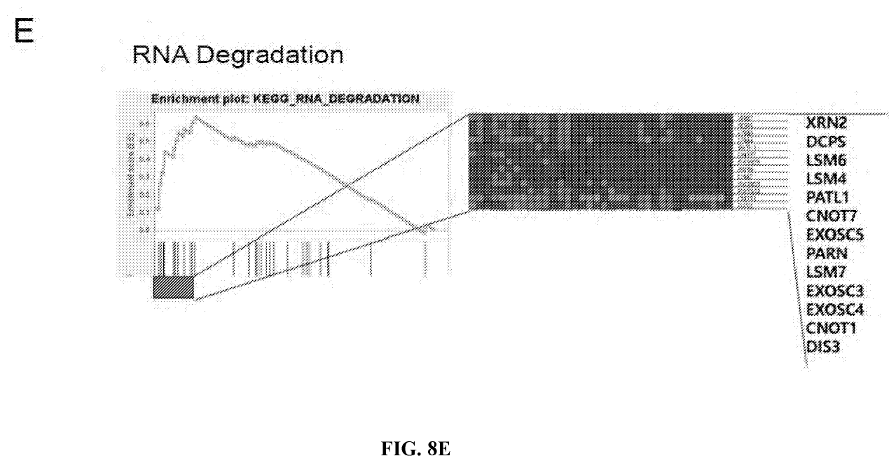

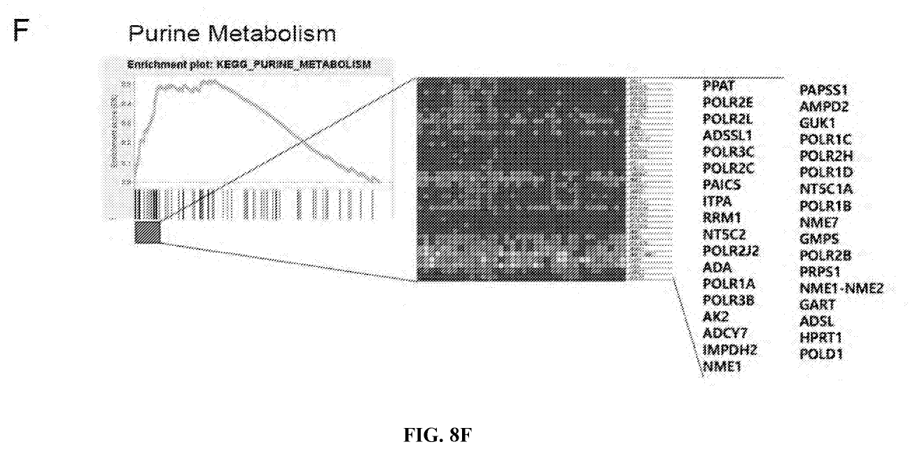

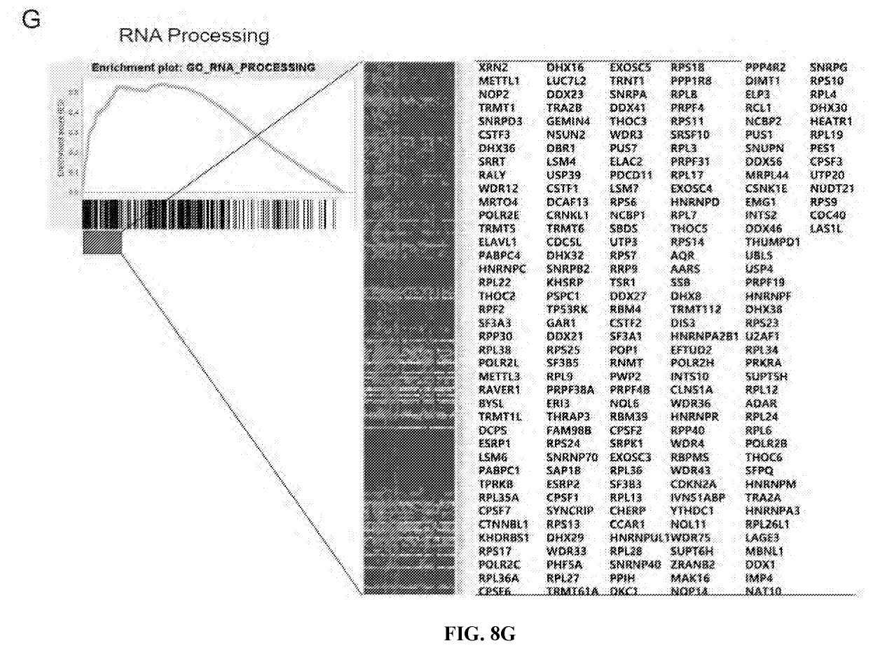

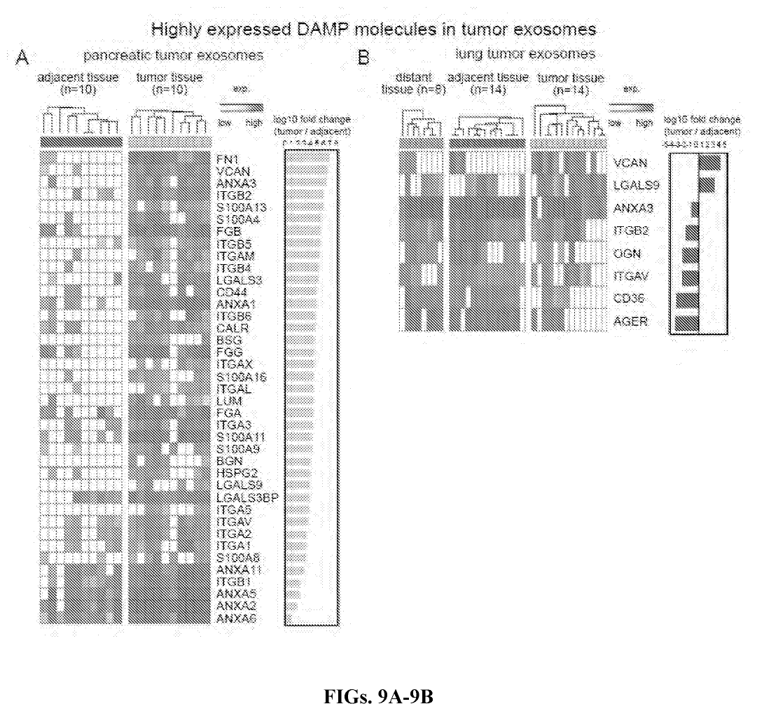

For LuCa, proteins related to Myc targets [small nuclear ribonucleoprotein Sm D3 (SNRPD3), AP-3 complex subunit sigma-1 (AP3S1), heterogenous nuclear ribonucleoproteins C1/C2 (HNRNPC) and 60 ribosomal protein L22 (RPL22)] and mRNA and RNA processing [5′-3′ exoribonuclease 2 (XRN2), tRNA (cytosine(72)-C(5))-methyltransferase NSUN6 (NOP2), SNRPD3, cleavage stimulation factor subunit 3 (CSTF3), ATP-dependent DNA/RNA helicase DHX36 (DHX36), serrate RNA effector molecule homolog (SRRT), RNA-binding protein Raly (RALY), ELAV-like protein 1-A (ELAVL1), HNRNPC, RPL22 and THO compels subunit 2 (THOC2)] were highly represented in TT-derived EVPs (Table 22; FIG. 8).

TABLE 22
Gene Set Enrichment Analysis (GSEA) of enriched proteins in lung cancer EVPs

| | | | | | FDR q |
|---|---|---|---|---|---|
| | | | | | value |
| | | | | P | (cutoff < |
| Name | Size | ES | NES | value | 0.2) |
| HALLMARK_E2F_TARGETS | 80 | 0.50 | 1.80 | <0.001 | 0.006 |
| HALLMARK_G2M_CHECKPOINT | 75 | 0.50 | 1.75 | <0.001 | 0.006 |
| HALLMARK_MYC_TARGETS_V1 | 170 | 0.44 | 1.73 | <0.001 | 0.004 |
| HALLMARK_MYC_TARGETS_V2 | 40 | 0.54 | 1.71 | <0.001 | 0.004 |
| HALLMARK_UNFOLDED_PROTEIN_RESPONSE | 58 | 0.48 | 1.63 | 0.003 | 0.011 |
| HALLMARK_GLYCOLYSIS | 98 | 0.37 | 1.36 | 0.031 | 0.187 |
| HALLMARK_DNA_REPAIR | 64 | 0.40 | 1.35 | 0.049 | 0.167 |
| KEGG_SPLICEOSOME | 83 | 0.56 | 1.98 | <0.001 | 0.001 |
| KEGG_RNA_DEGRADATION | 32 | 0.64 | 1.93 | <0.001 | 0.001 |
| KEGG_PURINE_METABOLISM | 67 | 0.52 | 1.81 | <0.001 | 0.009 |
| KEGG_RIBOSOME | 73 | 0.51 | 1.80 | <0.001 | 0.008 |
| KEGG_PYRIMIDINE_METABOLISM | 45 | 0.56 | 1.79 | 0.001 | 0.007 |
| KEGG_RNA_POLYMERASE | 15 | 0.71 | 1.79 | <0.001 | 0.007 |
| KEGG_AMINOACYL_TRNA_BIOSYNTHESIS | 24 | 0.52 | 1.49 | 0.028 | 0.181 |
| (TOP 40 GO PATHWAY) | | | | | |
| GO_RNA_PROCESSING | 389 | 0.55 | 2.28 | <0.001 | <0.001 |
| GO_MRNA_PROCESSING | 195 | 0.56 | 2.25 | <0.001 | <0.001 |
| GO_NCRNA_METABOLIC_PROCESS | 240 | 0.55 | 2.25 | <0.001 | <0.001 |
| GO_MRNA_METABOLIC_PROCESS | 317 | 0.54 | 2.22 | <0.001 | <0.001 |
| GO_RNA_SPLICING_VIA_TRANSESTERIFICATIO [symbol] | 149 | 0.56 | 2.20 | <0.001 | <0.001 |
| GO_RNA_SPLICING | 183 | 0.55 | 2.20 | <0.001 | <0.001 |
| GO_NCRNA_PROCESSING | 193 | 0.55 | 2.17 | <0.001 | <0.001 |
| GO_NUCLEOLUS | 314 | 0.52 | 2.13 | <0.001 | <0.001 |
| GO_TRNA_PROCESSING | 37 | 0.68 | 2.11 | <0.001 | <0.001 |
| GO_RIBOSOME_BIOGENESIS | 165 | 0.53 | 2.10 | <0.001 | <0.001 |
| GO_RNA_MODIFICATION | 40 | 0.65 | 2.08 | <0.001 | <0.001 |
| GO_RNA_CATABOLIC_PROCESS | 144 | 0.54 | 2.07 | <0.001 | <0.001 |
| GO_RIBONUCLEOPROTEIN_COMPLEX_BIOGENI [symbol] | 234 | 0.51 | 2.07 | <0.001 | <0.001 |
| GO_TRNA_METABOLIC_PROCESS | 72 | 0.58 | 2.06 | <0.001 | <0.001 |
| GO_RRNA_METABOLIC_PROCESS | 145 | 0.53 | 2.06 | <0.001 | <0.001 |
| GO_SMALL_NUCLEAR_RIBONUCLEAOPROTEIN_ [symbol] | 36 | 0.67 | 2.06 | <0.001 | <0.001 |
| GO_RNA_PHOSPHODIESTER_BOND_HYDROLY [symbol] | 46 | 0.64 | 2.05 | <0.001 | 0.001 |
| GO_NUCLEOLAR_PART | 31 | 0.68 | 2.04 | <0.001 | 0.001 |
| GO_SPLICEOSOMAL_COMPLEX | 97 | 0.55 | 2.03 | <0.001 | 0.001 |
| GO_RIBONUCLEOPROTEIN_COMPLEX | 351 | 0.49 | 2.03 | <0.001 | 0.001 |
| GO_TRNA_METABOLIC_PROCESS | 72 | 0.58 | 2.06 | <0.001 | <0.001 |
| GO_RRNA_METABOLIC_PROCESS | 146 | 0.53 | 2.06 | <0.001 | <0.001 |
| GO_SMALL_NUCLEAR_RIBONUCLEOPROTEIN_ [symbol] | 36 | 0.67 | 2.06 | <0.001 | <0.001 |
| GO_RNA_PHOSPHODIESTER_BOND_HYDROLY [symbol] | 46 | 0.64 | 2.05 | <0.001 | 0.001 |
| GO_NUCLEOLAR_PART | 31 | 0.68 | 2.04 | <0.001 | 0.001 |
| GO_SPLICEOSOMAL_COMPLEX | 97 | 0.55 | 2.03 | <0.001 | 0.001 |
| GO_RIBONUCLEOPROTEIN_COMPLEX | 351 | 0.49 | 2.03 | <0.001 | 0.001 |
| GO_RNA_PHOSPHODIESTER_BOND_HYDROLY [symbol] | 16 | 0.78 | 2.02 | <0.001 | 0.001 |
| GO_RIBONUCLEASE_ACTIVITY | 33 | 0.66 | 2.01 | <0.001 | 0.001 |
| GO_EXONUCLEASE_ACTIVITY | 26 | 0.68 | 1.99 | <0.001 | 0.001 |
| GO_RNA_3_END_PROCESSING | 51 | 0.60 | 1.98 | <0.001 | 0.001 |
| GO_NUCLEOPLASM_PART | 199 | 0.49 | 1.98 | <0.001 | 0.001 |
| GO_NUCLEIC_ACID_PHOSPHODIESTER_BOND_ [symbol] | 85 | 0.54 | 1.95 | <0.001 | 0.002 |
| GO_NUCLEOTIDYLTRANSFERASE_ACTIVITY | 47 | 0.59 | 1.94 | <0.001 | 0.003 |
| GO_RNA_POLYMERASE_COMPLEX | 36 | 0.62 | 1.94 | <0.001 | 0.003 |
| GO_SNRNA_METASOLIC_PROCESS | 28 | 0.65 | 1.94 | <0.001 | 0.003 |
| GO_TRANSFERASE_ACTIVITY_TRANSFERRING_ [symbol] | 58 | 0.57 | 1.93 | <0.001 | 0.003 |
| GO_MRNA_BINDING | 76 | 0.54 | 1.93 | <0.001 | 0.003 |
| GO_NUCLEASE_ACTIVITY | 56 | 0.57 | 1.93 | <0.001 | 0.003 |
| GO_METHYLATION | 76 | 0.54 | 1.91 | <0.001 | 0.004 |
| GO_RNA_METHYLATION | 15 | 0.75 | 1.91 | <0.001 | 0.003 |
| GO_TRNA_BINDING | 25 | 0.66 | 1.90 | <0.001 | 0.004 |
| GO_RNA_POLYMERASE_ACTIVITY | 16 | 0.72 | 1.88 | 0.001 | 0.006 |
| GO_S_ADENOSYLMETHIONINE_DEPENDENT_M [symbol] | 41 | 0.60 | 1.87 | <0.001 | 0.006 |
| GO_EXONUCLEASE_ACTIVITY_ACTIVE_WITH E [symbol] | 17 | 0.71 | 1.86 | <0.001 | 0.008 |
| GO_NUCLEAR_TRANSCRIBED_MRNA_CATABOL [symbol] | 27 | 0.64 | 1.86 | <0.001 | 0.008 |
| GO_DNA_TEMPLATED_TRANSCRIPTION_TERMI [symbol] | 49 | 0.56 | 1.85 | <0.001 | 0.008 |

[symbol]

indicates data missing or illegible when filed

Additionally, Gene Set Enrichment Analysis (GSEA) revealed that EMT, coagulation and actin signaling pathways were highly enriched in PaCa while cell cycle, metabolic and RNA processing pathways were significant in LuCa, respectively (FIGS. 7 and 8). Although the EMT pathway was found to be highly represented in PaCa EVPs (P<0.001), it was not significant in LuCa EVPs (P=0.49) and vice versa for RNA processing pathway in PaCa EVPs (P=0.77). The finding showing that PaCa and LuCa TT EVP cargo was enriched in proteins involved in distinct pathways demonstrates that EVP packaging is heterogenous across tumor types and reflects tumor biology.

In addition to examining EVP proteins overrepresented in TT, EVP proteins that were exclusive to TT versus AT/DT were also mined for and generated a list of proteins detected in ≥50% of either PaCa or LuCa TT samples but never found in AT or DT (FIG. 6C). Although over 50 proteins were identified, including ECM-related and pro-inflammatory proteins [e.g., periostin (POSTN), S100A13], exclusive to PaCa TT-derived EVPs, only two proteins, HIV-1 Tat interactive protein 2 (HTATIP2) and methyltransferase like 1 (METTL1), that were unique for LuCa TT-derived EVPs were found (FIG. 6C). Notably, among the top 30 EVP proteins enriched in PaCa TT (FIG. 6B), four proteins [flotillin 2 (FLOT2), TPM3, Fc fragment of IgE receptor (FCER1G) and G protein subunit alpha Q (GNAQ)] overlapped with proteins solely found in tumor EVPs (FIG. 6C). In LuCa, one protein identified in TT versus AT/DT comparison, HTATIP2, overlapped with EVP proteins exclusively present in tumor EVPs (FIG. 6B and FIG. 6C; Tables 4 and 5), further validating these proteins as having PaCa- and LuCa-specific biomarker potential. Taken together, these data suggest that cancer EVP proteins may reflect selective packaging which could potentially discriminate different types of cancer.

### Example 4—Pancreatic Tumor Tissue-Derived Extracellular Vesicle and Particle Expression of Mucins and Serpins

Human patient samples. Tumor samples from surgically resected primary pancreas ductal adenocarcinomas were from patients treated at Memorial Sloan Kettering Cancer Center (MSKCC), and at Shaare Zedek Medical Center, and Sheba Medical Center at Tel-Hashomer; consent to study the tissue was obtained via MSK TRB protocols #15-015 for the exosome analysis (Cohort 2; Table 23). This Cohort included fresh samples from 26 patients from which tumor tissues and/or normal adjacent controls were collected (Table 23). FFPE whole tumor sections and deeply annotated demographic, clinical, pathologic and genomic (MSK-IMPACTTM) data were collected for all patients in the study. In addition, fresh-frozen tumor tissue was collected for a subset of 12 patients

Proteomic analysis of human exosomes. Fresh pancreatic cancer tissue and peritumoral non-involved pancreas tissues were cut into small pieces and cultured for 24 hours in serum-free RPMI, supplemented with penicillin (100 U/ml) and streptomycin (100 μg/ml). Conditioned media was processed for exosome isolation. Exosomes were purified by sequential ultracentrifugation as previously described (see Bojmar et al., “Extracellular vesicle and particle isolation from human and murine cell lines, tissues, and bodily fluids,” STAR Protoc, 2(1): 100225 (2021) and Hoshino et al., “Extracellular Vesicle and Particle Biomarkers Define Multiple Human Cancers,” Cell 182(4): 1044-1061 e18 (2020), which are hereby incorporated by reference in their entirety). Briefly, cell contamination was removed from resected tissue culture supernatant by centrifugation at 500×g for 10 min. To remove apoptotic bodies and large cell debris, the supernatants were then spun at 3,000×g for 20 min, followed by centrifugation at 12,000×g for 20 min to remove large microvesicles. Finally, exosomes were collected by ultracentrifugation twice at 100,000×g for 70 min. Five micrograms of exosomal protein were used for mass spectrometry analysis (Hoshino et al., “Extracellular Vesicle and Particle Biomarkers Define Multiple Human Cancers,” Cell 182(4): 1044-1061 e18 (2020), which is hereby incorporated by reference in its entirety). High resolution/high mass accuracy nano-LC-MS/MS data was processed using Proteome Discoverer 1.4.1.14/Mascot 2.5. Human data was queried against the UniProt's Complete HUMAN proteome.

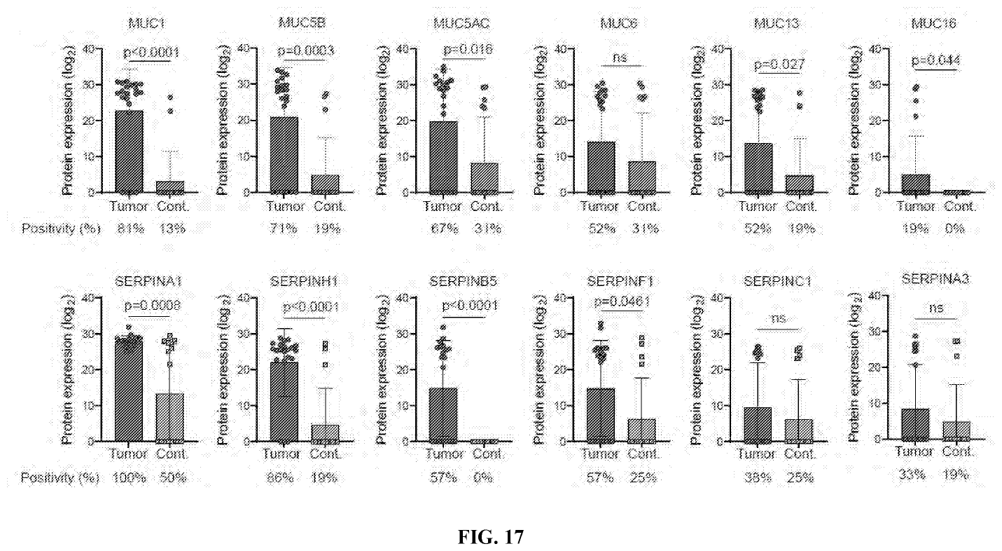

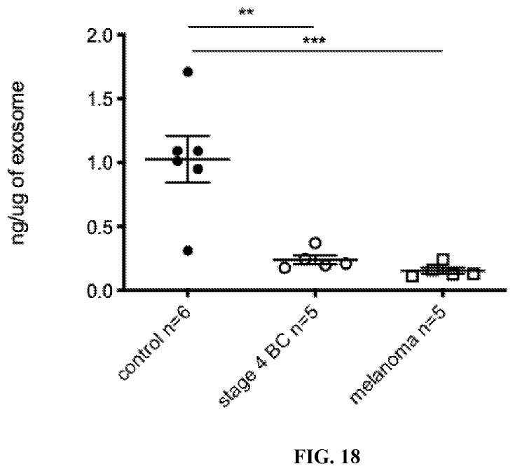

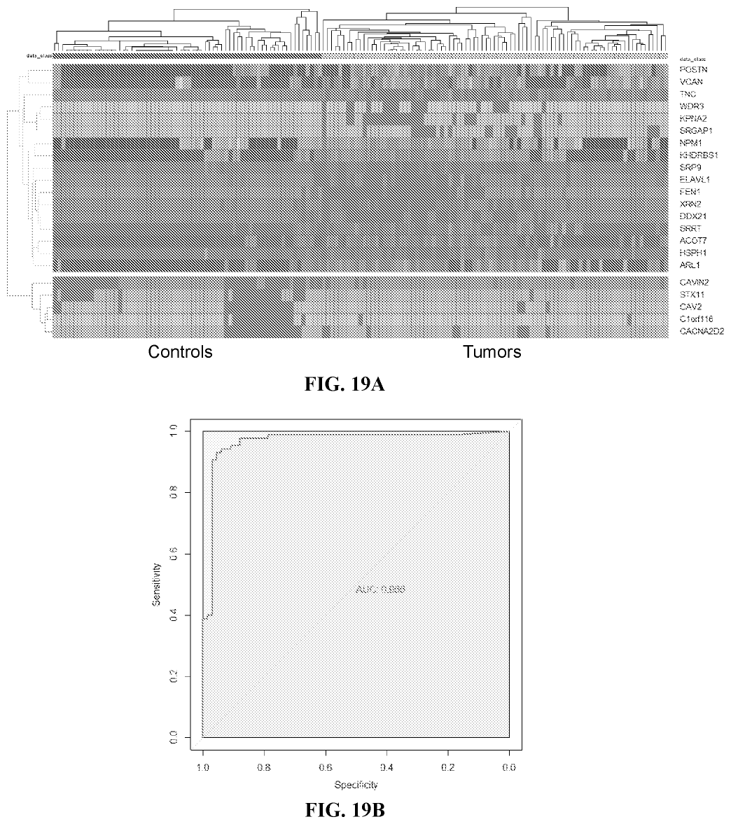

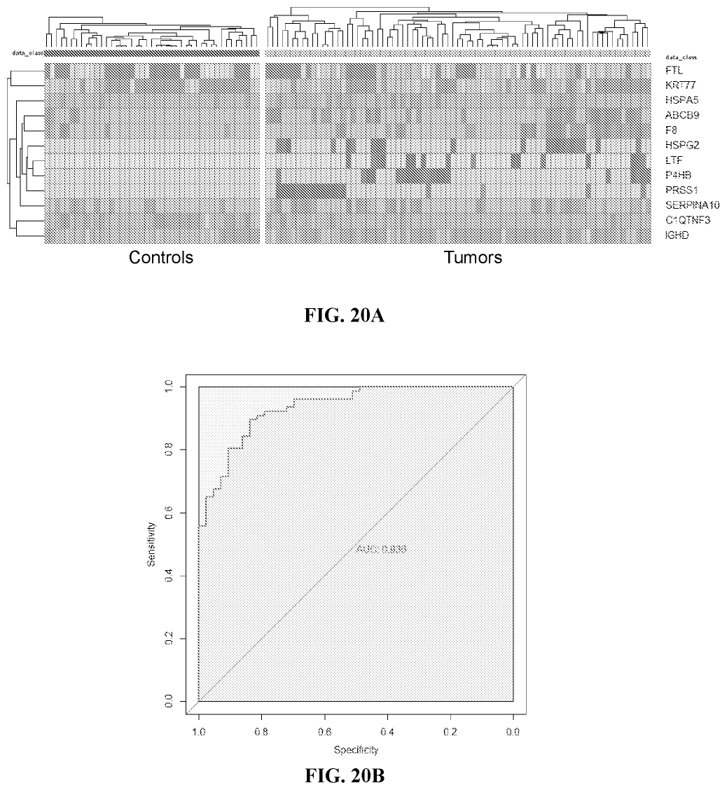

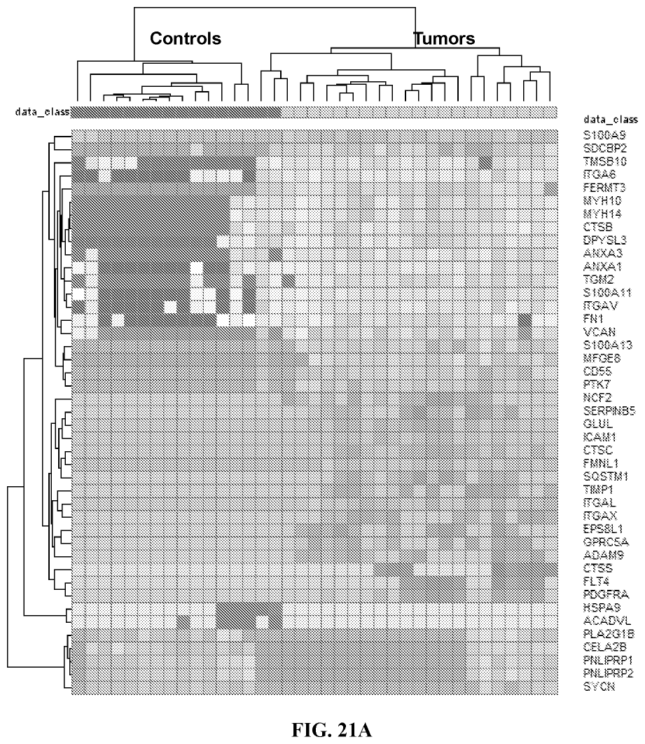

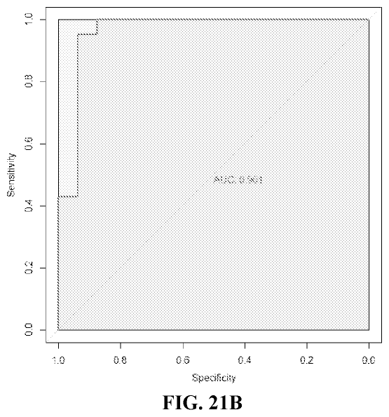

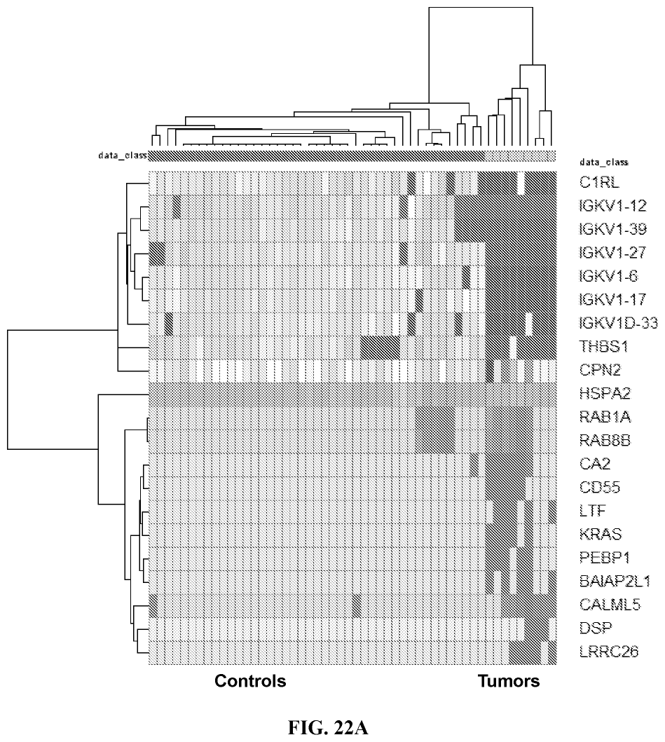

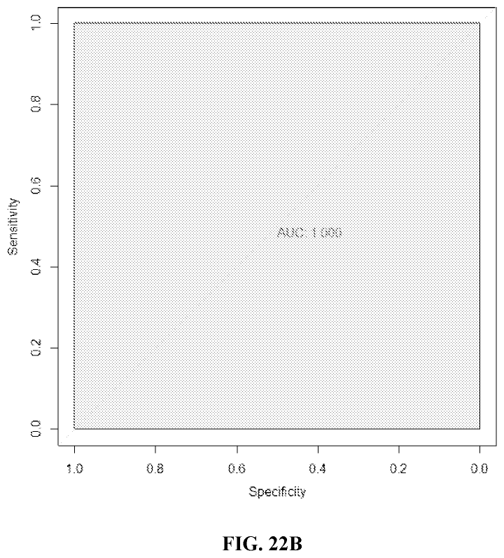

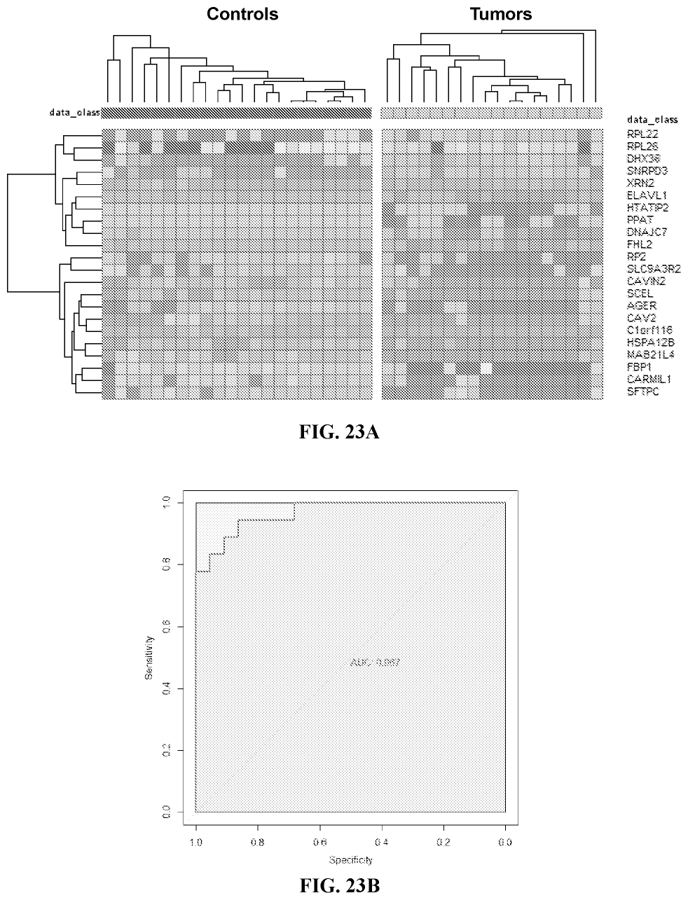

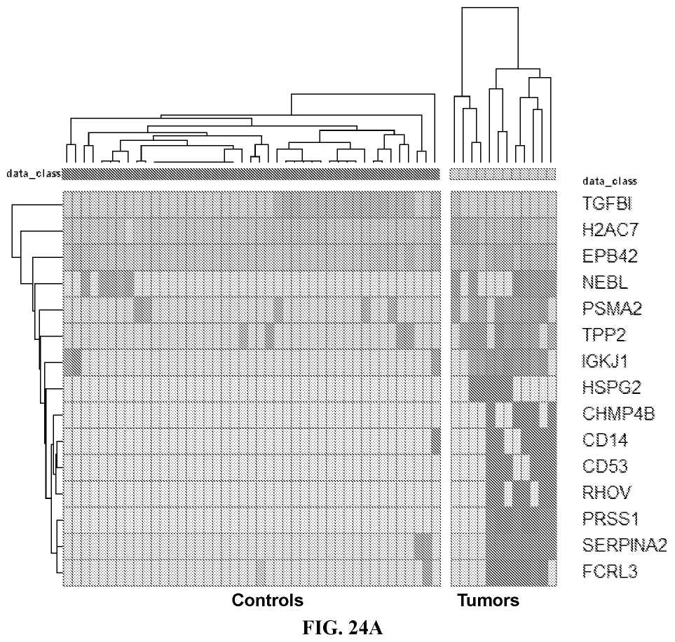

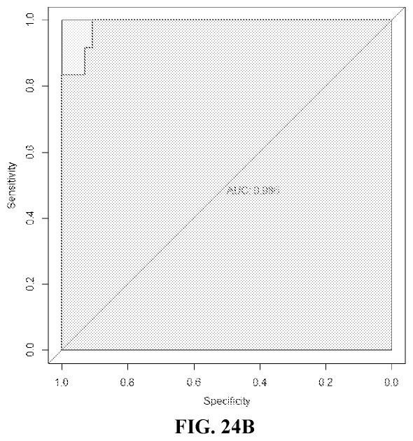

To test whether mucin and serpin proteins are indeed secreted by PDAC human tumors, the exosomal content of 21 PDAC specimens and 16 normal adjacent controls was assessed in an independent patient cohort (Table 23). This analysis revealed multiple mucin and serpin proteins that are highly expressed in tumor exosomes, compared to normal adjacent tissue-derived exosomes including IVUC5B and SERPINA1. Specifically, IVUC5B was detectable in 71% of PDAC-derived exosomes, compared to 1900 of adjacent pancreatic tissue-derived exosomes. SERPINA1 was evident in 100% of PDAC-derived exosomes, but was less specific as it was found in 50% of the control tissues (FIG. 17).

TABLE 23
Clinical Data of Patient Cohort Used
for Pancreatic Exosome Analysis

| | | | | | Tumor | Adjacent |
|---|---|---|---|---|---|---|
| ID | OR date | Gender | Age | Stage | (n = 21) | (n = 16) |
| 1 | Apr. 9, 2015 | M | 74 | 3 | + | + |
| 2 | Apr. 28, 2015 | F | 77 | 2 | + | |
| 3 | Apr. 29, 2015 | M | 72 | 2 | + | |
| 9 | Jul. 20, 2015 | F | 51 | 2 | + | + |
| 1 | Jul. 22, 2015 | M | 74 | 3 | + | |
| 11 | Jul. 23, 2015 | M | 66 | 3 | + | |
| 21 | Dec. 3, 2015 | F | 57 | 2 | + | |
| 23 | Dec. 21, 2015 | M | 67 | 2 | + | |
| 27 | Jan. 12, 2016 | F | 66 | 2 | + | |
| 29 | Jan. 25, 2016 | M | 82 | 2 | + | |
| 32 | Feb. 24, 2016 | F | 64 | 2 | + | |
| 37 | May 16, 2016 | F | 76 | 2 | + | + |
| 41 | Jul. 11, 2016 | M | 83 | 2 | + | |
| 42 | Aug. 24, 2016 | F | 66 | 2 | + | + |
| 43 | Sep. 10, 2016 | M | 74 | 2 | + | + |
| 44 | Sep. 14, 2016 | M | 53 | 3 | + | + |
| 45 | | | | | | + |
| 54 | | | | | | + |
| 58 | May 17, 2017 | M | 65 | 2 | + | + |
| 59 | | | | | | + |
| 65 | Aug. 17, 2017 | M | 69 | 2 | + | + |
| 67 | Aug. 28, 2017 | M | 74 | 2 | + | + |
| 68 | Sep. 5, 2017 | M | 71 | 1 | + | + |
| 71 | Sep. 13, 2017 | M | 66 | 3 | + | |
| 84 | Dec. 11, 2017 | M | 65 | 2 | | + |
| C2 | | | | | | + |
| C5 | | | | | | + |

### Example 5—Specific DAMP Molecules are Packaged in Tumor Tissue-Derived EVPs

Since it is well known that exosomes interact with the immune system (Becker et al., “Extracellular Vesicles in Cancer: Cell-to-Cell Mediators of Metastasis,” Cancer cell 30:836-848 (2016), which is hereby incorporated by reference in its entirety), it was asked whether specific proteins involved in eliciting immune responses, such as damage associated molecular pattern (DAMP) proteins, which have key roles in cancer development and tumor progression (Hernandez et al., “Damage-Associated Molecular Patterns in Cancer: A Double-Edged Sword,” Oncogene 35:5931-5941 (2016), which is hereby incorporated by reference in its entirety) (Table 24), could be packaged in TT-derived EVPs.

TABLE 24
Damage-associated molecular pattern (DAMP) molecules used in this study.

| Type | # of molecule | Proteins |
|---|---|---|
| S100s | 18 | S100A1, S100A10, S100A11, S100A12, S100A13, S100A14, |
| | | S100A16, S100A2, S100A3, S100A4, S100A6, |
| | | S100A7, S100A7A, S100A8, S100A9, S100B, S100G, S100P |
| TLRs | 6 | TLR2, TLR3, TLR4, TLR6, TLR7, TLR8 |
| Annexins | 13 | ANXA1, ANXA10, ANXA11, ANXA13, ANXA2, ANXA2P2, ANXA3, |
| | | ANXA4, ANXA5, ANXA6, ANXA7, ANXA8, ANXA9 |
| HMG molecules | 12 | HMG20A, HMG20B, HMGA1, HMGA2, HMGB1, HMGB2, HMGB3, |
| | | HMGN1, HMGN2, HMGN3, HMGN4, HMGN5 |
| Integrins | 28 | ITGA1, ITGA10, ITGA11, ITGA2, ITGA2B, 1TGA3, ITGA4, |
| | | ITGA5, ITGA6, ITGA7, ITGA8, ITGA9, ITGAD, ITGAE, |
| | | ITGAL, ITGAM, ITGAV, ITGAX, ITGB1, ITGB1BP1, ITGB2, |
| | | ITGB3, ITGB4, ITGB5, ITGB6, ITGB7, ITGB8, ITG8L1 |
| scavenger receptors | 7 | scavenger receptors, MSR1, SCARA3, SCARB1, SCARB2, |
| | | SCARF1, SCARF2, SSC5D |
| chemokine receptors | 6 | CGR1, CCR7, CCRL2, CXCR1, CXCR2, CXCR4 |
| Galectins | 11 | CLC, LGALS1, LGALS2, LGALS3, LGALS3BP, LGALS4, LGALS7, |
| | | LGALS8, LGALS9, LGALS9B, LGALSL |
| Fibrinogen | 5 | FGA, FGB, FGG, FGL1, FGL2 |
| ECM glycoproteins | 43 | ACAN, ASPN, BCAN, BGN, CEMIP, CHAD, CHADL, CSPG4, DCN, |
| | | FMOD, HABP2, HAPLN1, HAPLN3, HAPLN4, |
| | | HAS1, HAS2, HAS3, HSPG2, HYAL2, HYAL3, IMPG2, KERA, |
| | | LUM, LYVE1, MGEA5, NCAN, OGN, OMD, PAPLN, |
| Fibronectin | 12 | Fibronectin, ELFN2, FANK1, FLRT1, FLRT2, FLRT3, FN1, |
| | | FNDC1, FNDC3A, FSD1, FSD1L, FSD2, IGFN1 |
| Thioredoxin | 15 | TMX1, TMX2, TMX3, TMX4, TXN, TXN2, TXNDC12, TXNDC17, |
| | | TXNDC5, TXNDC9, TXNIP, TXNL1,TXNL4A, |
| | | TXNRD1,TXNRD2 |
| Purinergic receptor | 5 | P2RX1, P2RX3, P2RX4, P2RX7, P2RY2 |
| Others | 14 | A2M (CD91), AGER, BCL2, BSG, CALR, CALR3, CD14, CD2, |
| | | CD38, CD44, FPR1, HAVCR2, IL1R1, IL6ST |
| Total | 195 | |

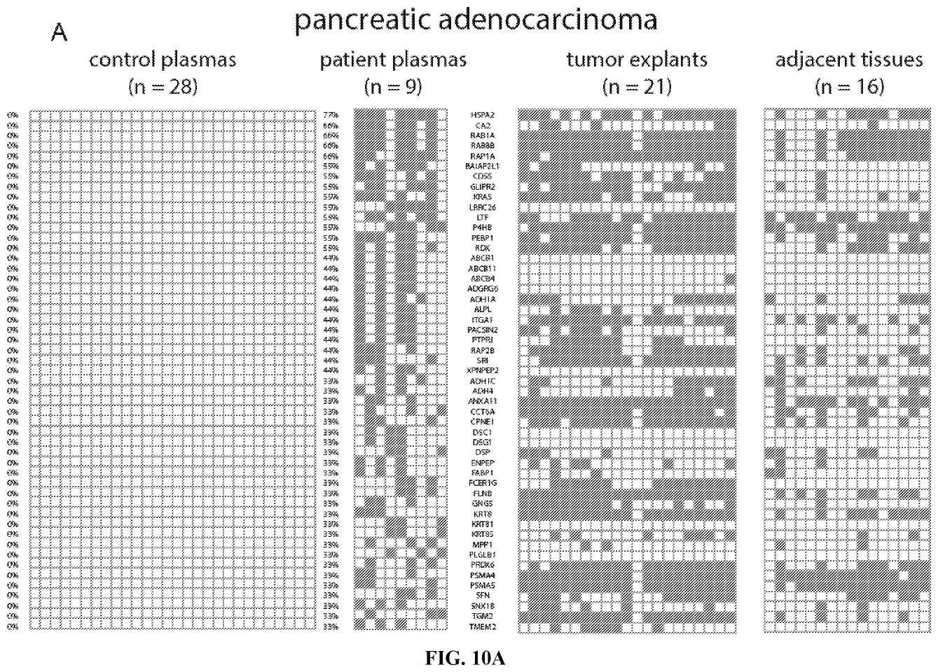

By assessing DAMPs and their receptors in PaCa TT compared to PaCa AT, 39 EVP DAMPs were found (i.e., versican) that were highly enriched in TT-derived compared to AT4 derived EVPs (FIG. 9A). Within this list, six proteins were present in TT-derived but never found in AT-derived EVPs. These included S100A13, basigin, galectin 9, biglycan (BGN) and integrins α5 and αX (FIG. 9A). Similar analyses performed for LuCa EVPs revealed two abundantly expressed DAMP proteins, versican and galectin 9. These were described as effective proinflammatory molecules (e.g., galectin 9, S100A13, biglycan) or receptors for proinflammatory cytokines (e.g., basigin and integrins) (Hernandez et al., “Damage-Associated Molecular Patterns in Cancer: A Double-Edged Sword,” Oncogene 35:5931-5941 (2016), which is hereby incorporated by reference in its entirety). Notably, versican and galectin 9 were highly enriched in both PaCa and LuCa TT EVPs, suggesting that they represent EVP inflammatory response markers shared across cancers (FIG. 9A and FIG. 9B). Interestingly, certain DAMPs, such as annexin A3 (ANXA3), and several integrins (e.g., ITGB2, ITGAV) were enriched in AT/DT EVPs in LuCa, but not in PaCa. This finding may reflect the presence of cancer-associated stroma in AT/DT (FIG. 9B) and further emphasizes that the non-tumor-derived EVP proteome is as informative as the tumor-derived EVP proteome in identifying specific cancer types. Collectively, unique DAMPs present in cancer or non-cancer EVPs may help delineate the pro-tumoral versus immunogenic roles of DAMP molecules.

### Example 6—Analysis of Tissue-Derived EVP Proteins Across Multiple Cancers Identifies Tumor Associated EVP Signatures

TT-specific EVP proteins have been identified. Therefore, it was next elucidated whether comparing TT-derived and non-TT-derived EVP proteomic information could be used to distinguish cancer from non-cancer, in general. A total of 131 tissue explant- and 20 bone marrow-derived EVP samples were analyzed. Eighty-five samples were isolated from TT, while 66 were classified as non-TT (FIG. 6D). Random forest classification was employed, which is robust to noise and overfitting, to identify a subset of proteins that could accurately discriminate between healthy controls and patients with tumors. To train and subsequently test the model, samples were evenly partitioned based on sample type (ie. control sample or tumor sample) and 75% of samples were used as a training set with the remaining 25% representing the independent test set. Applying 10-fold cross-validation to the training set yielded a sensitivity (true positive rate) of 95% and specificity (true negative rate) of 92% (FIG. 6E). When applied to the independent test-set samples, the model achieved 90% sensitivity and 94% specificity (FIG. 6E). This result is likely driven in part by tissue-specific field effects, as sensitivity and specificity improve when focusing on individual tissues. Analysis of larger sample sizes would be necessary to further validate this and inform on tissue-specific tumor-associated EVP signatures. Despite the inherent tissue-specific variation, a combination of proteins that were most predictive of differentiating cancer from non-cancer were identified (FIG. 6D; Table 25).

TABLE 25
Proteins defined as predictive for distinguishing tumor versus
non-tumor based on tumor tissue-derived EVPs (n = 85)
versus non-tumor tissue-derived EVP (n = 66) comparison.

| | | log₁₀ | | |
|---|---|---|---|---|
| | | fold | posivitiy | posivitiy |
| | | change | in normal | in tumor |
| | | (tumor/ | tissue | tissue |
| protein | protein name | normal) | (n = 66) | (n = 85) |
| THBS2 | thrombospondin 2 | 5.1 | 12% | 74% |
| VCAN | versican | 5.1 | 17% | 75% |
| SRRT | serrate, RNA effector molecule | 4.1 | 9% | 62% |
| TNC | tenascin C | 3.5 | 9% | 54% |
| DPYSL2 | dihydropyrimidinase like 2 | 3.6 | 55% | 98% |
| AHCY | adenosylhomocysteinase | 3.2 | 62% | 96% |
| DNAJA1 | DnaJ heat shock protein family (HsP40) | 3.3 | 45% | 85% |
| | member A1 | | | |
| PGK1 | phosphoglycerate kinase 1 | 2.3 | 79% | 100% |
| EHD2 | EH domain containing 2 | 1.0 | 88% | 87% |
| ADR1B | alcohol dehydrogenase 1B (class I), | −0.6 | 67% | 64% |
| | beta polypeptide | | | |
| CAVIN1 | caveolae associated protein 1 | −1.1 | 53% | 42% |
| FGGY | FGGY carbohydrate kinase domain containing | −1.2 | 20% | 4% |
| ABCA3 | ATP binding cassette subfamify A member 3 | −1.7 | 32% | 12% |
| STX11 | syntaxin 11 | −2.8 | 38% | 1% |
| CAVIN2 | caveolae associated protein 2 | −3.8 | 53% | 7% |
| CD36 | CD36 molecule | −4.3 | 71% | 20% |

Notably, thrombospondin and versican, EVP proteins highly enriched in both PaCa and LuCa TT, were predictive in identifying cancer, suggesting that these proteins could be used as pan-cancer EVP markers. Together, specific EVP adhesion markers [e.g., CD36, tenascin C (TNC), THBS2, VCAN] and metabolic enzymes [e.g., all-trans-retinol dehydrogenase [NAD(+)] ADH1B/alcohol dehydrogenase 1B (ADH1B), adenosylhomocysteinase (AHCY) and phosphoglycerate kinase 1 (PGK1)] can be used as pan-cancer markers (Table 25).

### Example 7—Tumor, Peritumoral Microenvironment and Distant Stroma EVP Proteins Contribute to Tumor-Associated EVP Signatures in Plasma

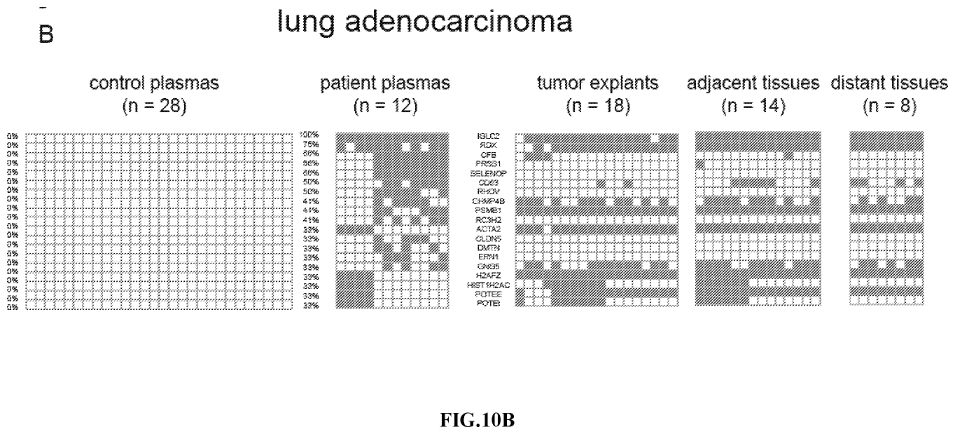

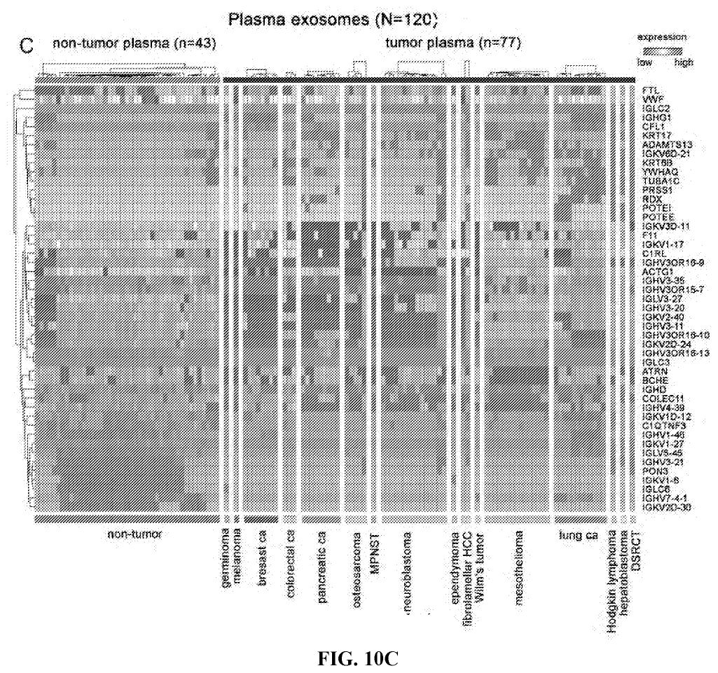

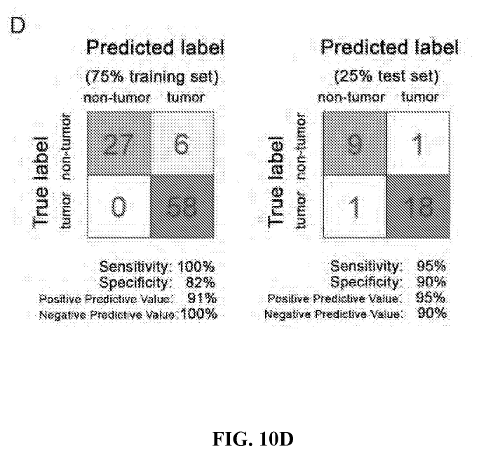

For clinical use of liquid biopsies, analysis of plasma-derived EVP data is needed. First, it was sought to determine which cancer-associated EVP proteins are present in the plasma of PaCa and LuCa patients and then it was addressed whether these proteins originated from the TT, AT/DT or elsewhere. The plasma EVP proteomes of 9 patients with PaCa (78% stage 2 and 22% stage 3) and 12 with LuCa (50% stage 1, 42% stage 2, and 8% stage 3) were analyzed and EVP proteins found in more than 30% of patient plasma but never in the plasma of any of the 28 healthy adult controls were selected. Using these criteria, 51 and 19 plasma-derived EVP proteins unique to PaCa and LuCa were found, respectively (FIGS. 10A and 10B). In an attempt to identify the likely source of these EVPs, these plasma-derived EVP proteins were compared with the TT, AT and DT-derived EVP proteomic data for PaCa and LuCa (FIGS. 10A and 10B). Interestingly, there were proteins, such as brain-specific angiogenesis inhibitor 1-associated protein 2-like protein 1 (BAIAP2L1), alkaline phosphatase, tissue-nonspecific isozyme (ALPL), receptor-type tyrosine-protein phosphatase eta (PTPRJ), high affinity immunoglobulin epsilon receptor subunit gamma (FCER1G), and cell surface hyaluronidase (TMEM2), which were present in both plasma- and TT-derived PaCa EVPs but which were packaged at extremely low levels or undetectable in all of the 16 AT-derived EVP samples, suggesting that these proteins most likely originate from pancreatic tumor cells (FIG. 10A). KRAS, an oncoprotein that drives pancreatic cancer, was frequently packaged in TT EVPs (76%) and could be detected in plasma EVPs of patients with PaCa (FIG. 10A). Surprisingly, many proteins, such as leucine-rich repeat-containing protein 26 (LRRC26), ATP-dependent translocase ABCB1 (ABCB1), bile salt export pump (ABCB11), adhesion G-protein coupled receptor G6 (ADGRG6), desmocollin-1 (DSC1), desmoglein-1 (DSG1), keratin, type II cuticular Hb1 (KRT81) and plasminogen-like protein B (PLGLB1), were absent or packaged at low levels in both TT- and AT derived EVPs but were found exclusively in PaCa patient plasma-derived EVPs, suggesting that these proteins originate from distant organs (DO), including immune cell-derived EVPs. The fact that these proteins were never found in plasma EVPs from healthy controls reinforces the idea that cancer is a systemic disease that alters EVP cargo at DO sites (FIG. 10A).

For LuCa, 19 plasma EVP proteins present in more than 30% of patients (FIG. 10B) were identified. In contrast to PaCa, all of the proteins detected in LuCa TT were also found in AT and most of DT (FIG. 10B). Proteins such as selenoprotein P (SELENOP), rho-related GTP binding protein RhoV (RHOV), roquin-2 (RC3H2), claudin-5 (CLDN5), dematin (DMTN), and serine/threonine-protein kinase/endoribonuclease IRE1 (ERN1), were only detected in plasma, but not in TT, AT or DT, supporting the systemic nature of cancer. Specifically, the liver-derived protein selenoprotein, was never detected in EVPs derived from the organ in which the cancer originated, but was frequently found in plasma-derived EVPs from LuCa patients (FIG. 10B), suggesting lung cancer affects liver function.

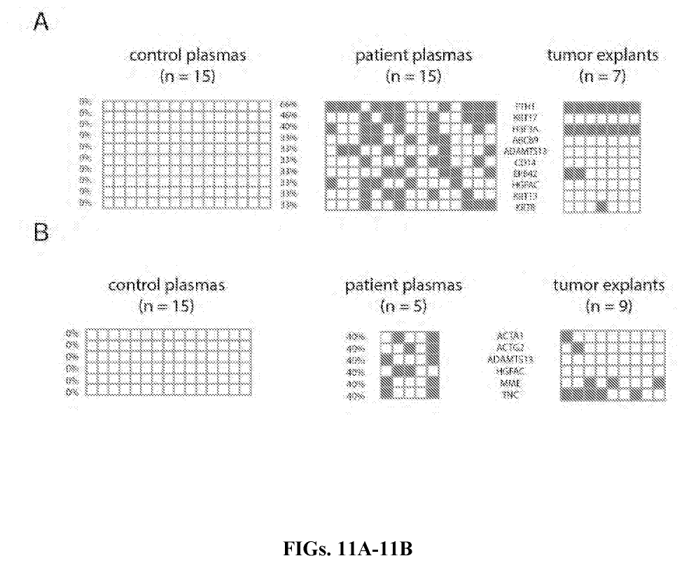

To demonstrate that these observations were not restricted to lung and pancreatic cancer, or adult cancers in general, TT- and plasma-derived EVPs isolated from advanced stage patients with two of the most frequent pediatric solid cancers: neuroblastoma and osteoblastoma (Table 2) were examined. Pediatric cancers are fast-growing, overtaking the organ where they originate, therefore rendering AT harvesting very challenging. TT-derived EVPs were analyzed from 9 neuroblastoma and 7 osteosarcoma patients and plasma-derived EVPs were analyzed from 15 neuroblastoma and 5 osteosarcoma patients (Tables 5 and 6). Plasma-derived EVPs from a total of 15 age-matched healthy controls also were assessed in these comparisons. (FIGS. 11A-11B). Analyses was focused on EVP proteins detected in >33% of plasma samples from cancer patients but never detected in any of the control subject plasma. In neuroblastoma, 10 plasma EVP proteins, ferritin heavy chain (FTH1), keratin, type I cytoskeletal 17 (KRT17), histone H3.3 (H3F3A), ATP-binding cassette sub-family B member 1 9 (ABCB9), a disintegrin and metalloproteinase with thrombospondin motifs 13 (ADAMTS13), CD14, erythrocyte membrane protein band 4.2 (EPB42), hepatocyte growth factor activator (HGFAC), keratin, type I cytoskeletal 13 (KRT13), and KRT8 (FIG. 11A), related to cellular proliferation/cell cycle and differentiation were found. In osteosarcoma, 6 plasma EVP proteins, actin, alpha skeletal muscle (ACTA1), actin, gamma-enteric smooth muscle (ACTG2), ADAMTS13, HGFAC, neprilysin (MME), and TNC, related to tissue morphogenesis (FIG. 11B) were identified. Interestingly, EVP protein cargo reflected the cell of origin of each cancer (osteoblast versus neuroblast). These analyses further underscore that the EVP proteome reflects the tumor origin, type and its progression.

Taken together, the data demonstrate that plasma-derived EVPs are derived from various sources, and EVP proteomic analyses can identify protein profiles in plasma EVPs that are cancer type-specific in resectable and advanced disease. By comparing plasma-derived and tissue-derived EVP proteins, it was possible to distinguish between tumor-derived EVPs, adjacent tissue-derived EVPs and distal organ EVPs. Furthermore, plasma EVP protein signatures of cancer patients were distinct from those of control subjects and were cancer-type specific, suggesting that EVP protein profiles could serve as a liquid biopsy tool to detect cancer and differentiate among cancer types.

### Example 8—Analysis of Plasma-Derived EVP Proteins Across Multiple Cancers Identifies Tumor Associated EVP Signatures

Employing random forest classification, in the same manner described for tissue samples, tumor-associated EVP signatures derived from plasma were explored. A total of 120 plasma-derived EVP proteomes from 77 cancer patients with 16 different cancer types, including breast carcinoma, lung carcinoma, pancreatic carcinoma, mesothelioma and neuroblastoma, and 43 healthy control subjects (FIG. 10C) were analyzed. Ten-fold cross-validation of the training set yielded a sensitivity of 100% and specificity of 82% (FIG. 10D). The model achieves 95% sensitivity and 90% specificity and showed a combination of different immunoglobulins related proteins as being the best predictive biomarkers for detecting cancer (FIG. 10C and FIG. 10D; Table 26).

TABLE 26
Proteins defined as predictive for distinguishing tumor versus non-tumor based on tumor plasma
exosomes (n = 77) versus healthy control plasma-derived exosomes (n = 43) comparison.

| | | log₁₀ | | |
|---|---|---|---|---|
| | | fold | posivitiy | posivitiy |
| | | change | in normal | in tumor |
| | | (tumor/ | plasma | plasma |
| protein | protein name | normal) | (n = 43) | (n = 77) |
| IGLC2 | immunoglobulin lambda constant 2 | 4.6 | 12% | 56% |
| KRT17 | keratin 17 | 3.0 | 7% | 43% |
| IGHG1 | immunoglobulin heavy constant gamma 1 | 2.8 | 12% | 36% |
| KRT6B | keratin 6B | 2.4 | 7% | 35% |
| FTL | ferritin light chain | 2.2 | 49% | 70% |
| RDX | redixin | 1.9 | 0% | 25% |
| CFL1 | cofilin 1 | 1.8 | 26% | 48% |
| PRSS1 | protease, serine 1 | 1.8 | 0% | 22% |
| TUBA1C | tubulin alpha 1c | 1.8 | 7% | 30% |
| ADAMTS13 | ADAM metallopeptidase with thrombospondin | 1.8 | 12% | 35% |
| | type 1 motif 13 | | | |
| IGKV6D-21 | immunoglobulin kappa variable 6D-21 | 1.7 | 16% | 38% |
| YWHAG | tyrosine 3-monooxygenase tryptophan | 1.7 | 16% | 38% |
| | 5-monooxygenase activation protein theta | | | |
| POTEI | POTE ankyrin domain family member I | 1.4 | 0% | 17% |
| POTEE | POTE ankyrin domain family member F | 1.2 | 0% | 14% |
| VWF | von Willebrand factor | 0.9 | 98% | 97% |
| ACTG1 | actin gamma 1 | −2.1 | 93% | 64% |
| IGLV3-27 | immunoglobulin lambda variable 3-27 | −2.1 | 86% | 57% |
| IGKV1D-12 | immunoglobulin kappa variable 1D-12 | −2.2 | 58% | 25% |
| F11 | coagulation factor Xi | −2.3 | 91% | 58% |
| C1RL | complement C1r subcomponent like | −2.4 | 86% | 64% |
| ATRN | attractin | −2.4 | 84% | 53% |
| BCHE | butyrylcholinesterase | −2.4 | 84% | 53% |
| IGHV3-35 | immunoglobulin heavy variable 3-35 | −2.5 | 70% | 43% |
| IGKV1-17 | immunoglobulin kappa variable 1-17 | −2.7 | 100% | 86% |
| C1QTNF3 | C1q and TNF related 3 | −3.0 | 53% | 25% |
| IGHV3-20 | immunoglobulin heavy variable 3-20 | −3.2 | 86% | 55% |
| IGHV3OR15-7 | immunoglobulin heavy variable 3/OR15-7 | −3.3 | 79% | 39% |
| COLEC11 | collectin subfamily member 11 | −3.4 | 84% | 40% |
| IGHD | immunoglobulin heavy constant delta | −3.5 | 77% | 35% |
| IGKV3D-11 | immunoglobulin kappa variable 3D-11 | −3.6 | 100% | 81% |
| IGHV3OR16-10 | immunoglobulin heavy variable 3/OR16-10 | −3.9 | 86% | 47% |
| IGKV2D-24 | immunoglobulin kappa variable 2D-24 | −4.1 | 86% | 39% |
| IGKV2-40 | immunoglobulin kappa variable 2-40 | −4.1 | 88% | 44% |
| IGKV1-27 | immunoglobulin kappa variable 1-27 | −4.4 | 79% | 22% |
| IGHV3OR16-9 | immunoglobulin heavy variable 3/OR16-9 | −4.4 | 88% | 44% |
| IGLV5-45 | immunoglobulin lambda variable 5-45 | −4.4 | 72% | 18% |
| IGHV3OR16-13 | immunoglobulin heavy variable 3/OR18-13 | −4.6 | 77% | 27% |
| IGHV1-46 | immunoglobulin heavy variable 1-46 | −4.7 | 72% | 16% |
| IGHY4-39 | immunoglobulin heavy variable 4-39 | −5.0 | 93% | 38% |
| IGHV3-11 | immunoglobulin heavy variable 3-11 | −5.0 | 95% | 43% |
| IGLC3 | immunoglobulin lambda constant 3 | −5.2 | 77% | 27% |
| IGKV1-6 | immunoglobulin kappa variable 1-6 | −5.5 | 67% | 1% |
| PON3 | paraoxonase 3 | −5.5 | 72% | 5% |
| IGHV3-21 | immunoglobulin heavy variable 3-2.1 | −5.7 | 70% | 9% |
| IGHV7-4-1 | immunoglobulin heavy variable 7-4-1 | −5.8 | 74% | 10% |
| IGKV2D-30 | immunoglobulin kappa variable 2D-30 | −6.0 | 67% | 0% |
| IGLC8 | immunoglobulin lambda constant 8 | −7.1 | 70% | 1% |

Notably, predictive proteins that discriminate cancer versus non-cancer relied not only on plasma-derived EVP proteins in cancer patients, but also on proteins found in EVPs derived from normal plasma but which are absent or present at low levels in cancer patient plasma EVPs. These proteins included immunoglobulins (immunoglobulin kappa variable 1-6 (IGKV1-6), immunoglobulin heavy variable 3-21 (IGHV3-21), immunoglobulin heavy variable 7-4-1 (IGHV7-4-1), immunoglobulin kappa variable 2D-30 (IGKV2D-30), immunoglobulin lambda constant 6 (IGLC6) and paraoxonase 3 (PON3), which were found in 67-74% of plasma derived EVPs from healthy controls but in less than 10% of plasma-derived EVPs from cancer patients (Table 10). Of note, IGKV2D-30 was only found in non-tumor plasma, further encouraging the notion that cancer and non-cancer discrimination should take into account not only EVP proteins enriched in/unique to cancer subjects, but also those EVP proteins that are lost in cancer-associated settings (FIG. 10C). Consistent with the findings in FIG. 10A and FIG. 10B, radixin (RDX), found exclusively in the plasma of PaCa and LuCa patients, was one of the proteins with the highest predictive value for defining cancer (FIG. 10C). The results suggest that plasma-derived EVP proteins could be useful as liquid biopsy tests for cancer detection.

### Example 9—Patient Tumor Tissue-Derived EVP Proteomics Classify Cancer Types

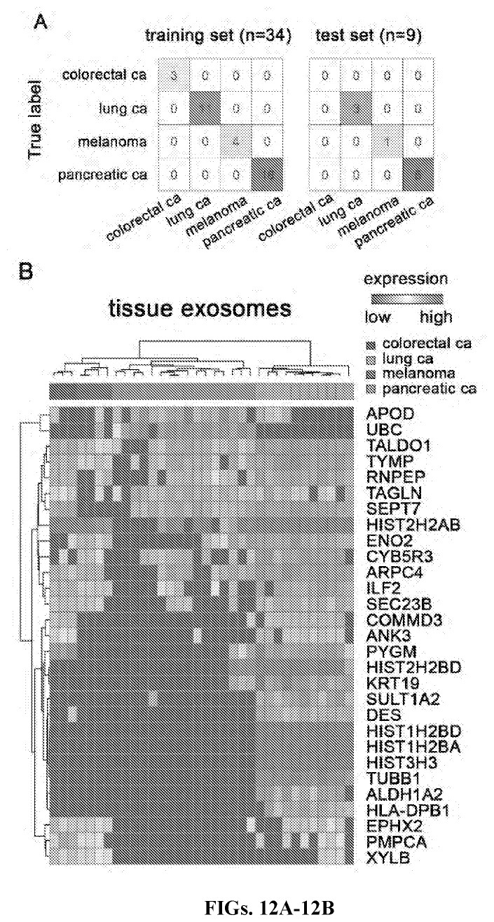

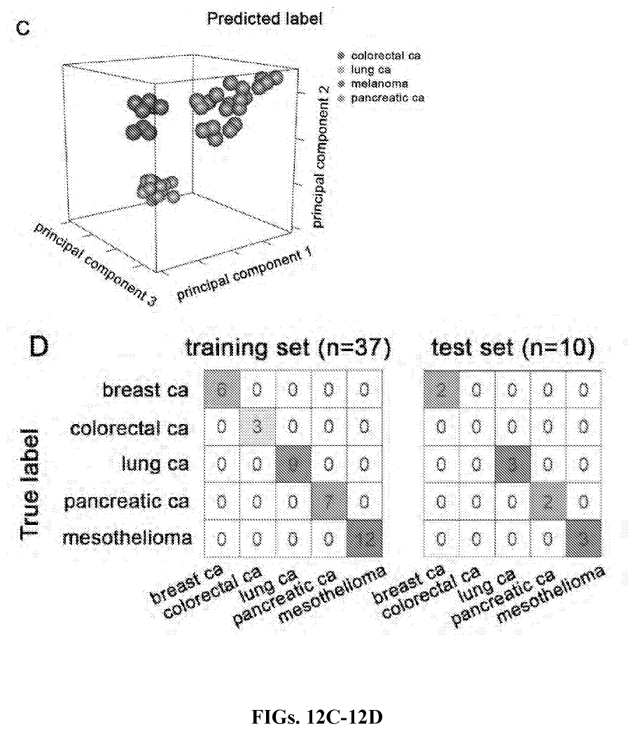

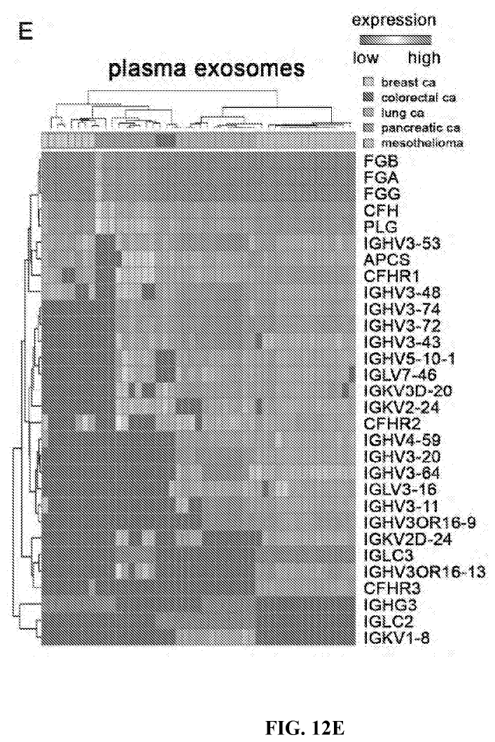

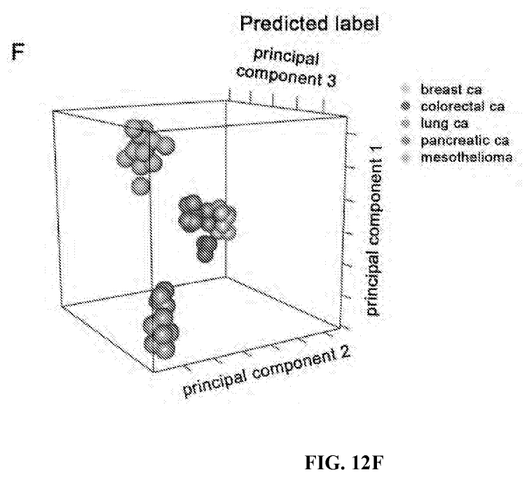

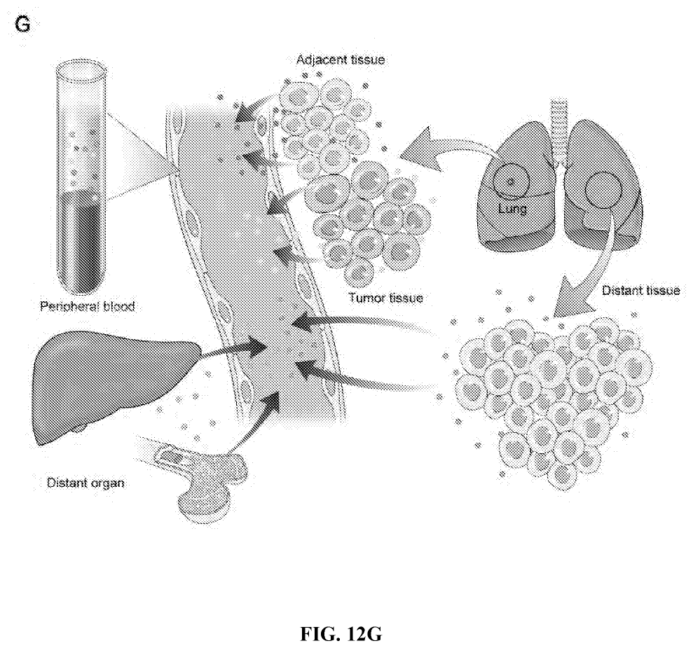

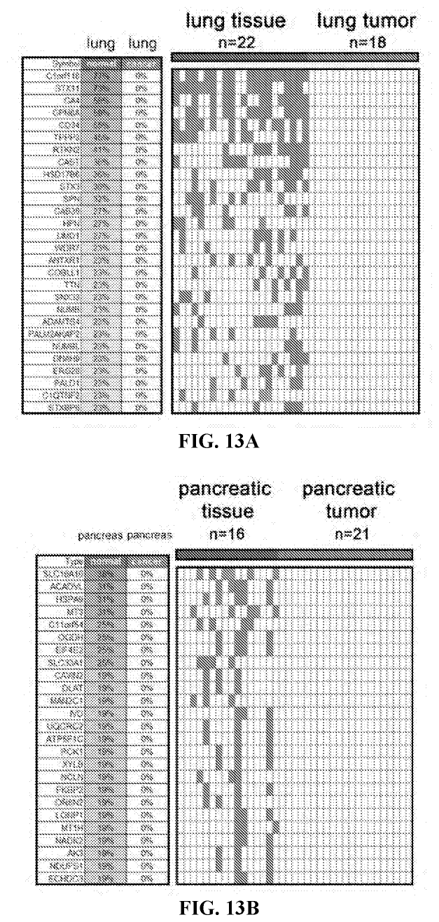

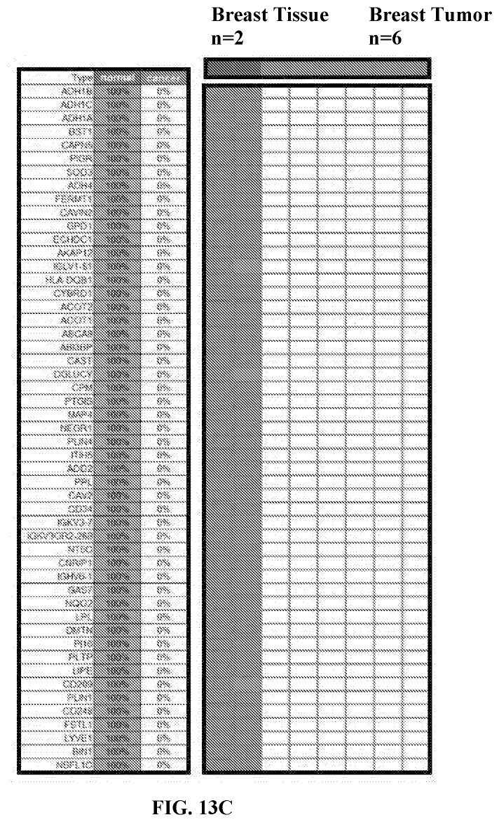

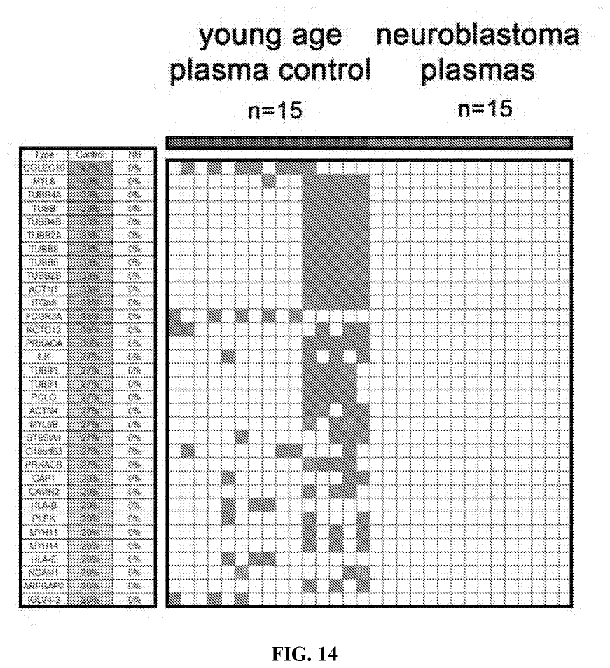

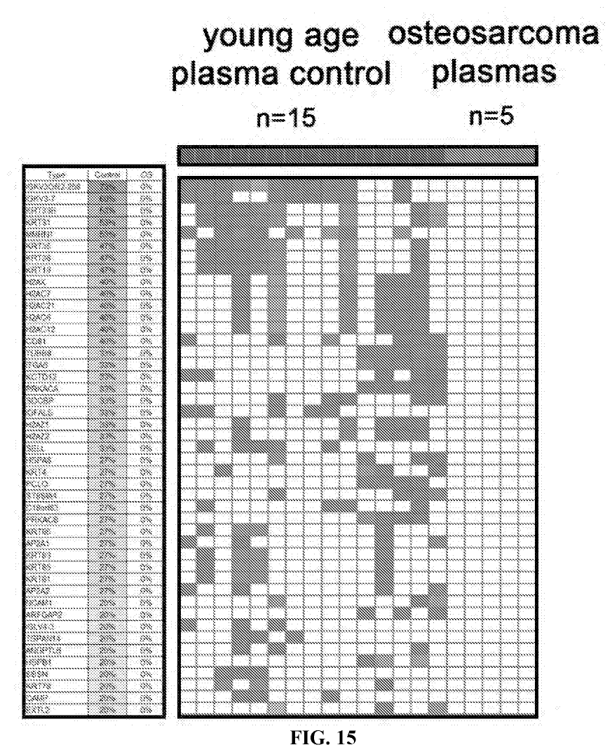

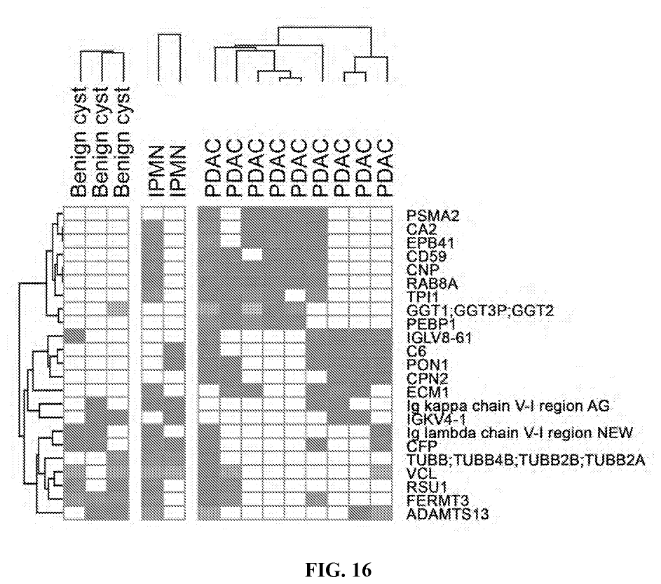

To take the previous analysis examining tissue-derived EVPs further, it was next sought to determine if a patient's EVP protein signature could be assigned to a particular cancer type. EVP proteins derived from tissues obtained from the primary tumor or sentinel lymph nodes of patients with four different cancer types: melanoma, colorectal, pancreatic and lung cancer (FIG. 12A) were analyzed and plotted. To explore the association between EVPs derived from different primary tumor types and to identify combinations of protein signatures that can discriminate between the four tumor types, random forest classification and t-Stochastic Neighbour Embedding (tSNE) for visualization was employed. It was possible to correctly discriminate every tumor sample, as summarized in a confusion matrix (FIG. 12A). Feature selection by random forest identified 29 proteins, some of which are immune-related proteins, as having the highest predictive value for distinguishing the four cancers analyzed (FIG. 12B). A supervised three-dimensional tSNE projection was used to visualize the differences among samples (FIG. 12C). Based on these 29 EVP proteins, samples clustered together tightly based on primary tumor type (FIG. 12B). Interestingly, based on the tSNE visualization and random forest classifier results, the differences in EVP signatures among tumor types were independent of cancer staging within that tumor types and could be applied even at early cancer stages, especially in PaCa and LuCa (FIG. 12C). Thus, EVP tissue profiles from tissue biopsies (i.e., lymph nodes) could help aid in classifying cancer types supporting a diagnosis that can be assigned an improved clinical treatment plan for patients.

### Example 10—Patient Tumor Plasma-Derived EVP Proteomics Classify Cancer Types

Since tissue biopsies are not always available and for further confirmation of a tumor type, a similar analysis was performed using plasma-derived EVP proteomic data from patients with five different cancers, including breast, colorectal, lung, and pancreatic cancers and mesothelioma. Even though the majority of plasma-derived EVPs are of hematopoietic origin (Caby et al., “Exosomal-Like Vesicles are Present in Human Blood Plasma,” Int Immunol 17:879-887 (2005), which is hereby incorporated by reference in its entirety), feature selection of EVP proteins by random forest revealed a strong association within the same tumor type, as demonstrated by the training versus test set classifier results, heatmap and 3D-tSNE projection (FIGS. 12D-12F). Similar to the analysis of cancer versus non-cancer plasma, immunoglobulins were the top classifying proteins found at high frequency in most plasma derived exosome samples, especially in mesothelioma and lung cancers (FIG. 12E). Importantly, it was found that samples cluster based on primary tumor type regardless of cancer stage for all five cancer types analyzed. These findings constitute proof of principle that plasma-derived EVPs contain proteomic content representing bonafide tumor-specific signatures capable of distinguishing cancer types, independently of their stage. Overall, tissue- and plasma-derived EVP proteomes can be beneficial in determining tumor type for a diagnosis in patients with tumor of unknown primary tumor origin.

Ficolin-3 expression in plasma-derived extracellular vesicles is also a negative marker of breast and melanoma cancers. As shown in FIG. 1, ficolin-3 expression in extracellular vesicles and particles is significantly higher in control (i.e., healthy subjects) than in subjects having breast cancer and subjects having stage 3 or 4 melanoma. Thus, detection of ficolin-3 in a patient plasma derived exosome sample would indicate the subject does not have breast cancer or melanoma. The absence of ficolin-3 in a patient plasma derived exosome sample indicates the presence of breast cancer or melanoma in the subject.

### Discussion of Examples 1-10

Liquid biopsies based on simple blood tests show promise as non-invasive approaches for early detection, differentiating tumor type, and for monitoring treatment responses. Circulating EVPs, present in the order of billions in blood plasma and other bodily fluids, could represent an essential component of the liquid biopsy test on which clinical care decisions would be based. Many studies suggest that exosomal proteomes could serve as potential markers for cancer detection (Castillo et al., “Surfaceome Profiling Enables Isolation of Cancer-Specific Exosomal Cargo in Liquid Biopsies from Pancreatic Cancer Patients,” Ann Oncol. 29:28 223-229 (2018); Gangoda et al., “Proteomic Profiling of Exosomes Secreted by Breast Cancer Cells with Varying Metastatic Potential. Proteomics,” Proteomics and Systems Biology 17(23-24) (2017); Hurwitz et al., “Proteomic Profiling of NCI-60 Extracellular Vesicles Uncovers Common Protein Cargo and Cancer Type-Specific Biomarkers,” Oncotarget 7:86999-87015 (2016); Ji et al., “Proteome Profiling of Exosomes Derived from Human Primary and Metastatic Colorectal Cancer Cells Reveal Differential Expression of Key Metastatic Factors and Signal Transduction Components,” Proteomics 13:1672-1686 (2013), which are hereby incorporated by reference in their entirety); however, a consensus on appropriate exosomal markers is lacking. This is primarily due to limited data sets for human-derived samples and a paucity of appropriate controls.

Here, a large-scale, comprehensive analysis of EVP proteomes from 426 human cancer and non-cancer samples derived from various cells, tissues and bodily fluids was performed. Several standard exosome markers, including CD63, TSG101, flotillins and ALIX, are not well represented in human plasma, suggesting a need for additional pan-exosome markers for EVP purification and detection. The analyses identified new markers, such as moesin, filamin A, stomatin and the Ras oncogene family member RAP1B, that were found at high frequency and could be used as novel biomarkers for EVP isolation, especially in a liquid biopsy setting. Based on the cellular localization of the EVP proteins identified by the unbiased proteomics approach, the majority of proteins detected were membrane-associated and cytosolic proteins rather than nuclear proteins. Importantly, tumor tissue, non-tumor tissue and plasma extracellular particles are heterogeneous populations that include exosomes and newly identified exomeres (Jeppesen et al., “Reassessment of Exosome Composition,” Cell 177(428-445):e418 (2019); Zhang et al., “Identification of Distinct Nanoparticles and Subsets of Extracellular Vesicles by Asymmetric Flow Field-Flow Fractionation,” Nature Cell Biology 20:332-343 (2018); Zhang et al., “Asymmetric-Flow Field-Flow Fractionation Technology for Exomere and Small Extracellular Vesicle Separation and Characterization,” Nat Protoc 14:1027-1053 (2019), which are hereby incorporated by reference in their entirety); therefore, future work will focus on determining the relative contribution of each of these particle populations to the proteomic signatures described here.

The proof of principle analysis in patients with PaCa and LuCa identified proteins that were expressed at significantly higher levels or found exclusively in TT-derived EVPs, as compared to AT- and DT-derived EVPs. Some of these proteins were linked to EMT, coagulation and actin signaling pathways in PaCa and cell cycle, metabolic and RNA processing pathways in LuCa. More than 40 EMT-related proteins (e.g., ECM molecules, integrins and proteases) were selectively packaged in PaCa EVPs. Noticeably absent were the EMT nuclear associated proteins SNAIL, SLUG, ZEB, and TWIST. This may be due to the predominance of secreted, membranous and cytosolic proteins versus nuclear proteins and transcription factors in exosomes. While the EMT pathway was highly represented in PaCa EVPs, it was largely absent in LuCa EVPs, again illustrating the tumor specificity of EVP protein packaging. Interestingly, proteins involved in clotting/thrombosis, such as Factors II, III and IX and thrombospondin 2 in PaCa and thrombosponin 2 in LuCa, were also highly packaged in tumor EVPs, consistent with the high incidence of life-threatening thrombotic events observed in both PaCa and LuCa patients.

Among the proteins highly enriched in PaCa and LuCa TT, 11 shared tumor specific EVP proteins including ECM molecules (basigin and versican), fibulin 2 and immunomodulators, such as galectin 9, were found while the vast majority of highly enriched TT EVP proteins were exclusive to each tumor type. This highlights cancer heterogeneity across tumor types at the EVP level. By expanding the analysis to 18 different cancer types compared to various control samples (e.g., AT/DT, breast reduction tissues), 16 specific proteins were identified that best defined cancer in both adult and pediatric cancers, many of whom represent adhesion molecules (with versican and thrombospondin 2 being higher in tumor samples, for example). These findings underscore the importance of adhesion proteins, such as ECM molecules, in a wide range of cancer types. Interestingly, it was found that both tumor and non-tumor EVPs packaged many DAMP molecules, which provide essential normal immune functions, such as sterile inflammation associated with tissue repair (Wolchok et al., “Ipilimumab Efficacy and Safety in Patients with Advanced Melanoma: A Retrospective Analysis of HLA Subtype from Four Trials,” Cancer Immun 10:9 (2010), which is hereby incorporated by reference in its entirety). However, DAMP molecules were also identified that were specific to tumor EVPs. These included various S100 family members, particularly S100A4 and S100A13, as well as basigin and galectin 9 (FIG. 9), which may be involved in tipping the balance towards immune suppression, contributing to the tumor promoting pro-inflammatory local and systemic milieu. These data are consistent with the previous findings in murine models of metastasis, demonstrating that tumor EVPs transfer their cargo to recipient cells at distant sites, reprogramming them to generate pro-inflammatory pre-metastatic niches that support future metastasis (Costa-Silva et al., “Pancreatic Cancer Exosomes Initiate Pre-Metastatic Niche Formation in the Liver,” Nature Cell Biology 17:816-826 (2015); Hoshino et al., “Tumour Exosome Integrins Determine Organotropic Metastasis,” Nature 527:329-335 (2015), which are hereby incorporated by reference in their entirety).

In LuCa, both enriched and exclusive protein analysis of TT-derived EVPs revealed that the HIV-1 tat interactive protein 2 (HTATIP2), which is secreted following HIV infection and associated with HIV-associated neurocognitive disorders, was specifically packaged in tumor EVPs. Given that tumor EVPs disseminate systemically and have been shown to penetrate and disrupt the blood-brain barrier (Chen et al., “Elucidation of Exosome Migration Across the Blood-Brain Barrier Model In Vitro,” Cell Mol Bioeng 9:509-529 (2016); Rodrigues et al., “Tumour Exosomal CEMIP Protein Promotes Cancer Cell Colonization in Brain Metastasis,” Nature Cell Biology 21:1403-1412 (2019), which are hereby incorporated by reference in their entirety), it is possible that exosomal HTATIP2 may in part be responsible for the paraneoplastic syndrome often described in newly diagnosed LuCa patients. Furthermore, since epigenetic changes have been shown to drive cancer progression in general and LuCa progression in particular (Duruisseaux et al., “Lung Cancer Epigenetics: From knowledge to Applications,” Semin Cancer Biol 51:116-128 (2018), which is hereby incorporated by reference in its entirety), it was perhaps not surprising to find that methyltransferase-like 1 (METTL1) was also an EVP protein exclusively detected in LuCa TT. This finding indicates that tumor-derived EVPs may drive epigenetic changes in the tumor microenvironment, as well as in AT and distant organs.

Moreover, the approach described herein took advantage of the selective packaging of EVP cargo, which is protected from degradation in circulation and reflects not only tumor-derived proteins but also microenvironment and immune system-derived proteins. Examining plasma-circulating EVP proteins may offer an advantage over circulating tumor DNA, as these EVPs represent the systemic effects of cancer, the cancer-associated changes occurring in the developing primary tumor, the tumor microenvironment, distant organs, such as the immune system and liver. In support of this hypothesis, it was determined that in the plasma of patients, tumor-associated EVPs were derived from all three sources, representing differential signals in early-stage cancers (FIG. 12G). Approximately 50% of the tumor-associated EVPs were derived from the TT and AT/DT, representing the tumor microenvironment, while the other 50% were derived from distant organs and immune cells. Interestingly, most of the plasma-derived EVP proteins from the organ where the cancer originated were shared by both the TT and the AT and/or DT from the same organ, implying that the tumor microenvironment could make a major contribution to the tumor-associated EVPs observed in the plasma.

By examining cancer-associated plasma EVPs from a diversity of cancer patients with disease stages ranging from stage I to stage IV, distinct tumor-associated EVP protein signatures were detected, prior to distant metastasis. Collectively, these findings suggest that plasma-circulating EVP proteins could be used as biomarkers for early cancer detection. The proof of principle studies provide a rationale for a concerted effort to rigorously screen patients. Future studies are needed to include those with genetic anomalies (germline BRCA1 mutations) or those who present with pro-inflammatory conditions (i.e., pancreatitis, ulcerative colitis, Crohn's disease) that predispose them to cancer development. Screenings should include specific tumor-associated EVP protein profiles in tissues and plasma as part of a standard-of-care monitoring strategy.

For up to 5% of patients admitted at major cancer centers, the primary site of tumor development and thus the origin of the tumor cannot be determined despite extensive evaluation and despite the presence of cancer in other organs (Stella et al., “Cancers of Unknown Primary Origin: Current Perspectives and Future Therapeutic Strategies,” J Transl Med 10:12 (2012); Varadhachary et al., “Cancer of Unknown Primary Site,” N Engl J Med 371:757-765 (2014), which are hereby incorporated by reference in their entirety). These patients are diagnosed with ‘cancer of unknown primary origin,’ and their treatment consists of a combination of several highly cytotoxic therapies meant to destroy possible sources of secondary lesion(s). Although it was shown that tumor-associated EVPs were largely shared by tumors, it was found that various cancer types, including PaCa, LuCa, breast cancer, colorectal cancer and mesothelioma, can be distinguished through distinct combinations of EVP proteins, or ‘signatures’ from either tumor tissues or plasma. These cancer type-specific EVP protein signatures could be used as a liquid biopsy tool to help identify the primary origin of each patient's cancer and to establish a diagnosis and guide treatment decisions.

Collectively, the comprehensive analyses of EVP proteins from human tumor and non-tumor tissue and fluid sources created a large body of data on additional EVP markers that could be used to improve or develop new EVP isolation technologies. Furthermore, tumor-specific EVP proteins were identified that could serve as early cancer biomarkers or tools for diagnosing tumors of unknown primary origin.

Further analyses are needed to evaluate the sensitivity, specificity, and robustness of EVP protein as a liquid biopsy tests especially in comparison with existing tests such as circulating DNAs and circulating proteins. The results revealed that cancer-associated circulating EVPs were derived from the TT, the tumor microenvironment, and distant organs in cancer patients, and that not only cancer-derived EVPs are informing us of cancer presence. Moreover, it has been shown that EVP proteins were able to identify cancers in early stage patients. Furthermore, the findings can lead to the development of a method for isolation of tumor derived EVPs using tumor-specific proteins. Therefore, improved EVP isolation and implementing plasma EVP-based screening may benefit patients in the clinic. For example, screening for pancreatic cancer in individuals with genetic and inflammation-mediated cancer predisposition, may lead to early diagnosis, prior to clinical manifestations, allowing for the administration of potentially curative radiation/surgical therapies. Taken together, the findings reported here support the idea that tumor-associated EVP proteins could be used as biomarkers for early-stage cancer detection, modulators of treatment response and potentially for diagnosing tumors of unknown primary origin.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method for screening a subject for the presence of cancer, said method comprising:
obtaining a liquid biopsy sample from the subject;
separating extracellular vesicles and particles from the sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting of ferritin light chain, von Willebrand factor, immunoglobulin lambda constant 2, keratin 17, immunoglobulin heavy constant gamma 1, keratin 6B, radixin, cofilin 1, protease, serine 1, tubulin alpha 1c, ADAM metallopeptidase with thrombospondin type 1 motif 13, immunoglobulin kappa variable 6D-21, tyrosine 3-monooxygenase/tryptophan 5-monooxygenase activation protein theta, POTE ankyrin domain family member I, POTE ankyrin domain family member F, and combinations thereof, and
(ii) a protein selected from the group consisting of actin gamma 1, immunoglobulin lambda variable 3-27, immunoglobulin kappa variable 1D-12, coagulation factor XI, complement C1r subcomponent like, attractin, butyrylcholinesterase, immunoglobulin heavy variable 3-35, immunoglobulin kappa variable 1-17, C1q and TNF related 3, immunoglobulin heavy variable 3-20, immunoglobulin heavy variable 3/OR15-7, collectin subfamily member 11, immunoglobulin heavy constant delta, immunoglobulin kappa variable 3D-11, immunoglobulin heavy variable 3/OR16-10, immunoglobulin kappa variable 2D-24, immunoglobulin kappa variable 2-40, immunoglobulin kappa variable 1-27, immunoglobulin heavy variable 3/OR16-9, immunoglobulin lambda variable 5-45, immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6, and combinations thereof, thereby detecting the presence or absence of the protein of (i) and the protein of (ii) in the extracellular vesicle and particle protein sample.

2. The method of claim 1, wherein the protein of (i) is immunoglobulin lambda constant 2.

3. The method of claim 1, wherein the protein of (ii) is immunoglobulin kappa variable 2D-30.

4. The method of claim 1, wherein at least two proteins of (i) are detected as a result of said subjecting.

5. The method of claim 4, wherein the at least two proteins of (i) are immunoglobulin lambda constant 2 and keratin 17.

6. The method of claim 4, wherein the at least two proteins of (i) are ferritin light chain and von Willebrand factor.

7. The method of claim 1, wherein at least two proteins of (ii) are detected as a result of said subjecting.

8. The method of claim 7, wherein the at least two proteins of (ii) are immunoglobulin kappa variable 2D-30 and immunoglobulin lambda constant 6.

9. The method of claim 1, wherein at least two proteins of (i) and at least two proteins of (ii) are detected as a result of said subjecting.

10. The method of claim 9, wherein the at least two proteins of (i) are ferritin light chain and von Willebrand factor, and the at least two proteins of (ii) are immunoglobulin kappa variable 2D-30 and immunoglobulin lambda constant 6.

11. The method of claim 1, wherein the liquid biopsy sample is selected from the group consisting of whole blood, blood serum, blood plasma, urine, saliva, sputum, breast milk, ascites fluid, synovial fluid, amniotic fluid, semen, cerebrospinal fluid, follicular fluid and tears.

12. The method of claim 1, wherein the subject is a healthy subject.

13. The method of claim 1, wherein the subject is undergoing treatment for cancer.

14. The method of claim 1, where the subject is in remission for cancer.

15. A method for screening a subject for the presence of cancer, said method comprising:
obtaining a tissue sample from a subject;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting thrombospondin 2, versican, serrate, RNA effector molecule, tenascin C, dihydropyrimidinase like 2, adenosylhomocysteinase, DnaJ heat shock protein family (Hsp40) member A1, phosphoglycerate kinase 1, EH domain containing 2, and combinations thereof, and
(ii) a protein selected from the group consisting of alcohol dehydrogenase 1B (class I), beta polypeptide, caveolae associated protein 1, FGGY carbohydrate kinase domain containing, ATP binding cassette subfamily A member 3, syntaxin 11, caveolae associated protein 2, CD36 molecule, and combinations thereof, thereby detecting the presence or absence of the protein of (i) and the protein of (ii) in the extracellular vesicle and particle protein sample.

16. The method of claim 15, wherein the protein of (i) is thrombospondin 2.

17. The method of claim 15, wherein the protein of (i) is versican.

18. The method of claim 15, wherein the protein of (ii) is CD36 molecule.

19. The method of claim 15, wherein protein of (ii) is caveloae associated protein 2.

20. The method of claim 15, wherein at least two proteins of (i) are detected as a result of said subjecting.

21. The method of claim 15, wherein at least two proteins of (ii) are detecting as a result of said subjecting.

22. The method of claim 15, wherein at least two proteins of (i) and at least two proteins of (ii) are detected as a result of said subjecting.

23. The method of claim 22, wherein the at least two proteins of (i) are thrombospondin 2 and versican, and the at least two proteins of (ii) are caveolae associated protein 2 and CD36 molecule.

24. The method of claim 15, wherein the subject is a healthy subject.

25. The method of claim 15, wherein the subject is undergoing treatment for cancer.

26. The method of claim 15, where the subject is in remission for cancer.

27. A method of determining the presence of lung cancer in a subject, said method comprising:
obtaining a tissue sample from the subject;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting of Four and a half LIM domains protein 2 (FHL2), 5′-3′ exoribonuclease 2, EC 3.1.13. (XRN2), Glutaredoxin-3 (GLRX), Vigilin (High density lipoprotein-binding protein, HDL-binding protein) (HDLBP), Serrate RNA effector molecule homolog (SRRT), Regulator of chromosome condensation (RCC1), AP-3 complex subunit sigma-1 (AP3S1), Small nuclear ribonucleoprotein Sm D3, Sm-D3 (SNRPD3), NOP2, 60S ribosomal protein L22 (RPL22), DnaJ homolog subfamily C member 7 (DNAJC7), STE20/SPS1-related proline-alanine-rich protein kinase, Ste-20-related kinase (STK39), Signal recognition particle 54 kDa protein (SRP54), ATP-dependent DNA/RNA helicase DHX36 (DHX36), ELAV-like protein 1 (ELAVL1), Thrombospondin-2 (THBS2), Aconitate hydratase, mitochondrial, Aconitase (ACO2), Acyl-CoA-binding domain-containing protein 3 (ACBD3), Signal recognition particle 9 kDa protein (SRP9), THO complex subunit 2 (THOC2), Heterogeneous nuclear ribonucleoproteins C1/C2 (HNRNPC), Eukaryotic translation initiation factor 5B (EIF5B), RNA-binding protein Raly (RALY), Ubiquitin carboxyl-terminal hydrolase isozyme L5 (UCHL5), KH domain-containing, RNA-binding, signal transduction-associated protein 1 (KHDRBS1), Splicing factor 3B subunit 6 (SF3B6), WD repeat-containing protein 44 (WDR44), BRISC and BRCA1-A complex member 2 (BABAM2), Cleavage stimulation factor subunit 3 (CSTF3), HIV-1 Tat interactive protein 2 (HTATIP2), methyltransferase like 1 (METTL1), and combinations thereof; and
(ii) a protein selected from the proteins listed in Table 8 or any combination of proteins thereof; thereby detecting the presence or absence of the protein of (i) and the protein of (ii) in the extracellular vesicle and particle protein sample.

28. The method of claim 27, wherein the protein of (i) is selected from the group consisting of HIV-1 Tat interactive protein 2 (HTATIP2) and methyltransferase like 1 (METTL1).

29. A method of determining the presence of lung cancer in a subject, said method comprising:
obtaining a liquid biopsy sample from the subject;
separating extracellular vesicles and particles from the liquid biopsy sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting of subjecting the exosomal protein sample to a detection assay suitable for detecting the presence of selenoprotein P (SELENOP), rho-related GTP binding protein RhoV (RHOV), roquin-2 (RC3H2), claudin-5 (CLDN5), dematin (DMTN), serine/threonine-protein kinase/endoribonuclease IRE1 (ERN1), IGCL2, radixin (RDX), complement factor B (CFB), trypsin-1, EC 3.4.21.4 (PRSS1), leukocyte surface antigen CD53 (CD53), charged multivesicular body protein 4b (CHMP4B), proteasome subunit beta type-1 (PSMB1), actin aortic smooth muscle (ACTA2), guanine nucleotide-binding protein (GNG5), histone H2A.Z (H2AFZ), histone H2A type 1-C (HIST1H2AC), POTE ankyrin domain family member E (POTEE), POTE ankyrin domain family member I (POTEI) and combinations thereof, and
(ii) a protein selected from immunoglobulin heavy constant delta (IGHD), collectin-11 (COLEC11), immunoglobulin lambda variable 4-69 (IGLV4-69), Thrombospondin-2 (THBS2), Immunoglobulin kappa variable 1-27 (IGKV1-27), Immunoglobulin lambda variable 4-60 (IGLV4-60), Complement C1q tumor necrosis factor-related protein 3 (C1QTNF3), Probable non-functional immunoglobulin heavy variable 3-35 (IGHV3-35), Immunoglobulin lambda variable 2-18 (IGLV2-18), Immunoglobulin kappa variable 3D-15 (IGKV3D-15), Immunoglobulin kappa variable 3D-11 (IGKV3D-11), Immunoglobulin kappa variable 1-6 (IGKV1-6), Immunoglobulin kappa variable 1-17 (IGKV1-17), Attractin (ATRN), Immunoglobulin kappa variable 3/OR2-268 (non-functional) (IGKV3OR2-268), Immunoglobulin lambda variable 3-27 (IGLV3-27), Cholinesterase (BCHE), Immunoglobulin heavy variable 3/OR15-7 (IGHV3OR15-7), Thrombospondin-1 (Glycoprotein G) (THBS1), Immunoglobulin kappa variable 1-8 (IGKV1-8), Multimerin-1 (MMRN1), Probable non-functional immunoglobulin kappa variable 3-7 (IGKV3-7), Immunoglobulin lambda variable 3-16 (IGLV3-16), Immunoglobulin lambda variable 9-49 (IGLV9-49), Apolipoprotein M (APOM), Immunoglobulin kappa variable 2-29 (IGKV2-29), Immunoglobulin lambda variable 1-44 (IGLV1-44), Sushi, von Willebrand factor type A (SVEP1), Collectin-10 (COLEC10), Integrin alpha-IIb (ITGA2B), Complement C1r subcomponent-like protein (C1RL), Immunoglobulin kappa variable 1-39 (IGKV1-39), Immunoglobulin lambda variable 5-45 (IGLV5-45), insulin-like growth factor-binding protein complex acid labile subunit (IGFALS), putative hydroxypyruvate isomerase (HYI), Mannose-binding protein C (MBL2), Platelet factor 4, PF-4 (PF4), Coagulation factor XI, FXI, EC 3.4.21.27 (F11), Transforming growth factor beta-1 proprotein (TGFB1), Probable non-functional immunoglobulin kappa variable 2D-24 (IGKV2D-24), Immunoglobulin kappa variable 2-24 (IGKV2-24), Immunoglobulin kappa variable 2D-29 (IGKV2D-29), Mannosyl-oligosaccharide 1,2-alpha-mannosidase IC (MAN1C1), Charged multivesicular body protein 4a (CHMP4A), SERPIN4A, C-type lectin domain family 3 member B (CLEC3B), Platelet factor 4 variant (PF4V1), Immunoglobulin kappa variable 1-16 (IGKV1-16), Immunoglobulin kappa variable 1-12 (IGKV1-12), Immunoglobulin heavy variable 3/OR16-12 (non-functional) (IGHV3OR16-12), and any combination thereof; thereby detecting the presence or absence of the protein of (i) and the protein of (ii) in the extracellular vesicle and particle protein sample.

30. The method of claim 27 or claim 29, wherein detecting the presence of a protein from (i) and the absence of a protein from (ii) identifies lung cancer in the subject.

31. A method of determining the presence of pancreatic cancer in a subject, said method comprising:
obtaining a tissue sample from a subject;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting:
(i) a protein selected from, Myosin light polypeptide 6 (MYL6), EH domain-containing protein 1 (EHD1), Myosin-10 (MYH10), Fibronectin (FN1), Tropomyosin alpha-4 chain (TPM4), Flotillin-2 (FLOT2), Apolipoprotein A-I (APOA1), Thrombospondin-1 (THBS1), Tropomyosin alpha-3 chain (TPM3), Versican (VCAN), Dihydropyrimidinase-related protein 3 (DPYSL3), Actin-related protein 2/3 complex subunit 3 (ARPC3), Cathepsin B (CTSB), Thrombospondin-2 (THBS2), Coagulation factor XIII A chain (F13A1), Rho-related GTP-binding protein (RHOG), Myosin-9 (MYH9), Actin-related protein 2 (ACTR2), F-actin-capping protein subunit alpha-1 (CAPZA1), Actin-related protein 3 (ACTR3), Annexin A3 (ANXA3), Vimentin (VIM), Transitional endoplasmic reticulum ATPase (VCP), AP-2 complex subunit beta (AP2B1), Cytoplasmic dynein 1 heavy chain 1 (DYNC1H1), Vacuolar protein sorting-associated protein 35 (VPS35), High affinity immunoglobulin epsilon receptor subunit gamma (FCER1G), TB/POZ domain-containing protein KCTD12 (KCTD12), Guanine nucleotide-binding protein G(q) subunit alpha (GNAQ), Serpin H1 (SERPINH1), Ras-related protein Rab-31 (RAB31), Cytochrome b-245 heavy chain (CYBB), Protein S100-A13 (S100A13), Tropomyosin beta chain (TPM2), Milk fat globule-EGF factor 8 (MFGE8), Periostin (POSTN), Platelet-derived growth factor receptor beta, PDGF-R-beta (PDGFRB), Histidine-rich glycoprotein (HRG), Interferon-induced GTP-binding protein Mx1 (MX1), LIM and senescent cell antigen-like-containing domain protein 1 (LIMS1), Acyl-protein thioesterase 2 (LYPLA2), Inactive tyrosine-protein kinase 7 (PTK7), Ras-related protein Rab-22A (RAB22A), IST1 homolog (IST1), Raftlin (RFTN1), Plexin-B2 (PLXNB2), Vacuolar protein sorting-associated protein 28 homolog (VPS28), C-type mannose receptor 2 (MRC2), Neutrophil elastase (ELANE), Formin-like protein 1 (FMNL1), Cyclin-dependent kinase 4 (CDK4), Cyclin-dependent kinase 2 (CDK2), AP-2 complex subunit sigma (AP2S1), Prolyl endopeptidase FAP (FAP), Basigin (BSG), NADH-cytochrome b5 reductase 3 (CYB5R3), Fibulin-2 (FBLN2), Beta-hexosaminidase subunit beta (HEXB), Cyclin-dependent kinase 17 (CDK17), Tyrosine-protein kinase Lck (LCK), Retinoid-inducible serine carboxypeptidase (SCPEP1), Integrin alpha-X (ITGAX), Complement C1q subcomponent subunit B (C1QB), Macrophage-capping protein (CAPG), Osteoclast-stimulating factor 1 (OSTF1), Syntaxin-7 (STX7), Ectonucleoside triphosphate diphosphohydrolase 1 (ENTPD1), Neutrophil cytosol factor 2 (NCF2), Intercellular adhesion molecule 1 (ICAM1), Kinesin light chain 1 (KLC1), S-phase kinase-associated protein 1 (SKP1), Polyunsaturated fatty acid 5-lipoxygenase (ALOX5), Anoctamin-6 (ANO6), Metalloproteinase inhibitor 1 (TIMP1), 5′-AMP-activated protein kinase subunit gamma-1 (PRKAG1), Unconventional myosin-If (MYO1F), Mucin-5B (MUC5B), Alpha-1-antitrypsin (SERPINA1), and a combination thereof, and
(ii) a protein selected from the proteins listed in Table 9 or any combination of proteins thereof; thereby detecting the presence or absence of the protein of (i) and the protein of (ii) in the extracellular vesicle and particle protein sample.

32. The method of claim 31, wherein the protein of (i) is selected from the group consisting of FLOT2, TPM3, FCER1G, GNAQ, RAB31, CYBB, S100A13, TPM2, MFGE8, POSTN, PDGFRB, HRG, MX1, LIMS1, LYPLA2, PTK7, RAB22A, IST1, RFTN1, PLXNB2, VPS28, MRC2, ELANE, FMNL1, CDK4, CDK2, AP2S1, FAP, BSG, CYB5R3, FBLN2, HEXB, CDK17, LCK, SCPEP1, ITGAX, C1QB, CAPG, OSTF1, STX7, ENTPD1, NCF2, ICAM1, KLC1, SKP1, ALOX5, ANO6, TIMP1, PRKAG1, and MYO1F.

33. A method for determining presence of pancreatic cancer in a subject, said method comprising:
obtaining a liquid biopsy sample from the subject;
separating extracellular vesicles and particles from the liquid biopsy sample;
isolating protein from the separated extracellular vesicles and particles to form an extracellular vesicle and particle protein sample;
subjecting the extracellular vesicle and particle protein sample to a detection assay suitable for detecting:
(i) Heat shock-related 70 kDa protein 2 (HSPA2), Carbonic anhydrase 2 (CA2), Ras-related protein Rab-1A (RAB1A), Ras-related protein Rab-8B (RAB8B), Ras-related protein Rap-1A (RAP1A), Brain-specific angiogenesis inhibitor 1-associated protein 2-like protein 1 (BAIAP2L1), Complement decay-accelerating factor (CD55), Golgi-associated plant pathogenesis-related protein 1 (GLIPR2), GTPase KRas (KRAS), Leucine-rich repeat-containing protein 26 (LRRC26), Lactotransferrin (LTF), Protein disulfide-isomerase (P4HB), Phosphatidylethanolamine-binding protein 1 (PEBP1), Radixin (RDX), ATP-dependent translocase ABCB1 (ABCB1), Bile salt export pump (ABCB11), Phosphatidylcholine translocator ABCB4 (ABCB4), Adhesion G-protein coupled receptor G6 (ADGRG6), Alcohol dehydrogenase 1A (ADH1A), Alkaline phosphatase, tissue-nonspecific isozyme (ALPL), Integrin alpha-1 (ITGA1), Protein kinase C and casein kinase substrate in neurons protein (2PACSIN2), Receptor-type tyrosine-protein phosphatase eta (PTPRJ), Ras-related protein Rap-2b (RAP2B), Sorcin (SRI), Xaa-Pro aminopeptidase 2 (XPNPEP2), Alcohol dehydrogenase 1C (ADH1C), All-trans-retinol dehydrogenase [NAD(+)] ADH4 (ADH4), Annexin A11 (ANXA11), T-complex protein 1 subunit zeta (CCT6A), Copine-1 (CPNE1), Desmocollin-1 (DSC1), Desmoglein-1 (DSG1), Desmoplakin (DSP), Glutamyl aminopeptidase (ENPEP), Fatty acid-binding protein, liver (FABP1), High affinity immunoglobulin epsilon receptor subunit gamma (FCER1G), Filamin-B (FLNB), Guanine nucleotide-binding protein G(I)/G(S)/G(O) subunit gamma-5 (GNG5), Keratin, type II cytoskeletal 8 (KRT8), Keratin, type II cuticular Hb1 (KRT81), Keratin, type II cuticular Hb5 (KRT85), 55 kDa erythrocyte membrane protein (MPP1), Plasminogen-like protein B (PLGLB1), Peroxiredoxin-6 (PRDX6), Proteasome subunit alpha type-4 (PSMA4), Proteasome subunit alpha type-5 (PSMA5), 14-3-3 protein sigma (SFN), Sorting nexin-18 (SNX18), Protein-glutamine gamma-glutamyltransferase 2 (TGM2), Hyaluronoglucosaminidase (TMEM2), and combinations thereof, and
(ii) a protein selected from Immunoglobulin heavy constant delta (IGHD), Collectin-11 (COLEC11), Immunoglobulin lambda variable 4-69 (IGLV4-69), Thrombospondin-2 (THBS2), Immunoglobulin kappa variable 1-27 (IGKV1-27), Immunoglobulin lambda variable 4-60 (IGLV4-60), Complement C1q tumor necrosis factor-related protein 3 (C1QTNF3), Probable non-functional immunoglobulin heavy variable 3-35 (IGHV3-35), Immunoglobulin lambda variable 2-18 (IGLV2-18), Immunoglobulin kappa variable 3D-15 (IGKV3D-15), Immunoglobulin kappa variable 3D-11 (IGKV3D-11), Immunoglobulin kappa variable 1-6 (IGKV1-6), Immunoglobulin kappa variable 1-17 (IGKV1-17), Attractin (ATRN), Immunoglobulin kappa variable 3/OR2-268 (non-functional) (IGKV3OR2-268), Immunoglobulin lambda variable 3-27 (IGLV3-27), Cholinesterase (BCHE), Immunoglobulin heavy variable 3/OR15-7 (pseudogene) (IGHV3OR15-7), Thrombospondin-1 (THBS1), Immunoglobulin kappa variable 1-8 (IGKV1-8), Multimerin-1 (MMRN1), Probable non-functional immunoglobulin kappa variable 3-7 (IGKV3-7), Immunoglobulin lambda variable 3-16 (IGLV3-16), Immunoglobulin lambda variable 9-49 (IGLV9-49), Apolipoprotein M (APOM), Immunoglobulin kappa variable 2-29 (IGKV2-29), Immunoglobulin lambda variable 1-44 (IGLV1-44), Sushi, von Willebrand factor type A, EGF and pentraxin domain-containing protein 1 (SVEP1), Collectin-10 (COLEC10), Integrin alpha-IIb (ITGA2B), Complement C1r subcomponent-like protein (C1RL), Immunoglobulin kappa variable 1-39 (IGKV1-39), Immunoglobulin lambda variable 5-45 (IGLV5-45), Insulin-like growth factor-binding protein complex acid labile subunit (IGFALS), HY1, Mannose-binding protein C (MBL2), Platelet factor 4 (PF4), Coagulation factor XI (F11), Transforming growth factor beta-1 proprotein (TGFB1), Probable non-functional immunoglobulin kappa variable 2D-24 (IGKV2D-24), Immunoglobulin kappa variable 2-24 (IGKV2-24), Immunoglobulin kappa variable 2D-29 (IGKV2D-29), Mannosyl-oligosaccharide 1,2-alpha-mannosidase IC (MAN1C1), Charged multivesicular body protein 4a (CHMP4A), SERPIN4A, Tetranectin (CLEC3B), Platelet factor 4 variant (PF4V1), Immunoglobulin kappa variable 1-16 (IGKV1-16), Immunoglobulin kappa variable 1-12 (IGKV1-12), Immunoglobulin heavy variable 3/OR16-12 (non-functional) (IGHV3OR16-12) and any combination thereof, thereby detecting the presence or absence of the protein of (i) and the protein of (ii) in the extracellular vesicle and particle protein sample.

34. The method of claim 31 or claim 33, wherein detecting the presence of a protein from (i) and the absence of a protein from (ii) identifies pancreatic cancer in the subject.

35. A method of determining the presence of neuroblastoma in a subject, said method comprising:
obtaining a liquid biopsy sample from the subject;
separating extracellular vesicles and particles from the liquid biopsy sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting ferritin heavy chain (FTH1), keratin, type I cytoskeletal 17 (KRT17), histone H3.3 (H3F3A), ATP-binding cassette sub-family B member 9 (ABCB9), a disintegrin and metalloproteinase with thrombospondin motifs 13 (ADAMTS13), CD14, erythrocyte membrane protein band 4.2 (EPB42), hepatocyte growth factor activator (HGFAC), keratin, type I cytoskeletal 13 (KRT13), and KRT8 (Figure S13A), and combinations thereof, and
(ii) a protein selected from the proteins listed in Table 3 or any combination of proteins thereof; thereby detecting the presence or absence of the protein of (i) and the protein of (ii) in the extracellular vesicle and particle protein sample.

36. The method of claim 35, wherein detecting the presence of a protein from (i) and the absence of a protein from (ii) identifies neuroblastoma in the subject.

37. A method of determining the presence of osteosarcoma in a subject, said method comprising:
obtaining a liquid biopsy sample from a subject;
separating extracellular vesicles and particles from the liquid biopsy sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting actin, alpha skeletal muscle (ACTA1), actin, gamma-enteric smooth muscle (ACTG2), ADAMTS13, HGFAC, neprilysin (MME), and TNC, and combinations thereof and
(ii) a protein selected from the proteins listed in Table 4 or any combination of proteins thereof; thereby detecting the presence or absence of the protein of (i) and the protein of (ii) in the extracellular vesicle and particle protein sample.

38. The method of claim 37, wherein detecting the presence of a protein from (i) and the absence of a protein from (ii) identifies osteosarcoma in the subject.

39. A method of cancer sub-type identification, said method comprising:
obtaining a liquid biopsy sample from a subject;
separating extracellular vesicles and particles from the sample;
isolating protein from the separated extracellular vesicles and particles to form an extracellular vesicle and particle protein sample;
subjecting the extracellular vesicle and particle protein sample to a detection assay suitable for detecting levels of at least three proteins selected from the group consisting of Fibrinogen beta chain (FGB), FGA (Fibrinogen alpha chain), Fibrinogen gamma chain (FGG), Complement factor H (CFH), Plasminogen (PLG), Immunoglobulin heavy variable 3-53 (IGHV3-53), Serum amyloid P-component, SAP (APCS), Complement factor H-related protein 1 (CFHR1), Immunoglobulin heavy variable 3-48 (IGHV3-48), Immunoglobulin heavy variable 3-74 (IGHV3-74), Immunoglobulin heavy variable 3-72 (IGHV3-72), Immunoglobulin heavy variable 3-43 (IGHV3-43), Immunoglobulin heavy variable 5-10-1 (IGHV5-10-1), Immunoglobulin lambda variable 7-46 (IGLV7-46), Immunoglobulin kappa variable 3D-20 (IGKV3D-20), Immunoglobulin kappa variable 2-24 (IGKV2-24), Complement factor H-related protein 2 (CFHR2), Immunoglobulin heavy variable 4-59 (IGHV4-59), Immunoglobulin heavy variable 3-20 (IGHV3-20), Immunoglobulin heavy variable 3-64 (IGHV3-64), Probable non-functional immunoglobulin heavy variable 3-16 (IGHV3-16), Immunoglobulin heavy variable 3-11 (IGHV3-11), Immunoglobulin heavy variable 3/OR16-9 (IGHV3OR16-9), Probable non-functional immunoglobulin kappa variable 2D-24 (IGKV2D-24), Immunoglobulin lambda constant 3 (IGLC3), Immunoglobulin heavy variable 3/OR16-13 (IGHV3OR16-13), Complement factor H-related protein 3 (CFHR3), Immunoglobulin heavy constant gamma 3 (IGHG3), Immunoglobulin lambda constant 2 (IGLC2), and Immunoglobulin kappa variable 1-8 (IGKV1-8).

40. The method of claim 39, wherein the presence or absence of all of the proteins are detected as a result of said subjecting

41. The method of claim 39, wherein the subject has a primary tumor of unknown origin, said method further comprising:
identifying, based on said subjecting, the primary tumor type.

42. The method of claim 41 further comprising:
administering, to the subject, a therapeutic drug based on said identifying.

43. The method of claim 39, wherein the at least three proteins are Immunoglobulin kappa variable 1-8 (IGKV1-8), Immunoglobulin lambda constant 3 (IGLC3), and Immunoglobulin heavy variable 3/OR16-13 (IGHV3OR16-13).

44. The method of claim 43 further comprising:
identifying lung cancer in the subject when IGKV1-8 is detected and IGLC3 and IGHV3OR16-13 are not detected during said subjecting.

45. The method of claim 39, wherein the at least three proteins are selected from immunoglobulin lambda constant 3 (IGLC3), immunoglobulin heavy variable 4-59 (IGHV4-59), immunoglobulin heavy variable 3-20 (IGHV3-20), immunoglobulin heavy variable 3-64 (IGHV3-64), immunoglobulin heavy variable 3-16 (IGHV3-16), immunoglobulin heavy variable 3-11 (IGHV3-11), complement factor H-related protein 3 (CFHR3), immunoglobulin heavy variable 3 or 16-9 (IGHV3OR16-9).

46. The method of claim 45 further comprising:
identifying pancreatic cancer in the subject when IGLC3 is detected and CFHR3, IGHG3, IGHV4-59, IGHV3-20, IGHV3-64, IGLV3-16, IGHV3-11, IGHV3OR16-9, or any combination thereof is not detected during said subjecting.

47. The method of claim 39, wherein the at least three proteins are selected from IGHG3, IGHV3-74, IGHV3-72, IGHV3-43, IGHV5-10-1, IGLV7-46, IGKV3D-20, and IGKV2-24.

48. The method of claim 47 further comprising:
identifying breast cancer in the subject when IGHG3 is detected and IGHV3-74, IGHV3-72, IGHV3-43, IGHV5-10-1, IGLV7-46, IGKV3D-20, IGKV2-24, or any combination thereof is not detected during said subjecting

49. The method of claim 39, wherein the at least three proteins are selected from IGHV5-10-1, IGLV7-46, IGHG3 and IGLC2.

50. The method of claim 49 further comprising:
identifying colorectal cancer in the subject when IGLC2 is detected and IGHV5-10-1, IGLV7-46, IGHG3, or any combination thereof is not detected during said subjecting.

51. The method of claim 39, wherein the at least three proteins are IGLC2, IFKV1, and CFHR3.

52. The method of claim 51 further comprising:
identifying mesothelioma in the subject when CFHR3 is detected and IGLC2 and IFKV1 are not detected during said subjecting.

53. A method of cancer sub-type identification, said method comprising:
obtaining a tissue sample from a subject;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting at least three proteins selected from the group consisting of Apolipoprotein D (APOD), Polyubiquitin-C (UBC), Transaldolase (TALDO1), Thymidine phosphorylase (TYMP), Aminopeptidase B (RNPEP), Transgelin (TAGLN), Septin (SEPT7), Histone H2A type 2-B (HIST2H2AB), Gamma-enolase (ENO2), NADH-cytochrome b5 reductase 3 (CYB5R3), Actin-related protein 2/3 complex subunit 4 (ARPC4), Interleukin enhancer-binding factor 2 (ILF2), Protein transport protein Sec23B (SEC23B), COMM domain-containing protein 3 (COMMD3), Ankyrin-3 (ANK3), Glycogen phosphorylase, muscle form (PYGM), Putative histone H2B type 2-D (HIST2H2BD), Keratin, type I cytoskeletal 19 (KRT19), Sulfotransferase 1A2 (SULT1A2), Desmin (DES), Histone H2B (HIST1H2BD), Histone H2B type 1-A (HIST1H2BA), Histone H3.1t (HIST3H3), Tubulin beta-1 chain (TUBB1), Retinal dehydrogenase 2 (ALDH1A2), HLA class II histocompatibility antigen, DP beta 1 chain (HLA-DPB1), Bifunctional epoxide hydrolase 2 (EPHX2), Mitochondrial-processing peptidase subunit alpha (PMPCA), and Xylulose kinase (XYLB).

54. The method of claim 53, wherein the tissue sample is obtained from a metastatic cancer site.

55. The method of claim 53, wherein the presence or absence of all of the proteins are detected as a result of said subjecting

56. The method of claim 53, wherein the subject has a primary tumor of unknown origin, said method further comprising:
identifying, based on said subjecting, the primary tumor type.

57. The method of claim 56 further comprising:
administering, to the subject, a therapeutic drug based on said identifying.

58. The method of claim 53, wherein the at least three proteins are selected from histone H2B type 1-D (HIST1H2BD), histone H2B type 1-A (HIST1H2BA), histone H3.1t (HIST3H3), tubulin beta-1 chain (TUBB1), retinal dehydrogenase 2 (ALDH1A2), HLA-DPB1, and polyubiquitin-C (UBC).

59. The method of claim 58 further comprising:
identifying lung cancer in the subject when HIST1H2BD, HIST1H2BA, HIST3H3, TUBB1, ALDH1A2, HLA-DPB1 or any combination thereof is detected and UBC is not detected during said subjecting.

60. The method of claim 53, wherein the at least three proteins are selected from apolipoprotein D (APOD), polyubiquitin-C (UBC), bifunctional epoxide hydrolase 2 (EPHX2), mitochondrial-processing peptidase subunit alpha (PMPCA), and xylulose kinase (XYLB).

61. The method of claim 60 further comprising:
identifying pancreatic cancer in the subject when UBC, APOD, or any combination thereof are detected and EPHX2, PMPCA, XYLB, or any combination thereof is not detected during said subjecting

62. The method of claim 53, wherein the at least three proteins are selected from SEPT7, COMMD3, ANK3, PYGM and XYLB.

63. The method of claim 62 further comprising:
identifying melanoma in the subject when XYLB is detected and SEPT7, COMMD3, ANK3, PYGM, or any combination thereof is not detected during said subjecting.

64. The method of claim 53, wherein the at least three proteins are selected from SULT1A2, KRT19, HIST2H2BD, COMMD3, and ANK3.

65. The method of claim 64 further comprising:
identifying colorectal cancer in the subject when COMMD3 and/or ANK3 are detected and SULT1A2, KRT19, HIST2H2BD, or any combination thereof is not detected during said subjecting.

66. A method of identifying a primary tumor of unknown origin, said method comprising:
obtaining a tissue sample from a subject, wherein the tissue sample is from a primary tumor of unknown origin;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting one or more proteins independently selected from the proteins of Tables 12, 13, 14, and 15.

67. The method of claim 66 further comprising:
identifying the primary tumor of unknown origin as a pancreatic tumor, when one or more proteins from Table 12 is detected in the extracellular vesicle and particle protein sample during said subjecting.

68. The method of claim 66 further comprising:
identifying the primary tumor of unknown origin as a lung tumor, when one or more proteins from Table 13 is detected in the extracellular vesicle and particle protein sample during said subjecting.

69. The method of claim 66 further comprising:
identifying the primary tumor of unknown origin as a breast tumor, when one or more proteins from Table 14 is detected in the extracellular vesicle and particle protein sample during said subjecting.

70. The method of claim 66 further comprising:
identifying the primary tumor of unknown origin as a colon tumor, when one or more proteins from Table 15 is detected in the extracellular vesicle and particle protein sample during said subjecting.

71. A method of identifying a pancreatic lesion in a subject, said method comprising:
obtaining a liquid biopsy sample from a subject;
separating extracellular vesicles and particles from the biopsy sample;
isolating protein from the separated extracellular vesicles and particles to form an extracellular vesicle and particle protein sample;
subjecting the extracellular vesicle and particle protein sample to a detection assay suitable for detecting one or more proteins selected from Proteasome subunit alpha type-2 (PSMA2), Carbonic anhydrase 2 (CA2), Protein 4.1 (EPB41), CD59 glycoprotein (CD59), 2′,3′-cyclic-nucleotide 3′-phosphodiesterase (CNP), Ras-related protein Rab-8A (RAB8A), Triosephosphate isomerase (TPI1), Glutathione hydrolase 1 proenzyme (GGT1), Putative glutathione hydrolase 3 proenzyme (GGT3P), Inactive glutathione hydrolase 2 (GGT2), Phosphatidylethanolamine-binding protein 1 (PEBP1), Immunoglobulin lambda variable 8-61 (IGLV8-61), Complement component C6 (C6), Serum paraoxonase/arylesterase 1 (PON1), Carboxypeptidase N subunit 2 (CPN2), Extracellular matrix protein 1 (ECM1), Ig kappa chain V-I region AG, Immunoglobulin kappa variable 4-1 (IGKV4-1), IG lambda chain V-1 region, Properdin (CFP), Tubulin beta chain (TUBB), Tubulin beta-4B chain (TUBB4B), Tubulin beta-2B chain (TUBB2B), Tubulin beta-2A chain (TUBB2A), Vinculin (VCL), Ras suppressor protein 1 (RSU1), Fermitin family homolog 3 (FERMT3), and A disintegrin and metalloproteinase with thrombospondin motifs 13 (ADAMTS13).

72. The method of claim 71 further comprising:
identifying the presence of a cancerous pancreatic lesion in the subject when one or more proteins selected from IGLV8-61, CD59, CA2, CNP, EPB41, C6, CGT1, PON1, TPI1, RAB8A, ECM1, PSMA2, CPN2, and PEBP1 are detected in the extracellular vesicle and particle protein sample during said subjecting.

73. The method of claim 71 further comprising:
identifying the presence of a cancerous pancreatic lesion in the subject when one or more proteins selected from PSMA2, CPN2, and PEBP1 are detected in the extracellular vesicle and particle protein sample during said subjecting.

74. The method of claim 71 further comprising:
identifying the presence of a pre-cancerous pancreatic lesion in the subject when one or more proteins selected from VCL, CFP, and FERMT3 are detected in the extracellular vesicle and particle protein sample during said subjecting.

75. The method of any one of claims 1-74, wherein said separating comprises:
subjecting the sample to at least three sequential centrifugations, wherein the extracellular vesicles and particles are separated from the sample based on said subjecting.

76. The method of any one of claims 1-74, wherein said separating comprising:
contacting the sample with one or more binding molecules, wherein each binding molecule is capable of binding to a target extracellular vesicle and particle protein selected from the group consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin;
subjecting the sample, after said contacting, to conditions effective for the one or more binding molecules to bind to its respective target extracellular vesicle and particle protein in the sample to form one or more binding molecule-target protein complexes; and
selecting for the one or more binding molecule-target protein complexes, thereby separating the extracellular vesicle and particles from the sample.

77. The method of claim 76, wherein the sample is contacted with at least two different binding molecules.

78. The method of claim 76, wherein the sample is contacted with at least three different binding molecules.

79. The method of claim 76, wherein the sample is contacted with one or more binding molecules capable of binding to alpha-2-macroglobulin, moesin, and galectin-3-binding protein.

80. The method of claim 76, wherein the sample is contacted with a binding molecule capable of binding alpha-2-macroglobulin, a binding molecule capable of binding moesin, and a binding molecule capable of binding galectin-3-binding protein.

81. The method of any one of claims 1-80, wherein the detection assay is an immunoassay.

82. The method of any one of claims 1-80, wherein the detection assay is mass spectroscopy.

83. A method of isolating extracellular vesicles and particles from a biological sample, said method comprising:
obtaining a biological sample from a subject;
contacting the sample with one or more binding molecules, wherein each binding molecule is capable of binding to a target extracellular vesicle and particle protein selected from the group consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin;
subjecting the sample, after said contacting, to conditions effective for the one or more binding molecules to bind to its respective target extracellular vesicle and particle protein in the sample to form one or more binding molecule-target protein complexes; and
separating the one or more binding molecule-target protein complexes from the sample, thereby isolating extracellular vesicles and particles from the sample.

84. The method of claim 83, wherein the sample is contacted with at least two different binding molecules.

85. The method of claim 83, wherein the sample is contacted with at least three different binding molecules.

86. The method of claim 83, wherein the sample is contacted with one or more binding molecules capable of binding to alpha-2-macroglobulin, moesin, and galectin-3-binding protein.

87. The method of claim 83, wherein the sample is contacted with a binding molecule capable of binding alpha-2-macroglobulin, a binding molecule capable of binding moesin, and a binding molecule capable of binding galectin-3-binding protein.

88. A kit suitable for detecting the presence of cancer in subject, said kit comprising reagents suitable for detecting:
(i) a protein selected from the group consisting of ferritin light chain, von Willebrand factor, immunoglobulin lambda constant 2, keratin 17, immunoglobulin heavy constant gamma 1, keratin 6B, radixin, cofilin 1, protease, serine 1, tubulin alpha 1c, ADAM metallopeptidase with thrombospondin type 1 motif 13, immunoglobulin kappa variable 6D-21, tyrosine 3-monooxygenase/tryptophan 5-monooxygenase activation protein theta, POTE ankyrin domain family member I, POTE ankyrin domain family member F, and combinations thereof, and
(ii) a protein selected from the group consisting of actin gamma 1, immunoglobulin lambda variable 3-27, immunoglobulin kappa variable 1D-12, coagulation factor XI, complement C1r subcomponent like, attractin, butyrylcholinesterase, immunoglobulin heavy variable 3-35, immunoglobulin kappa variable 1-17, C1q and TNF related 3, immunoglobulin heavy variable 3-20, immunoglobulin heavy variable 3/OR15-7, collectin subfamily member 11, immunoglobulin heavy constant delta, immunoglobulin kappa variable 3D-11, immunoglobulin heavy variable 3/OR16-10, immunoglobulin kappa variable 2D-24, immunoglobulin kappa variable 2-40, immunoglobulin kappa variable 1-27, immunoglobulin heavy variable 3/OR16-9, immunoglobulin lambda variable 5-45, immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6, and combinations thereof.

89. A kit suitable for detecting the presence of cancer in a subject, said kit comprising reagents suitable for detecting:
(i) a protein selected from the group consisting thrombospondin 2, versican, serrate, RNA effector molecule, tenascin C, dihydropyrimidinase like 2, adenosylhomocysteinase, DnaJ heat shock protein family (Hsp40) member A1, phosphoglycerate kinase 1, EH domain containing 2, and combinations thereof, and
(ii) a protein selected from the group consisting of alcohol dehydrogenase 1B (class I), beta polypeptide, caveolae associated protein 1, FGGY carbohydrate kinase domain containing, ATP binding cassette subfamily A member 3, syntaxin 11, caveolae associated protein 2, CD36 molecule, and combinations thereof, thereby detecting the presence or absence of the protein of (i) and the protein of (ii) in the extracellular vesicle and particle protein sample

90. A kit suitable for identifying the origin of a tumor from a liquid biopsy, said kit comprising reagents suitable for detecting at least three proteins selected from the group consisting of Fibrinogen beta chain (FGB), FGA (Fibrinogen alpha chain), Fibrinogen gamma chain (FGG), Complement factor H (CFH), Plasminogen (PLG), Immunoglobulin heavy variable 3-53 (IGHV3-53), Serum amyloid P-component, SAP (APCS), Complement factor H-related protein 1 (CFHR1), Immunoglobulin heavy variable 3-48 (IGHV3-48), Immunoglobulin heavy variable 3-74 (IGHV3-74), Immunoglobulin heavy variable 3-72 (IGHV3-72), Immunoglobulin heavy variable 3-43 (IGHV3-43), Immunoglobulin heavy variable 5-10-1 (IGHV5-10-1), Immunoglobulin lambda variable 7-46 (IGLV7-46), Immunoglobulin kappa variable 3D-20 (IGKV3D-20), Immunoglobulin kappa variable 2-24 (IGKV2-24), Complement factor H-related protein 2 (CFHR2), Immunoglobulin heavy variable 4-59 (IGHV4-59), Immunoglobulin heavy variable 3-20 (IGHV3-20), Immunoglobulin heavy variable 3-64 (IGHV3-64), Probable non-functional immunoglobulin heavy variable 3-16 (IGHV3-16), Immunoglobulin heavy variable 3-11 (IGHV3-11), Immunoglobulin heavy variable 3/OR16-9 (IGHV3OR16-9), Probable non-functional immunoglobulin kappa variable 2D-24 (IGKV2D-24), Immunoglobulin lambda constant 3 (IGLC3), Immunoglobulin heavy variable 3/OR16-13 (IGHV3OR16-13), Complement factor H-related protein 3 (CFHR3), Immunoglobulin heavy constant gamma 3 (IGHG3), Immunoglobulin lambda constant 2 (IGLC2), and Immunoglobulin kappa variable 1-8 (IGKV1-8).

91. A kit suitable for identifying the origin of a tumor from a tissue biopsy, said kit comprising reagents suitable for detecting at least three proteins selected from the group consisting of APOD, UBC, TALDO1, TYMP, RNPEP, TAGLN, SEPT7, HIST2H2AB, ENO2, CYB5R3, ARPC4, ILF2, SEC23B, COMMD3, ANK3, PYGM, HIST2H2BD, KRT19, SULT1A2, DES, HIST1H2BD, HIST1H2BA, HIST3H3, TUBB1, ALDH1A2, HLA-DPB1, EPHX2, PMPCA, and XYLB.

92. A kit suitable for identifying the origin of a metastatic tumor from a tissue biopsy, said kit comprising reagents suitable for detecting at least one or more proteins selected from the proteins listed in Tables 12, 13, 14, and 15.

93. A kit suitable for isolating exosomes from a human sample, said kit comprising at least one binding molecule capable of binding a protein selected from the group consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin

94. The kit of claim 93, said kit comprising at least three different binding molecules, each binding molecule capable of binding a different protein in the group consisting of alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin.

95. The kit of claim 93, wherein the at least three different binding molecules comprise a binding capable of binding to alpha-2-macroglobulin, a binding molecule capable of binding moesin, and a binding molecule capable of binding galectin-3-binding protein.

96. A kit for identifying a pancreatic lesion, said kit comprising reagents suitable for detecting at least one or more proteins selected from PSMA2, CA2, EPB41, CD59, CNP, RAB8A, TPI1, GGT1, GGT3P, GGT2, PEBP1, IGLV8-61, C6, PON1, CPN2, ECM1, Ig kappa chain V-I region AG, IGKV4-1, IG lambda chain V-1 region, CFP, TUBB, TUBB4B, TUBB2B, TUBB2A, VCL, RSU1, FERMT3, and ADAMTS13.

97. A method of determining a treatment regimen for a subject having a tumor, said method comprising:
obtaining, from the subject having the tumor, a biopsy of tumor tissue and a biopsy of tissue adjacent to the tumor;
separating extracellular vesicles and particles from the obtained samples;
isolating protein from the separated extracellular vesicle and particles to form extracellular vesicle and particle protein samples;
subjecting the extracellular vesicle and particle protein samples to a detection assay suitable for detecting proteins differentially expressed in exosomes from the tumor tissue versus adjacent, non-tumor tissue; and
treating said subject based on said subjecting.

98. A method of identifying drug targets for cancer therapy, said method comprising:
obtaining, from one or more subjects having a particular tumor type, a biopsy of tumor tissue and a biopsy of tissue adjacent to said tumor;
separating extracellular vesicles and particles from the obtained samples;
isolating protein from the separated extracellular vesicle and particles to form extracellular vesicle and particle protein samples;
subjecting the extracellular vesicle and particle protein samples to proteomic analysis to identify proteins differentially expressed in the tumor tissue versus tissue adjacent said tumor; and
identifying drug targets for cancer therapy based on said subjecting.

99. A method of treating a subject having a tumor, said method comprising:
administering a Sam68 inhibitor to a subject having a tumor, wherein exosomes from the tumor tissue express Src-associated in mitosis 68 kDa protein (Sam68; KHDRBS1).

100. The method of claim 99, wherein the Sam68 inhibitor is CWP232291.

101. The method of claim 99 or claim 100, wherein the tumor is a lung tumor.

102. A method of treating a subject having a tumor, said method comprising:
administering a nucleolin inhibitor to a subject having a tumor, wherein exosomes from the tumor tissue express nucleolin (NCL).

103. The method of claim 102, wherein the nucleolin inhibitor is AGR100 (AS1411).

104. A method of treating a subject having a tumor, said method comprising:
administering a tenacin inhibitor to the subject having a tumor, wherein exosomes from the tumor tissue express tenacin (TNC).

105. The method of claim 104, wherein the tenacin inhibitor is an F16-IL2 fusion protein.

106. A method of treating a subject having a tumor, said method comprising:
administering an inosine-5′-monophosphate dehydrogenase 2 inhibitor to the subject having a tumor, wherein exosomes from the tumor tissue express inosine-5′-monophosphate dehydrogenase 2 (IMPDH2).

107. The method of claim 106, wherein the inosine-5′-monophosphate dehydrogenase 2 inhibitor is selected from the group consisting of mycophenolic acid, thioguanine, mycophenolate mofetil, imatinib/thioguanine, VX-944, pegintron/ribavirin, mycophenolate mofetil/prednisone, methylprednisolone/mycophenolate mofetil, interferon alfacon-1/ribavirin, 6-mercaptopurine/prednisone/thioguanine, cytarabine/daunorubicin/thioguanine, cytarabine/thioguanine, IFNA2B/ribavirin, and ribavirin.

108. A method of treating a subject having a tumor, said method comprising:
administering a glutamine amidotransferase inhibitor to the subject having a tumor, wherein exosomes from the tumor tissue express GMP synthase (GMPS).

109. The method of claim 108, wherein the glutamine admidotransferase inhibitor is azaserine.

110. A method of treating a subject having a tumor, said method comprising:
administering a DNA topoisomerase I inhibitor to the subject having a tumor, wherein exosomes from the tumor tissue express DNA topoisomerase I (TOP1MT).

111. The method of claim 110, wherein the DNA topoisomerase I inhibitor is selected from the group consisting of capecitabine/cetuximab/irinotecan, irinotecan/leucovorin, cetuximab/irinotecan, gemcitabine/irinotecan, aflibercept/irinotecan, capecitabine/irinotecan, cetuximab/irinotecan/vemurafenib, and irinotecan.

112. A method of treating a subject having a tumor, said method comprising:
administering an ATIC inhibitor to the subject having a tumor, wherein exosomes from the tumor tissue express bifunctional purine biosynthesis protein ATIC (ATIC).

113. The method of claim 112, wherein the ATIC inhibitor is selected from the group consisting of pemetrexed, pembrolizumab/pemetrexed, and gemcitabine/pemetrexed.

114. A method of treating a subject having a tumor, said method comprising:
administering an aldo-keto reductase family 1 member B1 inhibitor to the subject having a tumor, wherein exosomes from the tumor tissue express aldo-keto reductase family 1 member B1 (AKR1B1).

115. The method of claim 114, wherein the aldo-keto reductase family 1 member B1 inhibitor is selected from the group consisting of pemetrexed, pembrolizumab/pemetrexed, and gemcitabine/pemetrexed.

116. A method of treating a subject having a tumor, said method comprising:
administering a cytokeratin-2e inhibitor to the subject having a tumor, wherein plasma tumor derived exosomes of the subject express cytokeratin-2e (KRT2).

117. The method of claim 116, wherein the cytokeratin-2e inhibitor is selected from the group consisting of CIGB-300 and silmitasertib

118. A method of treating a subject having a tumor, said method comprising:
administering a coagulation factor VIII inhibitor to the subject having a tumor, wherein plasma tumor derived exosomes of the subject express coagulation factor VIII (F8).

119. The method of claim 118, wherein the coagulation factor VIII inhibitor is drotrecogin alfa (recombinant human activated protein C) or recombinant coagulation factor IX.

120. A method of treating a subject having a tumor, said method comprising:
administering a peptidyl-prolyl cis-trans isomerase A inhibitor, to the subject having a tumor, wherein plasma tumor derived exosomes of the subject express peptidyl-prolyl cis-trans isomerase A (PPIA).

121. The method of claim 120, wherein the peptidyl-prolyl cis-trans isomerase A inhibitor is selected from the group consisting of cyclosporine A/sirolimus/tacrolimus, N-methyl-4-Ile-cyclosporin, alemtuzumab/cyclosporin A, cyclosporin A, cyclosporine A/tacrolimus, and cyclosporin A/methotrexate.

122. A method of treating a subject having a tumor, said method comprising:
administering a carbonic anhydrase I inhibitor to the subject having a tumor, wherein plasma tumor derived exosomes of the subject express carbonic anhydrase I (CA1).

123. The method of claim 122, wherein the carbonic anhydrase I inhibitor is selected from the group consisting of benzthiazide, ethoxyzolamide, brimonidine/brinzolamide, dorzolamide, diazoxide, dichlorphenamide, methazolamide, hydrochlorothiazide, sulfacetamide, dorzolamide/timolol, brinzolamide, topiramate, chlorothiazide/reserpine, chlorothiazide, chlorthalidone, acetazolamide, quinethazone, and trichloromethiazide.

124. A method for screening a subject for the presence of cancer, said method comprising:
obtaining a liquid biopsy sample from a subject;
separating extracellular vesicles and particles from the sample;
isolating protein from the separated extracellular vesicles and particles to form an extracellular vesicle and particle protein sample;
subjecting the extracellular vesicle and particle protein sample to a detection assay suitable for detecting:
(i) one or more proteins selected from the group consisting of Ferritin light chain (FTL), ABC-type oligopeptide transporter ABCB9 (ABCB9), Protein Z-dependent protease inhibitor (SERPINA10), Coagulation factor VIII (F8), Lactotransferrin (LTF), Basement membrane-specific heparan sulfate proteoglycan core protein (HSPG2), Protein disulfide-isomerase (P4HB), Trypsin-1 (PRSS1), Keratin, type II cytoskeletal 1b (KRT77), Endoplasmic reticulum chaperone BiP (HSPA5); and
(ii) one or both proteins selected from the group consisting of Complement C1q tumor necrosis factor-related protein 3 (C1QTNF3) and Immunoglobulin heavy constant delta (IGHD), thereby detecting the presence or absence of the protein of (i) and the protein of (ii) in the extracellular vesicle and particle protein sample.

125. A method for screening a subject for the presence of cancer, said method comprising:
obtaining a tissue sample from a subject;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting of tenacin (TNC), Periostin (POSTN), Versican core protein (VCAN), signal recognition particle 9 kDa protein (SRP9), Nucleophosmin (NPM1), Serrate RNA effector molecule homolog (SRRT), ELAV-like protein 1 (ELAVL1), Cytosolic acyl coenzyme A thioester hydrolase (ACOT7), 5′-3′ exoribonuclease 2 (XRN2), Flap endonuclease 1 (FEN1), ADP-ribosylation factor-like protein 1 (ARL1), Heat shock protein 105 kDa (HSPH1), Nucleolar RNA helicase 2 (DDX21), Src-associated in mitosis 68 kDa protein (KHDRBS1), Importin subunit alpha-1 (KPNA2), SLIT-ROBO Rho GTPase-activating protein 1 (SRGAP1), WD repeat-containing protein 3 (WDR3), and combinations thereof, and
(ii) a protein selected from the group consisting of Voltage-dependent calcium channel subunit alpha-2/delta-2 (CACNA2D2), Specifically androgen-regulated gene protein (C1orf116), Caveolin-2 (CAV2), Syntaxin-11 (STX11), Caveolae-associated protein 2 (CAVIN2), and combinations thereof, thereby detecting the presence or absence of the protein of (i) and the protein of (ii) in the extracellular vesicle and particle protein sample.

126. A method determining presence of pancreatic cancer in a subject, said method comprising:
obtaining a liquid biopsy sample from a subject;
separating extracellular vesicles and particles from the tissue sample;
isolating protein from the separated extracellular vesicles and particles to form an extracellular vesicle and particle protein sample;
subjecting the extracellular vesicle and particle protein sample to a detection assay suitable for detecting:
(i) a protein selected from the group consisting of Calmodulin-like protein 5 (CALML5), Carboxypeptidase N subunit 2 (CPN2), Carbonic anhydrase 2 (CA2), Heat shock-related 70 kDa protein 2 (HSPA2), Lactotransferrin (LTF), GTPase KRas (KRAS), Complement decay-accelerating factor (CD55), Brain-specific angiogenesis inhibitor 1-associated protein 2-like protein 1 (BAIAP2L1), Phosphatidylethanolamine-binding protein 1 (PEBP1), Ras-related protein Rab-1A (RAB1A), Ras-related protein Rab-8B (RAB8B), Desmoplakin (DSP), Leucine-rich repeat-containing protein 26 (LRRC26), and combinations therefore, and
(ii) a protein selected from the group consisting of Thrombospondin-1 (THBS1), Complement C1r subcomponent-like protein (C1RL), Immunoglobulin kappa variable 1-6 (IGKV1.6), Immunoglobulin kappa variable 1-17 (IGKV1.17), Immunoglobulin kappa variable 1-39 (IGKV1.39), Immunoglobulin kappa variable 1-27 (IGKV1.27), Immunoglobulin kappa variable 1-12 (IGKV1.12), and Immunoglobulin kappa variable 1D-33 (IGKV1D.33), and combinations thereof, thereby detecting the presence or absence of the protein of (i) and the protein of (ii) in the extracellular vesicle and particle protein sample.

127. A method of determining the presence of pancreatic cancer in a subject, said method comprising:
obtaining a tissue sample from a subject,
separating extracellular vesicles and particles from the tissue sample;
isolating protein from the separated extracellular vesicles and particles to form an extracellular vesicle and particle protein sample; and
subjecting the extracellular vesicle and particle protein sample to a detection assay suitable for detecting:
(i) a protein selected from the group consisting of Protein S100-A9 (S100A9), Protein 5100-A11 (S100A11), Protein 5100-A13 (S100A13), Integrin alpha-6 (ITGA6), Integrin alpha-V (ITGAV), Versican (VCAN), Fibronectin (FN1), Annexin A1 (ANXA1), Annexin A3 (ANXA3), Cathepsin B (CTSB), Protein-glutamine gamma-glutamyltransferase 2 (TGM2), Complement decay-accelerating factor (CD55), Thymosin beta-10 (TMSB10), Syntenin-2 (SDCBP2), Fermitin family homolog 3 (FERMT3), Myosin-10 (MYH10), Myosin-14 (MYH14), Dihydropyrimidinase-related protein 3 (DPYSL3), Lactadherin (MFGE8), Inactive tyrosine-protein kinase 7 (PTK7), Dipeptidyl peptidase 1 (CTSC), Serpin B5 (SERPINB5), Epidermal growth factor receptor kinase substrate 8-like protein 1 (EPS8L1), Neutrophil cytosol factor 2 (NCF2), Metalloproteinase inhibitor 1 (TIMP1), Cathepsin S (CTSS), Glutamine synthetase (GLUL), Integrin alpha-L (ITGAL), Formin-like protein 1 (FMNL1), Intercellular adhesion molecule 1 (ICAM1), Vascular endothelial growth factor receptor 3 (FLT4), Platelet-derived growth factor receptor alpha (PDGFRA), Integrin alpha-X (ITGAX), Sequestosome-1 (SQSTM1), Retinoic acid-induced protein 3 (GPRC5A), Disintegrin and metalloproteinase domain-containing protein 9 (ADAM9), and combinations thereof, and
(ii) one or more proteins selected from the group consisting of Syncollin (SYCN), Pancreatic lipase-related protein 2 (PNLIPRP2), Inactive pancreatic lipase-related protein 1 (PNLIPRP1), Phospholipase A2 (PLA2G1B), Chymotrypsin-like elastase family member 2B (CELA2B), Stress-70 protein, mitochondrial (HSPA9), Very long-chain specific acyl-CoA dehydrogenase, mitochondrial (ACADVL), and combinations thereof, thereby detecting the presence or absence of the protein of (i) and the protein of (ii) in the extracellular vesicle and particle protein sample.

128. A method of determining the presence of lung cancer in a subject, said method comprising:
obtaining a liquid biopsy sample from a subject;
separating extracellular vesicles and particles from the tissue sample;
isolating protein from the separated extracellular vesicles and particles to form an extracellular vesicle and particle protein sample; and
subjecting the extracellular vesicle and particle protein sample to a detection assay suitable for detecting:
(i) a protein selected from the group consisting of Putative alpha-1-antitrypsin-related protein (SERPINA2), Immunoglobulin kappa joining 1 (IGKJ1), Protein 4.2 (EPB42), Histone H2A type 1-D (H2AC7), Proteasome subunit alpha type-2 (PSMA2), Nebulette (NEBL), Tripeptidyl-peptidase 2 (TPP2), Monocyte differentiation antigen CD14 (CD14), Fc receptor-like protein 3 (FCRL3), Charged multivesicular body protein 4b (CHMP4B), Rho-related GTP-binding protein RhoV (RHOV), Leukocyte surface antigen CD53 (CD53), Basement membrane-specific heparan sulfate proteoglycan core protein (HSPG2), Trypsin-1 (PRSS1), and combinations therefore, and
(ii) transforming growth factor-beta-induced protein ig-h3 (TGFBI), thereby detecting the presence or absence of the protein of (i) and the protein of (ii) in the extracellular vesicle and particle protein sample.

129. A method of determining the presence of lung cancer in a subject, said method comprising:
obtaining a tissue sample from the subject,
separating extracellular vesicles and particles from the tissue sample;
isolating protein from the separated extracellular vesicles and particles to form an extracellular vesicle and particle protein sample; and
subjecting the extracellular vesicle and particle protein sample to a detection assay suitable for detecting:
(i) a protein selected from the group consisting of Small nuclear ribonucleoprotein Sm D3 (SNRPD3), Four and a half LIM domains protein 2 (FHL2), 60S ribosomal protein L26 (RPL26), 60S ribosomal protein L22 (RPL22), ELAV-like protein 1 (ELAVL1), 5′-3′ exoribonuclease 2 (XRN2), ATP-dependent DNA/RNA helicase DHX36 (DHX36), DnaJ homolog subfamily C member 7 (DNAJC7), Oxidoreductase HTATIP2 (HTATIP2), Amidophosphoribosyltransferase (PPAT), and combinations thereof, and
(ii) a proteins selected from the group consisting of Caveolae-associated protein 2 (CAVIN2), Na(+)/H(+) exchange regulatory cofactor NHE-RF2 (SLC9A3R2), Protein mab-21-like 4 (MAB21L4), Fructose-1,6-bisphosphatase 1 (FBP1), Heat shock 70 kDa protein 12B (HSPA12B), Sciellin (SCEL), Pulmonary surfactant-associated protein C (SFTPC), Caveolin-2 (CAV2), F-actin-uncapping protein LRRC16A (CARMIL1), Advanced glycosylation end product-specific receptor (AGER), Protein XRP2 (RP2), Specifically androgen-regulated gene protein (C1orf116), and combinations thereof, thereby detecting the presence or absence of the protein of (i) and the protein of (ii) in the extracellular vesicle and particle protein sample.
